SEQUENCE LISTING (150 sequences)

SEQ ID NO: 1 - DNA, 136137 bp, Homo sapiens
  misc_feature 1..136137: note = Description of sequence: ELAV Like RNA Binding Protein 2 (ELAVL2)
  source 1..136137: mol_type = genomic DNA
aagacgtcctcccccctatttctgttcttagttagagatgcagcagcttactcctgtagcgacctactaaaaagcaacaaggagaaagacatcgtctttttgaaaaaacgttctttcgtctcttttctttgttccttggtttgtttttcttgcccctttttgtttagttgaacggcaataggagggtagtctctccgtctttttaaactcttttttaagtttcccctcccctttcatattttttttcgccatttcttttagcattggactttggggtcgaaagcgtttctttttatttgcttcttttaagccgagcacagtttaggtttcgtgctgtcttaagagaactatccagcagcttcttgctcatccttattgggagaactgcaccgttactttaaaaacacacatacacaaaaaccttaagggagaaaggtaagtttcggtttggtttctttaatcattagttgcagcgatgcgctggcaaacccagaaaacctgtaagtgagcggaatacagaatatgaagttaattgcacatgtttgtctcatttcaaagctactgttctgcttttttaattttaaagagctagactaaaagacggtggacgcatgcttagcgtactgaatattcgtaaatgtccatggcagaaacttaagtggtaaatgtggccaggggggagtaggggtacaatgggaggttagatgtggagtgagcttcaagattgagatcggaagttcatttcgtggggagagagtgataacttgggtgttggggatgcatgcctctactttggctcagcaacggaaggacgggagaggctttacaatcattaaggggaatcagaagcctggactggcagattagaccagcgaaaaccacaggcttaattgcattaaccagatagcatgtgctggggagatgggattgccagatggaatttagatgctgcaccaggcagcgagttgtagttgactgaaaaaagtacatgccgcctgagggctggggctggggctgggctgaggctagggttgttagaagctgtcagaactcatcattgtatgcgggctctgattcacggactccaattatcataattcagttttaaaagaagatagccaatttggggctgaattcttaagcctgtaccggatatgagattccctttgttcagaaaagcgtaaataagatccttggagctgtgtctatgaagtgccggctgcttgggagcctcgctgtgctgggcgctgggaagtggattccaagccctgtccttggtcctacgcggctgacttcgccttcggggagagggggaacccctctgcagccgcgaggcttcgcagccaccccaggagccaactcggccacgtagccctcaccccctccttaccgcccccacttcctcgaatagctattgttaaattgacgccaggagaaagatggttggggatgacaagctgggcagataggcacacgcttcattttaacttttgtgatttctcagggtttaggagaaatcatttggctttctactgccaaagagaggatccagggtagatgtgggaaagggggaggatgttcgtgggccggggagggcggaaggggtggtggtggtggaacaagctgctggctttcctaagagaagtttaaattattggcacgagggggtgtgggtgagtgtgtgcgtgagagcgctcatgtggtgctcgagaggcagaggatgggaaaaagctacaaagccggctggggttaacgtctcgttgctcctcaccccacccactctaccggctgggaaagccagcgcaccgtcagcctcccggactggactctgcgaagggcggtgggggctagggatggcaaggtggcagtgcccaccactggagctccgcggcggacgagacctcgcgtggccattgttgcttaatttatgcccagcacgttgctacgtacccacctatttcacgtctgtacagaattaatacgaaagctatacaactgtgacaaattggaggttggataagttccccgtttcccattttcaagtaccggttagcaaaactgatggcgagactgggtattttctcgtaattaccggctgcaggtctggttttattatggcgagggtgagagaaatagccaaccatttagtatttactgttaacactgctggacagggatggaggtctctgtactttctccccgaaatgcacggattctgtcccttccccctctcatgtacaaacactccagttggttaagattctaaaccttcagccgttctccgcacccctatttattatacccatcctctgggggtgggagggagattaatgttttatttcccaggcctacactattaactgcaacctataaaacgtagacttacaatgaaaatttttagaaagtacacagcagacaatagtaactaaagaaattattttccaaattgaaaatcagtttctaaaggtcagaaaaagggtgttcgtgttttggggaacagaagggtaggaaatgtgtttgggccttaagacttccttgtaaaatggcttcttaaatgggacatctgaaagatttgtcagactatgaacagataagcccgcaaaaaaatatgttgagagtttcgaagttgttatggggttaacattttattttttaactctcgcccttacttattgctgtcattgcctgaatgtgtcttacagcaggcacttcttccccgttccctcttttaaccaagtgcttgggcaccccgcttcataaagctaacctgttctggagctcttcacaaaaaacgctacggttgtaggaagaagtggtttggaagacttggggaactcttctgagagctctcccataatttagtcagcgtcaggaagtagggcgtagggcattctaagagttttaggtttggattgacaaattaagtaggcccattttcgcctaacgttttggaaataggcatttttagcaatcccgcgtgcctgggatgagaatattataattaactatttactgagaggtcaaagaggcgcaggcgctttgagcagttttcccaggaacccacacaaagtcaattaaggtcatttaattgcaaatcggatttggggtgacacggaaatgctgatatagaatcagttagctgtatccggtgtccccagactgggtcgcacaaattaagggatggcctatctcgcttttgaccttggttgagccccctacttcactatgctgattagacttcgcccaagagggcaaatttgcctctcccacacccgccctgggttagacgagtcccctcccttccccccaaccccgctccgcccgtcgcatgggggcggggagcgctggacgcccttagcggaccttgctgcatcaaacctgcatcatgcccttgaagctgtaacctcggtcacgttcttgcggcgactgggtcgcccactgctcggttcctttctcttacctcctacaatttcaggtaggtatgacaggtgattttttaaatgtagtattttaggagaagggctaaaatcgatgcctttgcaatgaaaaaaatcccaactacaggtgctctcgtaattaagcggccgagcaggtgtaaaaacgtgctccagccgcgtggctccggggtgggttttgacttccagccgtggggccgggtctgaggacaggcacaggtttttctggggaaaagggattgagcttggcgaaggggcgggccaaggcaaggcagcgatgggacgccccctcggtgaagagttggccatttcgggtcttgagttggggggcgatagaaggatgcggctagcggcaagcgtttgcatagtcgagtgcaggatttcctactttgcgtttgaagcagagtacagcaccccgcacaggtactctgggcttatttaaacgtcaggttctctctagagaggcagggaggggcagcagggggctaagcctttattttctgtccagggacaggaccccttggctgcgaagtagggggaaaaagagaatttttaaaagagacacacatatgtgtgtatacttatatatatttataaatacatttatgaacacataaagtaacccccacgttagctgctctgactccccgttagcgccccgggcctcgggatttgcaaacttgcacctggaatcagcctcctctgggcgctcaacgctctccaccgagtccagagaaaccttgtcggggaagcctggtgagactcggaagtccggagggacggagctgagctgctgaagcccggcatctccctgccggcacggacccgaagggcggtcgggcagggcacgcgccggcggcggccgcggcgccgcgaactcgccgccggccccgcctcctggcgctcttccaagcaagcccctccttcccgccccgctacaaacggcgggaccgcggcgcctgggcgtcactgaggcagtagccggccgggtgaggagggcggttgccggcgcggcgcggcgcggcgcgggtggggcgggggttccgccggcttccagtcccctttcccgccgccgccgccgccaccgcctctccgcggagctcgccccgagcgactcctccgcggcagtgctgacggccagcggcacgagccgtagtagctgcagcttcgagtcacagcaggcaagtgctgggggcgtgggggcggggacggcgtccgcgccttgcccctggtccattttggacgccgaggctaaggtctgtgcgcgcccccgctgcgtctagcagggtaggggtggtgcgccctagtccgaactctgggcgggaacactggtgggggcggcggaggttgtgcccgcgaagttcctagagctcagcccgttgcggcgggagtagagagaattgggcgcctcgggaggtggcaccgcccctcccgtgggcacaagcaggttgggggcggcgggagccgagcggggacagtcgcgcctggcagcgtgcacgggcgtggacgtgcccgggtgcggccgcgtgtagcgcaagaaggaaactgttgagacgcaggtgagtgtattggagggaaagtgggggagggggcttttctagaaaaagggcaggattcctgtgttgcactggcaaccccgccttgcgggaggatgtgtgcactggcttctcccgcggaacacgccggggtggtgtctggtgtgtgcccacttgctacggcgccttcgcgcgcgcggtccttggagctctcggggcgtttggagacttaacaccggccgcgttgtgtgcggaggtggtgtagggccgcgggcgcccagggcaggagcaatgatgatcccctcgtgcgcgttaggtgaggcccgtcgcagccgcgcgaggtcgcagctcgcggtcacatctcgggtttgcgcgcgaggcgtgcgctgcgcgggaagcgcgggcgctctgccgggcgaggccggctctgggatttcagttcgcttgccgtggttgcggcagctgctcctccagcttggctggtagggcctgccagcggcggccggggtgcacggactagtgtgcgtgcactggtgagactgggggagccagcgtcggttcactggcaggtgctcctccagtggccgtgacaatgcttagccagcagtctttctcacggcatttccttaaaagcctccttgttatccttcagggccagagcgtaaaaccatgcctctagatgcgagctatgagcatgcagttgtagctgcccagatggtcatttagaattgtgtgcatttttaaagaccatctttatgacaaagattttcaagtcctatcgtctctacattttctgaaaacatgggaaaaggatttgctacgagttgtccactctcctttattccctccctcaaagaacggttcttagaagtgccaaaatgtaataccttcggcaaggacaaaagcgcttttaaaacttaattttctagtggccctattcctaataattcttcataaaaatttagctcagcctcaaaacttacggcgaatcagtccatttaaaaccattcgaatatcaattgctgtgtaatgtccccattccctcaagtaaaatctgtttgtttgaaatattgttttgttgcccttgaagagttttctcaagaatcgtttcgccagagtcgcttgaatttcccatttcttgtggaagctgacctggagtttccattttggtgagcgggtggaaaagctgtcctgcatctaccttgtcttagtaacctttgggtatatataaatggtgactgtattgctgagtggacccgaatgttactgcatttgtggtaggggccttctttataggaggtttgtagggatgctaaggtaacaacacttaaggttgtggggaggatatacttacgggaagttcgcttgcaaactatggggagagaaagaagaggaagaaaacatctggaagataaaggccagaatggggtaggaaaaattagaatatgaagaaacatgttacattaaattttctgtgattcctcctttgtaattttcctttttgagagagtatttttatttttgtgtggttaaaaccagccccttgttgcctgttatttgggtaactttgtgctgccacctttcagggagttagttgtttttttcttcctttgaaagacaactttaggtgttaagcccctccctcttccttatctgtaggtgtttccttcattgtgtagtgtcttctttcaggaacaatcaagtcagctatattcagtgctcacacactttccctgtggttcaagataattttagttataggatatgcttcttgtttagagcaagtacccactgctggtcagtgtctaccactcttttgacactcagtgtcaaaaggttactatggaaccttttttcttttctaggcctaagatgtgtttttatagatcctctctgatttagtttttttggttgatttgttctggttaccgcctcttcacaagagaaaaacaaagcctttctgtcccagtttcagtggttttagttaagactcagctcctggtaggttaagggagaatgatgctgtgagtgtcatataccatcattccttgaatcaaccttttgattatttatacacagacagatgctgctagctgaatccttgtaaccaaccgctcttggctcttccagccttccctgcttatcttttaaaaataatgtgcctcccctcctgccaccccacttctattctagaagcaagtcttaggttctctttaaggacacagcaaagccttccagaatatcctgacttttaagctgggatcaaccttttcctctttctttgctgaaagacatttttttaaaaaacctgtgacgcttttcaggcattattggcattgcacaagagcaccacagtggcaaattagccttcatgttctttttccccaaaggactagatactctgaaatagcttcttaagcttcttatcaagactgactttaccattcccctttttgatgacttgtctgcatggtagcatcagtattttcgctcccatttgatgtcttattaaatctatcacatcgtcacattcttattgccacttttatcactcataatgtttatttggaattgagagaggctagaatctgtggtacttagcttggtttggttacttttttgttgacattttaagggggctagatggtgtgccaccctgtggaaaacctgcccatctcagtctacccccagctctttgtttcttggctactcagctggacaggtcaaaaatgatagtaagctaccacatttgagtgtgtgtggggggaggttaattttatttttggcgtgagtacttagttatgggcaggactgcttttccagcttgtaaccggtatggttgcacatcctacccaatcttgaaattcttaagaatttttggacaaggttaccctgcattttcatcttgcactgggacccacagattacgtagcttttcctggttagttgccttcccacttgttagtatttagctgtcagtattgctgctttgttgatatattatagaactgtttgaaggctttatgtctagtgatgccacacctgactatcaaattcactatggaggacataaaatgcataaaattttttcttgctttggccattttcattttacaggtgtagaagtcttcaccggttctggttacgcacagagcccacatattgtagttcagtgccaaatccacacttttgtatgttctaatttgggaagaacatttattgtatatttatttggttcgagatactagacggtttatagtgctagttaattatttaaggatttcacataacgtaaatttgccaagtggaaacagaagacacttaagcttgaaaggcagtgcttattttaaggcttttacatattaaaagataagtgttttattatgatttaaatgataacttttggagtctgaattaaaagtcaaagtttgcgtaataaaatcttagatttttgtggggtacttaggacatttttcactagtaaatttaacagatttggctttcagcttgtgtgatgtgtatcatatgtttcattttagtaaggctagaagaattttttatttcaagggaagaattttttttatctctaagcttgtattccattgtgcaaaacaaaacaaaaggtaactcttgtgtgagcaacgctgtgctaaatgcattctcatagacatcttcatctttaatatcaatgttaagtttctagagattggtcttaataaaaataactatttttgcaacgagcattcttttctcagagctttgagttaggctctcaatgcgtgaactgaattgatatgcttaatgacatggtacctttttgaatattgctgaatagttctgtgtgacaacagatacaggttttcgttgtaagaaatctttatgaagagaaggatgggggctttgaggagtaaaataggtaaaataacccagaaagtcataagaataggtggaatggggataaaactaaaccagtttatagaatgccttacagtgtcttttttttaaataaacttgcagtttcttagtatatatttatattactgatatctattacatcagtttctgactgaaaatgaataggagataaggtctgacatgcaaaatcccctttcccacttttaaaaatgtattttaaaaaacttgacaaatttaaaattatacttactgtaagacagtaacagtaaaatgataggtttttttttttaagacacagtgaaggttttgctttagcactggtatattttgaaccaggtatgttatctacagtttgatgtacctgtatatttttccgttggccaatctaatacagatgctcctgataaacccattgcaagttgaaaatggcattaagttaaaaatgtatttaatacgcctaacctactgaatatcagagcttagtctagcctaccttaaacatgctcagaacatttatgttatagttgggcaaaatcatctggcaacacagagtatccatttttcactctcatgatccattgcctgagtgggagctgtggcttgctaccattgccctatatcgagagagtctcttaccatagactgctagcccgggaaaggatcaaaattcaaaatatagtttctcctaaacgtgtgttacttttatatcaccatacagttgaaaaatcgtaagtttgaaccatcataaattggggaccatctgtagtgatagatttaatccttgggtttttcctttgacttaaaaagtttatcatgaaagggaaatttatattctaaatgttcttttcttccagcattttgtttttgtgggttgttcattattggagattatttcagcaactttaaagactatgttaataggatatcacaaaaatcttttataaatatcaaagcaataaactattccttcaagatggatttttatcccctttttttttttttttttttttttttttttactgaaagcttttagtacacactttcttgactaatttcatctctttatgggactagggaatgatagttgttttgagattcttagctttcctgcatttctttagagtcattgtaaaattggaagttcttgttaatattgaggggctagaatctctttaagcatttgaagaaagctgtagctgtctcttcctagagaaatatatacaaataagaaatttccatacagtttttagtattttgcactttggaagaaagcgtgttaaaatgaggttccatttgggaaataggactttggcaattagagttgtcttcttatactagtgagaactgggtgaagtcattcagaggttcttgtctgctgttgagttagtgatgtgggcctttagcattctttgaaggatgactgactctaagctttcctgtctcatagtttgttttaaaatccattctgtttttgtaaacaaataatcacacacataaggtcttgtgatcaccgtgcccgtgcggccaatttgctgagttgtctcagtaggttttgtcacttaggttgaaattcttaatttagcacagccaggtggaaaatagtaggctagaagtctgaagttttcagcatctggtttggtttaattacttatatccttggtttcttaacctttgattgtgtataaaatcttaccctccccatctcaaggaccacgtaaagatgagcatattgaagtacaagattttttgacaatgaaatgacattgactgaattggaaaggtggtagtaggtggttactgcttgattagttttatatttttggctttcaatgtgttatgaaccatttttagtgattttgtttttttataatacaccatcatatgtttatttctgccatgaagtaaaagaaaaatcccagactgcagggaggatttgggagaattgtgactttggacatttcagtatggaaatagacatatttatacctatgatgatgggacttggaaagtttaaaatgcattttccttgaaaatataaataatgctaaaagattacatatgagactgagaatttagccagctaacattccaggaatataaatttcttaatttaaatttctcatatttttttcagtgaaaaatcaaaattttaaatttaaccatttatattaaaatagtccgcgttttctctgatttgaattttatttaacctcttatcaaggatccattcctatacatcacaaaatggagtttacatagaaatacatttttttcccctcattgctgatggtgtgaacatcattgatggatgccctgtcaagtaagttattggaaattgctcacttgttcagcttttaaaggatgtttagcactttttttattctttaggcatcatttgtgtatatcagaataaagcaaatcacagtgtatctgaaaataaattcagtgaattgggtaaggatgggaaaatatttctggagcacttgatcttgttttctctgagctgcttttcaaaagtaagaatttcttgttatactgatattgctttattggaggtgatatggcaggtgagggggtcatccacctttattgcagaggtatccagaatgtgttaaatatacaacatttattatagagggagcaagattttataacctgttcccaggtaaggcctatttctatttgagtttattcaacaccacttttgatctccccaggtgctatttactgattgctggcttattttgtttaaaaacccttggggtcttttctgtcctgtgcctctgaatgacccatggacttttcccaaatacgaggtcatcagtgcttttgactttcatctcttcccttctccatagtctccgcaaggaatttgaactacccacagccttcctcccctctttgggatgcgtctgtacttgccctctgtcccttgtttatccttttagccttgtgcttagttccaggtcctggtaatttggggtcaggtgcaacctgctcacttgtccctttattttcttatcctgcttgggcttttgcattggctcttatgcactttgttacattttacttactgaagaatatcttctgtgaccaagtgaatcactgataccagaaggagcagttggtatcagtggacaaattatttgtgccacaggtcctgaagggtgatccgtgagcttcatatggcctataggtgttttgattgccctgaactgtgttttaaatagttgtgagttgttgatttacggtttggtaatatataaagatctggagccaaatgtattgcttcttgaaaacttggaaggatggagcaacaacaatggagtccacatttatcgactgcgccttaaggatgaagcatggcttgtttcactttgaccttggtttgtttgcctggccttggtgtctctaatgctatacttcctaaagccacgtgaattaaaaagttaagtgtaaagacaaaggcagtagttctaaaggttttatattcgtttgatctcttgtatttacagataatgtttgaatcatgttaaactgctccttacatatttagatcttatagtctcctttacttcagagaagatttgtcttgggtctctccttccttaacccgtggttgaaaaacagtttctccttttataaaatgagcttctctcagcttaacttaccttttctctcctccccccctgggattttagaactgtccccattgggagtagggatttctccctgccagttgcctgcttctatttattcagatgctttggggtcaacattgctacttgatgctgtaaaaactgataggatgttatgagctgtttaaaagaggcctgtggcccacctgctaacctcattgctgatgttgtgagcttataaatgacaacagatttataagattgttgtaaaaaaaaaaaaaaaaagccgatatgagacttccaggtgtgtggtttatgtgtgtgtgttatttctctccctttcccctttatttttttcagtcttcagccttttctcccccactccagaagcagcagggcagccttagcaaggccctctcctatgtgtgccgtagagcagactgatgcaccagcagaagtagaaagaggctccacatgtcatgtttggaccaatgctgtaattttattttacaataaggaaggtggataatattaatctactcctgagcataatagcacaatttgaatagcagagagcaactttggtgtgtgttaatgtctagtgagatgaatatagttgaatgctaagtaggtagaaaatgtcagtgtggagccttccttttggtgactttaagcacttacatttctagttaacttttgttcagcctggtaaatagatatgaagatctgttgtatcaacccagcattaatgtagcttcttaggatggaacaatgcccataaaaccctgctctcctggcagccacttcagctaggccagttataacgatgaatcagaatgtgcccctgtattgaacatcttagcaaaacccttcagaataatagcttaatgttggcagttttaagcattggcttactctacaacccagccaattttttttttttttttgatatttggagtgttagtataagtgtatgctcacttaaaaacttaattttgaattaactgtgtatgattttaaaaaatcattgtaggtacatttggaacttatttttcagaggcacttaaaaattcttggtaccaagcatcttctattctatgattatatgattcttttaggtgaacgtgttgcttgtttttgatgctccctgcagcatctcattgcggcacctcagggtggcttagttactgggttggggtggggaattgaatatcagtaaaaacaagcatgttttatccccctaaagattgggagtcattcccgtggagcttctactgtgcaataagaatgactttgatatatgtgggctgctagttaacttgaagcttagggcttttccaaattaatctaattttgtttttgagtttgttgaaggatcaataaaaatattcttctaggttttgtgttgagattatgttcagaagccaggaagattgtgtaatacttattgtttcatcacaaattactgagtgctggatactgagaacgttgagacttgtataaagtgccctttacctcatattccttggtctctacctaaggagatgaggcagaaaatgataacttaaaaaaaaatcacacactccccactcttgcatttaatagtagaaactgagtgcttgaggaattaagcaaagagatatatccctgttgtctgggaatcaagggatcaggccaggttgttgcttattgctttagggacaggactgtgtcttttcttggtccattttgtctattttttgctttttatgtcttatttccctcccccgttcctcccatattgtttttgttacctttgattttcttttatggtatgagtaactatttcctagtttgccaggagctcagtcacaggtgaaaagtatttctgcaaaacctactcaagtttgagggtgtggctagagagagacaagctgtgactgtcttaattccactgttgtcccagaagagtagatctgaggcacgttgtgtttcaaactacatgaagctgggtgtttcccccagagttttattctgttgacaacattatgtgccagcagcctcctctgggtataacatttatttttcaaaacacctaggaaaaagtcaaaataattacaacttttttgacttcctcccttttgggaatatagttgcaggagcaagaagcaagtaacaggacagaagctggggtgcttttcagagcattgatctatgccttctccttgcgtcatcttcatggctaaggccatgtcttccaggagcatgtgttgtctgggctgctgactatggctttctcatgccagtgcttggtgttctgaggcctggccactgcttttctcccttaatggcatttgcattactgtttcgtgggtttttttttttttccttgaaaggtgatcgaacagcttgtacgctgcgttacctttttgatttagtggagaggctcattccttcagttggaactggctgttcggggaataatttgtgttcttgtttcttggactttttgaaaaaattttttcccaaacagtttggctgacttataaacattgttttgtggatggtagggtgaaatctttggttttagagtgaataatagtttgtgacctagacaggaagagtgaagacatctgttttggttttccttttgtacaacttctatggtaacttccaaatgactctcccttgagaatgcaacagtaagaaattttgtgttggacagtcaagttagcagtggattgggatggggcagcagggatctgccactttttagttgttgacttaccattaggaagatagaaaaatacgtggagttggcaattgaagatggtgaactaccacaaggtgatggaaccaggagacacggagcatttcctctagtaatggtgattaacaccaagtttttctgtccttatagaaacttgccacagctccctgggaatgatgttttataggtgaggaaaatgaagctcagaggtcaggtggtttgcccaaggatacaaccaagtggggaagtaggatcaggtcagactcctagttctgtacctctttccatggatcccagaatgatgttctttctacttttggtagttctgctggctgtagtttaaggtggctgaaatcagatccaagaagcaactcagttttcatgtttgagaatgtattgcttctcttaggggtgttttgaaataaaatggtgtgactccagcagcaattgtatctcacctccatattaaattgctttagaggctgtttggttccccagtggtggggttgttgtaccaggattattttttatttttataattttttttaaagcttcactgtggtccctgtagcactcctagcagtatgcccacaacttgttcctcagggccaggattctgaccttggattattttaggtccatgactgggtgacttctggaggcatttccatcacaggctggtgactttctgatgccagctggcatccttgacttgcaggattactttttggaaccatacaaagtatggattggttattttctgtaagcaaggaaatgaagacactgtcttcaggctttttttaggatgctgttaaaatcaagaaaaaggtgaaagtgacattgagataaccctggctcattgtagtagataatctgtaagggctgttttttgaacttgttaagtttatgaaaaaaaatgtgttcccaatcaggcaggattttagaaatactactttttgttctgagaaaaaaatggcaggtcaagtgttcaataggatgtttgatatccaaattggaataaacaaaatattcacccagtaaagattgttcttgattcacctttctccagtaagttttcagtatttctccaactttactgtttctccaactttactgtcagaaaaattggttgggagtggatgacaaagaattacagattcaaaatgacctttttgctcttccctagaaagaggccatgtgctctttttcattacttgatctctcacatagtgtgtttttaggagaaattcaggaaaatttgtggaggtcactggaacagcagaactcaaaagtctggaatagagataataatttaaagaaagaatatagtacatgagactgataatcaattgaaaattatttgcatatgtaagtaaatcatgaatgataggtaatcttactagaatttcaatctctacagggaaatttttgcaaaaactgccttaactaatcccatgttttctcatgtcttagatatgcaatttttatattacttttgagtctcttaagtttacttaatgtcctgattctatgtcccccaaagctgtgaggttagtgggtgcatctttcagtccaaatgtcttagaattgagaagctgaaatagcttcatgccaacatgttctctgatttcttatctctgcaaatattttactcttcctagagtataaatattgtccttgtgctatggaaagctggtgtaaacgtgagaaaataaggtcatcagattgtctctacatatttcaagcaggtgcctctggtataaaattggccaacaggctatgcacaaagcagtcaggtgggatcctgacaaggctgctccgtactggttagctgtaatttagaatatcacaggatgaagcagaagttaattgttgagggtgaaagctcaatagcattttactcatcatcacattttataggacatagtaaccttaaactttgaccttaatacgtatcatttctctttaatttttgttgtgtttccttaaatttggcgaagactaccagagattttgttgggtaaaatgctttgtagctccccagttccctatggaattcagaagcattaagagatccagggactttgttaagttttgggtgttatagttcagactccttaaggaattttctggaactttacccataaggaatttttcattatggctaacatttctttctctctctcattccctgtgattattctctgttatcgtctcaaagaaatgtggcaatttgatgacagtttattaaatagcctatgaatatttgaaatgtttacatctaaagtttttattagtagccactttaaaactttaaacttatctctcttttcctagcatttaacattttattattttattgtgtctggcctcctgacatgaatggcattttgctctttttgattgtccttctattcagtttttgatttgctgatccagccttttctgccccccagtactttaaaagcaattattttgccagtttacatgtgaacatttcttggtttcctgtgcagattttcatttgtttagtaaatatttgtgatcttgctatatgccacatatactaccttaggtattaagactatagtagtgaatacagtatactccctgctgatatgaaacttagagtttattggaggagatattatgttaaagttctacaaattattacatataattgtgatttgtgctgtgaaggaaaataacctttaaggcttataacacagtgtgggaggtgattagtgaagatcatttgttgatttccttcctagctaccttatttttactgtacataaaaacagactagtttttaattttataggcataaggatagacagaggtcctaaaattaaactctattctccttgataaattgatggtgaactttgagtcctagagctgattcctgccacccagcttctatcccaccatggatctggctttttatattttttacgagaaaccgattttaatgttctttgcaggacaacgtaatttggcaacttttactctgaaagctatgtggtgaatgcaaagcccagcagtgagatctgcattgtctaaatcctttattctctttacccatggctttgaaaatgtgttggattttgaaatattatcagtatttgtttaaataacactttctaatgtattaattgatgcagtaagaacttaggaatatttgttaggctttttgcttggcggaaattagaatttcactaattcatccacagttgtgttttgtcattgattggattatttggtttgtttatgaatgaccttttagataatccctttgacagatcaccccattgctttaccatattgtgttccttccagtattcatttaaattataactaaaaggaaataccatattaattggaattgtttttaaaatatttgttggagtggaagagacttaggcagttttttttttgtagaaactttggaagacatttgttgtttaagagtgtcgaaaaatactttttaaaaaaatgtcagtggcttctgtggagttccttaatcctaaagggctaggtatgttcgtaagagatgggtagaatatgggaaattactctaagcaattggaaaagacacggaaaaaaggttaaggaaaatgaggaagtgaattttccatctggctagtttcctcattcacacagatcaccagacctaaacagtacagtgaatgcatggcaaggatatgatattttaaaatagtgtctttctgaccaccttattagattattaagcttacttgactaggctcttagtttcctcagagaggtggactctggcaaacagttttatttttaatataagctggtctttaggaagataataaaaactttgcatgccttttagcttataccatttataattgtttgatttaacatggatatagcatttttttgtaggtggatattatttcatcacagcttttagatttcagccccttttccttaggtgcagaaggaagctactgaaaatatagacagtaacctctactcttgatggaatttggagagaagtaaaccaagttttagttcaggagaggagaattttaattttacttctggcattacagggacagcaggtatgagagttaaggagctgttacggaatgggcacttggggtaacaacttcccattgcctttggtgtctggtctctccttaatagctttttatattggtttaaattttcagggcacaacttaaatgttactttcaactacattggaagctctttgatgtcagtgattcttataactttgttttctgaatatagtgggtcttattgtcttattactgatttttttttttaagagatttagtctcactgcagtggcatgatcatagttaactgtaactttgaattcctgggcttttagggatcctccagcatcagcctcctgagtagttgggtctataggcatgctttactgcaactggctaatttttaaatttttttgtagagatggggtctcaccatcttacccaggcttgtcttgaaatcctgggctcaagggatccttctgctttggacttccaaagtgctgggattacagacctgagccacctcacctggctagtctgatttcttgaatctaataggtcctatgtctttaaaatgaagattaaaattatatatgctgaaggtaaatgttacatgcaagtttttttcttttaattgtgtattcggccttggtattttggttgctatgaagttgtataacttacatatttaaccaattggagttaataaaaattgttaatatttaaactatagcaaacatttctatgttttttagatatcactactatatgatggtaattaagtgctggatactgaataatattttaagatgactttattaaagtgctatttcttttcatccatacctgtgaataattaagaatttatggtatctgtttgcagtctagttggtgagtttaatttacttggaccacccagaaaaaaatatacaatgatgcttctgtaaccactttgactaagctgaaactatgctttttacatgtatttatgacctgttaataccgcttcaaagtgtgctgtattgaaaagatattggctgcttcaaggttggactgaaatgactgtgtttgccaggaataaggaggggtctccatggatgaccactgggccagtcatttaatagccatgtggaaccctcttcggctatcagaagaatgttttctgctctacactgagtatctttggaagcagatagagaagttgaggtctaaggtagatgctctaatcatggatggctaactttaagaagcgttattgggccaggttctcctgtggtatatgccaagctcatgctcgtgcatgggtacacacacaagcccaaggtacagtagcagctccatctgttttgtatgttgtaggcctgccttgacatagaactgttcgttctaaagcaactcattttaaagctgcagtatgttaaatgggcaacccttcattgaagaccttgggtatgttggcagtttatcagcagcagggttgtgatcctgtcacttcttgttaatgaaggcttttatttagcttagcaaatcatatctcttgttagtatccactctctcaaatggtagacttgaaagggatgtttgtaccgttttatttattagtagccatgagggtgctttggcccttacttgttatagccatttagccatatttcttttacggagatgtggtttgttttaaactaggaaaatccccaaatgattaacgtctttcttttgtttatcatttttgaaatggctttttatatttcctattaggtggtctttttcttcagtctcaatttcacttccattttgaaattcctagcaggggtttgtcagagcatttatgttcccagtgttgcagctgactttcccagcatttatttgttgtccttgggtagtccaagaggataagcattctgtcttcatatttaatgtacatttcttatatataacaagttcaaggacatcagagaatgttagagctggaaaggccctacccatagtgttgaacagcagtctcatttcatatacattcagaactgatgctaagatgtattgtctaaagtcttaggtataatgattgtggttggtgactgtgaccttgtggctggttcccgacttgcctttacttcccccagtgccctctctacactgctgcattatttcctttttaaaaatgtaggttatgctttggtgggactgcaaactgataagttttcagaagttttttttggcagtgactagcgtaattaaaaatgcaaatagactacaaaccagcaatttctatctttaaatttacatttttgctttactgtataaggtatctattgtaagcactgtttgaaatagcaaaaccttggaatgttcatcagtaggggattggttaaattaatcagggtatatatctctattatatagtatctgtaaataaagagtatgtgctgatcttgaaatgtaaaaagcaaggttcagagagcattcaaaaagatctatgtattaagtatttacttttcctcacctgtgccattacatgtttgtattttctttgaaagtgttcaaagtgttcatgaaattgttaacctctcaggtgtagatttgaagaaattaaatatttctgtcttttattgcttgaacccaggaggtggaggttgctgtgagccgagatcatgccattgcactcctgcctgggcgacagagcgagacgccgtctcaaaaaaaaaataaataaataaataaataataaatccatcttttaaacagttctgtggttatctttttgcatcaaatatatattttttaatttgcacaaattattataatagaattactaattacttcaaaagctgatcccccttgggcttttcagagcattttaatagatttgcacttttcctacatatgagctgtttgttagcattcagtgcctgccttgagctgagcgttgtgcatggtcgttgggtgctcagaaacactaggatgtggatgccactctcaagttctccaagtctggttgacagtccagaggaagaaaacgtgagtttatgggagtttggtgaggtctgctgcaaacattgggaaaaggttcctgaaaccaggtagatgtagtagcacttgtgaagctcttttatacagatagggggacttgtccccccaccccacccgcaaccccccaggagaagaggattaagtgtggtgtactcctagcatgtaaggaaaaagttttgattattggaacatgggtgggtttctagaggacactgaaatccatacaggaacatttctcttattttgtttgagagagccagccttgggacaattttacaggatggcttagtgtgaggaaaactgaagcaaaagacatgggctgatgtggctaaaggggccttgaggaggaacgcaccagggctttgagaggaagatggagcatgcatggcttcctgactagggtgtaaacccccaaatgaaagtgaaaaactgcttctggcatgctttgtattgtgtaggagagactcataagatactcaagcatttttctaaaccagtgtaagtatgcaacatgaaacatgtaatagcttgttatgctttataaagcatcttttctgagaaccagaatatacagttatattcatgtttccccgacaatgtttatgtaaaaattttacattaaaggcagttgagatgattcacgtttttttcttataaatcagggtagtactgggcatgagaagtagtttacatagtcccagagctggaggaacatccctttgaaggtaaggagagtagttggaagctgttctaaggtttatctatatgggctaaagggattcaccataaaggcatgcttatgtagcagtgaattagaccagtaattctcaaagttcttgatcagaggatccctttgtacacttaaattattgaggaccccaggaccttttgtttgttatgtgtataaatatttactgtataagaaattaagatgagaaagtttaaaaacctgtttaaatagcaaacccattacatattaaaaaatatttttaaagtgaggaatatgtatatttttaaaaagtaaggctggcatagttttacattttacatttctttaacgtctggcttaatagatctagtcaactgtattctcattgcaacagactgaatgcagaagcaagtgtcacatataacctttagaaagctctactgtacatttatgagaggaaaaggcattctcttgacttcccggatgtatgaggaagtctcagaggactcttaggttcccaggctatgctttgagaaccactggattagcctaatggattcttggaaggcagggctaggcccatctgagtcttaaaacttggaatctgtattgtagggaggagtgggcagtcatctttctaggggaggggtaaatttaagaataaagctgtggaattggagtactttggaggtaagattgtcttcaggagttgaaaacttgaatgctatgaccagattctatcactagcaaattctatcactagaaagtttggccaagtcttggggcttgctttctctgtctaaaaattgaggctgtgggctagcgtttggaagtcccttgtagcactaacattattaatgtattttttatagcgctcttgaccctttctataaatacatgcccacacatctacatacaacatacttggagcacgttggaaggtcacatgagctaaaaatgcacctcggagtagttcacctgctccatgcattttcttcttcctgtattggtttcattgtcctagtttgttgctcaagtagacattgactaaatggtacccaggatgtgtatagaatcttaacctctttcttcaattgtggaacacatctgcacttaaaaaaacaaaacgaaacagataaacaatcttccactgtatttatttcagcttcccgcattagtcgtgagattaggtgggtaaggaggagggaggtgaggtgtcatctaaaaccaccattgtcaatcagggctccacctgtcaattcctatttgcagcgcttgttagaagaaaactctgtgggagtcccttccccctttaaaatgcaaaatggggtgttggtagtgaagggtgcccatttatttttctgtgtatgattccacatttaatattgtaatagaattaatttctttctagtttcaaatgtgctgatttagacattttctgcattttagtttggataactaatgcgtgaacattctgttgtcagatgttgacctttatacaccctaactggatcaagaaatgtcagtaacagtgacatctagcattcttaatgatgttgatttctgtttaaaactaaagtcccttagctaggacacttctgttggtgagatcattgatactctgggatttctgcttgcaagaaatttacttgtctgaaaactgagtagcccttgaaggcaaagacacactttatattccaatagctcctgtacgtagagcttaatatttattgaatgaatgacagtataacttttcctttcaagcacaacattcaagagaagaaatattagtttttcattttgaaatcccttgtagctttaaaaaatcttctacctgagaaaatttctttgagtttaaaagaaataaagcagcagttaagttttaaatgttattttaaaacatattgagcctatttccagtaatgtttttgtattttgttctaggctcttaatatttatttcatacttcagtattcctgggtgcgtgtacctacttccaaacagaaagttttgagaaatagtctggagggcttctgtgtctgttaggtagctccaaattgaaataaaatataaaaatggaagacgaaggacagttgctgtacagttcacaattagccttaatttgtagatctctgctgggcccagctgtaaagtttattggtataaaatgtgttcttgatctggaaagccctcaaataaggtgccttgctttccaacaagcaggaaggaaatgttaatgccagttttcctgtggcttctgtttaatgtgaataggatcctctgaggagatttaaaaagcggctcattgcccttgaaggcacccgtatcagatgaagatagatctaaattcgtatttgaataaaaaatttcatacgtcatctgttttcccaaactaaatttaaagtttagttttaaacttttactgtaagttattttaagtaagtcaaattttctaattttgtagattcatttgcttgagaagcattctatactgattctctactccccaaaaccaagtaatctgaagtagttttccaaagttggggataatgacctatgttctttgaaagaattcttaaaatttccatctgatatcttaagcatatagggctgtagtgcatttcacttcatccttgaataagagggaagggggctaggctggacatgttctgcagcttgtgaggtccttctcctcatgtaagcatggtgccatttgctcattctggcttagtagagctgctcttgggcactaccatcccgggttgtgaagggtgcacttaggcccctgcatgagcagataccctgggttacacatttggtgttaaaataagctgagaagaggaataggaactctactgttttaggacttgattgttacacctccagttcagaatttggggtccctattcaaagtcactccacctatccaagccttcgatgtctgacctacagaatgggaataatgaaaggttgattgtgatgctttatggtgaagccctcattattagctactttcttttctcatttttgaattgggattctttggaagaccaaagaaaacttgccttaatgaggttattttaggagaaatattggactttaaggcttttgtttctttaatgctttgtttctctaatactcttcaaatcttgcatagggaaagattaggtctgtcccctgcacccacccacagagtccttggacagctgtgtttaaaagtgagggcttggtttaggtttgctgggagacctcttaaagcttttgggaaaggaaagagaatgggaaactacaaagggcttaagatttttcttttcagtggtaaatagagccctttcagggttttctaattcttaatagatttcttgcccaactgtggtctcttatttggtgtgttaggtctgtaaagaagcttagtctagcaggaagcaccttgggtgtggtaaggcaattaggggctctcaatagaagatgcactggtgacctgcatgtctttcaaactagggtgttggctgctgggagtagcaagagttactattgagcatcagtgacaaagaattaatcttattaagttggaaatggggctggatattgtaattagatgtgagagacccagtgatcagacttgaggattgtaggccctcaaaactctgcttttccccactcacgtggtggggtggagtaagagtacaaaatcttgttttttatggagggacatctaggttctagtccaggtttcaccctgagtgccttaggaaaatactcacttttcaggtcctgggtttcttatctcttaaaatgcgggatgggaaacaaggtaacagataggattgcttttctcctctaaagatctgactaggtgatgttagaaccaggggaaggtttggtgctactcctgacttcgcttgttaagaagtcataggcgagaggtactcctttgtattttgggttttttggtaggtagtttggagatagagatggaaaaaaattttaaaaagcttctgtgagattgcattagattttcttaggctttctagtcatattatgtctgtcttaattgtgtaataattagcactttaggaaggccattatgataagggttttaatagttcttccatccttcactttacatgaccaccacctgggagaaaattttgtacacatatcaaggaagaaaatgatcaagttttggatttaagaacctaattgttaagtcttgattaaatgatgcctattaggttgtaattatatctatctataaaaaacagatggtggcagatttgattgctgcatttcctgaagcagttttttaagataatgaatttaattacacgtgtgcctgggaaccattttaaaccatcgagaagggaaaaatggacaaggcctattggaaatggaaagtgttcacctaggtgtatagcttttagcttgtggttttgaaggataggaacagaaaaatctgactcctccctcgtctcccttcccacacacaccccctacaaaggcagaatccatattctgatactttatatgaaatgttacagtaacatttatcagatgtcaaagggtatggcatagaagtggttgtgtcagcataatttgaaggctgcttctctgtgccaggcactgttctcagcagtctacatggattcactcacttgagttcttacaaaactctgagatgtaggtggtataattatctccccacccccgccattttataggtaaggaaatggcacgtagagtgatttaagttacaggcccaaagatgcagtaaatgacagagctggaattagaactcacgcagtctggcttcagagtgcacagtaacatctatactggaattctctctgcccaggtgttagaactaaattatttaatcaaataaagggtcctgttttgctgttgcgacctttttgcactaatattatgaactgtctcctcagataaataaccttgcagaatttaggcacatttgagttagaatcttgtaagagacagttaaaggacagctttggctctgtcatcctgcctgaaggtttcagtggctccctattgcttacagaacagatttcaaacactttcaggcattcagagttctcccttctttgccggatgcaatgtttacttcatgaactagccaaataactttttctcttgatcatgacccatgaattcttatcccatactttttttttcttttcttttctttttttttcttttgagacagagtttcactcttgttgcgcaagctggagtgcaatggcgtgatttcagcttaccgcaacctccgcttcccaggttcaagagattctcctgcctcagccacccgagtagctcggattacaggcatgcgccaccacgcccacctaattttgtatttttagcagaggcggggttttctccatgttggtcagattggtctcaaactccagacctcagatgatccacctgcctcagcctcccaaagtactgggattacaggcgtaagccaccacgcccgcatcccatactttttattcactcaagtatatcattatggcaaacctaaatcgtacacctggtgtcctttaaaaaatctcctctcagtccctacctttaattatttctgtcattatctaccacccatgcccctcctgctaccaagcaccctttctgttttaaattcccaatagctggcctttgggattttattgaagtgtatcatacatgagatttaccagataacatgaagatgacttacacatttcttcatttagtttgtgtttatatttacactgttcctgtgatgtgctagggacttagcatgcattacaatttaagatcagtttaatgatatttagaaagccgtgctgtttttgtagtgcctttactggttgtatttttttgaaaacgcctgtgtttttcttttaaacagaggatagaggttcacttttgggcttaagtagaattagtaggattcattctggattttggttcatttttgtctcgaatcttaaactcttcaaatcctgttgttagctgctcaggactcagctgggcagatggtagctattgtacaaattaggacattgaagcataaaggtcaggttaattcaacctgtgtccatttagctaaattggctaccatgtcatgcttgtgagttggactctgcatcaaccagctttgcagtccattgtccctgtcctcattctttatagttagctatctctcaaatgcatccttactatttggaatgaggaaaatggggttaaaatgaagagataagcaagtcagaatctattttctctagtatattccatgactgatattgaatcagtagaataacctatacaaaggacgaagggcaagggcaattgttctcaggatatcttttacatatttgggatattactctgttttgaaaaaaaattatagagctataattaaatatcttcttgtgcagggaactaactctacgtatagatttggcaataatagtgtcaaggctctccgtaaggatggtgtaaactctagatatcttacttatagacagccattgccctagtgaggcaagtcatgcatctttggatcataatgactaagcagcagaattttaatctggcaattttgtgggtttaccgttagagtgtgaagctgtttttgccttttggcagtattttgctttcctcgagatgatctcagctttcattaagatttcatctgactgaaaagattgatttttgagtaggtgagagtccttttatgtgatgtctcaaaggacagagtatgggtcaaatcgctcttcaaatggacagtaattgtgaggaagaggctttttaaatggagattgcaagttacccaaaggttattagaagtaagagcttttatatgtaataagcttccacatttgtaaataacaagaactgtaataacccagtgaaagccccataaccagggcttgaaccagagttgtatgttaacaatgatcacccttctaactctagtgttttatagtaggacctagaaagttcatcatttgtcttttggtttaattcttctttggattgcagaagtatattactgttcaaatttttgtttctcataaaaattaagaaacaattgaaggtccaaggaccaaaatgggtacacttcgttttcataatattccaaaatctggtgggaaacaatattgatcaagaaagtaatttaaacatccacttatgcatttttcccccggtttgtaagaaggtggtggtgctgcagagcgggctgtgaagaggagtcaatattaaaaatgattgtttagctcttagaaattatttcctggaaatgctaaatagcaataaaatcaactttcagggaggagacacattgccattaaatgtcttgtttttgtctcagctttgtgatgaacaaaggaatattcgttcttaaatatgtggaatcttgagattttaaattccattttaatagtggcaacttaaaatttcttctctgtaagaggttctgtattttatgacagtcctattttttgagacattttatatatcaatgactatggacgtagtattttccagtacttacaatgttgttttccccatttattttttatttttatttttttgagatggagtcttgctctgtctcgctcaggctggacagcagtggcgtgatctcggctcactgcagcctccgcctcccaggttcaagcgattctcctgtcacagcctcgcgagtagctgggactacagccatgtaccaccacacgaagctaattttgtatttttagtagagatgggattttaccatgttggccacgctgttctcgaactcctgacctcaggtgacccacccacctcagcctcccaaagtgctgggattacaggcatgagccaccacgcccaacattttctccatttcttaaatagacactggcaggatcttaggttcttagggaatcttatataaaatataaacaaactacccacttaattataatgagcctgtggtctataaagataaggacttaatttttcaaagttgttgactttgaaaatattgcttagaaatgtgttttttaatgtctgaagagcagtaatattattttactcctttagtttgttttgatagcagttgatcaaaagctattataaggcatgtatacagatgttttgtaggtgtgcataagataaaataaggtgggaaatttggttaaagaagtgttcatcttctttagctttagttaaattttatttttctaggagctgtatatacctaatttgtatggtcctggctctctacttattattaaaacaagagaagatggaaggattaatgcatactctgccatattttatctgtattccattggtcaggaaatcttcccagattgtagggagcattgccaagtggttggtgtactggatctgaaaagattagactggtttccccacacaaattttttcatgattttcctaaacattaaggattgatttgtacaagctttgtgtaattttaataattagtctggtttgactaatgtagttatttgatgtaaaaatggttgatactttggaattctcaaattctaggaatgtttaaaactgaatgtatcttattcagccttgaatttgaaaaatgggggctgtctatccagtatggattagggtatatattttcactccctaagaaaaattgttgtcctcagaagcttttaggaactctggcaggacagcttgccttttctcctgcaagaggcctgctgtacgtgtgtttaacagtcaatctgttgtagaaagtgggtggttcttttcccctctttaggggaagctgatggcccagcccttttgggcactcagtgttctaggaaagtgtcactgaagtccctgaatgttacactatcagtttgcttttgattccaggccctgattgtagatgtcattggtgtcatttctttgccctaccaaccctgaagttagaaggggaaagataagctcatgaagcagcttgaatgctgttggtatgctagtcagtgacattggagtatctctgggtgctgctgactgcagtgccctttgatttagatcacaatgatgaaagaactaaagctcttctagaattttatgtctccattctggttgggtttctatataaagatgaccacatacaggtggagtcattgatggggccttacaaatggctgtatgatatagtgcatgctaagttaccaggaaagagctcttggaggactgtttttaaaggggtaagcaaaatttattagagaagatcagcaaagcttataatgaagagcatgcgtgtaaattggaaccagtagggagaagatcatgagtggagaaaatagttataaaagtttggaggtggaaagtccactgagtgggacagaggctattttgggggcatgtaaaaatcaagagccaggtattcaggttgttggaaatcttagtatattactgtgttttgtcactcacatgatgaaaatcaataaggatccagtataggttacgaagctcaggctctcataaagagagtgttgaagaggcttgttatgatgttctttggaggaatgataggggaggggatcgttgaaaattgggaaaatctgagagactctattagttttgttgctgggatttaggtgatagctttagaaaaaaagttgagggtttgagagaagaactgattgagctcagatcaaatctatatgtgtgtctcatggtctctagaccttacttaggatgataatacttagggggagatgagagcctctgaaaactctgaatttcatttgtgtcagatcttcccattttctatttatgtggagctgaaattggtaccatctttcagatattgtgtaagtacctggcatgatttggtggaggtaaaagcctatttgaataaaatcagtatgtggcagagcagcagttgagggtagaaggttttgccataacaagccccttgtgattgtgtaatgttcttcagaacttctttcttccagctgccatttagaaaggaaataagcagagaggacattccaaattgtcacaacaataggaataaaagcaaggaaacaggccaggcagtggtgcctcactcctgtaatcccagcactttgggaggccttgcggggagggattgcttgaggccaggagttcaagaccagtctcaacaacatagtgaggaccctgtgtctacaaaaagtaaaaaataaaaaataactgggtgtggtgatgcatgcctgtagtcccagatacttggaaggccgacgggggaggatagcatgattctaggagttggaggccaccgtgagctgtgaccacaccgctgtactccatcctgggcgacagagtaagaacttgtgtctaaaaaataaaagaaaaatatgaaaaaaggaagtaaataaaataatgcaacagattcagaggatggttatgggtcattttgaccttatgcagggttttggcttgagaagttgggagaaaacattagggatggattagaaagttaaaaaggcccttaaatgaaggtctgtatgtatacaattgatggtatttgagcaagtgatagcatgagatgtttttgggaagttgaagctagccattatataaagatgagcatgttggaggaggagacagatttgaagcagtgtatatgatgataaaggcttgccctggtctcatgggaattggaggatgaggggtagatattggtagacactcattttagtgaaggtaaattagcagttatggatagatgacagtgttgattcaaaagcgagggggcatggttcagtggctacaagaaggtgctctgacttatgatggggcttatgtcctgataaacccatctttaagttaagttgaagatgtcataagtagaaaatacatttaacatacttaacctgcttaacatcatagctttcgcctagcctgccttaaatctgcttagaacacttgcattagcctataattgggcaaaaccatctaacacaaagcctgttttataataaaagtgttgaatgtttcatgtaatttactgaatactgacagtgaaaaatagaatgatggtgtagttacttgaagtgctgtttctactgaatgtgtatcgatcttgcaccattacaaagtttagaaactgttaagtcaaaccatgtaagttggggactatttgcatacctaaggttcagattccaggaaaagattgggtggcagcaatgagaggaaaagacaactgagagattaagaattggtgtgggggttgtattagtttgcttaggctgccataacagaataccatagcctgggtagcaaaaacaatagaaatttattttctcatggttctggaggctggaagaccaagatcaaggttttggtagtgttggtttctggcaagaccactgtttcaggcttgcagaaggccaccttctcactgtgtcctcacttagcttttactctgggtatgtaaagaaggagaaatcattggtgtctcttccttttcttatatggacaccaatcctattggattagggccccatacttgtgacctcatttattcttaattatttacacttatgacctcatttagccttatttcctgaaataattaaggaaatagagtgaaaaacagagtggttgtgtagttacttgaagtaccgtttctactgaaactactgaactacacaaccctgttttcaaatatagtcacgttggagattatggtttcaacatgttaattttgggagatacacttcagttctctaacaggggtaagggtatttaatttctcgaggcatgaacagaaatgattcttttcccctgaatttctgatctacagttttagtttttcattccagaagggaaatgctttctcatctttacgattggtcaagactccttgctcccacccctgcgattagcatttgccgttttcaacctggagatttcttttccatttaccctgtctgtacagtagtttgtgaccacctctgccttttaaagttgagttttgacaattgatatacaattgatttcttctctaacctcttcagttttatagtttgacctcttgttacaaactgcttttaaaggcaccatgcttatgctttaaaagccacttaaacagaacatatatttagaagacaggatgtttttgccacagatgtcaagagggtcttggcagtgttttcacagaatgtctgctggtgtctgtgcagctctgagactctaggcactctgtacttagagaacatgtagaggttaagtgttcatcaggagacctgcgatgtttgtgttggaaatactaagagatgtgaggtttaggatgttttgtgttgtgttgctctgtgtgctcgtccagagcagacatcctcaagtcagaacttaatgaggtatgcggaaaatcaccaggggaaccgtaatatataaaacataaaattaaagctctgggatgaatggctaatgcctagttacattagatagtcagtttatagttatttgctccagcctaatttggctgtctattcaaggtaatcagcagagagtcaaacctttaactttttcttgagggactgcagtgagccttttagctttctttacgttgagctttatggtgacagatgagatgaaggatactatttcacttccttcaagtttttagtgtggcctcattatatttgaaagtgaattcccagagcagccgatatgcctgtaactaatcctaagtagaaaatctattccaaagcagaaaatatattttggctttacctgtgcagtcacagactgggaaagcgggtattggggtgtctaataatgtggatttaaatggtttggttagaatatctactctcctttcatccaaccaggcatttccatatatttttgtattccctctctttttttctaccacagaagtttaatttcaagattttctgtctcatctctctctggtattcattacacctgttacatctgttttgtttccagtacaaaacagcactcggacctgcctcttttacattggtcctctaaccaaagaatgatttaggagaaaatttgtcatttgttgaaaggttgttttttttttttccccttttttaccatcaacctgtcattttgtgtatcttaatggattattcaagcatttatagccctttgaaaactttggtttcaggcattagaaattaatgtaatttaggaacatggttaccccttaccttacctctgagtgtcctacctgtatacattcaggaaagggtttaaaaagcatagaacacttaaaaacttcaaatatgttttctcatttataagtgacatttagtaaagtcttgaaagatataagaagctaactgttttgcaaccatactgttttcctctgggctgtctttaagaatgtttatttacttttctggggaagggagatatgtgatagcttgtataatacgatacattatttaggaagcaatttcttgccattcagattgtgggtttcggatcatttagctaaaagattaccctgctgagtgcgtggaaatcatcttaagacaaaataaggtttgtgaaaatgaaagttgaataggaagttgtgcagatgaccaagctttatcattcagcaggtgtcagattttaagttgtgtggattcttttgagaaataattaaaagttttaccgaattgtattactattttgaactgtttcctcaatactatgcctcaagaattcagttgaggacacatatgcattttaatacgtaattttgtatgtagcataaatttaataaatcacttaatatataaattcactatttgtgaaatgacggtcacttcagtagggttaggggacatacattccaccttaccatttaggaaaacttgcatttcttgtggctgcgtctagtgtctgggcactggcctagactgatatagagggtcactgaccctctgtatagggactgaataagcagtccctctatatagggactaataggaactgaataagcaggccaaacttctattccccatttactgagtctttaacaaggccatctaaagttgtaatggtaagaaggaaaaaattgggggactatattcttacactgccctctataagaatacagaagccgttggtctcttttcttttgcttggatggcagtagttcctccttacccacagtttcgctttccatgatttccgttacctgcagtcaactgtggttcacaaatattaaatggaaaattccaggcataaacaattcacaagttttacactgcgtgtacttctgaatagtgtgatgtaccctcatgctgaccagtcaggatgtgaatcctccctttgtctagtgtatccatgctgttagtcccttagtatagccatctcagttataagattgactttcgaggtattgcactgctcgtgttcaagtagcccttcttttactttcagcggccccaaagcacaagagtagtgatgctggcagttctgacatgccaaagagaggatgttaaagtgctccatgttccctttaagtaaaaaggtgaaagttctcgacttaaagaaaactaaatacgctgagagatcacatatacataacttttattacagtgtactgttacagcagttcttagttattgttaatgtcttactgggcccagtttagaaattaaattttatcataggtatgtgtaggaaaaaacatggtaggggtcagttctatctgaagtttcaggcatctattggggcttttgagtgcctggtaaaaaggggactgccatatctcatatatttattccaataactgaatgagatgataggattattttctccatttcctaaatgaggaaatcaagacttaggatggcaaatgctagaagggatcaccgtagagttaacagttgtaactactaggctatatccgattgctcagcaagctctgtttattgtcactcacttgttatattttatatcctggtccattggctttaggttagtcaactgttattttttccttattttcctaaaattcatacccattggttacattagctgtaaaagtcattttgtgctcccttcttacctacagtcttaaaagtaaaaattttctaacctgtagttcattctctcagcaaaatgattccaacctggttttttgcgcctttttaatcactgcttggactaaagcctgtgttcagactttccctgaagttagtctggatcagctctgcctcagcacctccactgtttttgaacttctgtttgccctctaaaagctggatgaattccccagtctctgattatttgactcttgtttgtttggcactacctagctgatagtccacttgtcttacaaaagtcttttacagtggttctcatccttgcttgcagattagaatcaaatcgggtgctgataaaaatgccagtatctgggcccttaggagagattcttatttaattggtctggggtgaagcaagatattggttattttagctgcttcattgatgtgtggtgcaaagagcatgggctctggagtttgttagacccacattggcaggttttgtgagttctggaaaagttaatcttttttttttgtttttttaagcctatttcctcatttgaaaaatagggatgaggccaggcgtggtggctcatgcctgtaatcccagcactttaggaggctgaggcggttggatcacctgaggttaggagttcgagaccagcctggccaacatggtggaaacccatctctattaaaaatagaaaaattagctgggcatggtggcgggtgcctgtaatcctagctactctggaggctgaggcagtttgcagtgagctaagatcatgccattgcactccagcctgggagacaagagcgaaactccgtctcaaaaaaaaaaaaaatggggatgaaatgacccattttattaagttattaggatcacaggggggtaatgccctttaaaacggcttagagcatggcctagcatatcctaaattaaattaatagctgtcatcctaccctgtccaacaatgttgtcttccttttccaaatgctcatattctttctcacatttgccgtatatatatctgttgccctttcctcatgtttgaattcttgtctttcccattaaaatttatttacaggtccaagttcctcagtgatgttttattccattgtgtgcttaaataaaagtgtaccttttatcttttgcaatatattggacatgaaaaacttccaaagagaaatagagaaacagcatcagaacttctttgaatagaaaagtaaactgggtcatgtgggtgtcactttaaaattccagtggcttagatttttaaaaggctttcctttctctctctctcttttttttttttttttttttttgccactaaaatctgttactgagactactgcaggtttcctgcttatatactttttgaacattaatgtcttcgttttctgtcttaacaggctcctggtcctatctgtaccttgaagtttaatattttcctccaagcttcttatagcttcagggtaactttctgagtgatacagtctatctccagtaataaagggcagatcaacaccagttgaaagggataactgtctttctatttttaataggtacatttttattgagctgcattttttttttaaagtgtgaaagagactctcgccctcaagtttcattgttattcttctgatatgtcagtaatcacctttgggttgttgtcatttcagtagctcagggtcaatatatattgctaagtcattcatgtcctgtaaatttcagtatgaaataagcatttactggttccttgcctgaatagtctgagcctatatagtttatctcaatataaattagatctttgttgcttacatcattttgatgaaattaggtattatggttataatttttttaaagttctccttgactttcctggtatgttaaacagcttttctatgttactttttaaaaaatagtgcacacgatgtgggctttcacagcaattcttctggaaggtgacttttcttcattgttaacagagataaatactctgcatttatcacaaatgtttcaaatgtccttggatagacttagaagtctctcaagttcagctagcttgagacagagtacaaaatagaatgtagcatcagtgaagaccagtcaggctgttcttgcagtagatcactatgccaggtgacctcatagtgaccttcaagtaccgaaaagtctgggattcttacttattttacaatctattctttgggaatgtaaattcagtgaaaaagttgagatactttatttttgagtgggacccagtgtctcttcctctatcaaaccgatcaactacttttcccattgtgtgtcctctccagttttgatctgttattctgctcagactggagctggtcatatgctttggaaatacctgtttacagattgatgcagacctggagcagctgctcattgacagaataggtctagatttctatgtcgtgactgattcgtactcatactgggagttttgggttaacagacgtgagacagtgaggattgagctatagtggcttcaatttgaaggggattgtttcctcccttgttatttttgttttcacactatccttaaagtaatgcaaaatttgggagatatctgattaaaaatgtttcattttgcctatatttgacttgtaggtgcagctgtgataactaaaagatcctaagtccttttttgtcctccaggacactaagttaaattcatcaaaattctcattttataatatgctttggtctatgtgtgggctcttacttggctgctgaagaacattgaattgtgtgaacttgtatgactttatcttcatatctttcttgttcatcttccttgataaacactttgttttcatttcttgtgctttgatgaacagtgctgctgtgaactctgggagtggctccctctactgcctcaaccctcattgtgacagttgttccttgatatatgtacttctcagggaagtacaataagaagtaaaactgtacattgtgggaggggaataataggtttgtcaactctgttttgccaaggaaaggcatttaagaccatctgacacattatctattatcactgtcatttcctggtttgcttagatgccccaaagtaggatgcacgtaacttcacaagagaaattgaatatatttcactttacaaaaaactcattacatttcttattttgcctggaaccaaataagatttttcccacaatgtctagtgaagtacttggcatttaggcccaagtctacttatttttccctacggatattaagtgtttcttgtacatgatcaatttactgaattctgacattccacataaattctgattaaaaagttaattggcagtgtgatgtaatgggagttacaggttgtgccccacctctgcacctacctagttcagaaacactgagccagcctctgggtttcatttccttcatttgataaaaaggaatggttgaggatagatgggtttcaagatctctgtagcttaagttttatccataattgtaaaactctgcctgctgggttccacatattggatgtaattgcatccctaggatgacattcctcagggtacaaattatgtcttattcaccagtcccactgcccccaacactacctagtatgtgtgtggcaccatcctttaccttccaaagaccaagtattacatatatgtagtcatgtttatctttagatctcagcttaaatgtcacttaatctgcagaaggcttttcttctgatcactgtcccagttttattcccacagcatccttttttccttcatggcatgaactgcaattctaaacattagctttattctttggcttgtttaatatgtctctcccactaagttcccttagtacctgtcttgtttatgctatagtctctgtgcccagcatttttgtttttgttttttgagacggagtcttgctctgtggccccaggctggagtgtcgtggcgcaatctcggctcacagcaagctctgcctcccaggttcacgccattctcctgtctcagcctcctcagtagctgggattacaggcgcccgccaccatgcccggctaatttttttgtattttttttagtagagacggggtttcaccgtgttagccaggatggtctcgatctcctgacctcgtgatccacgcgcctcagcctcccaaagtgctgggattacaggtgtgagccaccgcgcccggcctgtgcccagcattttaaaaatgattgcaactatgtgtactttgccctttatttgaacctatgacaacagagagcctagtgcacatggcttttctctgttctgagcctgatttttttttttcatctgtcatccagatacaatctctccttctcacacttaccagattgcgtcctgtgttgtggttctgcatgtgttcccttctttgcttagacagtaagtgggaactccttatctgagtcatcttggttatccagcacagtgccctgcacataggatttgcatgttagaatgaatgcagatgtctgtccttcaaatgtgttgatatttgaggcatatctgttttcttctgtgtccttttagtggtgactaaagccttatttgaaagctccatctccaagaattcctgatgcctttattcaataatgaacctcgttgttttcaggttagtgcatggccactttgtagttgtgcagaattgtaccagtttttcatgctctctgaagttacatgtttttcttcagtttaactttcggttttccttggagttactccatttaagtggcttcagactgtgagctgagtgagatcttgctgtcttccagtcttggcaccttgaatatttacttttgtttacaagctagtttctctttggctgtttttaatttggaggactaaagtgtaacaaatgaggtaaggataattctgcttattaatgtcttatctggaagtatgatgacttcacgcctccggagagtagagtttggctacctttcacaggtattggagtaaattccaggtatgcttattgcccttttgagataagagtttgttggatgcatagaaaatgaagtgggctctcactctagccaaatagttaccagttagtatcttgggatatctgcagactcagctgagctctcaaatgggagaggggatgaaggcacaatgagcaactgagatgggaagctgggattggaaagagaagaggctatcaaagaaatttgtcttccttcctggtttcacttcagctcacttcttgtgccccggggtctccatccttagctatgcttccacatcgggcccaagtctgtttaccaaaccttggtggtcaagcaggctggtaaatagtttattaataaattcaggtagaatcgcctcattggcagaatataccaagaagtatttgagcatataaagtggttttcttggtatgggatggaacgtacgctaattgtcttcaggctaaatcttagttcatggttgcagctctttaaaatggcttaagctcttgcatggttttttgttttggtttggtttcttttcgtttgtttgtttgttttttggagacagtcttgctctgtcacccagtctggagtgtggtggcaggatcttggctcattgcaacctctgcctcccagattcaagtgattctcctgcctcagcctcctgagtagctgggattacaggtgtgcactaccacacgcctgtaatttttttttttgtatttttaattttttgtatttttaatttttgtatttttagtagagctggggtttcaccatgttggccaggctggtcttgcatgtttttgagaatgctaagactctgcgttaagcactatgctagcctctgagcaaatgcttacgatgtactttgattttgaatttttcaggaatagctatagcgtgtgattcttaaattgccaactaaaacatcaccgtctgctgttcagtaataaaagtgttttgacttggttatctgcttggacatttttatgttaaagaaacttcttggtcgggcatggtggctcacgcctgtaattctagcactttgagaggctgaggtgggaggatcctttgaacctaggagtttgagaccagcctgagcaacatggcaaaacctcgtctccacaaaagattttaaaaaattagccaggcgcggtggctcacgcctgtaatcccagcactttgggaggccgaggcgggcggctcacgaggtcaggagatcgagaccatcctgactaacacgctgaaaccccgtctctactaaaaatacaaaaaattagccgggcgtggtggcggttgccggctactcaggaggctgaggcaggagaatggtgtgaacccgggaggcagagtttgcggtgagcagagaatcccgccactgcactccagcctgggttgcagagcaagactccatctcaaaaaacaaacaaaaaaagatttaaaaaattagcggggtgtggtggtgtgtacctgtggtcccagctactcaagaggctgaggtaggaggatcccttgagcccaggaggctgcagtgagctgtgatcacaccactgtactccagcctggttggtgacagaattagaacctatcttcaaaaaaaaaaaaaaaaggaaagaaaaaaaagaaacttttcttaaatatgctattctgctgtatgttttggtggcttaaatatttggtacttggaacgtagtatattgtagaattcctgatctgtttggtgaaaacatgaccttttgagggaaagaagaaagcaaagcctggagtgtgtgtgtttatgtgtatctttgaccattaatttgaatttctcaggttgcagtgataatggcagtttgaccttaccataaattaaagatgtgtttttaggagagcatgaaagcagaaaacaattctcaagaaggttagtgccttgagaacaataccctactttgtatagtgcctgaaattttttataagccattcttttatctgtaatttgttataacttatttcttttaataaccctgttcgctagcagagtgagaaaaatctgttcttaatagatgagggaagttatgaacataacaaacttgttgagggtcacagaatcattaagaaatagatttaggaccagcagtgattgtctttggactttatgtaggcttttaccttcgttctagtctttttttccatatccctgttagtttgttagcctgggacttgggaatgagataattgcttctctgaggccactcctaaccctgactgttttatgtgaaagcttaaatttgctccaaatttcctgaatgctacatagtgttgtgacttgcccttttttggccttaatttacctggccatgtcttaactctcctgaagagctgaactaaaatcttcattttggtataaatggatgccttaggttctgatgttttagttatggaaaatagtgcttatgttttgggtgctgctgtgtgttacacatgatcatttgatttgagtttagaccaaatactcttatttctttaaccttgaccattaatgaagaatataagaagaatgttggtataagccaacatagttatggatgtgagctaagaggtgtgtttaagttttacgaaggtcacaggaaattgaccacaaattttctaagaaaataattataaaaagactcataggattcaatattataaactttcaacaaattgaaaatatgcttaaaaagccattttccaagtttattaagttggaaatttatcttaagagttaataacaattgaatgtccagggggaaccctaaacactttttagtgtttatatttgaataaatgtgagagcttcacattctgtttagaaaattctctgttgttagactgctttcttatttttgaatctcatgcatgagattaggaaacaagtgtaaaatgattattataatttactattaataccaactgttgatctgttctcagttcaaaatgttttataatacagtcctggaaaagagaatcaacctaagttatgtgtgaggttttgtagaatagatgatattctctgcagatttaggaacccatactttactttgcaatagagattggacttggaagttcatgaagtttttttttttttttttttttttttttttttttttttttttttttttttttttttaaggaacactatcaccgattctggggaaaaaaaagaaaaagccaatagcattaacataatactctgcaaccttaaaggtttgcagttgataaggaataatttaatgtgccctataaaacccacagttttggaattgctccagctttttatttttagctgtggcttttagttaatgagaagaggtgaaaagtatagtttctcctggtctctgcttgaaatcacagaaactttccgcgctgtgaaagttcaggggattttgggagaccatccttatgaattggatggtggtgtctcctcctctttgttcaggcctctttttctagagaatagcaaagaatggtgagttgattaccttcagattttaaccaaactgtaattgctctgatattgtcaaatgcacctagggacagtgttgcagtattttgaaaccttacacaggaaacaaaatagcagctttggaggcagggaagaatgcattagtttctgagtctaaggaagccagaaggaagcccgcccacccactctctagctttcatattccctcagccacctcccagatcttccattgctgtgctataaatagccagtgtttaggcagggcgttgctttcttcccttgcctttgaagactttctgcaggccctggaattgcctttgttcctctgtctgcccactttgccagtgtgaagcaggttttcatgggattctactggggtaaaccaaaagcagcagaaaaagtgtgaagtaagacctcttatttacccttcagttccttccccacttacctcctgttttacctccaaaatacaatttgtggtttgtcatgagacagtttttagagcgttaacctggaaatttaatgttaattcaagggcttctggtgtagtgccaggtgatttttatcttttttgtgtgctgccctactcactgcctcatcctggggataaagcacttcattggcccctcctcttctttctcatagcaaaatatttgattaggtttgaattaactgtctcaaactcagcccccagaagggacaggatatacctaattctttgtggacaggatgactttctaatccaaatccatagtagtataaacaggccaaaatgagttatttagagtgctttgaatgtctgtggcagtcatattaggatgtttagtgacacagacttgatgcttttttgcctcattttttgttatgcatattcattagaatattttcacagatgattaggtctacaattaaagatcctcagtttagatgaccttggaaaaccccctccctcccccagtactctgacattctgaacctctccatcatggtagaaaagcatatactctttagcacatttgacatttaggctatgatactcttttacttaaaatccagcccttgcaggggccggaacattaaggctgtattgggagagctggaaagaaggcaccccatttatagggcaacctgctggctagtagctctgggaatatggactcttgccaaactttatttattaagcgaaatcagaaatatagttttatgaatgtgaaattgttacattggtgaaattttaaatgttgaacactactgtttttgtttgtgttttgttttgtttttaaggacgttggagaaaccacgtaacatgttttcaggctaaatctacccgctagctgccagtccgaactttggcctggaggttgaggagagttacagaatctgcttgtttttgtctatcccttgccacttttcctttgtccttcttttcaacctcgcacgtgatttatttaagcagagtccattgttggctttttattttttgtttttgcaagctggccctcggattctgccatttatttgaatgcttaatactttaagtagacttttgactcccatcccctcaatgttttcttcctgctggtcatagccactctggggggttttgttcaccatgcccttaggtatatgatgtactttgtatcaggaagaatgttcagagtaggccacctggctgctgctgtttatgttggccatgtttattagaataggtcttagttaatgtgtagtcaaccttggtggataaaaaagattcacttgtttccattttggttatttgtaatgtaggaggccagctgtctttattcttggcaagtaaaagtatgggtcattttccagttagagcaagaagttacttaagagaactaactgctgtcttggtagccagcgatgcagagcagttgttcttccttaattgatggcaggcaaatttggtctaattactgttccagtgcagtggaagttttcttcagttttaatttaccgttcttgcttctacctcatgataccccccgtttcccacttttgctctttagaaatggagaataaagaatagtgtcctgatgactcaactcaattccaaggcctgcagtctccagatctttgttgtgaatcctgggttttagcctgacaaaggatggatgtaaaaagcatgcttgggtggggcagaagggtatagaaagggcacgtttctaaaccatctatttcccttatagtgattctcaactagagactggcactcagaatcgtcaaggaactctttaaaatagtctaagatagagcgatagagtggcatttgagtagatctacactcttcacagaagaattgaaaaatttgggttcagggtcatggttccctttagaagagtggtttaaggatctttttgttcatacccatctcagattaaagtagtggatgatagcgagaatttctttttagctcgatctgaaagagattgcagtcctaactgctgtagactatattgcctggtacttagtaggtacttcctgaaagttttatcaacagttaaagactatttactgctaagcaatttacatgttaaatctactctactgagagttcttttaaagaaacttgtactttacataggatgaagttgtaacaggaaatgggtacctggaaaactctctgagcaattctaaatcaggaaaacttcagcctgctcttctttataatttaaaattttcttagtgaggcagaagtccactttaatgttcagtatcaatgctgtgcacagtctcctgttgtttagcactttaaacactgggaagcaaaatgcctcatttcttctcaaatacgtgtccacactcaaatattgaagttagaagtggtagtttccatagcagcagtgattttgccatctttttttttttttttttttttttgatagcaaactatttagcataggtcaggtagagaagggattatttctggctgcgggttcttaaaggtttccttgtgtgcgtttgacttctttggacaccttcttaggacacaggtgtgttttggagaagttccgtgaggggtggaatgagtggggttttgtacatctttgttttcccagcatctgagcacagcgcttggtacatgcagtagatcaggaatgttgcttttctagccttggggagtttctttactcctctttttggtattctacacaccagaccttggctgtgttggggagagagtataaacttcaggtttcctcttgattttacacattggtgatgagattggcttattttgaactgtgagatggtgacaatggaacacacctgtccctggttcctccctgaggtttatattgttttaacaaaggcttgtagctaggaattaaaaaatgctgattccacttgaagtttgagcttacaatatggaattagtagctatatgcggaaagagatgtgtgtgttagtggtaggacttgacagcctactttcccctttttccccttgacagtctaccttcctctttataaacaacctgagatgactttggctgctcctccagctatacagggttgcaatggaaatttttcccacttggcagggaacataaaaccacatggataagtaagggggttgttttttacttgccaccttgaatcacaattcatggcattcattctctttatggatctgtcttgatattttttactggggagataaaggtgagtcaggcatataggagcacacagagttatctggtccaactgagtggttcacagaaggcttcctcgaggtttgtctgctctcttccggtgctgaggatgtttccatgaggcaggagaagaaaggtaggctgttgcaggtggagaaaacaggttgggtgacactggaaaacactgggagtgggaagaactactgggagcttgttagagcttgtgtatatgatgcgggggtgctgggcatgtggatgaagaggtaggagctcggtcaagtcagctgcctcactagggagctgagatgttacttcataggcaacgtggagtgagcagagctttaaaagagatagtagtggagtgctgcccattattttagttagatccccacagccgtggaatggaggaaggattggagaaaggggcaagcctggaaatagagagatcagctaggaagctctagatggggtctcagcactgatttcaaatcccagtttctcacttaatacttgtgtgaactcttgaagttttgaaattctcatttgtaaattgaagagaatgtaatatagcttatagtgcataagatttgttttggagacatggtcttgctctgccacccaagctggagtgcagtggtgtcctccagccttccaaagtgttggaattacaagcgtgagtcaccttgcccgctccacaagaggattaaataagattttgtttgtaaatatctagcttgagactggcatggaaagaggaatcaataattgctagtttctttcccttgccatttatttttacttttagttgtaggattcgttggattctccagaaggcagggagatcatctgaacttctttactgtagcaaaagatactcaaatcccacggaaagttccttttctatttttcttgtaattaaatgtgattttaaagccacagtttaaattcaggtcatttttgaagatcatgataggagaaagatgataggagagtgtatgattttgtatgaatgtagatacatggatcatttgcctgatccattaattgtggaaacttgatcccatgtgatctcataatgtacacattattaatctagattctagggtataatcgtggattagatttctacactcttgttttctctgtattgtaaatctgaatataagtacaagatgcctggatcatctttggagttcttagccagaataaggaggtagcaatttctaatcttattaaaattccaagagcctgagcataagcaggctcaaagaggatgggagatgtgtatttttatggggagttggggaggggggtgggggtcacacacttttaaaaataaggtaaattaaaataggcaaggctgtaattctaatgtgaagtacttgggctgttagtactctgtatatttaaacatagtctttggaggtctggggttaaaaattgcagcattgcaagtaaaaatttttaacttccttagtcaccgtttgtgactgtgctaaatgtagtttaacatagcatctgcttcagcagtccaggacaggaattaacagtcatgattaggaaaagtctgcatactgtactttctgtccacagtctttcctttccatgttaaaaggctcccaacacgactctgcagccttgctgtccatttagatgtggggggaagaaaaactgacaggcttaatccaaaagacagataagcaaattttctttctttctttctttctttttttttttttttttttttttttgagatggagtctctcgcacttttgcccaggcttggggtgcagtggtgcgatctagactcactgcaagctctgcctcctgggttcatgccattgtcctgcctcagcctcccgtgtagctgggactacaggcgcccgccaccaagtccggctaattttttgtatttttaatagagatggggtttcaccgtgttagccagggtggtctcgatatcctgacctcgtgatctgcccgcctcggcctcccaaagtgcagggattagaagtgtgagccactgcgccaggcccagataagcaaattttctatgctaacattttaagagacatcttgttttgtccagcaataatgataataccctctgttcacttggcaccttagagtttgtaaagtgctttgacattatcagacaaaatatgtatatgtaattaaagcttttaaagagaacagacccctttacctgctcttttccttcctagagtggaaataattcaaaggctaatgcttttgtgatttgagtcttaagaataatttcctgttaaaatgtagcctgtaattgcgtatgtgctcccttctcctcttcagcctgtgggggatagatgtttctcatcaacacttgggaatatgtgtgtataatgtatatacaggttatacatatacatatatgctcatacacttgcttcatttttcatctgtaacatgggggtacttgccccacctactttttgagatccttaagcgttcgtttcaaatgtgctttacttgaatgatactaagacaacttgcaaagcgggaagtctgacaaaagtaggagtaagaggaagctctatgctgggtgagatggtgaggtcatcaatctaagcatattgacccctttctctgttagggggttgttcgaaattgggtgtatgtgcctgatgtggatataagtggataaggtctgaggctaagaagggccttacctgttctatagtgtctagcattaacttttttgaaaaacattattaactcttatatacattcttatgtagcagattaacaagacatttccttgagatacttctcgcccttagcaaccctttcctcactttggaatcactgaataaaaataacacttgcattgaatgtaaattctccccctcccccacttttttttcttgaaatgtagtaaataaccaggaacttaattaccataaaaagtcacagatgtacctaggtgtgttttgttctagctgggatgtcggtttttaaagaaagctgtctttgggggagcaagaggcagaggtggaccaaaggaaataaagttactatcttagggtgtgaatcttgtttttttaagatgcctcaaatttccatagtaaataaaacatagttgtgtctctaaatcacaagtatacatgtcttgggaatctcaggacgttacacatcaacagaaaagaaaaacacatttcataggtgaataccagtaattgagatgatccacagttcagagcagaaggaagctgtctgtattgatcaaggtttttagactccttgttttgctctccacaatacatcaatgaattctaagatatttatgtgccctgggatggcatccctttatttgtagcctcctaatgttactaaggaaataaatcttgttggttgacattttagatgcctttttttttttttttttttttttggtgtaagctgcttgaggttaaacagtgtagcatttggttttctatctctaagtcctgctaaataacagtgaaaaataaaatgttaacctcactggccaggaggagtgttgtggcctttataacatttgtgagttaaaaaacattatttttgtccataaaccttccttgtcttttattttccctttaagattagtcattctttctgctttggagtttgtacatgacagagtattttagtgtctgtatccaacagaggagctatgtttttaacctacctaatgaaaacactcaactactgggattaatttgaggatttccaatcagcctttcacaatttgtactaccctgagggggttcctgatagctgtgacttttgtagaagaatgtgaagttaatccaatttctgtgttgattactatgttaactgtcttaatttagcaattcagattaatctgcctataccaaacaaaacctctgccacaaaactgagggagtagttgattttattcttgttgtactcatatccctttttagattctattgccaaataatggcatggaaattaattgggtccacttaggttggaatgaaagaatggggttggaatgattcatcaccagaaggtaatgatttagtgtattccagcctataaccaagacagagttatctcatggcttaacctttactattgtagaggtatataaatgaccctgttgaaaaatacagattttaaagtttactcccattttacaaaatgtgacttaatctaatcttcatccactttgtttttaaacttttggggagagctttgagctattttactttgaattctgaaagttttaagggggctcacatagattacaaatcaattttctgatatactatgtttagtagtagttgcatgttttaagctactgatttgtgattttatatttgctcctaaatgaatcagttgattctttaagaagctggcagtggattcatttagtagcaaatataaagtgaatagtgaatatccaaccataaagtgaataactctggatttaatatttataaagtcaaaatttttgtatgttgggtttcctatatatatctatatatagatttatatatatatccttattttcttttaaggcacaactgtccataaaatctcttcctttgcttattcatcttgtgttcttagtgtccagagcatagtaaatgttttggtggtttgtaggaatgtgagcagttagtatctaaaccagttttttgacaatggatttcaacagtgtactgatgtaattaaaatgtagcaccttaataatgtgttttcttctgggtatatccagaatctgatgacttctccccaacttcaccctggtgggagtcactgtcatcctgcacctgaattgctgcagttgcttcttgactggtctgtttccaaactcactgagtcttcctgcacacccacacacaatttattttcacagtagccacatgctactcttttgctgaaaaccttttatagtttccttttttgttatggtggccttcaaggccctatgtgttccggcctcattacctcttagactcatctcctcctactctccctgttatctccttgtcattctgcacagcagacaagctttgcactggcaggtctctgcctagaatcttttcttctagatatctccatgggggctgtctttacttcctgttagacctctaacaaattactttaaatttactggcttaaaacaatacaagatttattgtcttaaaattccgaagaacatcagaagtccaaagtggttctcagtgggctaagattaaagtgttggcagggctgcattcctcccagagtctttatgagagattgtttccttgccttttgcagctcctagaggctgcttatattccttggctggtggccccatttaaagccagcaatgtcctttcgatttcttctcatgcttcatcaccctgacagtctcaccttcctctttcacttataaggaaccttgtaattaccttaggcccacctcagtaaccaaggataatctccctttctcaaggtgagctgattagcagctttaattctacaatcttaattcccccttgtcatgtaacataacatattcacacttctgggagtaggatgtagacattgaggggaaggggatttttcttcctatcacagcagccagcccatatctccttcaaaccttggttcaaagctcaccttttcattgaggcctgcttgtttaatgctggaaactacccgtcttcatctccacattttcaaccccccttattcttctctatatgtattccacagcactcttacttattgctgatgtactgttttcttctatgtgttcatacttgtatatgttctgcacacaccatccacagcaaatcagcactccaagatgcttcttcattgctgtacctcaggactctcctgaattcgtgggagctgcatggagatgttcgttgaatggatgaatgaacaaaacatgaaacttaagcagatctttttcgccagatctttactttcccattgctcaaactggatacctgtaatggacttagactgaagagaaggacttgggaccctaaactcattatcttacctctctaacctcaaagcttttgaacctgccagctctgaaactacaaaacctcagaaagactggcatgaaggagactgtgtactttgtccgtagaagtcataaagggccaagagacagaaaaacgttagtgtttgctgccaaagaacattctccttatgacgcgtgcagctgttcagtgcattgtctctgcaagttcttgggccgttgttagccccaaggctgaaaatgtgggtttgaggaattcgtagagctagtgagacagctaacatggtatgtgggtgggctggagaattgacaaggattctagattgaataaatcctttgacatttaaagctacaaacctttattttaggaatcacaaagcaaagagagttaaagaaaacaaactaatggcttgttcctgtaactgacattgttagccttgtgattttagcacttggttgactattctcttgcttaattaagtgaaaccacaaacatatcctgaaaataccacttagtgatttgaatgggaaagataaagcatttgctataggaatggtgtgtgtcgggcagatgatccctttgactgcctacccagctgtcatctataagctcatatgacttatttcaccaattttggagtgcttttgtccctgctgcaagttccccaaaggatggtgtggtaaaggttgtgatgcctggttttgttttttgtttgtttgtttgtgttttgtatgtttggctctaaaactttagagacaaatgtcttctgttcatctgtaacttggcatttagggagaggtttgatgtggtcagaaggaaggcctttgtactgggctgattctgtcctggtgtgctgtgtttgaatactcagggtattcaaagcttctagttgcaaataagtgtagagaccaagttcactctgtttaggctggacctttccagcctgagagataagcttgtctttgcctctagccatgattcttttaagagatgttatcgtttctgtttgcctaaacttttctagttagtatgaagatcactggtgatccacgacttaatcatgatgtgcttctaaatgccttcttcaagatctgtttaattttcctgcatttacctttgattttgccattttcctgaagttggacccaaattccacatgcccatatccataaccctgctggccagaggcttggatttgtcttgtcttccagggttttgtagttagtgtgatagattgttgaaataaaaatacacaacttttcacaaaggtagatggtttccttttaccatataataggaatttagatttcagtgggtgagggtggctgatggagtgcgtcaaaacttgaaaatatgctttcataattacttctggtttgaaatacttggtgggatagaaaaacaagttagagccactcatgattatttgtgtttccaatttatagtttaaatgtatttgtaaaaagtaaaaaaaaaaaaaaaagttacctagtttgtacacatttagggagggatttgctctttcagtctccaatttttaagaaactgttcttgccctttgctttttcaatgcttagtttgtacaaacccctgaaatggagatggggtgggtttggatggagccttgaataagggagatgggactgcataaacaaagctagaagagtgaaggtactggactgtttgagggacaaggagtagttcagatcttaaggtgcttgaataagtgggctgataaaagtagttgtacaggatttctgttgatacctagtagtttacacttcatttgataggcagcagggagctgtgggagggtttagaaagtctacctgggaacattgttaaggtagattaggatgaggagagagggaccttttggaggctttaatttgctggtcctgaagaagatgagagcccaggtcaggatgttgaccatggcaatggagtggctggagcaacaggcgcagcctcaccaggggtcattgtattggccctttcagcagccctgctatgactttaacccgcagaagtctggtccctaaatcaggtggtttctaggctatggaacctttttccctcctcgcaacattatactgagtgaacttaataacaactctcagaatatagttaggatatatttatttacttattttggagacagagtctctcactgtcacccaggatggagtgaagtggtgtgatctctgctgactgcaacctccccctcctgggttcaagccattctcctgcctcagcgtcctgagtagctgggactacagaccacgcccagctaatattttgtatttttagtagaggtggggtttcaccatgttgcccaggctggtcttgaacttctgacctcaagtggtccacctacctcagcctcccaaagtgctgggattacagacgtgagccgccgagtctggcctatttaagatgcttttgattaactacctggtttagtggctgtctgcaagaggtttgaaaatagtgaagaagctgattaccttagtttgcagaattttttaattgtaagattagaaaatatagactgcttttctctcatgtgaccaggaaactattggatcagtttgacttctcaaattcttaactcttcccagatatgagatataaagcatttattttctaaattccactattgtgaaaacagtagatgttaaaattcataaaggattatttaaataaaaagtttagaagtctttatggctttttggaagtacctgtgtgtataaaagtagagacaatcttctatttcccaataaaacctacgctttcaccatagtaacagccattttaccttggagcccagtgaaagtttagaatattctagattcatctatgctaaagtgccttttcgtaaattattacttggataataacaggaggaaaagcagtctgaattggcacaagtgatgtctaaattaagaggagaatatatattgaaacattatttttttaagccttaaaattaatggctctgtttcactggaatatggccttttttttctttccgaagagtttcagagccagtggaatctataaatagtacacagcccagttgtctgtggtagattgagtagagcagaattgcctgtccttaagtgaaggggaagtagctttacatttttagtaactgaattgttggccagcataaaggtaagggccatgtgagcaacttatttataacttcccataaaggtatgtgtctgcctcaagcggagaagactgtctggggtgcttgaaatgtaaaagtgcagtagaatcattttccatacacagccttcctatctagttggtgcaataaagtgattgttcacctccctggggctttcagaaagaggggtgtcaatagattagctcattagtgtccacctcctatgaaatagaagggactgaggcagttaaattaaaatctgttttatgtagtgagcaatgtttagattgaactcctgaaggtattcaggacatacatttatgcctgctttaccctgctgggtgtggtcctgctcatgtagtcttaactacagttgggttcttatgaaaaatctttcatgggtgtaaagcactttttcacaggttcagttaaactataagtagttttttgaaaaataacatagggtcagaccaaaatctcttgttacagtgggagaacggaagaagagtaaaatattttcatggcttcctgtaaaaaagtatgttaatcctttttgatagatgccagaaagtggaatccatcgttactggcggcattgctaatatcaagagtaaatcacagccaggattgcctctcaggataaaatttttataattatcaataagtaaaagacatttttggacctcttctgttattcactgtctatttttagaaactaagttttctgattatattgaccatttgaagcatctgcatatatggcaaaaaaagtaaactttggcttttgtgccaatatcatggctctgtttggtatttgggcaagtttcaataagtggtaaatggcccattaactatattggaggcctcatagcagcactaatttcaaatcgtgctgggtttacaatttacatattaaaaaatgttcacttgatttcgttcagggacaaagctgacatcctatgcgagcattactccaaacaatcctatttatcggtccaattatttcaattgaaagatgctggtttactgcctcttagtatatgtgagcaattctcattgaaactgttagaaaccgtttgactgtagtggcaaaataacctcacagggtttcgattcacttttaattttaagtcccaggattaaggatcatttatagtataatgtaacagtgtgggcaaatatagcttctcttaatgtctttctgatattttggggaaagagtgcttttatgcaaaccaatcacttaggaattgcatgatgactttttcccactataaaatttcattcacatttcctcccttctcccacatccttaagagataagacagcctgcagtattttgctttattttactttattttttaaatttttgaggtgtggtttacattagttcttcagaatagatgccaagataactagttaatactgttttaaaatcaggctcttaattaaaaatgcttatatttgtgcaagaaaactacatgttgaaatgaatttggaaagattagattggaattgcctcaatttcaattaagccacactgcatggacgtgctgggaatatcaatacaatcactgtaaattttgtacaaactgatttgttgcattatcttgctcatctaacatacggtactttttgtgtccatattgcagacggcaactccaaactaacatggcagtcagactgtgtgatgtggcttctctgcttagaagtggttcgtgggcagcagagccttggaccggggtctgtgggatttttcttaaattctgtaggctacttttttttagcatgttggaggagtgctgaggccataccaaacacttgaatctttcatttggcatactaattattcttacatttaacctattgtggtatggccatcattaggacttaaagcacattgcaattttaactgtagtggtgtatgtagcatcagaaccatgctctcgtatgtaatttttttacaatctaaattaggatttagcgcagactcaaaagagtgacaagcttttttaaaaaaacaaaaaaagttttggtatggtctgaatactaagttaccgtaattctatacattctgtttactatgtatttttttggttactaaattttgaagttatttacaattctttatgctccaaaaaaaaagttgaaattgtaaatctggctgaaaattatttgcccagtaactgttgaaagagtttgcattctgtgaacagttgtgttgagcctatcaatggaatatgcattatttgtaaaatatagcacattagtatcattttattctgagcagatggtcctatagcagagagatgctgtaattttcagcgcacacagcctactgcagctgttcacttgaatgttggcttaggagctcattcttgccacctgaacatggaaataaatgtgcaattaagtgaggagtgtttgttgtcctttctaaatcttgctgatttcagtcaatgaacactttgtatttcaaatgctatcccaatgagctttagtccgaatctcccattcgggtctcttgtagtttctacacccatccaaacttttcagtagtgatttaagtgtctttggccactttaatcttgtagctcattgtttccataactgctgggcaagggttaaccccagaagaatttttgctgtgaaaagagtgacccacagctaatattctacaaaccagaacagttatgtttaattaggaaatctgaaacttcccttttcaaattcaaatagatttgctaattataataaaacccatatcttgaattatttaagatttttaaatacagatttccaaaggtttattttccttcatccctttaagtattccctgagaaaacttagcttacgtagagaaacagcttgcagcttgtagaataaattcccacttctttgacaaggaaatgaagtcacagaggggttacaggttgttggatatttcaaaagctattctggattttgcctcatgtttttttatcatgacagcactgattcttgttcagtctgaccagtctcaatatgtccaacactgccttcttttgtaaacatctcctggcagtagagatttcttcctatatgtttatctgctaatgtgcccttttatttttcttaggcaatatgtgttttaaaagacagttttttctctttccttgtaaccctctttccctttactttgctttttttctttgcctcaagttctttgccttttctatgttaaagcttccctaaaactagcatcccgtcaattcagaattgtcagttttgagaagcagttctttgtggaggtgagagatgtatgcaatatgaaggataatcctttcagtgatgcgttgatttttagattccaccacagtcttggctgtccaaagataggtagttagtggaaaatgattttggaaaatttggggctgtggtttgtgactcttttgcataggattggttttaggaaactagatttaatactgcttaataagtgatttgtagagtagcctgaacagtttgaggccattttccccaattctgttctttatctggtgtttggagtttatagataccctattacttgcattttcaacaggcttatgtgctcagacgagaattccttgggctggtctgtttgcaaaagttgatatatttacattgttgttttcattgactggataggttttcttttttctcattaaaaagcagagtcaaggagaaacaccatatcgaagtgtttggggattttattttgatgttgtagatcagtcaccttgttggtaatttttagctgccaaggttgacaggagtgtcctattagattctaccagttagacaaagtattcatagctcattcccaagttaccttacagttagttcctttccactccctaaaatatagcatctgtttttttcttaccaatggtagagataagatattgtccatcaaaaacgttttcaatatgtaaccaaaacagtacatgttgacaaatttatatttttaaaagtactcttgctctttgatttttaagttaatggctatctgtcagttttagaatattgaacctcttaggataactgaaaatctcttcagagttaggtaggtcatttttttgttcatttttgtagcatgttaagcacttttattttctagtatgatttttagttagggttcaattctgtgccctttggtacttgattttctcatctgtggacactgcaggtgagtaaggatatgtgaatagacccagaatgtgattctagaaaatgtttaagatttaaattgtgcttatatgcatgtatggaatatgtatgtgaaaatatagttgtaaccttaagatgaataggttaatatgaagtgaaaattagtgtaggtattacactttttgttgaagcattgttgtaatttcattagaacgacttgtgtttagtgacaagtgtttaaattcttggatggagtctctaataggttgtgaaatatgaattctgacatttcttgttttctctgtttttagcaggtaattgctgccatggaaacacaactgtctaatgggccaacttgcaataacacagccaatggtccaaccaccataaacaacaactgttcgtcaccagttgactctgggaacacagaagacagcaagaccaacttaatagtcaactaccttcctcagaacatgacacaggaggaactaaagagtctctttgggagcattggtgaaatagagtcctgtaagcttgtaagagacaaaataacaggtaagcagttttatgtagatgatttatatttaacggatcgtgtctcaagataggagaaaataataagattactgcttgtaaataattcactgctgcaattttacatatctagattttctctctgttattacatcagcaatatgaaacttagtttaaaattttctttttaaatcataaactattttgggggcagtatatattttaactggactttaatgtactctgatctggacaggatctttggaaaatcttctaaaagtatatgtatctgggatagccagtgtcatgttaacttttatcttttagtaccctgagtgtatttaagttagcatttcaacagtgagatacaatatgtgtttgggagaggggaaaaaatcttaaaatgtgttttccttatgttaatataatcattttgcttatatatgataaaataaatggtaatgttcacattgtcagtgtataaacattttcactgatataataccaagcgtgatgagatcttatgttccgttccttaacaaggaaaggacatggtatatgattgtccttcttgttataaagataaagccttaattatagtacattttgaatattaggtgaaaatctccttagtattttttaagatttgactaaatttttagagtaaaccaaaacctaatttaaagagcacgttcttgggatagaagggttaatgcttcctgcaggagttgttttcaaacactaaaaatacatgaagttagacatacatatgaagttaaacactaaaaatatacgaaggtagacagactctgtggacttttcatgttaattaaatttgattgatagtgtataccaaattgctttacaaagcttataaccaaaaatataaaatggaatatttgaaatgagtaaaagatacttaaaggcagtgtgaagaatgagaccaggagtctgaactctcacctagtcatgtgacattggataagtcagccctagacttctggtttccattggcaaaataaggtaaaaggcagagaaaaagggggtaacactgggccatttctaagttcacttttgactttttcatacatgggactttgagctattccgttaatcgttagggctcattctctccaagatctgcaagtgagttctagagccagcccctggacttggctcataatcatatttcctcaagctacagattgagattcacagagacaacttactgaaatcaagttaaaaagctaagtacaaactgattgtcattgatccatagcaaacggccattatattttaattatgttgcttaaatgttcactatgatattaggaaggtggttcttatgtttctgaacactgataatcagatggatgagatttttcaatttcttttccaaaatgagtgtgataagcatatgtgaaatttatagtgtttttaataagttgaaaaaggtgaagaacaaaaggctgctcttacgtctctgtcgtgatgtttttaaacatctttttaacatgttttaaacatgttatcttttgtttttaaagtaaaacaacacccctgacatgtggattttgcaacctttttaatataaaagataaaaatatgataaatgacttgagggctatgacacaaatgatgcaatttctgattgcttagtagagctccccttactattaaaaagaaagatgcatgaacagagcacaaaagtcatgttttgttatgaaactgttacgaaatgatacattttatcattttataagatgaaaatattagacaaatcatacatctttcatattaggcatctgccacaggagacatcttacagatcctcacgtctcacctctagtcaatcttgaattaatcttactcttttgtgaatctttagagctgttatgtggcaaaatgggataaatttgagttctttatcttcacctcctttgcctctctaactttagtgtggtagacggtggtaatagaagcacaacacatgggtattctttaatacatacctagatactaaaaaactgtgtaaatatttctcatccatttccttcacctctgacccctgcctgttgtcttttcagtggcttgaggggaaggatgtaatggagatttccactgtggtaaaatttctattaaaaatatttggtaagcaagtagagtccccaagaaattccacaatggtcatgagtattgaaagatgtttctaaagatgtaagacgtttctatacattatatatatatatatatatatatatatatatatataatactatatatgatgtatacacatatatattacgtgtacactactatatatagtatatatttgtatatagttaacacataattgaacaaatttgtggagtacagtgagatgttttgatgctatatacattgtgtaatgatcaagcagggtattatagcatttccaaatctcaaacattcataatttctttgagagtgttcaaaatcctctcttctagctatttggaagtatactatacaatattgctaaccatagtcacccttctgtggaatagcacaccagaacttatttcttttaacagtgaccctgtactcattaaccatctggccccctccttccttcctcctctcttctccaccctctggtacccactattctgctctcgcctccaatgagatcagcatttttcgattctacctatgagatcgtgtggtatttggctgtctcacctcacttaacatagtgtcctctaagttgacccatgtttctggaaatgacagaatttcattcttttttgtggctgaatggtatttcattttgtatatatagcatattttctttatccatttattggttgatggacatttgggttgagtccatattttggctgttgtaaacattgctgcaataaatatggatatgcgtgcatcccttttgacaaattgatttcatttcttttgggttaacatgcagtagtggggatgctggatcatacggtagttctgtttttaattttttgaggaactttcataccatttttcatagtgtatttggtgtattaatttgccttcccacgatagtgtgtataagattgaaagatgtttttcaaccttcatttggtgagatactagtaattatttcttaacataagtattatattgacagacaagtttaggaagtgctgcactctgtagttctttatttaagatccatactcgataatcactgtattaagaactatgaaaaatccagcagtaaagaaaacctgtttgaattatagttccccggccttatttgatcatagagcctatttggggactgtgggaagagtgagtatctctccctgacagtagattacactgaacatggtgctataaaagcttttgaaatcatttgttgttaactggttatcagaccagtttaacctgttcacatcaataacatatgagcatgattcctaaaagaaacatatatgataataaattcaaaagtggagtaggtaaatctgaacactaggcatctcaacttctggtaatcttggtttgatttggaccactgacatatttgtaatatgggttaaaatggcttttagaaggatctcactcatttaacttaagcataaactgctttaggatatttttttttctgagtttcttactgaatacttctattgagtaaaatagtctagaaaatggtagtatctaaatctgggttctgagtggttgtactttgaaaagtaggcatacttcgtaagaggtagtactaatgtatctctagcaaggttttgaaagaatttgtggagctttggaccctgtcctgtggtgtgtagcattacccgtcttgtgcagtttctcagtacgtattttagggacagaccagagttctttggtccttctctaattatccagcttattctacctcctgggaagcagttgggacccatttctgtattccgattttgtaggagcatgtgcgttaattgggttacaggaaaactctggagtcactctacaggaagatttgaaatctttccttttcccttttcttaaatctttcttaatgctgaatcaggaaactatatctaaggaacaaaaatatctagttaggaaagtgtgtgcttcttaagtccttgaagagagtgtataacttttagactgtgtcagtgttagcaaattaatgtcactattttgatcgagtgaggattagggaagtacattaagaaatgtcttcagtagcaaatacaataatcttctctctagactaagttctatttattatgttatttgtcaggtccgtctctttgggcttctgcccagctgtatgcttcagcctgtttcctatctataggcattcatcatgagaagcttctgctgcttgtacagcttagctgactttagtccatccctttgcgtttttaaatgcacttgggagtccttgacacctgctgatgctgcattctagatgaacgaaatggattttatgatgcaaggaatgagtaataaagtgttacagtcagaaaagccctagaggtgaccttaaccaaccctctcccttctttgccccaaacatcataggtgatgagaggcccaggtttgtgaaatgactagttcaaggccacagagctattaagcggtagagtagggaccagaattctgattatcttatgctttacctggatgtttcctctttgccacttgttggaggcaagagctcactctttaaagatgaagcaaatggccaactgatgaatttatagagagatgtgtgatttttacagagttatccagagggcatcttctggcgagtgtgtcttctctggaaggagaatggaaggtttttgacagtaaggactgacgaatacaccaaatgaacaagaacatctgtgcttctctagaaacattataatctatggttgtgaacttgatattctagcccggtggtacccaaatttggctctacatcctcacctactcacctaagggagttttggaatctgttgtcagagagcttcccaggtgatcttgaagcccaaaaaagttttgggaatcagactgtgtctcatgaagtatggtcacaggattcccgcattggaatcacttgtaatgagtgctagaaatgcacggtcctgggctctgtccaggcctactgaatcagaatctgtagtggggacctggaaggtactcgttagctaggagttatcaatgtcttaaacattttgtagggagattgaaccagggatttccaggtttacctgaattctagatttagggcgttatggaagaggtgttaacaattgcagtacttaattctttggaaagtttaagtttaaagtgctgtcccctcttcagcattggtctctgagttaattcctagctttcagaactagacatgttgagtttacgtcactaggctgtgtccttactgatctgatatgttgctgaaagcacgtgtgcgagcatgtgtgtgtgtgtgtgtttaagtgtaattaggtttttgaaactccagggtagattgttctttcactgttatttttcctttcttctaaaagaatggctagaggagtagttggtggaagacctcaggtaatgagtatttcaccattactctcagttgtttgaggtaggtcttgtactgacctcatcagctgctaactagaaactttgacaaaaggggaaaaaacaaaaacccaaatataaattccagagggcccaaagagatgttgattacctctgagtaggtagtgccttgcatgataaggctcctgccatgccccagcacgccttagggctagtatcttaaaatattttttattaatatgagtgttcttagctaggtttatcttgtgtctctgcttgctgagaattttacagctagttttgatccttctttattctatcaaaataatgtgatttccaaactattttaatggcataggattctcttctttttaaacaaatacaatcttagctcctcaggattgaaacagataagtggtattttattagactaatgggaaccttcaaatttaatagcacttatttcctatgagtcaatcacagtgaacacaaagctgttttgatgaacattaatttgaaaatgtgattttgtatgtcagttacagttgtacagtgagcatgtgaaatgtccatagtatcaagagatcaagaattctgattcttgcagaccagtagttggaaacagcagggtggttgggtgggtggtaacttaataaacacctcactttgcaaactctgtatgctatttaattagaaagatggcagaaaggttgtgtgtgttcttaggtctgtaggaaaggagatgcctgcacaggggtaaatgctttggtccgcagtgcgttaactgtggaatacagtacatttgaatctatttttagattttagtgcccaagtttgtataaaatgaaatttgtgtcaagtttgtggaacctctatttttggaacatctctcataggcaaaaggtaatggagaaactttatttatatcttcattgatgtatctttaaacttaattattttttcattatatcttgtttctttgccaaaggtcttgcagtcatttaaaaaatttggtgttatcactgtagaagcttcatataaaattttggagaaagtctttaacatttattcttctaggttcactttttacttgaacctgggtatttttatagaacaattttcagtgtcagccttgattagtttagtggaaagattacaggatgatgaaactagaaatttgtaacattttaagaaatcaaatacagtacagttctttatcattgttaggaaaaacaacttctgtcctcttttgtttagtacctggggacttggaaattaattggagaaatggcacacaatttttgctcgtattttgtgtttgggagttgacagaaaagtattataaaactaaagtggtcagggttggggggcttatataccattttaacaaaggaaaggtttggactttgcggaataggaaatcatggggaagcgttcagaaaatatgtaggggtaagtaatgacaagataagggcacttttaacaaggttggtttatacagaactgtatgcataaacaatctgatgttgactgtctcaagtaatgagtcgcttacttctgaactggtacaggacagagagacatcttcccatgggaaatttatactctgcttttagggagaaaaggggagggcagagaactcatcttgcatctgttgacttcttattttcctcagcctaaaataatacttgatgccagccaggcgcagtgattcatgcctgtattctcagcactttggaaggccaatgcaggcgaatcaccggaggtcaggaattcaagaccaaccatggccaacatggtgaaaccctgattgtactgaaaatagaaaaattagccaggtgtggtggtgctgcacgcctgtagtcccaggtactcacaaggctgaggtgggagaattgcttgaacccaggaggcagaggttgcagtgaactgagatcgtgccaccgcacttgcatttcagcctgtgtgacagagcaagactccatttcacaaaaaaataataaaaaaaaaaaacttaatgccaaagtggaatattttggggtggcaaatctggaccttttcaccattcagtaagttaattcccagttaacatctcttcataaatcaaagagctgcttctttagataacctgtggtaatgaagcctttaatggctagttgctaggatgaagaaacatctatttgaggatacagaaaagtatgaaacaatattcttctctagtaattcattaatcatccagatttgtttctctgatttcctgatattgatatttgtgttacttggtattttgttccgttagaataaaatgtaaaaaaaaatatgtgtcactcccacgagacagttatacagttttccaacagctgtcttagagtgtttactgggaaaataaagttgaacctatggtgtaaatgaggtttaggagttttgattttgtgctttcctagcccttatcagaggaacttttagaatagccgtatttgccttaccagtagattaggaatagtcttcagtaacctttctctttgttgctttgctggtttgatagaaaacacaaactttgtaaacatattcaaattcatagaaaataaaattacagttgaaattctcttgtaacatttgcccctttataattcagcataagagtgcacgtgtgctgtttgctattccagaccttttctggggggtagtctttgtttactttgcatcaggagattggtggtatagaagagacaccattctcaatttaagcgcttgtcgtaggaatgatggaaaatgtcgatgaggaagatggtgaactaaaatggaaatagttcagttagaagataaaaaacagttaaattgtgatatgaacttgttgcttcttttttttgtagtttatagaattttaaaattctgtttctttggtatgaaaatgatttataccccccatttttatgatgtagtacaaaatcaacctgtttttgacagttttcattctatttcactaccttaactatgacacagctttggccaccagccttttattttttttttaactttgtttaaattcagaatagaccgaagcacttttattctgtacctgttggaacatttcctttcaaatattcctgaacagaatagttgattattggagctaaaactgtgcttcaaaatttgccaacttcttttgagcttgttctaagttaattgactttcagctgagtacactgctgctactgaacgttgagagctgtggaaaccctcagagtgtgcttttccagctcaaaaggtcaactttaacagatattttctggcattttatttgagtgtcattcgatttgtcaggtctatacagtggcttaattagcatgttttagtccttaaggtatttgttttggctctttaaagttttagattgtcaatatttcaaaacccttggatgccccttttattggtgttgggaaatgtaatcaaatattttttgaggggctgtaaatcacagagtagcatttgctgctctaagtgttgtgaagaaacagattcgcagttaaaaccatcttttcagactccactcagtaatttctgacaccatatatcatctttatgtaatcgaggggtgatcattctgtattgttctaaattagaacaaagatttaccttatgcctcctttaagggggaaaaaagcaagacagaagtatttaagctgtgtttataagagttccttggctgggtatggtggctcatgcctgtaattccagcactttgggaggccaaggtgggcagatcatctgacatcaggagatcaaggccagcctggccaacatggtgaaaccccgtctctactaaaaatacaaaaattagctagacttggtgctgggtgcctgtaatcccagctcttcaggaggctgagacaggagaatcgcttcaacccgggaggcggaggttgcagtgagccactgcactccagcctggatgacagagggagactctatctcaaaaaaaaaaaacaaaaaaaagtttcttaagtggtagtaggatttactaaacccctgtgtacactaggtttgttacttagtttgggatcaagttcttgggattaagtttgaatcaaaatacctttgcttcctcctgctagggaggtggcctactggacactaatcttagagcttttagcttagaacattgttacacttgggttccaattccagcctagccactcaatttgtgttgtgtcctgtgtgtgctttttgactctgaacgtcagtttctttcataaagtacctagcacacaggggtactgtctctctttcctgtcctcttcttaccagttaatacccattcagttacgtgacagttgcagacttttgtttttccccccataggtaaaaagtagtttttgaaaaacatcttgaagtagatctgttgtcctagatttcttggagtttgtcatgatgtgggaagaatgcaaagctcatcctccatcctttgctttctaggtgatttgaccaacataattgattttccaggggtggaacaaagtaagaatagaaagtgcgaggcaggacttgggagcccagaatgatgattaatgatattacaccagcctttatgaactagagcaccggagtgtggaattattgctctatgtggtattgtgtgaatgtttcagaatgaattagcttgacttaaaatatgaaggtgctctcaagtgagctttccagtgagctattactgggctttcgaaggtcagcaagttgttaaattgagccatgaagtgttggttagataaaaaccaactgcggcttatttttacttttattgacatcatcttccttaagatgagtatccgtttaatataaaacgtaaatactatactaaatagtctcattttatcaaagatgatgttcaagtctagatatacagagacctgtgttttgggatgttcaaaaacctaatattcctgcagtgttagcagatcagaaggctgaggaaaaaaattactatccctttgttttcttttccattgaaatattcattgaaaacatctaaagcatttcttcattcagtcatcacagactttacagatttcagtttgtgtgtatgggggagggggcagtttccatgagggtatggttaacttagcagatgcaattttacatcttgttttccttttttgttaaatccttatgcatattgtgaattagagtttatttttagctcttaatgagttcttttctcatcattgtactttttgttggaactctgttttgtacatactgctttgattttgtagggaaaatgtgtatggcgtacaagtcctgtatccaccacccccaactcccatcctctagctcaacaaaaatttgcatttcttggtgagggatgatagacttgaatgtttattaccttgtagagtagatctgatggggcccttgactctttacaacctgtttgacctttggacttaatactgagaaagtcaaatcctccttttaaagtaaagtttaagggaggcatagctgtgtgtaaactggtaaatcagaatcactgcactgtttttaaggtcactggagcctgagagaacagttttatttcctcacattttaatgctttaaatccagtcttaaggagttgcctccaagggtactgtcgtacgaatcctttttcctaaccctggtactaagtaagccatgggagcttttttcattccatttaaatgaattgtagtttgctcttattctactttaaatgtatctaaacattttgaatataaatgagagactgatggtgtatcaaatcaattgacatcttctcatgtagacaagggctctcctaaatgacttcccaaatcctgcctctaaaattgtcttttttttcttttgtgaacctgagtattaggttagctctttttaaacatggtgccaagtgattttgccattatatggtgcagacagtgtactaccttctcaagtagaatgatttgggatctaatatcttaaaatctaacaatgttataccaggtttgtttttagacctttatattctttattatttgttgtcctttttttatgggggtaagcccttttatacatattggaataggattaaaatacagaattgttaataccgatttgttactgtgttttatatcaatatgtgtatttttttaagtgctggaaattgggagcctttgcaaaaaggcgtgataacattccattatatccccagctctagaacatacaaagctagtcctgatgtgctacttaaaagtagaatatggaatcttctggggttagtttcttactctatttgatattttagttctctgaatgcttatagaaaacaatatatttggatatagccatcaaagtaaggaggatgtctaatattctttcctagcctttatagttggacaaccgacttttaaagagtcttttttttttttttccttttttaagacagtacccttggaggccactgtgttcatgaccagtgactccttgagactgggtttattaaatggacctgttagtatagtgaataacagctcttaggtaattttgcgtagttgttagtaggcaccaaaggaggaagggtgagcgttagttgctctatagattataatgaaagaaggtctcaggccagatcccaccacacatagaaacctctatctagcaagcaggaccaaattgatggagaagccttttctgaaagcttggtaaagtcccacttctctcttcttttttaagtgctttgaacacactttaaatgaaatgttcaccggaggctaacaatatcccaaaagtcacgttctgcaccctcactttgcatcatgcaaaaagaaatccctttaaacaaagactgaaacccaggaccatttaacatttaggggttctctccataaagatataaccaccttggattatgccaccttggattttgtgccctgtttatacgaaggtgggaagtgtctctaaactaatttagcccatctgcgtatcttgaactcctttaattttaccggtaagactccatttctaacactcgttatgctaattaacaaataactggcttacagaacaaaaccctattttgacatccattaggtgaatcagcagagaattggttgttgatttaggtatggatagtatgtaggcttacactagtcacttgaattttagcctacttgtattatactagttaactaatcctggattgtgcattaatgatagtatgttagatgaccacccagaattaaagcttaaatggctggtgcacacgtgtcatcagattaattctaaactttaaaaagaaatgtttttattgtggcaaaatataacataaaatttatcatttgaaccttttcaagtttacagttcagtgacattaagcacattcacagtattatgcagccgtcacccttatccatttccagtaccttgtcatcatcccagatagaaactctgtaaccatcaaacagtaactcctcacccacctcctttgctcccagcctttggtaaccattatattttctgtgtctctatgaatttgcctattctcggtaccccatataaatgaaatcatacaacatttgatataccaaagtattttgaaagtaaattatgataattgttttcaggagatactagtttttttccccttgggctgtagactcttctgtatgttttatttctcttcctctccaagtggctttagggaccaacccctccccctgaaataagtagcctggcttcaaatttagttcatgccacattccccgtaaaagaaacgtccaattaaaatactgaatttgtgcaatattttgccagattgctgactacttgcagggacttcatcttaccccctttgcagttgttctgcctggtttcaagcttgttttattcttttgaaaattgccttcaatctcttatcactccccatcttggcaagttacctggaggaagaggcagttggtgtatgtacagtgggtcagaaagaagaacccaagtagttttgctccctggaagtcttctctgccttcatacttttctactcttttctcccagagagtcagagcatccagggacaaacatccgtgtggggtcttgggacacaggacacagtgtgtaacatccccagactacttctcgttacctcacttcattctctgctgagcacttgaaggaaatcttggcagaaagccattcctcaatcagagtcaaagagcgacgagttagaaattctcttctgtagttttagttcttccatttttttctcccccttccctagtactctttccccctacccctcaaggaagccccccatcaatataacagtcaaccacccaaccagaaacccacaggacagagccatttcttttttttagaccactggaacaattattgtagttagagaaaacttaaatatctttctgaagcaaccttgggagacagatgctgatgtatgttgaagctagagaggcaggtggtttgtggcctatttctgcctttgcattatttcagattctgttactgagtagaacagtcctttttttaaaaaaaaaaatttaggtcttatatcttggcctcctgccttctctgttggcatcagccaaatgcatgttggctgcaagtacatcattccacacttagtgactggatttattttcagacgttggctattgacaactgaaaagcaatttcctgtgttggggcttaacagaaagcctcccctgggttgtaagagtttaaaataatgaaaaaggtgaatacttcatgggtaagtcttaatgcaagctttgtgtagacttatccccactgcctactttgcactcatgtttgtagggagtttccaaaagcctctgattgagatgaggaaggtcttctacctctggagctgtaaggtcttgtcttgattaaagatgactcaagcttaaccagttgttcagtagtctttgtctgagctttgggtgtgctggagaatatcagccagtgttaagttcttgatttcaagtggtgactatctgttgaagagagcctgtgccatgaaataccgtagtcctcccattatcaacagtttcgctttctgcagtttagttaactggtggtcaaccatagtgttaaaatattaaatggaaaatcccagaaataaacaatccataagttttgagttgctcaccattcagagtatcgtgattaaattttgcaccttcctgcccaggatgtgaatcatccctttgtccacattatccatggctggtacatgttccctgtcctttaatcacttagaatccatcttaacagatcggaaaaaaaaaagtagtgtatattatgttggattcagtactatctgtggtttcagttatccactgagggtcttgtaacccccaaggataaggtagggggtggaggactactgtactcggctagtggaacttcttgtggtatacctcatggttcaagggaggtagagaaggagggaattgtcatactgtgaaggtggaatgcaggatgttctaaattggtgcttgttttgttgagcgaagatgtaaagtatagtctggtccactaatgatttgttgattatttcatttgatgattaatattggatgcagtcatttatttgaacagaaatgagagaggcttttttttttttttttctttttcagtttacatggaaagacttctaaggatgttcgtggatccaagacaaacttgtctcagtcttacatttcattagggtccttctgtattggggaattctctttcttagtatattgctgatgggaaaaagctaattccagaagccagttgggtgggtcctctctctttctgcctgcctataggcaagcagccagtaaggaaccgaagtaaagtcaattaggcattttatctcaggacctagggtctcaaggaagtgatagaaggataagatggtgatgatgactcacctctctgttctgaggtgggggtagttttacttcctactacttaactccttgaagtctggttcccagcccttactcattctgaaaacgctctgtgactcttccagtgtgtttctttgtgccagttaggtagagtcagtgtacaatctagaactccattgaaacaatatttatgtaattcatattttttatctttttctatcagtgttttttaactcaaaagtaagcttattaattttgtattggtctcaatttgacctataagagaataacttttgtaattggttagaagcccctgttgtgaacttgccatagctattggtaaaggagtttgttttgagaggtgtgtatgtatgtatgcatattttgtaaataaagtcacttgtcagtgcctttcaaacaatttactcatatttgtgtcactttgttagtgttggaggaaagcaaagtttgaatttaattttgaaaatagtaggtgcagtgaagataccgatcctcccttccaagtcatctcttgtgacttcctcccccttttgattatgtttcttgggcactactttgctcactgagtgtgagcaagaacaacctgaagaatgactgagatgactactagcagtagcattcacacagcaggtctttttgaatgtttcatctctagcttttagtcttcagaggtcagtaatgtttggtgactcccattgcttttagtagctaaggagagccatatatcttgtggtaaaacagatttgcctctgaatgacctacatttatctcctctgcttatttagtggtcatttctttaaaacccttcagttaacgcttttggtttccagccatgtagggacatgtgtatgcttacaggcattatatttctgtgtcctatgggataagtagtatatgtcatcattaggtttttggacagtctttggttctccttgaaaagatgcaagtctagttttacctttattacagtatgttttttttcccagcaagtaagtcattgatggtgaattgtaaatgtaattgttcaaaaaagaatctagtcaattttgtgttcttctttgcaacctattaagatgtttgtgtacacctaaaggatttgaatgggatatctgttcaggtagcttaaagtgaaaagatggtggtgggtagggcaggggaatgacctggccaagataataccaggttatagaggattacacagaaaatctttgtataatatcttaatttggctgtagattatacattcagaaaacattcttctttggggattgtaaaaatctaccatagttcctggttatttgatttgtattttatttgtttcttgagacagagtcttgctctgtcactgcccaggatggagtgtggtggtgtgatcacagctcactgaagcctcaagctcccaagctcaagtgatcttcccaccttggcctcccgagtagctgggaccgcagacatgtgccattatgcccagctaatttatatattttgtagagacaggttctccctattgttgcctaggctagtcttgagctcctgagcttaagtgatcctctagcctcagactcccgaagtgctggtatttgggagccactgtgcccagcctgtttttttggtttgtttatttttaaaagaatttttttgcttcattttctttaagtcatagattacaatgacagagagtgcaaatgaaggaacaaattagctttgtagggaataatgaggtaaaagttctaattcctttactattagatttaattattatttttatttttattttttttcgtggggggtgcgcgagaaggataaagaagcccaaaatagtatagtaggcttttgtgttaagatccatttcagtatatatatgttttcttgctagaaagtactcttgcttgagaaaaataaagtttataatctaagtagaatccttaataatgttgttgtcaattaggataactagaataagcacttaataggttatattctgtttgggtacaggggctagtaagtgatctggcagcatacatatttaaagtttttccctgaataaaggagattagtttataatatataactattttattccttcattagaaatagcaaggaagaagttttcagcaacaaaataggaaaactcttgacagcataggctattgaatattggaatgagctgaaaaactagttatttagtaaaacattcatttcttcgagtggtgtacttgtaattgaatacctaacattttgtatggcattctacattgtaggaacctcttttaacaagagtgattctaacacgcacatcagaatctatttgtgaaacttaaggtggagacggtagaataataggtgggggcgagcatcagagagtacctttagtgcagtttcttttcccacagatgagatcccaatctcaaattcataaggctagttagctggaggccttaaactcctaacctccttttccttagggacccctttgtggtaataagatggattcttctgatccacttgtcttagccttaatcttaaaatgtgtttgccaagtgggcatgtaatgtggcccaaatagctgagaagctctgtgtatagaagttacttcccacatcagatggtccttcatattgtttaatctggtgccataaaaacatagcgccttccccaagagtgggtctcagatatgtgaataagcaaatcgagtaacttcagtaacagcagattaaagaaagcatcacattaaaagtttttaagatgaattaaaattgaagtgttgttgaataagatttttttaaccctctaccctgttatacatttaagagtacttgagatgataaaatggtaatatgttaaaattatttttgtgtttaggtttttcctgtttcataggaaagatgtatccagaattagcagcaatggtcagatgtactaagttgctgggtatatgatttagatagttcgtttttaatatactttaaagtcagtggtaaaagacaagacaagcctctgtgctctgctcattcggcaggataatgcgtttactataccatgctgatgtggatggtgccacattacttttgtgtcctaatgaatcattgtctaggtaaaatctttttagcaaaatcttcctcaaagtatccgtgaattttaaatttggaggattacaatagctaatcacttcaacttttggatgaggtgatttgggaagaaatgcttatgtgtgatatgaatgtggcaatcgtaggagctgcaccattttgtacttctctttaggagccatctgttaattggtgggcaagttgccctgtggtctcagagtggtttgctgtagcataagctgtccctgagcaaaaacaaaattgatacttatttctgtcagggtacctctttgacttaattcgtagccatggttcccatctgacccatagcctttacaactttaaccatgtacagctcaatttcctctggggactgtgttccattaggattccttttctttattgcttcagagagagtgttcttgaggtttgtgaagctgtgtctgtagtatttaaataggtttcctttatactcagcatttatacccccaccagcccaaacaccatcagtctgttttggaatttttcctaagcatgtaaaaatcagtgctgtgtgtgactattttaagtttagaggaaagaaagaaaaatgttcctcctaccctcttgattaactacctcttcattaactataaactaagcaacatgtcctgtacaaagagaagaaaaatcacgttatttatacttaatccttgtgtcgactggggacgatggatgtcctttggaaatttacttcctttgtctttgctaaaaatacctctaatttaattctgctctttctttttgatataaaaggaattacagtggacattttccccccggtggaagggttgtacatatcggtctatagcagatactttatgatcagtacacataaggtgaaatatgtttttattgaaacacctgatcatgtgatgctacccaatattcccttcttaatgactcagcttccctccctctttcactccttttctaacttgatgtcaccatattgtatgcgaaggacgtaggtatgtacgtgcattcacaaggcatgtgtaattttgcagttgctactcagttaaaaataaggtttgttaacacttgcagtagtttttccccatttactgtggtagttattaacactttgtccactcataatagcagcttagcttggtcggggggcagaaagaggattgaggtacttgtttttacagctgtgtaaaggaggtctcagaaaatcgtaagccctgcctgatgtagtcatcttgggtttagctttttggtggttggggttatccatatctgttagttttatagtgcaagaatactttcaaggctgctgagttggtgtgttgtattaatctctgggtattatggttcaaaaggtttcttgcccttctagttacttttacaaaggccagatgttatcatgccaaggaagtatggatcctcctgggacacaaggagttgctaggtttcaatcctggccattactctagaaggcctgtaaccgaacccaggttcagctgcttgccacttgaaagccagaacacaagaggcaaggattggtggaaggaaaagcatttttaatgggagccagcaaactgagaagatggcaaactagtattctaaagtaccatcttcaatttaaaaatttaccgtagggctttcaaagggagaaaggtatgggagacgtgcagaagtggtgcaggatctgtgtgtcttgttcagtggctctctcaggtgtctgaaggtctaattggcattatcttgactttggctcagtggtggtggactaattatgactcctcctaatcatgaggattttgcagtggggactctgttcctggtttgtttcaagattagcctgtgggatttcttaagagcctaagtagataagcatggattgcctaagggggagtgtctagagagggaaggaataaagagttgagaggggagggaagaaggaaaaagaatgtgggtgatttctaacactgaggtccctagttacagtcttacatctgcatctgctcagtgattataccaggccactaaggtgactgtccctgggtccctcgtgtgcctccttcctcattgtgcctcagtgcttggtgcatcctgcagtttgtctgctgtaaaccttcacatgggccagtcttgcttccaggatggggggtcggtttgcttacctctacatagatgccttacgttcagcgtagtatgcttttggcttgtctcttttatacttctttacaaatttgttcttctccatgttcctattaccttaatttgtttgtcaggaaacacctgaatttcatcttctctgttttgacacactaaagagttctctttttcaagctggtcctggttaagcctgaaacccctgcgatgcactttatgtatttcatgttcattaggtgcctgcctctctcccttgtttactgtgttgggcttaaaatttttgagcatcttagagggcagagaccatgtggtattgatcccatcttttttccaccgctatgaaatcatgctatgcgtatgaaagcattttaatgcaatctgctgaggagagaaatgtcttgtgtgggagaccgttttggcagtagcaaacatgtttggctttagccataagaactgctttctgagtttggaaaaggagtatgtaaatcaaggcccattttaaatggatctttttataatgcagatacctctcatctatctgttttgtaatgatggatttgttttgggggcttaagtggtttgtgcacctgcaggatgtccttccttttctttcaggtcattttttttttctttcacagatagtctcttggatggattaagaaagaattgaagttccagtcaatttatttcatgacaaagaacaagaaaaaaatacagatgtatttggagattataaatattctgattggaaagttgtaaactttggatgtacaggattctgccacaaagtaatttccacacatggccaaaggcaacaaagtgtacacttttgaaagactgagtttggaaaggttgattgtagtgtggaaacccagctgctcctttgagctagctgtgtgtgcccttgagcaagttaacttagcatctttgaacctccaactttatgtccgtaaaatggatacagatgtatttacatgtagccttgtggtgagaattaaatgtgatatatgtaatgtaaggatcctagttgcttgatagtttagtttggcagtggtactgttgactgccagacaggtagacatagcatttatggggcagtgggaatgggcatttttccccgacaaacttactgactttttcatactggaaggtttgggctggtgaacctttaacttttgggtgagagagtgatttttttaagaaaaatgtgttcaagaagaaaatttatctctaacgagaatttttgtttcacatggacttggtgagtttgttagagtctaattttatagtttcccgttcttgacaaaatggggagctctccatggtggtttgcagggactctgacatagtttcaaaacagggcctttaaattttttgtttcttgtggtatctgagttaaaaaatgatcttgtacagagagaaaaacacattaagaagctgtactgcctcttgatggactgcttagtggggggggtgtgtgcgtgtgtgtgtgcaggcacaggcaagagtgcatgtttgtgaagattgacaccagctggtcgcacaagcaacagcagatgccctttttactaccacagttaacttgttttcctctaccacaatactattttgaggaatacagttcctttgccttcatcccaaatggtatatacttgaacaactccattgtcttttaagtaaacacaagataaagcagataatataagactgttagtgtgtgtgggggtgggggtaggggcggtggcggatctgtgtagaattaattataattaacaaagaaatgagaaagttgcagcatacccaaacttttaattgaaagtagattggactagcctagagttgttaatgtcctaaccttttttgaactgtcaggtatgactttcaggctatctgaaggtattttggcatttgctctatcctggtggcagctccagtgtttgaaggtagaactgctgacctatcttttagagaaactacctatatcaataagtgatgagaaccgccttttgttttcattgtggtgctgagcgtgctacactatctcagtcactttctttcaggctggagaacctgctttcaggtagagtctcatcctgtgggttgtagaggaattggtttattttcctactgcacataatttttcctcatcttcctggtcttggatgcaagatatctttgtgtacccggtgtaatacttggcaagaatttagaggatttaatatgctgtttctgttctcaatagtctaagactgaattgtgtgggagttcttaatgttgtctgaaaatgtgatatattaagaacccacagtatcgccaacttaagcagggtcatgccgcattgccagcagattcataattactgggactggaactacaggaaaatagagctctaccagttgcataatcaggggacagatatggctattaaaattaacttgaagggaaaatgccctggtgctgaggacattaaccgctctctctagaattaggctgcttaggtaatatttaccaaatgatcactatttgcagctttgatttcagcagaaaagttatgatttaaactcttctatattagctccatgtatctgtaaagtcctgaaaatcatttagaaactttggggcctttcagtctcaggccagctttattagcatttgggtggaagatgagtcttcgagtgagccatctgttttctgtattatgtgagtgtctaggttgaattctccatccttgtgataatccctggtaatgtcctttgggaagctggtagaaatgctcccagctttcaggaaggctggcttggactgtagtaataggatctcaccagtcctccatctcacaatttgatcttactcattagagggcagcaaggaagacctgtttaacttgacccaccttaacttgagtgtttaaatggaaggacctgctgcatctggcatgctacatttgaggagactcaggctaagtcaggaattaaaatgtggagaaaaacgtgactagagagaactgcatccctctgttctgagattctctcaaaccttttccctgctgtgtggtacccacatggttttgctgtgtatcagatctgagatattaagaaagctcctttgtgaagtctggtgcgagttgctttggtgccagagttgcagtgcacagcctgtctgaacagacacacataatgagtgcccaagaacagtaagtccatctgaagaaatacaatatatttgcaactttattgtgtgtttgagggaggagagagaaggatatcaggaggcaacagggtctttattaaaaacaaaaattttccccctttgtaacgaaaacttgtttttgcaaatcaaattatttgcttttttttgtcgttaaggcatgcagttttatgcaagagctgtgtttggaggaagcgaatctttccattttgctttttaaaagtattgcattattaaacctcaggttgcaagtcttccttgacagagaggaccatagtgacctgtttctgctaactgtgggatttgaaaaagattcacacggcttttattcttttatttcataaaacattactactgctgtaaatggaataatagatacacatcaatcagtgtagtcaccttttattgttgattcaactaccaggtttttacagaattcactctgcattttatttgacttgtacatgaatagtctcagcagacttaattatattagtgaaaaagtactttcaacaaaattatatgctgacatcccataattcatacttaccgatttgtggaacagatttatgtaaatctatcaaattactctagatgacactttttaaaattgagacttcatcttaatggtatttgtagaaatttttcgtatgaacaattttacatgaactatttaaaactcgtatgacttccttatgaaggcagaattactataacatttgagcatcttgtctccacaagttctgtaatccatctggtacaatgtagggacatgaggaacatgtttataagggcaactatctcctaaaccacaggctcctggaaggcagatgctgctgctccttcatttgtttttcattcagtcattcacagaacatttaatgatctgtcttagggtcctccttgtcaggaactatgttctgcttgcccttgctcagcagtctagtgagggagcatccaaatggaccccagacacacataacaaagataacggaataatataagaagctgtgggaggactagtcgagaggagcactgtacagtgtgcactggggatggggcaagttgtggcaggaaatgacttcagagagtctcaaaggatacatgggagttggctagttggagagctgtgttccagacatagagaactgaatgtgcattttagggtgtatgtgtaggagtggtggaagatgagcgaaagtagaagtgagcaaaaaactgacagtgactggaaaccagagtatggttagatttgtgttttaagaagttctcctggacaatgttttggtggaggatggctgtttttggaattaatgggagtctaagcaagggttgtcctattaggatagtgtagcagtgatcagggccagaataatagagaaccaatgctaacgcagacaaggggccgatttagctggtaggactggcagaacgtgatgcgggtgtggcagaacttgggctggaacgtcttgcacatcttcactgttagacttgagctagttccttcaagccttggatcaaatgttacctgcaggatagggcctgctctgaccattgtatttaaacagcagccctgctcctcccctccccactctcaccccctgctgcttttttctccttagctctattttcttctaataccctgtagaatgtgttcctgtcagaatataggctttatgagaaagatctttttctcctttgcattctcagggcctagcaaaaagtatggttcccatagttattgaatgcttattgtagatgagaaatttgtagggtgactcttgttttttttaaatacaaaattttattttccacaattttgtgttgagggtcttgtttaatattggttggataaagttaatatacttttaatcaagaattacggtagtctattgcctttacctttaaacctgagggggtaatttgatttttctgaacattggtagtaatttaaaatagaaaagactaaataaagtgacctttgaatgaaacaggtccagaagattgtgtatttttaattaaaatatgcaattaaaattttaattaaagtagggtattaaaacaatgtaggtggcaggggaatagctgatgctttgaattacacacagtttgggatatattgccatttactgtgtttcccacagtagcactttgccactaaacgggttttcttcctgttttcctctccttgctctgaaccctgctctcagtagtctttggtcctcttcagaggcctctggagtgcttcagctctgtggtatagatcccttgaccatgatttctaccagcctgtttataactgttctccttgtgccaggagcatcttataaactatttttctatctcttgtgaagaagggtttcttttctgtggcatgtatgttagattaaaaagaagcgtgtgaaacaaaaggcattcatacacaacagtgcagcacagaatatagcaaaagttctagaatccctaccaaggccattaaaatcaagactttgggccaggtacggtggctcacgcctgtaatcccagcacattgggaggccgaggttggcagatcagttgaagccaggagttcgagacgagcctggccaacgtggcgaaaccctgtctctactaaaaatacaaaaaaatagctgggtgtggtgatgcatgcctgtaatcccagctacttgggaggccgaggcagaagaatcgcttgaacccaggaggtggaagttgcagtgatccgagatcatgcccactgccatccagtctggatgacagagagagagaccttgcctcaaaagaagaaagatttgaattaatggtttgaaaattcttagaacacttgaccacaggtagcttctgagaatagctgaggacacgtggcattgagcctcttctttatgttcagttaccaggcaaattttttggcatttaaagacaggatttgtttttcttaagggcttttttttttttttttttttttgaagagccttagctttgtcattttggtcagaatagtcttttaatcatgttggtttccataatggttgtacttggaatttaaatgtagcatcttataaaactttttaaagaaatgaatcttagtttggggattatcagtatgcacattctaaattcagagagacttgaaaaataggtttggatgatatggataagtgatgcttggtttccaagccttccctggtggcccatgacttactgatgaatataagagtgggaaaagggtaggtaaggtatttttttttgtaatgtttgccttgctactgcttttatttttaaaaactgtgcgctcatttataaccagaaatatgcactatataaataagatgcaatttatgttgtcataggaatgaataatggcactgaaattcatatatagtaaaactgatatttgtataaaaggtacagaagtttgagatagtaatcaaacaagaaacaacttttgcggtaaaagtgtcagaagtgcctggttacgttattattcaggcatattgggtacaaactagtcctgggcttatacgcaatatgcctgaagtctcagttcaggtataaactagttaattgagtggagtagagcacagcatttgaaagtcctgacttcaagtcctgactttactacttcctccctgcctttttgatttgtagaagctgattgaccttgccaagcttcagatatcatttctaccttgtaggagaagaatctgaataccgaacatggttaatgtgaagctacaattaaacagtaaaataccatgtagctaaaaggtgagaatggggaggcttgcctatcttggtatcatctgcgatacccttaatatattttaaacatccaataaatatttgctgccttgattacagattttcactgtaaagtaatttaaagttctaactcccttttgttatagtgccaacattttaatacaccaacgatggtaggtttaggaatgcagtcttttaatagcagataccccctttcatattattttgcttaggataataatacagttttgcttacctcaaacatattctgagtggcactcatcctaataatgccactaggcaaataagaaactttttataaggaggcaaagtcatttggggataaatatttctgagtataaacaacttaacagatactagatatttgactgactcatatagctagtcatgattgcttgagggtatcaggtggaagacccatttgatggttgggtcttctgatggtggtggtggtgatgatgataatacctttggttaaataaataacatcagtaagtttctcattctttcatttagcaaatgtttccatgcctacagcaggccaggagtaagggttcagaggctagagtgggaaacagctaagtaggaagtaagcaagacaggcttttcactttacccagtgatctaatctcagtaggcactcttgtgccccagacagacctcaggaggccaataaactgtttctttgaagtaataggtaacttgaagattttagaagaaacacatccacgtgtacctaaacaacgcaactgtgaaacagcagtcatccctgcaagacctttcttagacctcccagcagccccgttgtagccccagctaggttctcaccattaaggcagtgatttctcaacatggtggttgtacgttagtcaccaggattttaaaaagctagtaccaatgcttcatgacagactgataaaatcagagcccaaggtgggaggatcgcttgagtccaggagttcaagcatggcccagccaatgtagtgagatctcatctcatatacacatatatatacagacacaaacacacatatatgtatatatatctatctatttttgggagtgggattcggggatcaggtttttttgttttagttttttttttttttttttttaagctttctagatgattccattgcagtcaagtttgagaatgcactggggttagaagctgctaagatgcaggaggaggagtatctatcagtagaaagggcaattagactatgctgatacaattgatttattttgtcatagaatagcagtatgacaaaactatatttgtattgcccttttctttttctgttatatttgtggcatcttttttatttaactttaaatgaatatacttaaaatctttaacagtaagcatgttattttttctcatgttatcttttgactagggtgacgatccactttcatcattaatgaatattttcatcattgcatcagcaacctagttgggtaggaattaccctcgttttagagagaaggaagctgtggttcagagaggtgttaagtagcttcttcaaagttgcacagctaactgcgagccagtaaacaccagagccggtattggaagtctgtctttccactataagctggttccttgagcagattttgttacattcagccaatttaattctactcaatgacaataggactgcaacacattttcataagaggggcacaatgcatgcagtaaacacatgataaattttgaattaatgaaaagctgaatgcttaaaaatacagtggcttcacatgtgactctttactagaactgtaaaataaaacctcaattttagtgtgagatgaaattaacctaattagtttttagctctctattcctttctcttttttaaaaaggaaaaaatacattttgatagaggacatatattagcagtgattatccaaaattctgattgtggtcccaaaatataatcactagttttcatattgggcactgtatttagccttctcttttcgtttatgtggtaacaagtatccacattaggataaccagagtagctgtcctaaaaacccacattgataaccacttgcacctacggtgccccatcaagaaaaaatacctcacttccctatgaaattgatgcaacagtagtacttagtaaaagcatttggaccgtccttgggcatctgggaaatcgtcatttagtttgttttgacttttgtagactaaacttttcttatgccatttttgtcacctgtataatgtttcttgccatttcaaactgagaaacttgcttcattgaaaagttgagaccattgcgattgactttagttatctttgcattccattgatgcaactgcttttgatagctcccattgtctgagatctagggcatcagtgaaccaggagtgagagggctcagatgatggggggagggtggcaacaacaacaaacacttagaagcaacttttactctagactttttttctcctgttatttttgtttatgttctatttcttcttttcctatatgttctcattgtcttcgatgtcatccctgtaagtcttttgtgttcatattttcctgttcttatctagatcataaaaccctctttggagaaatttgattgagtgtatttgaagataagccctcgtctaaaataaacttacatgtatacacgcccacacatggttcttcaccattttagactccgacattattgtacttggtaaaggctgtaaatatgtcagggtacaatttactgaacactgacagattcttggttgatgttttctgtcttcccatttttatggctaaggaaactgaagctgaaaaggtattgcctgttaagccctctccatagcacagagatttatccccacagtttatgaagctgaccttgtcttgctgatggtatactctggctcaggcagggcatggatggtggaatcctttggggatgtgtgttttttttttcctaaacagcactgcttgctgagttctaaaacttgaaaatgtagtagacaggagacataaagacttcatgtatatttctagaattaatagagagttctttttactgttgaggaaatatgctggtgttaatgacaagaccatatgccaaaatgaaagttgatctctgtcaacagtatagaactaatatatgcctttttccccttccttttcattagggcagagcttgggatatggctttgtgaactacattgaccccaaggatgcagagaaagctatcaacaccctgaatggattgagacttcaaaccaaaacaataaaagtatgtttaaagtttttttatatctacttgcggaacagaagtttaaaaaaattaagatttatttgtagttctttcctttcttccttttgggcccatataaacagttacctgggagacatagtggaattttggtgtttgttgcaagatggaagaaactgtttgttcaagagtgctatgtataatcattcatgtattcgcaaagggggacagagcaacaactctcttagataaatgttataggaagcctacggaatcaagtctccttcccttatagagaattacttgccctataacttgaggaaaccttagacttgttcttctataaaggtctgggtagtgaataagcaaggttttgcgagccatgcattctgtcacaacttctcaactctgcctttatagtatgaaagcagtaatagccactacataaacaaatggatgcagccgttatccaataaaactttatttatgggcaatgaaacttgagattgataagatgtttatgtattacaaatattctttttattttttccaagttaaaaaatgtaaaaaccattcttagctcatgggttatacaaaaaggtggcaggctagatgtggtgcttgagttgtagtttgctctagctaattcagcttgctttgaggagactgactggaggaacttacatgacacaggttattttttctgttgtttttctttttaagacgtacagtaaataactttgtacatactttgctgcttttgtgaatctttgaaattcttatgtagattctctcttgagagggagctaatctaattcacagcatagatttcttttcctttagaaaataagagggatgtcgcctctagcaaattcttagttagctgtaccatcaatttgccctctcctgcccagtgcagacattgccagtcaatcatggcactctttctcacagagtctagatgtggctcccaaatatttttttcaaaatagtgttccagacaacttctatcaattgactcaagtttgaacctattttccacccttgatttaagaaaccagctcagactatagcctgaagtggtgaggggaagttagtcaaggctgactgtagtatccatcttgcttagagacttgtgtaaaagatttttgctcatttttttcaggtatctggaaatgaagtgtcatttttgtgctttaaagttgcttttcttagaattaccagttaaggtaatttattagccctgagtggttttgacacatcagtctttatatttattggcccctaagaaaaatgcctaccagccctgtcttatctaaatgaaagtatatagtggttcccaaatatactagggtatgagaggtccaagaattctagatacttcttgagcaagaaactgttctatgaaaagattcaccaatcagaaaatgaactggttactgtatattatcaaaatgtgaagggaaaaatgagatttccaataaagtgtaagacaagtttgttttgaagttttttcagtctgaactgctttttctcgttatctatatttctctgcatactttctgtgtcagagatcttttactagtactattaagtaacaataagtgttctttaaaaagacatccagtgttcagttaccgtcagaccgtgtgctctttatcctacagcagatttttgcaggtttcttttcctcaatttatattgatttccagcgttgctgctaatttgtcctagttctgaggtcctgagttgtagttgggaagaaaatggaataggtggtcaagatggcttgtcccaggaactatccttttctccattctagaaataattacccttatttcattctagtgtgtcagttcttttactaaatatcctcctgcacgaagatccagaagaaggtagaaatgcaggggaaataggtctccacaggagaaatgcccgtggccttcaaggagacagttgatgacagctgcttccttactgttcatcatccagccttttctctttagggcccagctgcttgggctgaatatgtggaagaaagcacagtaaagctttgtggcttttatagtgtgacatcagtttctgattttctgagatgtgggtcagggtaatgggcaaatagcaggctacttctttccccgcccctatttggttcctgtaaaaatatcagataacagctggcatcctcaatttggcccacatttgtctaagaggcaaagcagaccatttcccacaagtatatagcaccccctttgcctgtttgttctttctatgtttatttatttccattagacagattttgtgctcttgtacaatgtcttgagccaagtttttctagtattcacagctttaagaatgttttggaagttgagttaagccttttcatttcctgggatactattccataaataagtctttttccctcgtctaatctatactggtgatggctgtcagcaccactttcttacacagcaccactttactcgtgttactgctcactcaaaactctatggtgtttctctactgttttaccctatcatctggctgagttttgtaggagtgatgccagtgctattgccgttctccactgttctcagactggctctttacttgtaagctgatcttactcccagcgtccatgcatatgcttttgcagctccgactttttggtataccctttcacactcatttttagtttaactgtggtcatctcatcttttggagcccatctaaaggaattctgtccttcagcttctgtctttccaggttgttccagctcctgctgagttcccccttctttgaagtctatattctttacccttgaggaaccacagaagttttggaattacagggggacccatatgtaatttaggcttaagcactgttctctgtccacagtcagagtggagatcttggtggtcatacaatttagccaagaccaggcagttgaagaaagagctgcgactcaaacaaatccttgtcttctgatcgttatatcaaaactcttgccaggacactgtgctttactttagaaaggtacagaagattggttagtatatacaagtgtttgtgtactccaagccacttagaatttagtatgcgtttggtaaaattcctggttattgggatattgaaacagtgaatcaagtgctgtgaaaggtggtttatttagcagtttctcctggggctccctaatagcattagctcagtagggttttgtgcagtagttggtgtgttgaataaatgacagatgttcagtatttccatggtgagtgaagtgaagtccctcaggctctgtgaggacctaaagcatttgttcccaaactttggttgcatcttaatcccaagggaagtgagttagaaatacagtttctgggatccactgtagatctactcgatccctgtccacaggtgagattccgggttctgttttttattatgcttcccaggtaatttatgacagcaagtctaaggcctagcacttgggaatcaccaaagatgtttggcggttctctatacatttggtatttgtgtgtgcaaagttcagtttgtcttctagcattttaccttaataatcctcctgtggttcattctgttaaccaaaatattaatgttttttcttgagcatgctgggcgatcgtttagcattcagagttcaacgtggttcaccagcctttgcttataaaaaaacatggcatagaaattgaaaacaacgtatccgtctcatagtaggatgctgtttgtttaagtagtagcagatacaggttaaagtctcgagatacttgttctcttgtcctaaaggtcactttttttcctggttaaggagatgtcccagaggcataccttgctggtgaaatgacatcatccctgtactgcatgggaggcagctattttgggttaagcctgcccacagtatccccagtgttgacatggtaaataaagtgtgaatattacaaaaatgttttaagattctcattagggaaggtagtgaaaaggtgatatgtgtacagttcaggcatgggggtgggggagtaatctcatggaagtgtcaagttgtatgctggtgagaagaagagaggtttttctccaagatccttcaggtccctaaaggaatccttcctttggtgagttattcatgaagtgagtggttgtacctactgtgttcagtcttcatgctctttctctactcattttatttttaggggtcttaagtattgaaaaaaatctaaatagtatgtggaatttttatggcttaaagccaatacttttgacttgttaggttaacggtgtcagacttaaattcatgattagcaaactaaaatcattcatttccaagaatatgtgtagacttcctccctcctctgcataaagaactctggataggtgattatcacataacacagtgaaatggatacgtggctcccagcatctctctcagcttttccctcctctacaatgggcctaggatgctttatttttcctgaaactggggttgatttatctggaaagaaggagctgttatctcagggaatagaatgttttattgttattggtaagttctgacccctcaaaagaaaaaaaaattctgtctagaattaactaccatagaaaaactgcccgattttggtcttgattaaagaaatattttccttaatcattcagctatagaccaggctaaaatttacctttgcttaaaagttattaattggtatacaatcatgaaatttcttatactgaaataaaccacaatttaaccacatttggaaaattctattataaaaatgagaatattataaatagtattatctattttttgtgttcagtttgagagtgattaagcaaacctaagaaacctgtattctaagctactgtattgccattaactttttgttaaatttatacctttttctaactagcttatcctttcctcattagtaaatgtcatatttatacttttttttttctgttcagcatgagcttgacccttaacataggaatatagaactacatttctgaaatcttactctgtctaaattaccttactaaaatgatttaactggctgcttaatcttggacagagcttttaaccatttaagtttccatttatatcgttaaaataatttattcatcactgagaattttcattacctgctttttaggagaggaaaaaaagcttttcatggtctgctaagttgcatattaagaagaaatgttaactttttaaacaaattcatgtttattgcataaaatgatttgatgaaaagcagcaacctctcctgaagagagaataattttaattgccttcttctttcactcttgaaagtttaatagccactgtaaactttatatggggatttgagtttctagttcaacctccttttgcgacaggattattgggattgggttggtttttctttatcattataatttgttcttttataaaagttaatactggccagaaggaaatattcttagatgctgtgtaaaccagcccccactgtaaagaaggaggagagggtatgcaggcaggaatgtaagtaaagctacgctaagggcttgccacccagtaggtcctctgttaaaatacctgcagcacagttctgtgtttgagacgagtggctctaatagccataatgctgggagttcatgactttagtagggcatggaagttttaaagcagttgataggggaaccatatagaatttgcccaaaaagaaagcaggatgggaggcgagtgtgggaaatgagcctgagttatgcgttatagaagtgtgttctgggtgcgcatgtatgtgaaggcgctgatgttttgttctctaggtgaggaaggggcaccggagggttttgagcagagtagtgttgggattgaccttgagtatttattcggagaggaaggtttgttttgtatttaaacgccacatctttctcagcatttcaacccaccctagtgtttttctggcttgtgtagtttgttatttcctgtgatggtacaaagggtagaaatttataaatcatcgtctacaaagtacccctcatttaaaaacctttaccttgagcagatttggaaaatttagaagtaatagttggaagaacaggcctcaaattaactgacctacttatgagatgttagcctaataactgttatttatccagacaaaaaaaaaattgaaaaagtatatgccaggagtggggtaaaaattgaaacgttgagtaaattgttcctctgccttttaaaaaatacagactgaccgagtacatgttttcctaacttcattgttaacttgtattctggaaatttccacagccacatgtcaagttttaacagtatattacatctgattatcagcacctcttcaatacatttaaataaaatgcttctatctatattgagtcccatttacatagtccttatttgaagtgagaagtcacagatgacttttttattgttatttgtataatgcccttcatgtgttttgtagaatgctctcttgtcagctttaatttttttcatacaaaaagcccagaaatttcttactcatctaatgtggtctagggaatgtgggtaggtagcccaagtaaggtccagagcttgaatgtcctgtcctcaggcctcttgtgttacttctgttccttttagccaactccaaagtgagagttaattggaattctcaaagggcaataactaggcattcttccaatggatttcctttagattttcccacagttgttggagcagaggcagagcctacaaagagggaaggatgaggaacagggttcttggtggcagtgtcccctgcttacttcctgcctgtcagccacacagtggatagtgatggtagaagagtctcaacagttgactgaacccatctttattggaattgatggatcattttgttacctaagcatgctccacaggggtatcatagcccctcatggcagatgcttaaacttatgcaaagtatggtctataccagaaaagcctggctatttcatttctgtaaccagcatggagaatgtcaggttttatgaagacctccattggaattaaaagagtaactgcctccttgtgagtattgcttctgtgtaaggtagggggaaagccacagaagtaataacttgtataggtttaggaaattgttgctgtctgtgtgtgtgtgcacgtgtggaattgaaaggggcatgtggtggggatgtcactgaaacttactgtttatcagaaatttgagtcattgaggcagaacccttataacagctcacaggtctagaatgttaagtgcaaaattggtttccgaataataaggaggctgaggaagagattcaagcaaaccatgtgcatcatgatagaagacactgaaaatagttgaaccacatgttcaaatgtgtttgagtttgatttctaagataacagtgagataagataaattgaaactgatctggatggccaaaaatcttttcttttttttttttaaattattattatgctttaagttttagggtacatgtgcacaatgtgcaggtcagttacatatgtatacatgtgccatgctggtgtgctgcacccattaactggtcattcagcattaggtatatctcctaatgctatccctcccccgtccccacaacagtccccagggtgtgttgttccccttcctgtgtccgtgtgttctcattgttcagttcccacctatgagtgagaacatgcggtgtttggttttttgtccttgtgatagtttactgagaatgatgatttccaatttcatccatgtccctacaaaggacatgaactcatcattttttatggctgcatagtattccatggtgtatatgtgccacattttcttaatccagtctatcattgttggacatttgggttggttccaagtctttgctattgtgaatagtgccgcaataaacataccagaatctacaatgaactcaaacaaatttacaagaagaaaacaaaaatcttttctacctgaacatcacactgctagcccttgttgctttactttcccactgctttttaaggggaatgtggggtagggcttttctttcatttatcacctgggaaaagtggtgctaggtcagtttggcttcagtctgttctaccacatacctggcttgtgaagttaagatgattgtgttaacttcccaagaaggagcccttaattctttgctgtatctacttttgaaacaaatttcataggactattatctttttactgttttgaattcgtgcagaaacagcagtagtctctggtaggcaaatacttaatccctttgatgaaataaagctcaggttaaaagggatttgttcaaaatgaaatagctaataagcatcagaaatgtagtatcatgttggctgatgccgtagcttttgttgttttctttccatttcacattattgatgttaaactgttttgctcagataccactctaaataaaccaactgagagaggccttaatttgggcctttgagtgtctttgtcttgagactttctggaccagatctcttaggtgccagagagtggagctgtctttgcctaggagtgaagaactctgccctcttgcagagtgagcacttgagacagccagacgtacagtttgagaagaaaactggatctggaggtcatacttagcagataagtgagttaacattgattcagttatttcagttgtaatgaacagtaaaattattattatagtagtaacttcctgaaattattttttaggcagctacgcattcttcgacaatgtttttctggtaggttcatgtaagtcgtagatgctagtttaaaggttacagaaatgtgtgttaacattggtctctctttaaatttgcgtagactctttaaaaaagcatggatggtgcattttcttttattttcacttctgccagccattcctatcattacttttagtgtgtgttctcacccgcatgtactacattagaattacttgtggatgttttatttccagaagtgtcttgacatgctgcttttgggcaatgagacagagctgtacagttgacacttgaacaattcgggggctatgggtagcagcccccatatagcaaaaagtctaacttttgactcctcaaaaacctaactactagtagcctactgttgactagaagccttactgataacataaacagttatcacatattttgtatgtttcatgtattacatattatgtgttattacaatataaagtaagctagagaaaagaaaaggttaagagaatcataaggtagacaaaatatatttactgttcattaagtggaagtggatcatcataaggatccttcccccttgtcatgtttatgttgagtaggcagaggaagagggaggggttggtctaaaatcccatattaagtgcacccacacagttcaaacccatattgttccagggtcatctgtattttgaaaacagtacaggaaatctgatacttgcctgcctcctctcccttccttctgtgaagaactcttttgaatgctgcaataggacttggaggtgtaagtcttggatttgtgttctgttcttggtttcttgatcaggtgagctgcaggaagggcaaggaaaaatagaacaagcatgtagtcctggaaactaataattcagaaaaagtttcagtttctagttttggttgttaaatgtctatctagaaatttgtgcttgccttaaccaaataaccatattcatattcctagaaagagtcattgagagaagctcctaacttcatagaatcaagcacagtggcctttttgaagaggctaacccagttgagccacatccagccagtctgggttcttggaaatatctttggatgagtttcatgggctttattttacgtctgcatttttgcatttgcaggtgatttatattcctaaatgctttgctttcaagctacctggcatttgtcttatttagaaactaacgatcagtggatggcaaggaaaagggcttggcatccgtggttgttatgagctatctaggaaaccctaatatgcccagagttttgaggattatttcaaatgcctttacacaagagactgccaacactttaatgtttatcaggatcagtggggtggggtggttgattaaaaatgcagatttctacaccccaaccaggacttactgaatctgattatctgcagagagagactgaggaaattgtatttcaacagccaccacaggtgattcttaagcagatggtggcctgacctgagaaacactgttcagtggatcttaatgatttgcacttgggtaggtctgtggtgcttattttaaaacaagtctacttgtaaaaaataggattttcatttgtcggttacatcaagatatgttgaatggctgggattcactaatgtgtttttaatgaatatagtgctggatttacctaagaaataatgcacgttctcacacacaatttgctcattttcagggcatttgtgtgtcattaaatacttcaacattacaattatgctagcataatttatttgcttctgagggtatttggagaattttcagtatggttctttagggcctatccatttaaaatgggtgtcttggccaggcacagtggttcatacctgtaatcccagcactttgggaggctgaggtgggcagtcagaagttcaggatcagcttggccgccaacatgacgaaaccccatctctactaaaaagtacaaaaattagccgaacatggtggcgggtgcctgtgatcccagctactccggaggctgaggcagggagaattacttgaacttgggaggcggaggttgctgtgagccgagattacaccactgcaccctagcctgggtggatagagggagattctgtctcaaaaaaaaaaggtgaaagggtgtcttatagagattgggagttaagttttgaaagtggcttttgagccaggcgcggtggctcacgcctgtaatcccagcactttgggaggctgaggcgggtggatcacctgaggtccggagtttgagatcagcgtggacaatgtagtgaaaccacgtctctacttaaaatacaaaaaattagctgggtttggtagcaggtgcctgtaaacccagctactggggaggctgaggcaggagaatcccttgaacccgggaggcggaagttgcagtaagctgagatcacaccattatactccaacctgagcaacaagagcaaaactcccatctcaaaaaaaaaaaaaaaaaagtggcttttaaaatttaataaaaatttgtcttatttttatagcctgccaaccaaacccctcttccttccctacccagtgtcttgatatttgagctttctagccagggcctcaataaatgggaatttgctctattataagagcttccacacattttatgctataattttttaaaggaaaaaattttttgctatatgcaaacttaatttagaaacccttttgacctgtaaaccaaatgttgccccaaagctatgtgaaagcagtgcatactcttgccaaaaggcttacagtgttttccccctgctcagagttgcaaaaatgaattaataagaaaccaaggatttttcttcatgttcttcattaagcaatgtctgaggggaattgtaaaatatgcattgtctacagtatatgtatatcctggtgcacaaagtcagatgaactgtgaaggtgaggggtaaaaacacccctctcacctttgtactttgttctttggagtattaaagaacatttatctgccaagtggcaaaggggatactatttggcggtttaattacagccagtgagtgttattgatgctatcagtcagcaagacccagggaaagggcctggtaccaaatcattcagtgtaattaaatatctcaggacgggattctcaaggtgcttctggagatctgaagcaggagaacactttgtcttagaacaaagataaaggtggtagttcccctcctcgctgcccctctttaaatttctgcaggtgttgcatagatggcaatgatatggtctctttattgcgtacagtgaaggttgtccatgttttccttaaaaactctttcttttaaaatcttcacaactgtcacacaaagggttttgggaatacagtaattattttttgttaaagctgttagggggaatatacagctcaggtgtctaatagatacttggagaatgaaggattgtctttgaatgagactattagtcttgcaaatcagttcatttaaaaaaagctttccctatggttattctctaatgtctaatcatgttgtgagagaaatatcttggtagacttaaagtcataaaacaacactcactaatgtttcaggggttatccccttatgtattgaataaatttttcatagttgcataacttatgtcagccccacaaaatatgtgtaggtaaaacttcctttggctctgtggggagggcttttaataacatttctgttattctttttccttgctttctcctttatcatacgtgccttacacaaatttccatctctgcctgttaaatgccataagcaatctgcagtgtttgctgttactgctttttaaatttttaaaaaactggtacagtttctactgattgaggattaccaaagacaggctgcccttgaaagaaagtgagctggcttcctaacttctttgtgtatgtatacctgtatacatacattagatccctgataattgtgtcaaatcagggcccaaaacactaaatcttggatagcgtatggtttacttgtaatgtagatagggcttgcagattgtgttttaaatttaaaacaggtgtttaaatggggattgttgacataaaacatcctttaagaggaagattatttgttaggaatttttctctgggtgtttgaggaatcctatttttaaaatacgttaaagatttagtagaagtagttactttatgttcaagacttcccaccccttagggctttattatattatatggaaccataacaagactaataggcctgtctccaagtttgtaggaaaggtcagtagctttaaaagatgtgcatggtgctcaccttgcccacaaaacaaagggccaggataagcaaatgtaaaagtttttgagaagtatttaattgcaagttgtttttttgttttactgagaccatgtaatcttggctatacagtgttggacagggagtatctgtaattaaatattttataaacattttgagtgcttaatgtgggctagttagctttgctaaatgtttgaacagcattaaaataagacaagaggtagccttgtccttgatgtcattgtgtagtaagggagacagcacttagtttatatgaccattatttggatatatgatgagtaagtgcagaatacaatgaaaatggatatgccacttgctgtctagaggcctctgtgttgatttcagagttgatggtgttttacaagctaggttttattgggcggaaaaagaagaaatgtattacacataagggaatgtctcattcaaaagcacagaggccttcacgtgatgtgtaccttttaggagaagcttacatctgaacaccataaagtatacttattctatcaagtaataaccttctacaaaaatgattggcaggaaaatttgattaaacctagccctgtactttacatttatccccctttagtgaggacctaatttataagagaaacaataggaatgaattttgggtggatcaagtaatttgctcgtagaaacatatttgccttgctctgcaaagatccgtggagacccatgggactcatttaagtatttacaaatggtatcaagatgaagtttctctaagcagtatttagtgtacacacagaggaaaggatcattatattttcattactatatgaagtaggtcaaagaagacttgggtttctgaacattgttgcaaagcagtttttcttctagttatagtacaaagggaaacttaactgtagatggcatttacatttaactttactaccacactgattttgactgttctcctcacctgcgttatgatgggaaactgtctctgtattctccttccatacatttctgtctgataatacttaccaacatttagattgtcattcctctgctcataaaagttttgccctgacattcaaggtctctagactctgccctcaactcactgttctagtgttgtcagtgtcctacagaaatctttattctggctcagctgggctcttcatcattcagtatggaagcctaattctccattgccttggattattctctgcctacatggccctctaaccctgtcattgataaagttttatttggccataaaataaattaataaagtggataaaaatttcttaactatacatttcctttctcatctatttggtggtctgctctctgagggcaacgggatttttgtctttggagagcagcttgcagaatgtttttatttataacctctgcatgaggaagcttctgtctattgcttataagagacagtgaagacctgctctgtgtgctcaaagacgaaaaaagtcacagtttaagtgaaattaaaagttgagattcttggaggtttttcgttttgttttgtttttgttttaatgccatttttaactttgggtgtgtttttcaaaacttgttggtaggtgtcactatacattgaagatgtggtcggatgaaggatatgtaaaggacttaacagcttttttgtttcatacaaaaacaaagtttttttatatatacatatttttaaaattatactttaagttctagggtacatgtgcacaacgtgcaggtttgttatatatgtatacatgtgccatgttggtgtgctgcacccattaactcatcatttacattaggtatatctcctaatgctatccctcccccctcccccaacccgacaacaggccccggtgtgtgatgttccccaccctgtgtccaagtgttctcattgttcacttcccacctatgagtgagaacatgtggtgaaaaacaaagatttaagtaaaggtatcccgttatgtgagattttctacataatgtccacctacccgtctcttgtcttatccacttctccttttcatgtacttctctctgtcagatcatggatgaagaagtgtatgaaagtcctaggatgtttaattcaccaaaattggcttgatgagaaccttttttcttttttcacattaccaaccggtactggttgttttttattctaagacacctgcagtttatttatgccagccagtgtgtagtccttctttagtttaacattctaactcagcattttaatctgttgacagtgtcttaggcatactagacattcagatactacacaaattatatcttaagtacatttttggagataaagttgccatggttatcttgatagtattggcatctgttcgagatgtcattgggtcaagtttcagaatcttgaaagaatactgaagccattgctggagaatgggcttctaacaattgaatgaatatatctgattttctgcaagccaatttagcctgcgttgttttcagtgaagcagcaaaagccaagttatttgggcaaatgccacaacttctaatcatcttgcttgagaggtacttcagaacaacctcctagctgggcgtggtggctcacgcctgtaatcccagcgctttgggaggccgagacgggcggctcacgaggtcaggagatcaagaccattctggctaacactgtgaaaccccgtctctacaaaaatacaacaaaattagccgggcgtggtggcgggcgcctgtgttcccagctactcgggaggctgaggcaggagaatggtgtgaacccgggaggtggagcttgcagtgagctgagatcgtgccactgcactccagagtgagactctgcctcaaaaaagaaaaacctcctgcacaaggccatggaatgtatacgttcaacaggtagtagagtccctacttggtgggtgggactatgtgagataggcatcatataagacttagtttcaaccctccctctagaagtataaaacatagtaatataaaagctttgaatggtaatactgttaacttgggaggaagctttgaggcagtctagttctctggccgtatttaagaaggctcaaaatactgagtactgactagtgagtactgagatttttcttttagtctctcgaatgtattcatagtatatttaatgaaaatgtctaaagtgaaagttttatagtggtacttgagagtgtagcctcttcagtattttttttttaactgggaaatcatattgaatcatcaggtggccgattgcatggtagcttgtaacacatttcacatgcagaaacatacccataaccttcatctcaagatagaagttattccatgaagttcatttatatttctactactgtctatccaaaagaatcgctcattttacgttttactggcttaggttcaacaggtctctacaaaagagaattgaggtagtgagaggtttaatatgtaacagcttgatgggagaaataaaattttggggccttaactgttggcccatttatagatgttgaatgtttgctctctgtatgtcagtaagacagctccttccttatgttccctccatccaggctgcaggcttcccagcacactcatctgctgtcatcttacaaaatatttggaatattcaggaattaactttaataattatcaccatggatttggaagaagcctcatttatatatgtgatttttgaactgtttctgtgttagagtgcatacattgttcttgtgccacgtctcctcctctgtgtcatctttcctgacctcaaagataatagtattccttgagtttttgttccataaattggggatgatgatgctgcataaacttattgggaggagcaattgaattaacatatgttacatatccagaacagtgtctggcacatggtaaacaataagtgttaggagctatcattatctgaattgtttcctatgtatgtatataaaatgatgttatgattaaaacatttatggttcctctgctctttgctaatagttttattgttgcatttaatacttgtaaggtttccatgaggtaagtactgtgattaacatcaaattacaaataaagagatagaagacctaaggaaatgattaactggtaagtaacagagccaagacctggacctagctctgtgtgactccataccatatgtttttactccttaacatgggagggttctggtccaaacattccatcctaacctctagtttcatagtatgctgtgtagtggccagaactttaccagttacagtgtattctcttctgctttgtataatatatttttgaacctggtgctaagctaacacgtgcttgcctcagttgcagcctattcacattacttagtatttataaatattatgtggctaaataaatgcaggctcatttctgaagttaaaagtaagggagttagcattcttaaccactactgacaattttctaatgcagagagctaccggtaggaatagactctttggtggttacgctaaaactcccatctattttttatactactttagtgtaatcaccttctaaagtctgaaaaggctaagtgaaatatacaggattggaaatggaacatggaacattttgattgacaagttcctgcttagacttggaatggctagcctactgccactaccatagtgaatttgatttcactgcctccacctcagatgccatatccactaccattgctcaattgttttattttctgttaagtatctctgcaggtaaaaaatttaagaaatggtaaggtatttatggcctaagttgcatttcaagaatatctttaccatgtatcccatttgcctttaccagtatgagatgtccttgttgaaccaacttcatactagaaccaaggagaaacaacaataaaagggcagaagttatgtaccagttgtactttactaaaaacgagatgcctgtcagggcagcctatttagaccctctttgatacacatggatgcaactgtttgggtagcatttaataaaccacactcagtgcagtgtagtttgtcagaggctgtttaagatgtgtgaaaaaggtggtctaatttgtgaagtctgtagttgatgatcagttgcttgatatacagttcttacagccaaaacactttttatttttctgtggctattcagtagatttattattgtctttaacaatgtcataggatcacttatttttatgacattacttatttattcctaggtcgcctttaagttgaagctaagatcaactgccttgactaatatttaatgatgttgcactaaaacttgaaaattctatgaaaatgagaatctcccctagtgtaaacagccagttgccattctgttttcctgttaattggtttgtaatcttataaaagggagagacactatcaaaaagattctagctattgtaggttggtacgttttaattggtggaaagtactagtctcaggattcttagtttgatttttctcttcaactcccaaataattagatttttttctccaacatctcctaaataattctcaggcattactagtagaagaattataataactgtcctaagacagtttttttgatgtgttttattatttccttatgtgtatacaagaaaatattctatgtagcaaagctaaggttcttattttctaggaatagctcttctgatgtctccttggaaatgtctgattattgtcccagtcactgtcatttcacaggaagattttatgattttgaagactgtgttgcagctaatgatgtaatggaaaatcccgtgatgaagagctaagaccgcgagtcctacacccaactctttttctttgtgtctttggaaaagttgcttcacctttctgtgcctcagtcttcctatttcccagatacaaagaaattggactaaatgatcactgtattttctttttgctccaaaattaagactccccccgccccacagcctcctatcagcaccatattactttgttgaagattgtgtcgcctctggggacttgaatccccaatttgagaaatgaaatttcatttttgaaatgattaatagaattttcattgtagtctgtacacaaagtaccaatgcgtataatgttgggtatatgacaaagattgtggcttctgttagcttttaaacagtatttcagaaataaactctcaatttaatatgtgattataaatcatcttcattacagtgagtgtgtggtagttaccaggatgtacaaagtaggatgatgacaaggtttcttatttcaaggggttaagtcagtcacaagaaagggttaagagataagtacattttggttaagtcagtatttaagaaaacaaggcatataatgcagtgtgggtgcaggtaagaggcacccaactcagactgtagtggagaggactgattagtgttgactttagggactaggtgatacccatgaggtgtctgaaatgactgaaatttagctgattggaaggagggcagcttatttttaacacagagaagtgagagcaggaagcttggaatactaccgaaggttttgtatacttgtatggaagaggtagccagtgctctgaccataaaggaataagaattctaaaatttatctgaaagtcttagggagccctttaggtaatttaagttgaggatgacattatcaaatatattccgtggtatctctgaacttctcacttgaattaaagcaattgtttagtcttaaaacattcaacaaatccagtcacgataaaatttaaggttctgtgtgtattttccactgatagttttctaaatcctatattttggaatatccgttaaagataactgccctaacctttaataccttttataaaatttcctgtttggatgtgattatttcttttagctctaactttgctggagctaatacttaatttcttattgttatcttaggcttattcattttcaacccattgtgaaatatgcccttccttattgaaaacttaatattggactgtgatacatgtatgccttgatgtgtgtttgagacttgttcctggtaggagtttgaatttagaagcagatcaaagtacacatcttccttttgggtgaattttgaggtctctggcctgagttgtggttagagatcccccttcttctgtgtactatacttctgagctcattttggaaggtccgatgtagccacctgtccaaatatccatgatgctgtcacaagacctttgctactgcttacttagagcctagagctaacttttattgagttatagtgtcatttttcagatagtcccacgaagactccatctttaaaaaatatgaacagcaattaactgtccgtttaaataggcataactgtgacctttcagctggagagattgaaaggagctttttgaaaaaagtctaaagtggcaagtgtctaattttcaggtttaatgtttgtagtcctgggtcagtaattgacaagtgataaaagttgttcagtggtgatcttagcagtgggtgacttctcaaatgtttttagtttaaaaaagagggaacttctttgttcatttatagaccttatcactttaatctattggtattaatgtccactgtcactgatctttccattgatgaatcagagaaacgagagcagttgtgtgtttggacgaaagcatttagagggacttctgatagtgatgcactgggattttaaacattgctatgtacatttaataaagtactaattcataagatggttttgtttctttgtataattttgatcaacttgtgtttgatagcccactcttagtgtatgtgttactcatctttgtatgtttaatgtatcattagttttagctgctgatgaagaaatgagcttttaattacaagtaatacactatatttcaaacggttccattttcaagacataattcaataaatatattctatctttattttgtctagtctagtcttgtttgtgaagaacagtttgagaagccatgggccatttacttacctgtagtagcaagtacttactgcctccccgtccctccccttccctcatataatctcagtattgcttgtctttgtttattatatcactcgtagcttttgtgagaaattccagaataaacctgagttttaaattgctatctttggagtagattggtgttcagtgttgtactccctgtatcatctttaaatatttaaattgcagactctctggctgcctcttaaattagatccttcacttagtagttttgacatttcataaccttgcttctcatgcattaaatattcacctgtttaagtggcagtgaaaatttaaaatccaaactcatatattaatccagcaaatggaaaatggagaacagattgcagtggagaaagaggctattttgacctcagtaggacatatgctggaagtccattgttttgaagcatttctaaatagaatttgtcatttaaagcgaactcttttgggccttcaagggagtttagtcttgtagaaccaaaaagatacttaacactttctgaaaatagcccctgtatgattctgtaggtgcatatgaaggcacctgtaagatctaaatccacaggaaaaaaaaaaaatcggaatggagaaggttgctggtaatggtcagatacaaaatttagtttggtaactgatttgatctccagagtgtgagtccttaggtaactgatctccagagtgtgagtccttatcccactatcagccttcttcccacttaggtctaaatacattttctatttataatctttttagatgaaagaagaaatgttttccaggaagggaggattttgatgacagcgatatccttgatagtggaagggaaaggaagctgttttaattccaagaacagtaaataaagggataggggtaggaccttgagccttggtctgggaggaaggagaaggcaattaccctttcttacacctttatttcctgaaagtgaacgaaaatggcattgcttactcctgagaattagaaagtaacaaaaattaatttaaaaattctaatgatgatgtgtagtttctttggttaattgtaattagtggccacttctcttttcttagaaaaaattactcttaaaaatattaatggagccgggcgtggtggctcatgcctgtaatcccagcactttgggaggccgaggtgggctgatcacctgaggtcaggcattcgagaccaggctgaccaacatggtgaaaccctgtctctaaaaaatacaaaaaaagccagtgtggtggtacatgcctgtaatcccagctacttggaaggctgaggcaggagaatagcttgaacccgggaggcagaggttacagtgagccgagatcacccactgcactccagcctgagcaacagagtgagactccgtctcaaaataataaaaagtggaataacttttaactaatttctagcaacatcctgttttctgtctgtggccagtttctctgtgtatgcctttaagaaacagccaggattcatttaatttttcccaactcagaacaagtgaactgaaacactcacttaccatagtattattgaaggaaaactggatgaggagaccaggacacagaaagagaaagatcagagtttgaacccagaattcaacactagttgtgtataaccttaggcatgttatttaccttgtctgtgcctgtttccccagctataaaatactaatatgtatgctgcctacctcatggggttgttgtgaggcacagacaaaatattgtagtttgtgaaaatgccttgtaaatggtgaagtctcataatgccctaaatgtaagtttaacattaaacattgttaccgacgtaagtttatatataatctaatcactcctgtcctgcctgtggtagtggaaaatggttctttgacaatgtcactgaaagatactgtttttgagaaggtttgtcttggagcctttttaatgtggctgggaactaaagatacaatataaactgaagagtggacattggggaaaaaaaaaatcacataactgacctaggtatgttttcaaagcctttatataagcattaaatataaacccattttcctaaatatattttaggatgacttttaagacacaccacctacactcatctgaattgcacaaagcagtgaatgtcactgcatgagtaaattgttctagaacactgtaatggacccaggtttctctctactttaaggggctgggcatttacctaaattttcagatgcatgtagaggtaaataatatttataatgtcacatagattaggctctgaagcttttcatgtggattagaaattatttaattattcctaatagccatataaagaaggtagaattgtggtaataggcttgaagaacttagttctaagttcacagaactggttatgaattaaacctagttttgagcccaggcacaccgattagctgttacctttaaccacatttccactgcctcctgtgacacttgatctcatgcagagcctgtcacctgctgctggttgcttgatcagtcattggaagttagcctccttggagaatgtgtagatgcctgccctggagcccatgggacatggctgttcataatgggagtacttcagtcacatgatctggaacagagtaacagctgacttgttgggtgctccctcctgtggaaaaaaaagccatagaatcctagatagggatgcacgtcccagctggtgaggtttcaccttttctggataattgagaatcttacacgagaaaagaaattaagttttcattgattcatattactgaatgcatatataattcattggccttgtttcatcagtttatagcaatcccaagatttgattccatctaagttacattaatcacttgaaattggccacttgttcttcacttacatttaagttccccagtcttcattgttcttaagtatttaggcatagagaaaacaaatagcgtatgaatagttatgtatgttttctccaccactattccaaattccttgtttcttctgtagaacaggacagtgctaatataacttccaaacatagcctttggaacaacaggtatattttcacttgttaatgagcaaacggtttgaataaagaagagtattaaatgttaacaccattggggcaggggtgtgaaagataacgaggtctttataaagtagtataaaatgtggcttatatttttttctggaaagccaagaaaaagtggcaacagagagctgtgggcacaattcaaattatggggaaaatgtttggtgcttcatactaaaagttatttaagtattagtgtaaattgggaaagtttgttgttaactggattaaaggtgggtaccaataataaaatgagcaggcagtagttatcactttacctcagcaaatcaatcccagacacattgaccatttttagtaataatctgaggatggcgtatattgtgaaatttctgtcccttgaagtaggctagtccagaatattacagctaaaccaaagggaatgcgatcaggaatagctcatgagactaaatgttggcaaagataggttataggcagagagtcatcaattacaattggggcaggagatctggatgacttgaactgacccttaaatgctctggttcagtaagccgttgtggatctgcatgatcaatggggtattattggatgggctttactcaccaaatgtgttagcatctatcacatgtcaggcactcactaggcattgaggatacagtgatgaccaagaatgatgtggtttcttccctcacagagtttacatgaggaatgatgattataaacaaatacatgcagacctattttgtctttcgtccttgtgggatttggtataccgtatctcaaagacatgaaactttttgaaagtacatacagtggcaggcaaagttaattatagggacttaagaccttttgaaaatcaaggttaaaaagacctgtggatgagaagacaacagacaaaggataatgagggcttgttcacacattcaaatttctgggaatttatggacaatcatacagcatatatcaaaagagcatacttactgataagggtgaatttttttttagaaatgtagcactaccacccacacagatgtgagaggctagaaaacaaaaatgttccatgaatttggaatctttaagcttagaatgattgggcctaaatccctattctagagcaggtgttcccaacccctggccatggacccataccagtccgtggcctgttaggaaccagtgcacacagccagaggtgagcagcaggtgagccagcattattgcccaagctccacctcctgtccgattagctgcagcattagattctcatcagggcacaaaccctattttgaactgcacatgcaaggaatctagattgtgcagtttttatgagaatctaatgcatcccccaccatggtccatggaaaaattgtcttccacaaaaccagtccctggtaccaaaaaggttggggaccactgctctaggggattggccccacagaagacagcctattattgtgaacagtactgtgctgtatgcaccactggtctgttttaagaactttaaattttagtgaattaaaactggactaaaggtgggtacaaatgataaaatgagcaggcagttgttgaatcactatacctccgcaaattgatcccagacccattgaccatttttagtaataatctggaggatgggatatattgtgaaatttctgtcccttgcagtaggctagtccagcatattacagatactcttccctgtccacctaggggagagggaatgggaatactactacccatttgtctagaattttcctggaaatgaataaagcagttagcacctgacttgcttgaacttcagtgttctatgctgttcatgggaaagttaccgaggcagtttttgagtttctaaagggaggtgggtgagtgagcatgcatatacatttaattttattttccttttccaggtttcctatgctcgcccaagttcagcttctatcagagatgcaaatttatatgtcagcggacttccaaaaacaatgacccagaaggagttggaacagcttttttcacaatatggacgcattattacttctcgtattcttgtcgaccaggtcactggtaagtagacagccactccggacagtctttccctgtctctgactagcagattgtgaaatattctgtctttccatgtagcagtcttgcttctagaacatatcaaaggtatatgtgggccagaaaatgcaattttctgctggaattgttcataaatatgcatggataaaaatagattgtggagtatagtgagcgtgcttgagtcatttccaccctgaaggtgcaggattgaagcctgtttcatggcttgtaataaatttatagatgctataatcttcatttccaagccactgtgtgataaaagcccccatcctgcttatagagtactaataaacaacctttcttttgtcaaaaagcgaagtttaatgcagcggtatgcagctagttctccaagggaggattacactacatccacactggtaattgctgttcaggctgttttccaggttcatgctgtgctgtatgggcatatgtaagagtttgaaattttgaatgttaaaaatttattttattcctaatatcactgttagcccaactgaaatgggaagactaaaatgttcattaaaactcttgaatgtaagggagttgttctgccaaatcttagttaacaaagctatagcaatatagatttgattatgaattttaaaaataaaatacagatacaattttctattataaaagcaatacttggccaggcacagtggctcatgcctataatccaagcactttgggaggccaaggagggcggatcacctgaggctgggagttcaagaccagcgtgaccaacatagagaagccccatctctactaaaaatacaaaattagctgggtgtgatggcgcatgcctgtaatcccaggtactcaggaggctgaggcaggagaatcacttgaaccccagaggcggaggtcacggtgagctgagatcatgccattgcactccagcctgggcaatgggcaacaagagcaaaactccatttcaaaaaataagtaaaaaataaaatgaaagcaatacttgttctttaggaaacagttaaaaaatagagaaacattgtgaggaggaaatattagctgtagtaccatagactgaagcagccacatgaagatttgtgaggttgtttttgtttgtttttttaaggtagcttttctcctcactgtttgtttctctccccgccacccgtataataataataccttggaggcatgttgtatattacaatgtctaattcttttaaagcagtgacttcagaagaaaattgagccagatttttcattcatcaagtgaagatccatttagttgatctgaatctaatcagaggagcccatattacctgcctctgtgttgttaattggcttcataggaagcaccacctttagagttctgagtagagagagctagcactacatgcatgcctgggagaatagcctacagattttgctgtcatgagactatttagtcaagtgagagtatataaaatacagcctccttaattgtgttagttaattcataatacttatacattgccaccctgtgttgttatcacatagactaataatggtgttaaatagaaatccaggcctagaaataaagatgaatttgttcaaagcaacaaaaatattttttatttcatttgtgctttctaattcattcattctgagatcttgtgaattgagagtaggtttcacctatgacacaaagtgcgtctcatcttttacctgctagtccatcacaggtggacctagcttgttttagtcagcggttctcaagtatgtcccgcacctgcagtatcagcagtcttctggaaccctgctaaaaatgcaaattattgggcttcattcagaacctactgaataaatctctgagggtgagccacaggttgttcttaggtatgcttaagtaggaggaccaccgatttaggtgactctagctgtgtagaggctgttttttttttttttttttttttttttttttgctaatctgatgcttttctaatggaacagctaaattctagaggttattaattcttaataggcgctagtagataagtgaaaggatttgctatcatttcactatttattcttcatacttaaggctttcaaagtgaacttactcaatttacctgaaatgaaaaatgaaaccctgcaattttcttattcagccatcaggtggtaataatgtgcaatgcatgctctttaaatagaaaaggtaaagtgataggggctgtcttagatacgcatatgtggaaaaaatgctttgtctacattagaaaattcttagtcattgaccttacacagtattaattttaaagcaagtagaccttctttctttttttttttttaagctcctccaagtatgatggtccagtgctcatttacttgccaaaaaaaatttccctgccactgcttatctcatgatgaccagctgtggtcaccccactagcctgttcacgctaattaaatggaataagctgggtttccctctacaatttttcataacctggaaaggttcttggctatcctttcccatttgtgtctttcacactggggagaatgtggggcagggagtgatcttctgtagtcaggagtgatgctgctgaggtcagaaagctttgatgctttacttctagaaatttatttatttttaaaatctacgtcttttctttttctccatataatatttttctgtaataaaatatagctagtcagaagcacaagagagcttcagagaactggaagagcattttcaggcatttttttcctaaaggacctatatcctatgcaatattattaagctgaatatattatttaaaatttcaaccgaaatgtctttctaccacagtgtatgtatttttagcatagctttgtagcatttttagcaaattcttctttctgttctccttgtttcaaggacaggaactactgtgtccctcatttctgagctgcttatccaatagcacaaattgaaaaatggaactccggtacactattgacattaaaagagtgtacgggagggaggtgggagagagagaaaacatggacatgcttgtgttttcttgtcttagctgattggaatgcatgtgataatgggtactcagattatgtaagtgacacgaatttttctaaaaatgcaaaattgtgattctaaactgaatggctagtaagtaaaggggaaagtttcctcctggtgtgattttgtgggaaatgttatatgtaggtgaaaacatgttcttgagaaggaaaaattaatgtgtcctttcttctctccttcccttctccttctgttccctcttttccaaatctttactcaaatctaccataggcatatcaaggggtgtagggtttattcgatttgacaagcgaattgaggcagaagaagctatcaaaggcctaaatggccagaaacctcccggtgccacggagccaatcactgtaaagtttgctaataacccaagccaaaaaaccaatcaggccatcctttcccagctgtaccagtctccaaacagaaggtatccaggaccgctagctcagcaggcacagcgttttaggtaagtctggtttggttcttgtgcccagctactaaactgaaagagaatactcaggtttactgtccagtacttttattgacaggatctctggtttgcttttgtcagtgttgttggttttattactattaatttttcatctccatgggcacagagagaatctacttgaggaatagtgtcttttgctaatattgaaggtggggaccaaaggacagtaacctacagcagtgattcttaaccaagggtgatttttgcctcccagggaacatttggcaatgtctagaggtattcatgttggcacaacctgtgggggtgctactagcatgtaatgctagaggtcggggatgctgctaagctcctacagtgtacagcatagccccagcaaccagaagttacctggtccaaagcaccaataatgctgtggttgagaaattctaatctgtagctttctggggttatctgaattgtttccagggcttcagatttccttttttttttttcttaaaggattttattctcatgtgtcaatttttaggtcaagctgtttctgttacttcactttatagtatgtgcaccattaagggtatataacctatgtgactacgtgaagtttctacaaatgagttcctctcttccttttcaaaattgtttacaaacaggttggcagactttctctgtacagtaccagatagtaaatatttaactttatgggttatggtcactgttgcatctgtcttgtttttgtttttaaacaacctgttaaaaatgtaaaagccattcttagcttatagcccatacagcaacaggtgggaggcagtctccatttggcctgtcggttgtaatttactgacctctctgtagttcatagaatgaccgtggctttagcccaaagaagacactctatctcactgacaggtgtacatatgctaactgtttaagttttattctgatcctgggataacaaaccaggagtcattcaaaaaatctctaatctgcatgtgagaagaacagtgactttaagaagttaagcagagcagagagtttttggtttcataagcaagtcatgcagtttttattttccaggacatttttaaatatcctttttggccatatggtacactatacccagaatgctatctaaccccacactgatttttaaaaattatttccaagtgcatatcaattttatttatttcattcatgggacagaatagttaattaccaaaaatataatcacaaatataacctaataagatcaggagttatgttttctagcttaaatctatgcatttatttttttcattggaatatttgcagtgctgaagagagtggagataaaatcagttaaaaatagctccttttaaaaaaatactaaattatgttgcttgagatatgagaaggaaatttgttatgaatgccacattaaatttcaacttaagttggcctttgccgtgggagaagaggtggtactgtcaaaagcattgagccttgtacttcatgctctgctgtgaggaaaaggcattggtattaaaaaaaaaaaaaaaaaaaaaaaaaaaaaaaaaaaaaaaacctttgccacctttgacttcccatggtagtggtgatcagactagtgtgggagatgcgtggggctgcctgatgcttgcctttgtggagaaagaagtctcacaaagaaagccctttttcagctctgcacataggtttcctctgtgacatcttaagtgaataatatatgtataaatatacttttttaaattgcaaacgtaggatgaacaagcctgaaggaattttacaggtatttcctaggcagcttgtccagttaatttcagagctctttggggccttgaaaatgatctcgagtttagatagaacaaagttcttcataatgtattggttgatttttaacattgtgaagttataaaatgtttgctgttagtggaaagctatattagctactttttttttccagattgaagcaatgattttgtttgatagttattaagtaaaacaaagttcttggaatcaagattgaattcttaggaagattgagaagacaggatttgggagagtgcctgattattgaaatggcaaggcttgtaggtgactcaggtgctaggttgttgatgggtgtcagcccttaggtttacctagcctccatccctgcaggctctgtgctttttatttttggttgagtaaagctttacaggatgttcttatggtcaaatttaattatctttttctggcccattctgtgtttttatcttttagtaccatatgttatcattaacttgaaaattctttttccttcactcctatagaaagacattttggaagaagagaataagagattgtcagttgtaagggaaacttggtttcagaaaatgggagaaattgttattctctcttcagatttttaactgttgtcccattcaccccatttctcattcctcccatgatccctgtccagataataaaatatgaacgagttagtgtgctcagagatagtgtttgatctagattgggtgtgttggaagaaatcagatttcaggtttaacctgatgtttgatgcttttctttaatacagtatattgaacctggttgtcattatcttgatttccgccccactcccccttcttctttcacctgataaagaaacttgaattaagggcttgcttgttttgttggtattttgctgttttctgtagttcgagtctaaatcatcacacagatatattttgtccctgatatttgtgttttaatgctcttgccttgagtttttgtcacctttcagcagacatgtagaaaatttctcactgtatttttatgttctcatagtaccttgggagttgagagagattgagcaatataagttatatgtattatggtcaagaatacttttaataatcataaagtatgagccatatcttttatggtcaaaaagcttaaaaatccatgtaataagggttaagtttaaaggtgggtttgtgaggtttatggcttagtcaatctatgaataatcagagaattgttgtaatatgagcactgctggtctaatacgcttatttaattgactgcagttttcaaaataggacaagccttatttatttggatggatggtgtaattcaagccccttaagtatattcttgggacatgatatcagaattaaactttaagccaaattttttcatgttcttttctgtgaaacagctataattttattgaatatgatctagtgtaatgacctgtgtgcctgcttctatgagcctttatgcattttttaaaaagccttttaaatgagcacttgagttgcctttaaggcatgacgtactggttgcattatcacaatttccttttggaagaaaccggtatcaatccccttgaatacttttcctctttcttagaggacagatgataccatctaattgaataaggcagtaaggcagtaagaagatgatcttgttgtagataatttccaagcagttctcggttggcgctttggagagattttgataatcattatttgtgggattccgagtatattttcttacccatcaataacttatcttcctatttttatttctgactttaatcgggttcacatttccccttacttacatgtaggccttcccatagtccatcagctaatttttttagaaattaggcaggtactttatttttattttctacttatgaacacctagctttaaaataggaatgatctgatacatggttctgttcaaaattttgacaggttataattggagctttggtcctgttgatagaaaaatgtttatacacatctctctcctatgtgcctttttcataaattacatatgtacatgggcgtgtgctattcattaaattataaactctaatcaaacataaaaaaggataaagatgagacaatctgtattaaatgtcttgctaattactatactggttggcttcattttccatgtacctaatatcttaataaatgtcttttaaagcaagaccataaaataatttcagatatctcatctgacttcatgtgatgcatgttggattatcttgtgtctaaccttataatgtagcttgtactaagagtaggagaaataatcggctctccctttccttatttacttgtatttcccttactggtaccattctttactctgatttacatttgtagtaatctttttttaagttactttttctttaaatggaggtcattttaattaaagcaggttatccagtctaatcctccatatctgcttgctgtgttatttgtaaaatggattttgttcaggctaaagagcaggagagagtgccactcgccgcctctccccttcgatgattgctcagtcttcccccagggaggcaggtgatacacaggactgagaaggtactgggttcagtgcatctattaaatataagtaaaaaatactttattttaaatacatatttattttatattttattttaaatatatatttaatggatactttaatatttaccttccatatgtactacgttatcatccacacccctccctgagatattctggaggcagctagcttgctctccatttaaaaattgacttccatttataaatgaccataaataaaagatggctgggcgtggtggctcactcctgtaatcccagcactttgggaggccaaggcgggtggatcacgaggtcaggagatcgagaccatcatggctaaatggtgaaaccccgtctctactaaaaatacaaaaaaaattagccggacgtggtggttgggtgcctgtagtcccagctactcgagaggctggggccagagaatggtgtgaacccgggaggcagagtttgcagtgagccgagatcacgccactgcactccagcctgggtgacagagcgagacttcgtctcaaaaataaataaataaaataaaaagatgtccttgagcagtggacagacccagcagacatatttctaggggcttgttgtagtagtccttgtgtttgagctgctgcactattttctcttacaactgcctgtgttacagtttgccaactgctactggatttggagtgaatggtgcccatgtagagcacccgttgccttttctgagctcccatgggaaacatgaatctatggcaccgagaaagctaggaaatacttctgggctcagatgcatacagtcagggggcttcagcagtgcctgggcactgttttctaggttccatgcttctccctacactcgttggtgatggaggagaagaaagattagagtggataacttggcttagttccaaaggaccaatttttttctaaaatgagagagaagtgaaggaatcagtgaagatcatggtatcatgtaaaacttcacagatagggtaaaaaagatgccctatcataatttggagggtcctcggagatcctgttataaaaaattcgaaacctcctgtctggggatgccagagatggaagtgagggagatggaggatggaaaacgaaatgtttctccctcactgcatgtacagtcatgcatcacataacgtttcagttaatcacatgtacgaccgtggtaccataatatagtagactgcaaaaaattcctattgtctagtgatatcatagtcatcctaatattgtgcaatgcattaccttttctatgtttagatatggttaaatacacaatacttagcattgtgttagaattacctacagtattcagtacagtgacatgttatacaggtttgtagactgggagccatagcccataccatataactaggtgtgtggtaggctattaccatctaggtttgtataagtgcattctatgatcgtacaacaacgagatcacctagtgacacattttcagaacatatccccattgttaaaaaaggcctgacggtaattcacttcaggggcagatttggtttctaccctgtttgaaaattctgaaaactcatctgtctctgggccgtatgcttggtgtcctgagtgggtggtagcaaagcatgacctgtccctacatggatgcaattaatgggcatagatgatgtaacttttgagtgaattgataaggtgcctctgcattctgggtcttctagcactttggttggagacctcagtatgcggttctcttctacaggtagcatatgccaaatgcattctgtgttgatcattccagcaatttgataaaattgtattgttactttatgattggtggtgctaatatgtgtttgtctttattgctggtagttgtgatccttattcttgactataaaggcatggcatctttgactgtactcacctctgcaaaaaataaaaccccacatctttttacttatgtgcagacaaggtcatccaaccaatcctcttactagttttcattctggactaagttgaccataattttggtaacctcattgtctttatttggtcatcacatatttattataaactatcagtgaccctggcttgtgctcagtggtgcaatgagagggtggtgggtcaaggcacgcacacacacacacgcaccacgcacacagagacacactcacacacgaagatcttttgtcattgtgatctgttctgtttccttacactttgcataagctcagttcttctcatctttacaaaacagaattttcagtggagactgagggcagggatgtgggaggctatatatttccccattctgaaagaccagatgagatgtgttttggcttttacaaaactaaaaggtgttggcttttgcatgtatactttagcgtgacttgatgccacaggttaggtgataaggctgagacttttcaaacttacctttcacgggtgttaaagaaaaaattctcacccccacccccagtcccagagggacaaccctgggatgtctttggagagtgttgagcattgtagcagttgagggtggttgtggactatgtattaagttatgcctcctcctcagaacacataattagttggtaagcactgtggaaatttgggacattagagtggtgttttgaaactgataaagaagagtcaaatgctgtaaaactaatatttgatataggaaaaatgtagaaggttttaaagagtacttacacctccacccccacccctatttactttctataaacattttaagtgaggctcagattggcttgttttgtttcctacctggcctttgaaaactgagtaggacgacacttcctgtagcgtaatttgaatacagcatccagatcaggatctgaactcttggctttgttgcacactggagtgatggatttttcttttttccttctctagacctcagctgagcacatttgagaaatgggagggttagcttgggttgtccataaggttttttctacctgtaattatacctccttgattctaaaagatgaaagcccttcaaagttgggaaatgaaaggaaaatgtatagaattctgtaacctgcagagtttgagtatcaatgccataatttactagccctgtggtcttgagcaagccacttaaaccctgtgtgccttaggtatctgcaggcaccaacagagttgtgaaaatcaaaagtagtataaagaacatgtagctatacattgatcactttgaagctacctgatgagaaccctaaagttaactatcccagagctttcgcacttcctctctgaaaggtatcctcagatgaccgtcactgtgtcccatgtactgacccttgtagagagaaaccattcattggaagaactgtcctgtggcagtcggtgcccattagcttaaaaacgtaaaaagacccagcattctgagtcttcacatataatcagtgtacagatatcccctcggaagatggcagtaatgagcccaactttcttgaacatcaaggggaaaaattaaaaactgaagtttgctaaatgtgtgttttgcaggttggacaatctgctcaatatggcttatggagtaaagaggtaatgatagaggttcatgatactcataatccctttccacagttgattggtttctttgattttgggagtgctattcacacccctcataagtttttgttgggttttattttgcatgtgcaacctaatgaaaagctgctctttgaagcacaattgactcaagaaatttttataccaccggatgccacctggtcttttaaaaacccatcatttgtttcgtattgcgttacgacgttacagcatgtttacgtatgtgttgatcccttcccctgccaatcctgcttaattcatgaagccagttctaaatatatttctttgctattgtggatgatattcagtacagtgaataatggtcatttcataacttattaatatctcagtagtctagcatgtcactgcaaagccacacacagaccttggtcaaaacatgaaatagtttatatatttgttgggggaggagagttactaaaattcttggggatggaaaaggtatacgttcattgctataaccaacctctgtttttattttttgtgtgtatgtgtgtatttcctattctgtttagcaggttttctccaatgaccattgacggaatgaccagtttggctggaattaatatccctgggcaccctggaacagggtggtgtatatttgtgtacaacctggctcctgacgcagatgagagtatcctgtggcaaatgtttgggccttttggagctgtcaccaatgtgaaggtcatccgtgactttaacaccaataaatgcaaaggttttggatttgtgactatgacaaactatgatgaggctgccatggcgatagctagcctcaatggataccgtctgggagacagagtactgcaggtctcctttaagacaaacaaaacgcacaaagcctaatgagctcttgtcctcagtccatttatatatgaaaactatacaacaaaggcaagttaagagaaactttatacattagtaaatgtctttgtaagtcagtgttgagatggggataaaatgactacttagcatcctaagaaatatgtgagattttttattgctagtatttgaattaaaacttcttaaatatcttttatgtttgaatatggacaagaggtacagggtttttacctgtcacattgcattctattgccttctttgaagaaggtggaccttttaaagtgtttcagctaagggaagacatttcttttctttttacataactgccttgaacctgtgagtaaatattgaggctttgtgttgtaattcttcagttggttgtgtcttttttttcccccctttttttcctttttctgattagctttgtgtttggtttacatttaaagcattgctgttatgtctgtttaagaaaagtattttgaagtttacatttttatttatgaagtttaaaacagtatttattttgtaattatgatttgggttggggaagggggggctacattataaacgcttattgtaagaatactggagaacttttcgtaaagcagtaccttgccaaagagataagagcctctttgatgtgggtttaaaaaaagcatctatttttataaaaaagaaaatttggagaaactttttactggtcctggaacaaatattttgacttgaatactttgagaaatctcttcatatgacacctagtgagcttttaaaatttaccaggaaatttgcagcggttggaaaatttagaaagatttatggtgtagaaaatacttttgagatctttgtatgaaaggagtagaatcaatggggggaaacactgctggtttcatttttgtaatcaccagtggagcgtctgatcatcctggttattatgtgataggtggctcacattgatttgtgattttgaaacaaataaaaaaaatttacaaaagaatatataagagcaggcaagaaatttaaattaccgagagatgggggaaaaaatctgttcttcctaaagaaatcccttcagatagagctcatggtgtttagtgatgtacttgcagtattgtttgaagaattgttttgtcttaaggaaaaaagacgttgcacatgatttgtactgcagcaaatcagcaaaagtgatctgagttggatatatttgaaggtattttgaaagttacgttcaaggctaacacctgagctttgtgtaatgtaaataagaccttgtgtttatgaacctttcagctaatttaattttttttcccttacatgccaagtgatgttcaggttttgaatgtttttgtatcagttttttcctttgtaaatggcattaacattgttacttgaggtcttgcttaatcacttttgttgtcctgaggacttgaatttacagtgcatcagatttgttgcaaattttgtctgtagatagtctagcttcagctgtttatggtgatgctacattttggtttataaatatgtttgtggtataaaaaaatgagtataaccataggttttgaacaaatttccttacatttttcatacaaaaatcataaatatctgtatgctattgaaatttaactttgtatgatgcttaaaaaccactatttggggaaataataaaataagtctttaccatgtatgaaagaaattttaaaaaatacaaaatattttctgattagcatctagcttataataaattttcaaaaaagctgaaggcaaaaatgccttcatcaggatgcactgagaactatatagttacgtcctgctttttgtataaactgagatgctcacatgcttccccttagaacaggcaatgtgctatgcataacatagttgtacattatctttgcggttgctttgagttttattttttattatttaaaattgtagttataaaatttttcagtatagtacagtacatatactgtgaggcgcgtgctaaagtgaataagcgagttttcatgctgacccactcaatgctattcagaaatcaattggcttagcactttctcatatccttaggtgcatttagattgccagagttaaccttctgcgtttaaaaaaagaaaaacactaaaaaataaaatacatgtatatacttaaaaaaaaataataaggtttccctcaagggaaaacagcagctacatgcttctttcctatactactgtagcaaaccaaggcattgatgagagggcatgcaaattgtgcttcactttacagtgttttatcagagcacttaataaaatgtaaggctggtatttatttgaagttgtacagtatgacttaattcacatctgttggaatagaaaatatattctgttgagtatttaagaggctgtacatgttttcttttgtgtttggattctttgtactttttcatgttcagtacatcaataaacaaagttgaagggaaa

SEQ ID NO: 2 - DNA, 3983 bp, Homo sapiens
  misc_feature 1..3983: note = Description of sequence: ELAV Like RNA Binding Protein 2 (ELAVL2)
  source 1..3983: mol_type = genomic DNA
aagacgtcctcccccctatttctgttcttagttagagatgcagcagcttactcctgtagcgacctactaaaaagcaacaaggagaaagacatcgtctttttgaaaaaacgttctttcgtctcttttctttgttccttggtttgtttttcttgcccctttttgtttagttgaacggcaataggagggtagtctctccgtctttttaaactcttttttaagtttcccctcccctttcatattttttttcgccatttcttttagcattggactttggggtcgaaagcgtttctttttatttgcttcttttaagccgagcacagtttaggtttcgtgctgtcttaagagaactatccagcagcttcttgctcatccttattgggagaactgcaccgttactttaaaaacacacatacacaaaaaccttaagggagaaagcaggtaattgctgccatggaaacacaactgtctaatgggccaacttgcaataacacagccaatggtccaaccaccataaacaacaactgttcgtcaccagttgactctgggaacacagaagacagcaagaccaacttaatagtcaactaccttcctcagaacatgacacaggaggaactaaagagtctctttgggagcattggtgaaatagagtcctgtaagcttgtaagagacaaaataacagggcagagcttgggatatggctttgtgaactacattgaccccaaggatgcagagaaagctatcaacaccctgaatggattgagacttcaaaccaaaacaataaaagtttcctatgctcgcccaagttcagcttctatcagagatgcaaatttatatgtcagcggacttccaaaaacaatgacccagaaggagttggaacagcttttttcacaatatggacgcattattacttctcgtattcttgtcgaccaggtcactggcatatcaaggggtgtagggtttattcgatttgacaagcgaattgaggcagaagaagctatcaaaggcctaaatggccagaaacctcccggtgccacggagccaatcactgtaaagtttgctaataacccaagccaaaaaaccaatcaggccatcctttcccagctgtaccagtctccaaacagaaggtatccaggaccgctagctcagcaggcacagcgttttaggttggacaatctgctcaatatggcttatggagtaaagaggttttctccaatgaccattgacggaatgaccagtttggctggaattaatatccctgggcaccctggaacagggtggtgtatatttgtgtacaacctggctcctgacgcagatgagagtatcctgtggcaaatgtttgggccttttggagctgtcaccaatgtgaaggtcatccgtgactttaacaccaataaatgcaaaggttttggatttgtgactatgacaaactatgatgaggctgccatggcgatagctagcctcaatggataccgtctgggagacagagtactgcaggtctcctttaagacaaacaaaacgcacaaagcctaatgagctcttgtcctcagtccatttatatatgaaaactatacaacaaaggcaagttaagagaaactttatacattagtaaatgtctttgtaagtcagtgttgagatggggataaaatgactacttagcatcctaagaaatatgtgagattttttattgctagtatttgaattaaaacttcttaaatatcttttatgtttgaatatggacaagaggtacagggtttttacctgtcacattgcattctattgccttctttgaagaaggtggaccttttaaagtgtttcagctaagggaagacatttcttttctttttacataactgccttgaacctgtgagtaaatattgaggctttgtgttgtaattcttcagttggttgtgtcttttttttcccccctttttttcctttttctgattagctttgtgtttggtttacatttaaagcattgctgttatgtctgtttaagaaaagtattttgaagtttacatttttatttatgaagtttaaaacagtatttattttgtaattatgatttgggttggggaagggggggctacattataaacgcttattgtaagaatactggagaacttttcgtaaagcagtaccttgccaaagagataagagcctctttgatgtgggtttaaaaaaagcatctatttttataaaaaagaaaatttggagaaactttttactggtcctggaacaaatattttgacttgaatactttgagaaatctcttcatatgacacctagtgagcttttaaaatttaccaggaaatttgcagcggttggaaaatttagaaagatttatggtgtagaaaatacttttgagatctttgtatgaaaggagtagaatcaatggggggaaacactgctggtttcatttttgtaatcaccagtggagcgtctgatcatcctggttattatgtgataggtggctcacattgatttgtgattttgaaacaaataaaaaaaatttacaaaagaatatataagagcaggcaagaaatttaaattaccgagagatgggggaaaaaatctgttcttcctaaagaaatcccttcagatagagctcatggtgtttagtgatgtacttgcagtattgtttgaagaattgttttgtcttaaggaaaaaagacgttgcacatgatttgtactgcagcaaatcagcaaaagtgatctgagttggatatatttgaaggtattttgaaagttacgttcaaggctaacacctgagctttgtgtaatgtaaataagaccttgtgtttatgaacctttcagctaatttaattttttttcccttacatgccaagtgatgttcaggttttgaatgtttttgtatcagttttttcctttgtaaatggcattaacattgttacttgaggtcttgcttaatcacttttgttgtcctgaggacttgaatttacagtgcatcagatttgttgcaaattttgtctgtagatagtctagcttcagctgtttatggtgatgctacattttggtttataaatatgtttgtggtataaaaaaatgagtataaccataggttttgaacaaatttccttacatttttcatacaaaaatcataaatatctgtatgctattgaaatttaactttgtatgatgcttaaaaaccactatttggggaaataataaaataagtctttaccatgtatgaaagaaattttaaaaaatacaaaatattttctgattagcatctagcttataataaattttcaaaaaagctgaaggcaaaaatgccttcatcaggatgcactgagaactatatagttacgtcctgctttttgtataaactgagatgctcacatgcttccccttagaacaggcaatgtgctatgcataacatagttgtacattatctttgcggttgctttgagttttattttttattatttaaaattgtagttataaaatttttcagtatagtacagtacatatactgtgaggcgcgtgctaaagtgaataagcgagttttcatgctgacccactcaatgctattcagaaatcaattggcttagcactttctcatatccttaggtgcatttagattgccagagttaaccttctgcgtttaaaaaaagaaaaacactaaaaaataaaatacatgtatatacttaaaaaaaaataataaggtttccctcaagggaaaacagcagctacatgcttctttcctatactactgtagcaaaccaaggcattgatgagagggcatgcaaattgtgcttcactttacagtgttttatcagagcacttaataaaatgtaaggctggtatttatttgaagttgtacagtatgacttaattcacatctgttggaatagaaaatatattctgttgagtatttaagaggctgtacatgttttcttttgtgtttggattctttgtactttttcatgttcagtacatcaataaacaaagttgaagggaaa

SEQ ID NO: 3 - DNA, 18 bp, synthetic construct
  misc_feature 1..18: note = Description of sequence: antisense oligo
  source 1..18: mol_type = other DNA
actaagaacagaaatagg

SEQ ID NO: 4 - DNA, 20 bp, synthetic construct
  misc_feature 1..20: note = Description of sequence: antisense oligo
  source 1..20: mol_type = other DNA
tctaactaagaacagaaata

SEQ ID NO: 5 - DNA, 18 bp, synthetic construct
  misc_feature 1..18: note = Description of sequence: antisense oligo
  source 1..18: mol_type = other DNA
ctacaggagtaagctgct

SEQ ID NO: 6 - DNA, 20 bp, synthetic construct
  misc_feature 1..20: note = Description of sequence: antisense oligo
  source 1..20: mol_type = other DNA
tgctgcatctctaactaaga

SEQ ID NO: 7 - DNA, 20 bp, synthetic construct
  misc_feature 1..20: note = Description of sequence: antisense oligo
  source 1..20: mol_type = other DNA
tttagtaggtcgctacagga

SEQ ID NO: 8 - DNA, 20 bp, synthetic construct
  misc_feature 1..20: note = Description of sequence: antisense oligo
  source 1..20: mol_type = other DNA
tcccttaaggtttttgtgta

SEQ ID NO: 9 - DNA, 20 bp, synthetic construct
  misc_feature 1..20: note = Description of sequence: antisense oligo
  source 1..20: mol_type = other DNA
accttcttcaaagaaggcaa

SEQ ID NO: 10 - DNA, 20 bp, synthetic construct
  misc_feature 1..20: note = Description of sequence: antisense oligo
  source 1..20: mol_type = other DNA
tcaaagaggctcttatctct

SEQ ID NO: 11 - DNA, 20 bp, synthetic construct
  misc_feature 1..20: note = Description of sequence: antisense oligo
  source 1..20: mol_type = other DNA
aactatgttatgcatagcac

SEQ ID NO: 12 - DNA, 20 bp, synthetic construct
  misc_feature 1..20: note = Description of sequence: antisense oligo
  source 1..20: mol_type = other DNA
gtgcccagggatattaattc

SEQ ID NO: 13 - DNA, 18 bp, synthetic construct
  misc_feature 1..18: note = Description of sequence: antisense oligo
  source 1..18: mol_type = other DNA
acctaaggatatgagaaa

SEQ ID NO: 14 - DNA, 20 bp, synthetic construct
  misc_feature 1..20: note = Description of sequence: antisense oligo
  source 1..20: mol_type = other DNA
ctgctttacgaaaagttctc

SEQ ID NO: 15 - DNA, 18 bp, synthetic construct
  misc_feature 1..18: note = Description of sequence: antisense oligo
  source 1..18: mol_type = other DNA
cctcacagtatatgtact

SEQ ID NO: 16 - DNA, 20 bp, synthetic construct
  misc_feature 1..20: note = Description of sequence: antisense oligo
  source 1..20: mol_type = other DNA
cggtaatttaaatttcttgc

SEQ ID NO: 17 - DNA, 20 bp, synthetic construct
  misc_feature 1..20: note = Description of sequence: antisense oligo
  source 1..20: mol_type = other DNA
cactaggtgtcatatgaaga

SEQ ID NO: 18 - DNA, 20 bp, synthetic construct
  misc_feature 1..20: note = Description of sequence: antisense oligo
  source 1..20: mol_type = other DNA
ccaattgatttctgaatagc

SEQ ID NO: 19 - DNA, 20 bp, synthetic construct
  misc_feature 1..20: note = Description of sequence: antisense oligo
  source 1..20: mol_type = other DNA
agtcctcaggacaacaaaag

SEQ ID NO: 20 - DNA, 20 bp, synthetic construct
  misc_feature 1..20: note = Description of sequence: antisense oligo
  source 1..20: mol_type = other DNA
ttcaaaaagacgatgtcttt

SEQ ID NO: 21 - DNA, 20 bp, synthetic construct
  misc_feature 1..20: note = Description of sequence: antisense oligo
  source 1..20: mol_type = other DNA
ttcccttgagggaaacctta

SEQ ID NO: 22 - DNA, 18 bp, synthetic construct
  misc_feature 1..18: note = Description of sequence: antisense oligo
  source 1..18: mol_type = other DNA
gcaggacgtaactatata

SEQ ID NO: 23 - DNA, 20 bp, synthetic construct
  misc_feature 1..20: note = Description of sequence: antisense oligo
  source 1..20: mol_type = other DNA
cgcaaagataatgtacaact

SEQ ID NO: 24 - DNA, 20 bp, synthetic construct
  misc_feature 1..20: note = Description of sequence: antisense oligo
  source 1..20: mol_type = other DNA
aaaatatttgttccaggacc

SEQ ID NO: 25 - DNA, 20 bp, synthetic construct
  misc_feature 1..20: note = Description of sequence: antisense oligo
  source 1..20: mol_type = other DNA
accataaacagctgaagcta

SEQ ID NO: 26 - DNA, 20 bp, synthetic construct
  misc_feature 1..20: note = Description of sequence: antisense oligo
  source 1..20: mol_type = other DNA
gctggatagttctcttaaga

SEQ ID NO: 27 - DNA, 18 bp, synthetic construct
  misc_feature 1..18: note = Description of sequence: antisense oligo
  source 1..18: mol_type = other DNA
  modified_base 1: mod_base = OTHER; note = 2'-o-methoxy-ethyl-adenine
  modified_base 2: mod_base = OTHER; note = 2'-o-methoxy-ethyl-cytidine
  modified_base 3: mod_base = OTHER; note = 2'-o-methoxy-ethyl-thymine
  modified_base 4: mod_base = OTHER; note = 2'-o-methoxy-ethyl-adenine
  modified_base 5: mod_base = OTHER; note = 2'-o-methoxy-ethyl-adenine
  modified_base 14: mod_base = OTHER; note = 2'-o-methoxy-ethyl-adenine
  modified_base 15: mod_base = OTHER; note = 2'-o-methoxy-ethyl-thymine
  modified_base 16: mod_base = OTHER; note = 2'-o-methoxy-ethyl-adenine
  modified_base 17: mod_base = OTHER; note = 2'-o-methoxy-ethyl-guanosine
  modified_base 18: mod_base = OTHER; note = 2'-o-methoxy-ethyl-guanosine
  modified_base 1..18: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18
actaagaacagaaatagg

SEQ ID NO: 28 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-thymine
  modified_base 2: mod_base = OTHER; note = 2'-o-methoxy-ethyl-cytidine
  modified_base 3: mod_base = OTHER; note = 2'-o-methoxy-ethyl-thymine
  modified_base 4: mod_base = OTHER; note = 2'-o-methoxy-ethyl-adenine
  modified_base 5: mod_base = OTHER; note = 2'-o-methoxy-ethyl-adenine
  modified_base 16: mod_base = OTHER; note = 2'-o-methoxy-ethyl-adenine
  modified_base 17: mod_base = OTHER; note = 2'-o-methoxy-ethyl-adenine
  modified_base 18: mod_base = OTHER; note = 2'-o-methoxy-ethyl-adenine
  modified_base 19: mod_base = OTHER; note = 2'-o-methoxy-ethyl-thymine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
tctaactaagaacagaaata

SEQ ID NO: 29 - DNA, 18 bp, synthetic construct
  misc_feature 1..18: note = Description of sequence: antisense oligo
  source 1..18: mol_type = other DNA
  modified_base 1: mod_base = OTHER; note = 2'-o-methoxy-ethyl-cytidine
  modified_base 2: mod_base = OTHER; note = 2'-o-methoxy-ethyl-thymine
  modified_base 3: mod_base = OTHER; note = 2'-o-methoxy-ethyl-adenine
  modified_base 4: mod_base = OTHER; note = 2'-o-methoxy-ethyl-cytidine
  modified_base 5: mod_base = OTHER; note = 2'-o-methoxy-ethyl-adenine
  modified_base 14: mod_base = OTHER; note = 2'-o-methoxy-ethyl-cytidine
  modified_base 15: mod_base = OTHER; note = 2'-o-methoxy-ethyl-thymine
  modified_base 16: mod_base = OTHER; note = 2'-o-methoxy-ethyl-guanosine
  modified_base 17: mod_base = OTHER; note = 2'-o-methoxy-ethyl-cytidine
  modified_base 18: mod_base = OTHER; note = 2'-o-methoxy-ethyl-thymine
  modified_base 1..18: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18
ctacaggagtaagctgct

SEQ ID NO: 30 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-thymine
  modified_base 2: mod_base = OTHER; note = 2'-o-methoxy-ethyl-guanosine
  modified_base 3: mod_base = OTHER; note = 2'-o-methoxy-ethyl-cytidine
  modified_base 4: mod_base = OTHER; note = 2'-o-methoxy-ethyl-thymine
  modified_base 5: mod_base = OTHER; note = 2'-o-methoxy-ethyl-guanosine
  modified_base 16: mod_base = OTHER; note = 2'-o-methoxy-ethyl-thymine
  modified_base 17: mod_base = OTHER; note = 2'-o-methoxy-ethyl-adenine
  modified_base 18: mod_base = OTHER; note = 2'-o-methoxy-ethyl-adenine
  modified_base 19: mod_base = OTHER; note = 2'-o-methoxy-ethyl-guanosine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
tgctgcatctctaactaaga

SEQ ID NO: 31 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-thymine
  modified_base 2: mod_base = OTHER; note = 2'-o-methoxy-ethyl-thymine
  modified_base 3: mod_base = OTHER; note = 2'-o-methoxy-ethyl-thymine
  modified_base 4: mod_base = OTHER; note = 2'-o-methoxy-ethyl-adenine
  modified_base 5: mod_base = OTHER; note = 2'-o-methoxy-ethyl-guanosine
  modified_base 16: mod_base = OTHER; note = 2'-o-methoxy-ethyl-cytidine
  modified_base 17: mod_base = OTHER; note = 2'-o-methoxy-ethyl-adenine
  modified_base 18: mod_base = OTHER; note = 2'-o-methoxy-ethyl-guanosine
  modified_base 19: mod_base = OTHER; note = 2'-o-methoxy-ethyl-guanosine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
tttagtaggtcgctacagga

SEQ ID NO: 32 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-thymine
  modified_base 2: mod_base = OTHER; note = 2'-o-methoxy-ethyl-cytidine
  modified_base 3: mod_base = OTHER; note = 2'-o-methoxy-ethyl-cytidine
  modified_base 4: mod_base = OTHER; note = 2'-o-methoxy-ethyl-cytidine
  modified_base 5: mod_base = OTHER; note = 2'-o-methoxy-ethyl-thymine
  modified_base 16: mod_base = OTHER; note = 2'-o-methoxy-ethyl-guanosine
  modified_base 17: mod_base = OTHER; note = 2'-o-methoxy-ethyl-thymine
  modified_base 18: mod_base = OTHER; note = 2'-o-methoxy-ethyl-guanosine
  modified_base 19: mod_base = OTHER; note = 2'-o-methoxy-ethyl-thymine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
tcccttaaggtttttgtgta

SEQ ID NO: 33 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-adenine
  modified_base 2: mod_base = OTHER; note = 2'-o-methoxy-ethyl-cytidine
  modified_base 3: mod_base = OTHER; note = 2'-o-methoxy-ethyl-cytidine
  modified_base 4: mod_base = OTHER; note = 2'-o-methoxy-ethyl-thymine
  modified_base 5: mod_base = OTHER; note = 2'-o-methoxy-ethyl-thymine
  modified_base 16: mod_base = OTHER; note = 2'-o-methoxy-ethyl-guanosine
  modified_base 17: mod_base = OTHER; note = 2'-o-methoxy-ethyl-guanosine
  modified_base 18: mod_base = OTHER; note = 2'-o-methoxy-ethyl-cytidine
  modified_base 19: mod_base = OTHER; note = 2'-o-methoxy-ethyl-adenine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
accttcttcaaagaaggcaa

SEQ ID NO: 34 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-thymine
  modified_base 2: mod_base = OTHER; note = 2'-o-methoxy-ethyl-cytidine
  modified_base 3: mod_base = OTHER; note = 2'-o-methoxy-ethyl-adenine
  modified_base 4: mod_base = OTHER; note = 2'-o-methoxy-ethyl-adenine
  modified_base 5: mod_base = OTHER; note = 2'-o-methoxy-ethyl-adenine
  modified_base 16: mod_base = OTHER; note = 2'-o-methoxy-ethyl-thymine
  modified_base 17: mod_base = OTHER; note = 2'-o-methoxy-ethyl-cytidine
  modified_base 18: mod_base = OTHER; note = 2'-o-methoxy-ethyl-thymine
  modified_base 19: mod_base = OTHER; note = 2'-o-methoxy-ethyl-cytidine
  modified_base 20: mod_base = OTHER; note = 2'-o-methoxy-ethyl-thymine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
tcaaagaggctcttatctct

SEQ ID NO: 35 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-adenine
  modified_base 2: mod_base = OTHER; note = 2'-o-methoxy-ethyl-adenine
  modified_base 3: mod_base = OTHER; note = 2'-o-methoxy-ethyl-cytidine
  modified_base 4: mod_base = OTHER; note = 2'-o-methoxy-ethyl-thymine
  modified_base 5: mod_base = OTHER; note = 2'-o-methoxy-ethyl-adenine
  modified_base 16: mod_base = OTHER; note = 2'-o-methoxy-ethyl-adenine
  modified_base 17: mod_base = OTHER; note = 2'-o-methoxy-ethyl-guanosine
  modified_base 18: mod_base = OTHER; note = 2'-o-methoxy-ethyl-cytidine
  modified_base 19: mod_base = OTHER; note = 2'-o-methoxy-ethyl-adenine
  modified_base 20: mod_base = OTHER; note = 2'-o-methoxy-ethyl-cytidine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
aactatgttatgcatagcac

SEQ ID NO: 36 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-guanosine
  modified_base 2: mod_base = OTHER; note = 2'-o-methoxy-ethyl-thymine
  modified_base 3: mod_base = OTHER; note = 2'-o-methoxy-ethyl-guanosine
  modified_base 4: mod_base = OTHER; note = 2'-o-methoxy-ethyl-cytidine
  modified_base 5: mod_base = OTHER; note = 2'-o-methoxy-ethyl-cytidine
  modified_base 16: mod_base = OTHER; note = 2'-o-methoxy-ethyl-adenine
  modified_base 17: mod_base = OTHER; note = 2'-o-methoxy-ethyl-adenine
  modified_base 18: mod_base = OTHER; note = 2'-o-methoxy-ethyl-thymine
  modified_base 19: mod_base = OTHER; note = 2'-o-methoxy-ethyl-thymine
  modified_base 20: mod_base = OTHER; note = 2'-o-methoxy-ethyl-cytidine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
gtgcccagggatattaattc

SEQ ID NO: 37 - DNA, 18 bp, synthetic construct
  misc_feature 1..18: note = Description of sequence: antisense oligo
  source 1..18: mol_type = other DNA
  modified_base 1: mod_base = OTHER; note = 2'-o-methoxy-ethyl-adenine
  modified_base 2: mod_base = OTHER; note = 2'-o-methoxy-ethyl-cytidine
  modified_base 3: mod_base = OTHER; note = 2'-o-methoxy-ethyl-cytidine
  modified_base 4: mod_base = OTHER; note = 2'-o-methoxy-ethyl-thymine
  modified_base 5: mod_base = OTHER; note = 2'-o-methoxy-ethyl-adenine
  modified_base 14: mod_base = OTHER; note = 2'-o-methoxy-ethyl-adenine
  modified_base 15: mod_base = OTHER; note = 2'-o-methoxy-ethyl-guanosine
  modified_base 16: mod_base = OTHER; note = 2'-o-methoxy-ethyl-adenine
  modified_base 17: mod_base = OTHER; note = 2'-o-methoxy-ethyl-adenine
  modified_base 18: mod_base = OTHER; note = 2'-o-methoxy-ethyl-adenine
  modified_base 1..18: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18
acctaaggatatgagaaa

SEQ ID NO: 38 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-cytidine
  modified_base 2: mod_base = OTHER; note = 2'-o-methoxy-ethyl-thymine
  modified_base 3: mod_base = OTHER; note = 2'-o-methoxy-ethyl-guanosine
  modified_base 4: mod_base = OTHER; note = 2'-o-methoxy-ethyl-cytidine
  modified_base 5: mod_base = OTHER; note = 2'-o-methoxy-ethyl-thymine
  modified_base 16: mod_base = OTHER; note = 2'-o-methoxy-ethyl-thymine
  modified_base 17: mod_base = OTHER; note = 2'-o-methoxy-ethyl-thymine
  modified_base 18: mod_base = OTHER; note = 2'-o-methoxy-ethyl-cytidine
  modified_base 19: mod_base = OTHER; note = 2'-o-methoxy-ethyl-thymine
  modified_base 20: mod_base = OTHER; note = 2'-o-methoxy-ethyl-cytidine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
ctgctttacgaaaagttctc

SEQ ID NO: 39 - DNA, 18 bp, synthetic construct
  misc_feature 1..18: note = Description of sequence: antisense oligo
  source 1..18: mol_type = other DNA
  modified_base 1: mod_base = OTHER; note = 2'-o-methoxy-ethyl-cytidine
  modified_base 2: mod_base = OTHER; note = 2'-o-methoxy-ethyl-cytidine
  modified_base 3: mod_base = OTHER; note = 2'-o-methoxy-ethyl-thymine
  modified_base 4: mod_base = OTHER; note = 2'-o-methoxy-ethyl-cytidine
  modified_base 5: mod_base = OTHER; note = 2'-o-methoxy-ethyl-adenine
  modified_base 14: mod_base = OTHER; note = 2'-o-methoxy-ethyl-guanosine
  modified_base 15: mod_base = OTHER; note = 2'-o-methoxy-ethyl-thymine
  modified_base 16: mod_base = OTHER; note = 2'-o-methoxy-ethyl-adenine
  modified_base 17: mod_base = OTHER; note = 2'-o-methoxy-ethyl-cytidine
  modified_base 18: mod_base = OTHER; note = 2'-o-methoxy-ethyl-thymine
  modified_base 1..18: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18
cctcacagtatatgtact

SEQ ID NO: 40 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-cytidine
  modified_base 2: mod_base = OTHER; note = 2'-o-methoxy-ethyl-guanosine
  modified_base 3: mod_base = OTHER; note = 2'-o-methoxy-ethyl-guanosine
  modified_base 4: mod_base = OTHER; note = 2'-o-methoxy-ethyl-thymine
  modified_base 5: mod_base = OTHER; note = 2'-o-methoxy-ethyl-adenine
  modified_base 16: mod_base = OTHER; note = 2'-o-methoxy-ethyl-cytidine
  modified_base 17: mod_base = OTHER; note = 2'-o-methoxy-ethyl-thymine
  modified_base 18: mod_base = OTHER; note = 2'-o-methoxy-ethyl-thymine
  modified_base 19: mod_base = OTHER; note = 2'-o-methoxy-ethyl-guanosine
  modified_base 20: mod_base = OTHER; note = 2'-o-methoxy-ethyl-cytidine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
cggtaatttaaatttcttgc

SEQ ID NO: 41 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-cytidine
  modified_base 2: mod_base = OTHER; note = 2'-o-methoxy-ethyl-adenine
  modified_base 3: mod_base = OTHER; note = 2'-o-methoxy-ethyl-cytidine
  modified_base 4: mod_base = OTHER; note = 2'-o-methoxy-ethyl-thymine
  modified_base 5: mod_base = OTHER; note = 2'-o-methoxy-ethyl-adenine
  modified_base 16: mod_base = OTHER; note = 2'-o-methoxy-ethyl-guanosine
  modified_base 17: mod_base = OTHER; note = 2'-o-methoxy-ethyl-adenine
  modified_base 18: mod_base = OTHER; note = 2'-o-methoxy-ethyl-adenine
  modified_base 19: mod_base = OTHER; note = 2'-o-methoxy-ethyl-guanosine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
cactaggtgtcatatgaaga

SEQ ID NO: 42 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-cytidine
  modified_base 2: mod_base = OTHER; note = 2'-o-methoxy-ethyl-cytidine
  modified_base 3: mod_base = OTHER; note = 2'-o-methoxy-ethyl-adenine
  modified_base 4: mod_base = OTHER; note = 2'-o-methoxy-ethyl-adenine
  modified_base 5: mod_base = OTHER; note = 2'-o-methoxy-ethyl-thymine
  modified_base 16: mod_base = OTHER; note = 2'-o-methoxy-ethyl-adenine
  modified_base 17: mod_base = OTHER; note = 2'-o-methoxy-ethyl-thymine
  modified_base 18: mod_base = OTHER; note = 2'-o-methoxy-ethyl-adenine
  modified_base 19: mod_base = OTHER; note = 2'-o-methoxy-ethyl-guanosine
  modified_base 20: mod_base = OTHER; note = 2'-o-methoxy-ethyl-cytidine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
ccaattgatttctgaatagc

SEQ ID NO: 43 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-adenine
  modified_base 2: mod_base = OTHER; note = 2'-o-methoxy-ethyl-guanosine
  modified_base 3: mod_base = OTHER; note = 2'-o-methoxy-ethyl-thymine
  modified_base 4: mod_base = OTHER; note = 2'-o-methoxy-ethyl-cytidine
  modified_base 5: mod_base = OTHER; note = 2'-o-methoxy-ethyl-cytidine
  modified_base 16: mod_base = OTHER; note = 2'-o-methoxy-ethyl-adenine
  modified_base 17: mod_base = OTHER; note = 2'-o-methoxy-ethyl-adenine
  modified_base 18: mod_base = OTHER; note = 2'-o-methoxy-ethyl-adenine
  modified_base 19: mod_base = OTHER; note = 2'-o-methoxy-ethyl-adenine
  modified_base 20: mod_base = OTHER; note = 2'-o-methoxy-ethyl-guanosine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
agtcctcaggacaacaaaag

SEQ ID NO: 44 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-thymine
  modified_base 2: mod_base = OTHER; note = 2'-o-methoxy-ethyl-thymine
  modified_base 3: mod_base = OTHER; note = 2'-o-methoxy-ethyl-cytidine
  modified_base 4: mod_base = OTHER; note = 2'-o-methoxy-ethyl-adenine
  modified_base 5: mod_base = OTHER; note = 2'-o-methoxy-ethyl-adenine
  modified_base 16: mod_base = OTHER; note = 2'-o-methoxy-ethyl-thymine
  modified_base 17: mod_base = OTHER; note = 2'-o-methoxy-ethyl-cytidine
  modified_base 18: mod_base = OTHER; note = 2'-o-methoxy-ethyl-thymine
  modified_base 19: mod_base = OTHER; note = 2'-o-methoxy-ethyl-thymine
  modified_base 20: mod_base = OTHER; note = 2'-o-methoxy-ethyl-thymine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
ttcaaaaagacgatgtcttt

SEQ ID NO: 45 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-thymine
  modified_base 2: mod_base = OTHER; note = 2'-o-methoxy-ethyl-thymine
  modified_base 3: mod_base = OTHER; note = 2'-o-methoxy-ethyl-cytidine
  modified_base 4: mod_base = OTHER; note = 2'-o-methoxy-ethyl-cytidine
  modified_base 5: mod_base = OTHER; note = 2'-o-methoxy-ethyl-cytidine
  modified_base 16: mod_base = OTHER; note = 2'-o-methoxy-ethyl-cytidine
  modified_base 17: mod_base = OTHER; note = 2'-o-methoxy-ethyl-cytidine
  modified_base 18: mod_base = OTHER; note = 2'-o-methoxy-ethyl-thymine
  modified_base 19: mod_base = OTHER; note = 2'-o-methoxy-ethyl-thymine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
ttcccttgagggaaacctta

SEQ ID NO: 46 - DNA, 18 bp, synthetic construct
  misc_feature 1..18: note = Description of sequence: antisense oligo
  source 1..18: mol_type = other DNA
  modified_base 1: mod_base = OTHER; note = 2'-o-methoxy-ethyl-guanosine
  modified_base 2: mod_base = OTHER; note = 2'-o-methoxy-ethyl-cytidine
  modified_base 3: mod_base = OTHER; note = 2'-o-methoxy-ethyl-adenine
  modified_base 4: mod_base = OTHER; note = 2'-o-methoxy-ethyl-guanosine
  modified_base 5: mod_base = OTHER; note = 2'-o-methoxy-ethyl-guanosine
  modified_base 14: mod_base = OTHER; note = 2'-o-methoxy-ethyl-adenine
  modified_base 15: mod_base = OTHER; note = 2'-o-methoxy-ethyl-thymine
  modified_base 16: mod_base = OTHER; note = 2'-o-methoxy-ethyl-adenine
  modified_base 17: mod_base = OTHER; note = 2'-o-methoxy-ethyl-thymine
  modified_base 18: mod_base = OTHER; note = 2'-o-methoxy-ethyl-adenine
  modified_base 1..18: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18
gcaggacgtaactatata

SEQ ID NO: 47 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-cytidine
  modified_base 2: mod_base = OTHER; note = 2'-o-methoxy-ethyl-guanosine
  modified_base 3: mod_base = OTHER; note = 2'-o-methoxy-ethyl-cytidine
  modified_base 4: mod_base = OTHER; note = 2'-o-methoxy-ethyl-adenine
  modified_base 5: mod_base = OTHER; note = 2'-o-methoxy-ethyl-adenine
  modified_base 16: mod_base = OTHER; note = 2'-o-methoxy-ethyl-cytidine
  modified_base 17: mod_base = OTHER; note = 2'-o-methoxy-ethyl-adenine
  modified_base 18: mod_base = OTHER; note = 2'-o-methoxy-ethyl-adenine
  modified_base 19: mod_base = OTHER; note = 2'-o-methoxy-ethyl-cytidine
  modified_base 20: mod_base = OTHER; note = 2'-o-methoxy-ethyl-thymine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
cgcaaagataatgtacaact

SEQ ID NO: 48 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-adenine
  modified_base 2: mod_base = OTHER; note = 2'-o-methoxy-ethyl-adenine
  modified_base 3: mod_base = OTHER; note = 2'-o-methoxy-ethyl-adenine
  modified_base 4: mod_base = OTHER; note = 2'-o-methoxy-ethyl-adenine
  modified_base 5: mod_base = OTHER; note = 2'-o-methoxy-ethyl-thymine
  modified_base 16: mod_base = OTHER; note = 2'-o-methoxy-ethyl-guanosine
  modified_base 17: mod_base = OTHER; note = 2'-o-methoxy-ethyl-guanosine
  modified_base 18: mod_base = OTHER; note = 2'-o-methoxy-ethyl-adenine
  modified_base 19: mod_base = OTHER; note = 2'-o-methoxy-ethyl-cytidine
  modified_base 20: mod_base = OTHER; note = 2'-o-methoxy-ethyl-cytidine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
aaaatatttgttccaggacc

SEQ ID NO: 49 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-adenine
  modified_base 2: mod_base = OTHER; note = 2'-o-methoxy-ethyl-cytidine
  modified_base 3: mod_base = OTHER; note = 2'-o-methoxy-ethyl-cytidine
  modified_base 4: mod_base = OTHER; note = 2'-o-methoxy-ethyl-adenine
  modified_base 5: mod_base = OTHER; note = 2'-o-methoxy-ethyl-thymine
  modified_base 16: mod_base = OTHER; note = 2'-o-methoxy-ethyl-adenine
  modified_base 17: mod_base = OTHER; note = 2'-o-methoxy-ethyl-guanosine
  modified_base 18: mod_base = OTHER; note = 2'-o-methoxy-ethyl-cytidine
  modified_base 19: mod_base = OTHER; note = 2'-o-methoxy-ethyl-thymine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
accataaacagctgaagcta

SEQ ID NO: 50 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-guanosine
  modified_base 2: mod_base = OTHER; note = 2'-o-methoxy-ethyl-cytidine
  modified_base 3: mod_base = OTHER; note = 2'-o-methoxy-ethyl-thymine
  modified_base 4: mod_base = OTHER; note = 2'-o-methoxy-ethyl-guanosine
  modified_base 5: mod_base = OTHER; note = 2'-o-methoxy-ethyl-guanosine
  modified_base 16: mod_base = OTHER; note = 2'-o-methoxy-ethyl-thymine
  modified_base 17: mod_base = OTHER; note = 2'-o-methoxy-ethyl-adenine
  modified_base 18: mod_base = OTHER; note = 2'-o-methoxy-ethyl-adenine
  modified_base 19: mod_base = OTHER; note = 2'-o-methoxy-ethyl-guanosine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
gctggatagttctcttaaga

SEQ ID NO: 51 - RNA, 20 nt, synthetic construct
  misc_feature 1..20: note = Description of sequence: siRNA molecule: siELAVL2_15 mRNA
  source 1..20: mol_type = other RNA
ttagcatcctaagaaatatg

SEQ ID NO: 52 - RNA, 20 nt, synthetic construct
  misc_feature 1..20: note = Description of sequence: siRNA molecule: siELAVL2_16 mRNA
  source 1..20: mol_type = other RNA
tcagctaagggaagacattt

SEQ ID NO: 53 - RNA, 20 nt, synthetic construct
  misc_feature 1..20: note = Description of sequence: siRNA molecule: siELAVL2_18 mRNA
  source 1..20: mol_type = other RNA
tcagaaatcaattggcttag

SEQ ID NO: 54 - RNA, 20 nt, synthetic construct
  misc_feature 1..20: note = Description of sequence: siRNA molecule: siELAVL2_15 siRNA
  source 1..20: mol_type = other RNA
catatttcttaggatgctaa

SEQ ID NO: 55 - RNA, 20 nt, synthetic construct
  misc_feature 1..20: note = Description of sequence: siRNA molecule: siELAVL2_16 siRNA
  source 1..20: mol_type = other RNA
aaatgtcttcccttagctga

SEQ ID NO: 56 - RNA, 20 nt, synthetic construct
  misc_feature 1..20: note = Description of sequence: siRNA molecule: siELAVL2_18 siRNA
  source 1..20: mol_type = other RNA
ctaagccaattgatttctga

SEQ ID NO: 57 - RNA, 21 nt, synthetic construct
  misc_feature 1..21: note = Description of sequence: siRNA molecule: siELAVL2_15 formatted antisense (as) strand
  source 1..21: mol_type = other RNA
  modified_base 1: mod_base = um
  modified_base 2: mod_base = OTHER; note = 2'Fluoro-adenosine
  modified_base 3: mod_base = um
  modified_base 4: mod_base = OTHER; note = 2'Fluoro-adenosine
  modified_base 5: mod_base = OTHER; note = 2'Fluoro-uridine
  modified_base 6: mod_base = OTHER; note = 2'Fluoro-uridine
  modified_base 7: mod_base = um
  modified_base 8: mod_base = OTHER; note = 2'Fluoro-cytidine
  modified_base 9: mod_base = um
  modified_base 10: mod_base = OTHER; note = 2'Fluoro-uridine
  modified_base 11: mod_base = OTHER; note = 2'O-methyl-adenosine
  modified_base 12: mod_base = OTHER; note = 2'Fluoro-guanosine
  modified_base 13: mod_base = gm
  modified_base 14: mod_base = OTHER; note = 2'Fluoro-adenosine
  modified_base 15: mod_base = um
  modified_base 16: mod_base = OTHER; note = 2'Fluoro-guanosine
  modified_base 17: mod_base = cm
  modified_base 18: mod_base = um
  modified_base 19: mod_base = OTHER; note = 2'O-methyl-adenosine
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tatatttcttaggatgctaat

SEQ ID NO: 58 - RNA, 21 nt, synthetic construct
  misc_feature 1..21: note = Description of sequence: siRNA molecule: siELAVL2_16 formatted antisense (as) strand
  source 1..21: mol_type = other RNA
  modified_base 1: mod_base = um
  modified_base 2: mod_base = OTHER; note = 2'Fluoro-adenosine
  modified_base 3: mod_base = OTHER; note = 2'O-methyl-adenosine
  modified_base 4: mod_base = OTHER; note = 2'Fluoro-uridine
  modified_base 5: mod_base = OTHER; note = 2'Fluoro-guanosine
  modified_base 6: mod_base = OTHER; note = 2'Fluoro-uridine
  modified_base 7: mod_base = cm
  modified_base 8: mod_base = OTHER; note = 2'Fluoro-uridine
  modified_base 9: mod_base = um
  modified_base 10: mod_base = OTHER; note = 2'Fluoro-cytidine
  modified_base 11: mod_base = cm
  modified_base 12: mod_base = OTHER; note = 2'Fluoro-cytidine
  modified_base 13: mod_base = um
  modified_base 14: mod_base = OTHER; note = 2'Fluoro-uridine
  modified_base 15: mod_base = OTHER; note = 2'O-methyl-adenosine
  modified_base 16: mod_base = OTHER; note = 2'Fluoro-guanosine
  modified_base 17: mod_base = cm
  modified_base 18: mod_base = um
  modified_base 19: mod_base = gm
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
taatgtcttcccttagctgat

SEQ ID NO: 59 - RNA, 21 nt, synthetic construct
  misc_feature 1..21: note = Description of sequence: siRNA molecule: siELAVL2_18 formatted antisense (as) strand
  source 1..21: mol_type = other RNA
  modified_base 1: mod_base = um
  modified_base 2: mod_base = OTHER; note = 2'Fluoro-uridine
  modified_base 3: mod_base = OTHER; note = 2'O-methyl-adenosine
  modified_base 4: mod_base = OTHER; note = 2'Fluoro-adenosine
  modified_base 5: mod_base = OTHER; note = 2'Fluoro-guanosine
  modified_base 6: mod_base = OTHER; note = 2'Fluoro-cytidine
  modified_base 7: mod_base = cm
  modified_base 8: mod_base = OTHER; note = 2'Fluoro-adenosine
  modified_base 9: mod_base = OTHER; note = 2'O-methyl-adenosine
  modified_base 10: mod_base = OTHER; note = 2'Fluoro-uridine
  modified_base 11: mod_base = um
  modified_base 12: mod_base = OTHER; note = 2'Fluoro-guanosine
  modified_base 13: mod_base = OTHER; note = 2'O-methyl-adenosine
  modified_base 14: mod_base = OTHER; note = 2'Fluoro-uridine
  modified_base 15: mod_base = um
  modified_base 16: mod_base = OTHER; note = 2'Fluoro-uridine
  modified_base 17: mod_base = cm
  modified_base 18: mod_base = um
  modified_base 19: mod_base = gm
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
ttaagccaattgatttctgat

SEQ ID NO: 60 - RNA, 16 nt, synthetic construct
  misc_feature 1..16: note = Description of sequence: siRNA molecule: siELAVL2_15 formatted sense strand
  source 1..16: mol_type = other RNA
  modified_base 1: mod_base = cm
  modified_base 2: mod_base = OTHER; note = 2'O-methyl-adenosine
  modified_base 3: mod_base = um
  modified_base 4: mod_base = OTHER; note = 2'Fluoro-cytidine
  modified_base 5: mod_base = cm
  modified_base 6: mod_base = OTHER; note = 2'Fluoro-uridine
  modified_base 7: mod_base = OTHER; note = 2'O-methyl-adenosine
  modified_base 8: mod_base = OTHER; note = 2'Fluoro-adenosine
  modified_base 9: mod_base = gm
  modified_base 10: mod_base = OTHER; note = 2'Fluoro-adenosine
  modified_base 11: mod_base = OTHER; note = 2'O-methyl-adenosine
  modified_base 12: mod_base = OTHER; note = 2'O-methyl-adenosine
  modified_base 13: mod_base = um
  modified_base 14: mod_base = OTHER; note = 2'Fluoro-adenosine
  modified_base 15: mod_base = um
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
catcctaagaaatata

SEQ ID NO: 61 - RNA, 16 nt, synthetic construct
  misc_feature 1..16: note = Description of sequence: siRNA molecule: siELAVL2_16 formatted sense strand
  source 1..16: mol_type = other RNA
  modified_base 1: mod_base = cm
  modified_base 2: mod_base = um
  modified_base 3: mod_base = OTHER; note = 2'O-methyl-adenosine
  modified_base 4: mod_base = OTHER; note = 2'Fluoro-adenosine
  modified_base 5: mod_base = gm
  modified_base 6: mod_base = OTHER; note = 2'Fluoro-guanosine
  modified_base 7: mod_base = gm
  modified_base 8: mod_base = OTHER; note = 2'Fluoro-adenosine
  modified_base 9: mod_base = OTHER; note = 2'O-methyl-adenosine
  modified_base 10: mod_base = OTHER; note = 2'Fluoro-guanosine
  modified_base 11: mod_base = OTHER; note = 2'O-methyl-adenosine
  modified_base 12: mod_base = cm
  modified_base 13: mod_base = OTHER; note = 2'O-methyl-adenosine
  modified_base 14: mod_base = OTHER; note = 2'Fluoro-uridine
  modified_base 15: mod_base = um
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
ctaagggaagacatta

SEQ ID NO: 62 - RNA, 16 nt, synthetic construct
  misc_feature 1..16: note = Description of sequence: siRNA molecule: siELAVL2_18 formatted sense strand
  source 1..16: mol_type = other RNA
  modified_base 1: mod_base = OTHER; note = 2'O-methyl-adenosine
  modified_base 2: mod_base = OTHER; note = 2'O-methyl-adenosine
  modified_base 3: mod_base = OTHER; note = 2'O-methyl-adenosine
  modified_base 4: mod_base = OTHER; note = 2'Fluoro-uridine
  modified_base 5: mod_base = cm
  modified_base 6: mod_base = OTHER; note = 2'Fluoro-adenosine
  modified_base 7: mod_base = OTHER; note = 2'O-methyl-adenosine
  modified_base 8: mod_base = OTHER; note = 2'Fluoro-uridine
  modified_base 9: mod_base = um
  modified_base 10: mod_base = OTHER; note = 2'Fluoro-guanosine
  modified_base 11: mod_base = gm
  modified_base 12: mod_base = cm
  modified_base 13: mod_base = um
  modified_base 14: mod_base = OTHER; note = 2'Fluoro-uridine
  modified_base 15: mod_base = OTHER; note = 2'O-methyl-adenosine
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
aaatcaattggcttaa

SEQ ID NO: 63 - DNA, 20 bp, synthetic construct
  misc_feature 1..20: note = Description of sequence: siRNA molecule: hELAV-5 (w-2244) formatted sense strand
  source 1..20: mol_type = other DNA
  modified_base 1: mod_base = OTHER; note = 2'-o-methoxy-ethyl-cytidine
  modified_base 2: mod_base = OTHER; note = 2'-o-methoxy-ethyl-cytidine
  modified_base 3: mod_base = OTHER; note = 2'-o-methoxy-ethyl-adenine
  modified_base 4: mod_base = OTHER; note = 2'-o-methoxy-ethyl-adenine
  modified_base 5: mod_base = OTHER; note = 2'-o-methoxy-ethyl-adenine
  modified_base 16: mod_base = OTHER; note = 2'-o-methoxy-ethyl-uridine
  modified_base 17: mod_base = OTHER; note = 2'-o-methoxy-ethyl-uridine
  modified_base 18: mod_base = OTHER; note = 2'-o-methoxy-ethyl-adenine
  modified_base 19: mod_base = OTHER; note = 2'-o-methoxy-ethyl-uridine
  modified_base 20: mod_base = OTHER; note = 2'-o-methoxy-ethyl-uridine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  modified_base 16: mod_base = OTHER; note = t is uracil
  modified_base 17: mod_base = OTHER; note = t is uracil
  modified_base 19: mod_base = OTHER; note = t is uracil
  modified_base 20: mod_base = OTHER; note = t is uracil
ccaaaacctttgcatttatt

SEQ ID NO: 64 - DNA, 20 bp, synthetic construct
  misc_feature 1..20: note = Description of sequence: siRNA molecule: hELAV-7 (w-2246) formatted sense strand
  source 1..20: mol_type = other DNA
  modified_base 1: mod_base = OTHER; note = 2'-o-methoxy-ethyl-cytidine
  modified_base 2: mod_base = OTHER; note = 2'-o-methoxy-ethyl-guanosine
  modified_base 3: mod_base = OTHER; note = 2'-o-methoxy-ethyl-cytidine
  modified_base 4: mod_base = OTHER; note = 2'-o-methoxy-ethyl-cytidine
  modified_base 5: mod_base = OTHER; note = 2'-o-methoxy-ethyl-adenine
  modified_base 16: mod_base = OTHER; note = 2'-o-methoxy-ethyl-adenine
  modified_base 17: mod_base = OTHER; note = 2'-o-methoxy-ethyl-uridine
  modified_base 18: mod_base = OTHER; note = 2'-o-methoxy-ethyl-cytidine
  modified_base 19: mod_base = OTHER; note = 2'-o-methoxy-ethyl-adenine
  modified_base 20: mod_base = OTHER; note = 2'-o-methoxy-ethyl-uridine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  modified_base 17: mod_base = OTHER; note = t is uracil
  modified_base 20: mod_base = OTHER; note = t is uracil
cgccatggcagcctcatcat

SEQ ID NO: 65 - RNA, 20 nt, synthetic construct
  misc_feature 1..20: note = Description of sequence: siRNA molecule: siELAVL2_1 mRNA
  source 1..20: mol_type = other RNA
ttatacattagtaaatgtct

SEQ ID NO: 66 - RNA, 20 nt, synthetic construct
  misc_feature 1..20: note = Description of sequence: siRNA molecule: siELAVL2_2 mRNA
  source 1..20: mol_type = other RNA
tttaccatgtatgaaagaaa

SEQ ID NO: 67 - RNA, 20 nt, synthetic construct
  misc_feature 1..20: note = Description of sequence: siRNA molecule: siELAVL2_3 mRNA
  source 1..20: mol_type = other RNA
tttggtttataaatatgttt

SEQ ID NO: 68 - RNA, 20 nt, synthetic construct
  misc_feature 1..20: note = Description of sequence: siRNA molecule: siELAVL2_4 mRNA
  source 1..20: mol_type = other RNA
tggtttataaatatgtttgt

SEQ ID NO: 69 - RNA, 20 nt, synthetic construct
  misc_feature 1..20: note = Description of sequence: siRNA molecule: siELAVL2_5 mRNA
  source 1..20: mol_type = other RNA
tctgattagcatctagctta

SEQ ID NO: 70 - RNA, 20 nt, synthetic construct
  misc_feature 1..20: note = Description of sequence: siRNA molecule: siELAVL2_6 mRNA
  source 1..20: mol_type = other RNA
tgacggaatgaccagtttgg

SEQ ID NO: 71 - RNA, 20 nt, synthetic construct
  misc_feature 1..20: note = Description of sequence: siRNA molecule: siELAVL2_7 mRNA
  source 1..20: mol_type = other RNA
ttggtttataaatatgtttg

SEQ ID NO: 72 - RNA, 20 nt, synthetic construct
  misc_feature 1..20: note = Description of sequence: siRNA molecule: siELAVL2_8 mRNA
  source 1..20: mol_type = other RNA
ttcgtaaagcagtaccttgc

SEQ ID NO: 73 - RNA, 20 nt, synthetic construct
  misc_feature 1..20: note = Description of sequence: siRNA molecule: siELAVL2_9 mRNA
  source 1..20: mol_type = other RNA
cagctaagggaagacatttc

SEQ ID NO: 74 - RNA, 20 nt, synthetic construct
  misc_feature 1..20: note = Description of sequence: siRNA molecule: siELAVL2_10 mRNA
  source 1..20: mol_type = other RNA
cttcatatgacacctagtga

SEQ ID NO: 75 - RNA, 20 nt, synthetic construct
  misc_feature 1..20: note = Description of sequence: siRNA molecule: siELAVL2_11 mRNA
  source 1..20: mol_type = other RNA
tatgctattgaaatttaact

SEQ ID NO: 76 - RNA, 20 nt, synthetic construct
  misc_feature 1..20: note = Description of sequence: siRNA molecule: siELAVL2_12 mRNA
  source 1..20: mol_type = other RNA
tgtgtaatgtaaataagacc

SEQ ID NO: 77 - RNA, 20 nt, synthetic construct
  misc_feature 1..20: note = Description of sequence: siRNA molecule: siELAVL2_13 mRNA
  source 1..20: mol_type = other RNA
gctgttatgtctgtttaaga

SEQ ID NO: 78 - RNA, 20 nt, synthetic construct
  misc_feature 1..20: note = Description of sequence: siRNA molecule: siELAVL2_14 mRNA
  source 1..20: mol_type = other RNA
ggctacattataaacgctta

SEQ ID NO: 79 - RNA, 20 nt, synthetic construct
  misc_feature 1..20: note = Description of sequence: siRNA molecule: siELAVL2_17 mRNA
  source 1..20: mol_type = other RNA
gcagtattgtttgaagaatt

SEQ ID NO: 80 - RNA, 20 nt, synthetic construct
  misc_feature 1..20: note = Description of sequence: siRNA molecule: siELAVL2_19 mRNA
  source 1..20: mol_type = other RNA
ttggatttgtgactatgaca

SEQ ID NO: 81 - RNA, 20 nt, synthetic construct
  misc_feature 1..20: note = Description of sequence: siRNA molecule: siELAVL2_20 mRNA
  source 1..20: mol_type = other RNA
gctaacacctgagctttgtg

SEQ ID NO: 82 - RNA, 20 nt, synthetic construct
  misc_feature 1..20: note = Description of sequence: siRNA molecule: siELAVL2_1 siRNA
  source 1..20: mol_type = other RNA
agacatttactaatgtataa

SEQ ID NO: 83 - RNA, 20 nt, synthetic construct
  misc_feature 1..20: note = Description of sequence: siRNA molecule: siELAVL2_2 siRNA
  source 1..20: mol_type = other RNA
tttctttcatacatggtaaa

SEQ ID NO: 84 - RNA, 20 nt, synthetic construct
  misc_feature 1..20: note = Description of sequence: siRNA molecule: siELAVL2_3 siRNA
  source 1..20: mol_type = other RNA
aaacatatttataaaccaaa

SEQ ID NO: 85 - RNA, 20 nt, synthetic construct
  misc_feature 1..20: note = Description of sequence: siRNA molecule: siELAVL2_4 siRNA
  source 1..20: mol_type = other RNA
acaaacatatttataaacca

SEQ ID NO: 86 - RNA, 20 nt, synthetic construct
  misc_feature 1..20: note = Description of sequence: siRNA molecule: siELAVL2_5 siRNA
  source 1..20: mol_type = other RNA
taagctagatgctaatcaga

SEQ ID NO: 87 - RNA, 20 nt, synthetic construct
  misc_feature 1..20: note = Description of sequence: siRNA molecule: siELAVL2_6 siRNA
  source 1..20: mol_type = other RNA
ccaaactggtcattccgtca

SEQ ID NO: 88 - RNA, 20 nt, synthetic construct
  misc_feature 1..20: note = Description of sequence: siRNA molecule: siELAVL2_7 siRNA
  source 1..20: mol_type = other RNA
caaacatatttataaaccaa

SEQ ID NO: 89 - RNA, 20 nt, synthetic construct
  misc_feature 1..20: note = Description of sequence: siRNA molecule: siELAVL2_8 siRNA
  source 1..20: mol_type = other RNA
gcaaggtactgctttacgaa

SEQ ID NO: 90 - RNA, 20 nt, synthetic construct
  misc_feature 1..20: note = Description of sequence: siRNA molecule: siELAVL2_9 siRNA
  source 1..20: mol_type = other RNA
gaaatgtcttcccttagctg

SEQ ID NO: 91 - RNA, 20 nt, synthetic construct
  misc_feature 1..20: note = Description of sequence: siRNA molecule: siELAVL2_10 siRNA
  source 1..20: mol_type = other RNA
tcactaggtgtcatatgaag

SEQ ID NO: 92 - RNA, 20 nt, synthetic construct
  misc_feature 1..20: note = Description of sequence: siRNA molecule: siELAVL2_11 siRNA
  source 1..20: mol_type = other RNA
agttaaatttcaatagcata

SEQ ID NO: 93 - RNA, 20 nt, synthetic construct
  misc_feature 1..20: note = Description of sequence: siRNA molecule: siELAVL2_12 siRNA
  source 1..20: mol_type = other RNA
ggtcttatttacattacaca

SEQ ID NO: 94 - RNA, 20 nt, synthetic construct
  misc_feature 1..20: note = Description of sequence: siRNA molecule: siELAVL2_13 siRNA
  source 1..20: mol_type = other RNA
tcttaaacagacataacagc

SEQ ID NO: 95 - RNA, 20 nt, synthetic construct
  misc_feature 1..20: note = Description of sequence: siRNA molecule: siELAVL2_14 siRNA
  source 1..20: mol_type = other RNA
taagcgtttataatgtagcc

SEQ ID NO: 96 - RNA, 20 nt, synthetic construct
  misc_feature 1..20: note = Description of sequence: siRNA molecule: siELAVL2_17 siRNA
  source 1..20: mol_type = other RNA
aattcttcaaacaatactgc

SEQ ID NO: 97 - RNA, 20 nt, synthetic construct
  misc_feature 1..20: note = Description of sequence: siRNA molecule: siELAVL2_19 siRNA
  source 1..20: mol_type = other RNA
tgtcatagtcacaaatccaa

SEQ ID NO: 98 - RNA, 20 nt, synthetic construct
  misc_feature 1..20: note = Description of sequence: siRNA molecule: siELAVL2_20 siRNA
  source 1..20: mol_type = other RNA
cacaaagctcaggtgttagc

SEQ ID NO: 99 - RNA, 21 nt, synthetic construct
  misc_feature 1..21: note = Description of sequence: siRNA molecule: siELAVL2_1 formatted antisense (as) strand
  source 1..21: mol_type = other RNA
  modified_base 1: mod_base = um
  modified_base 2: mod_base = OTHER; note = 2'Fluoro-guanosine
  modified_base 3: mod_base = OTHER; note = 2'O-methyl-adenosine
  modified_base 4: mod_base = OTHER; note = 2'Fluoro-cytidine
  modified_base 5: mod_base = OTHER; note = 2'Fluoro-adenosine
  modified_base 6: mod_base = OTHER; note = 2'Fluoro-uridine
  modified_base 7: mod_base = um
  modified_base 8: mod_base = OTHER; note = 2'Fluoro-uridine
  modified_base 9: mod_base = OTHER; note = 2'O-methyl-adenosine
  modified_base 10: mod_base = OTHER; note = 2'Fluoro-cytidine
  modified_base 11: mod_base = um
  modified_base 12: mod_base = OTHER; note = 2'Fluoro-adenosine
  modified_base 13: mod_base = OTHER; note = 2'O-methyl-adenosine
  modified_base 14: mod_base = OTHER; note = 2'Fluoro-uridine
  modified_base 15: mod_base = gm
  modified_base 16: mod_base = OTHER; note = 2'Fluoro-uridine
  modified_base 17: mod_base = OTHER; note = 2'O-methyl-adenosine
  modified_base 18: mod_base = um
  modified_base 19: mod_base = OTHER; note = 2'O-methyl-adenosine
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tgacatttactaatgtataat

SEQ ID NO: 100 - RNA, 21 nt, synthetic construct
  misc_feature 1..21: note = Description of sequence: siRNA molecule: siELAVL2_2 formatted antisense (as) strand
  source 1..21: mol_type = other RNA
  modified_base 1: mod_base = um
  modified_base 2: mod_base = OTHER; note = 2'Fluoro-uridine
  modified_base 3: mod_base = um
  modified_base 4: mod_base = OTHER; note = 2'Fluoro-cytidine
  modified_base 5: mod_base = OTHER; note = 2'Fluoro-uridine
  modified_base 6: mod_base = OTHER; note = 2'Fluoro-uridine
  modified_base 7: mod_base = um
  modified_base 8: mod_base = OTHER; note = 2'Fluoro-cytidine
  modified_base 9: mod_base = OTHER; note = 2'O-methyl-adenosine
  modified_base 10: mod_base = OTHER; note = 2'Fluoro-uridine
  modified_base 11: mod_base = OTHER; note = 2'O-methyl-adenosine
  modified_base 12: mod_base = OTHER; note = 2'Fluoro-cytidine
  modified_base 13: mod_base = OTHER; note = 2'O-methyl-adenosine
  modified_base 14: mod_base = OTHER; note = 2'Fluoro-uridine
  modified_base 15: mod_base = gm
  modified_base 16: mod_base = OTHER; note = 2'Fluoro-guanosine
  modified_base 17: mod_base = um
  modified_base 18: mod_base = OTHER; note = 2'O-methyl-adenosine
  modified_base 19: mod_base = OTHER; note = 2'O-methyl-adenosine
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tttctttcatacatggtaaat

SEQ ID NO: 101 - RNA, 21 nt, synthetic construct
  misc_feature 1..21: note = Description of sequence: siRNA molecule: siELAVL2_3 formatted antisense (as) strand
  source 1..21: mol_type = other RNA
  modified_base 1: mod_base = um
  modified_base 2: mod_base = OTHER; note = 2'Fluoro-adenosine
  modified_base 3: mod_base = OTHER; note = 2'O-methyl-adenosine
  modified_base 4: mod_base = OTHER; note = 2'Fluoro-cytidine
  modified_base 5: mod_base = OTHER; note = 2'Fluoro-adenosine
  modified_base 6: mod_base = OTHER; note = 2'Fluoro-uridine
  modified_base 7: mod_base = OTHER; note = 2'O-methyl-adenosine
  modified_base 8: mod_base = OTHER; note = 2'Fluoro-uridine
  modified_base 9: mod_base = um
  modified_base 10: mod_base = OTHER; note = 2'Fluoro-uridine
  modified_base 11: mod_base = OTHER; note = 2'O-methyl-adenosine
  modified_base 12: mod_base = OTHER; note = 2'Fluoro-uridine
  modified_base 13: mod_base = OTHER; note = 2'O-methyl-adenosine
  modified_base 14: mod_base = OTHER; note = 2'Fluoro-adenosine
  modified_base 15: mod_base = OTHER; note = 2'O-methyl-adenosine
  modified_base 16: mod_base = OTHER; note = 2'Fluoro-cytidine
  modified_base 17: mod_base = cm
  modified_base 18: mod_base = OTHER; note = 2'O-methyl-adenosine
  modified_base 19: mod_base = OTHER; note = 2'O-methyl-adenosine
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
taacatatttataaaccaaat

SEQ ID NO: 102 - RNA, 21 nt, synthetic construct
  misc_feature 1..21: note = Description of sequence: siRNA molecule: siELAVL2_4 formatted antisense (as) strand
  source 1..21: mol_type = other RNA
  modified_base 1: mod_base = um
  modified_base 2: mod_base = OTHER; note = 2'Fluoro-cytidine
  modified_base 3: mod_base = OTHER; note = 2'O-methyl-adenosine
  modified_base 4: mod_base = OTHER; note = 2'Fluoro-adenosine
  modified_base 5: mod_base = OTHER; note = 2'Fluoro-adenosine
  modified_base 6: mod_base = OTHER; note = 2'Fluoro-cytidine
  modified_base 7: mod_base = OTHER; note = 2'O-methyl-adenosine
  modified_base 8: mod_base = OTHER; note = 2'Fluoro-uridine
  modified_base 9: mod_base = OTHER; note = 2'O-methyl-adenosine
  modified_base 10: mod_base = OTHER; note = 2'Fluoro-uridine
  modified_base 11: mod_base = um
  modified_base 12: mod_base = OTHER; note = 2'Fluoro-uridine
  modified_base 13: mod_base = OTHER; note = 2'O-methyl-adenosine
  modified_base 14: mod_base = OTHER; note = 2'Fluoro-uridine
  modified_base 15: mod_base = OTHER; note = 2'O-methyl-adenosine
  modified_base 16: mod_base = OTHER; note = 2'Fluoro-adenosine
  modified_base 17: mod_base = OTHER; note = 2'O-methyl-adenosine
  modified_base 18: mod_base = cm
  modified_base 19: mod_base = cm
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tcaaacatatttataaaccat

SEQ ID NO: 103 - RNA, 21 nt, synthetic construct
  misc_feature 1..21: note = Description of sequence: siRNA molecule: siELAVL2_5 formatted antisense (as) strand
  source 1..21: mol_type = other RNA
  modified_base 1: mod_base = um
  modified_base 2: mod_base = OTHER; note = 2'Fluoro-adenosine
  modified_base 3: mod_base = OTHER; note = 2'O-methyl-adenosine
  modified_base 4: mod_base = OTHER; note = 2'Fluoro-guanosine
  modified_base 5: mod_base = OTHER; note = 2'Fluoro-cytidine
  modified_base 6: mod_base = OTHER; note = 2'Fluoro-uridine
  modified_base 7: mod_base = OTHER; note = 2'O-methyl-adenosine
  modified_base 8: mod_base = OTHER; note = 2'Fluoro-guanosine
  modified_base 9: mod_base = OTHER; note = 2'O-methyl-adenosine
  modified_base 10: mod_base = OTHER; note = 2'Fluoro-uridine
  modified_base 11: mod_base = gm
  modified_base 12: mod_base = OTHER; note = 2'Fluoro-cytidine
  modified_base 13: mod_base = um
  modified_base 14: mod_base = OTHER; note = 2'Fluoro-adenosine
  modified_base 15: mod_base = OTHER; note = 2'O-methyl-adenosine
  modified_base 16: mod_base = OTHER; note = 2'Fluoro-uridine
  modified_base 17: mod_base = cm
  modified_base 18: mod_base = OTHER; note = 2'O-methyl-adenosine
  modified_base 19: mod_base = gm
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
taagctagatgctaatcagat

SEQ ID NO: 104 - RNA, 21 nt, synthetic construct
  misc_feature 1..21: note = Description of sequence: siRNA molecule: siELAVL2_6 formatted antisense (as) strand
  source 1..21: mol_type = other RNA
  modified_base 1: mod_base = um
  modified_base 2: mod_base = OTHER; note = 2'Fluoro-cytidine
  modified_base 3: mod_base = OTHER; note = 2'O-methyl-adenosine
  modified_base 4: mod_base = OTHER; note = 2'Fluoro-adenosine
  modified_base 5: mod_base = OTHER; note = 2'Fluoro-adenosine
  modified_base 6: mod_base = OTHER; note = 2'Fluoro-cytidine
  modified_base 7: mod_base = um
  modified_base 8: mod_base = OTHER; note = 2'Fluoro-guanosine
  modified_base 9: mod_base = gm
  modified_base 10: mod_base = OTHER; note = 2'Fluoro-uridine
  modified_base 11: mod_base = cm
  modified_base 12: mod_base = OTHER; note = 2'Fluoro-adenosine
  modified_base 13: mod_base = um
  modified_base 14: mod_base = OTHER; note = 2'Fluoro-uridine
  modified_base 15: mod_base = cm
  modified_base 16: mod_base = OTHER; note = 2'Fluoro-cytidine
  modified_base 17: mod_base = gm
  modified_base 18: mod_base = um
  modified_base 19: mod_base = cm
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tcaaactggtcattccgtcat

SEQ ID NO: 105 - RNA, 21 nt, synthetic construct
  misc_feature 1..21: note = Description of sequence: siRNA molecule: siELAVL2_7 formatted antisense (as) strand
  source 1..21: mol_type = other RNA
  modified_base 1: mod_base = um
  modified_base 2: mod_base = OTHER; note = 2'Fluoro-adenosine
  modified_base 3: mod_base = OTHER; note = 2'O-methyl-adenosine
  modified_base 4: mod_base = OTHER; note = 2'Fluoro-adenosine
  modified_base 5: mod_base = OTHER; note = 2'Fluoro-cytidine
  modified_base 6: mod_base = OTHER; note = 2'Fluoro-adenosine
  modified_base 7: mod_base = um
  modified_base 8: mod_base = OTHER; note = 2'Fluoro-adenosine
  modified_base 9: mod_base = um
  modified_base 10: mod_base = OTHER; note = 2'Fluoro-uridine
  modified_base 11: mod_base = um
  modified_base 12: mod_base = OTHER; note = 2'Fluoro-adenosine
  modified_base 13: mod_base = um
  modified_base 14: mod_base = OTHER; note = 2'Fluoro-adenosine
  modified_base 15: mod_base = OTHER; note = 2'O-methyl-adenosine
  modified_base 16: mod_base = OTHER; note = 2'Fluoro-adenosine
  modified_base 17: mod_base = cm
  modified_base 18: mod_base = cm
  modified_base 19: mod_base = OTHER; note = 2'O-methyl-adenosine
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
taaacatatttataaaccaat

SEQ ID NO: 106 - RNA, 21 nt, synthetic construct
  misc_feature 1..21: note = Description of sequence: siRNA molecule: siELAVL2_8 formatted antisense (as) strand
  source 1..21: mol_type = other RNA
  modified_base 1: mod_base = um
  modified_base 2: mod_base = OTHER; note = 2'Fluoro-cytidine
  modified_base 3: mod_base = OTHER; note = 2'O-methyl-adenosine
  modified_base 4: mod_base = OTHER; note = 2'Fluoro-adenosine
  modified_base 5: mod_base = OTHER; note = 2'Fluoro-guanosine
  modified_base 6: mod_base = OTHER; note = 2'Fluoro-guanosine
  modified_base 7: mod_base = um
  modified_base 8: mod_base = OTHER; note = 2'Fluoro-adenosine
  modified_base 9: mod_base = cm
  modified_base 10: mod_base = OTHER; note = 2'Fluoro-uridine
  modified_base 11: mod_base = gm
  modified_base 12: mod_base = OTHER; note = 2'Fluoro-cytidine
  modified_base 13: mod_base = um
  modified_base 14: mod_base = OTHER; note = 2'Fluoro-uridine
  modified_base 15: mod_base = um
  modified_base 16: mod_base = OTHER; note = 2'Fluoro-adenosine
  modified_base 17: mod_base = cm
  modified_base 18: mod_base = gm
  modified_base 19: mod_base = OTHER; note = 2'O-methyl-adenosine
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tcaaggtactgctttacgaat

SEQ ID NO: 107 - RNA, 21 nt, synthetic construct
  misc_feature 1..21: note = Description of sequence: siRNA molecule: siELAVL2_9 formatted antisense (as) strand
  source 1..21: mol_type = other RNA
  modified_base 1: mod_base = um
  modified_base 2: mod_base = OTHER; note = 2'Fluoro-adenosine
  modified_base 3: mod_base = OTHER; note = 2'O-methyl-adenosine
  modified_base 4: mod_base = OTHER; note = 2'Fluoro-adenosine
  modified_base 5: mod_base = OTHER; note = 2'Fluoro-uridine
  modified_base 6: mod_base = OTHER; note = 2'Fluoro-guanosine
  modified_base 7: mod_base = um
  modified_base 8: mod_base = OTHER; note = 2'Fluoro-cytidine
  modified_base 9: mod_base = um
  modified_base 10: mod_base = OTHER; note = 2'Fluoro-uridine
  modified_base 11: mod_base = cm
  modified_base 12: mod_base = OTHER; note = 2'Fluoro-cytidine
  modified_base 13: mod_base = cm
  modified_base 14: mod_base = OTHER; note = 2'Fluoro-uridine
  modified_base 15: mod_base = um
  modified_base 16: mod_base = OTHER; note = 2'Fluoro-adenosine
  modified_base 17: mod_base = gm
  modified_base 18: mod_base = cm
  modified_base 19: mod_base = um
  modified_base 20: mod_base = gm
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
taaatgtcttcccttagctgt

SEQ ID NO: 108 - RNA, 21 nt, synthetic construct
  misc_feature 1..21: note = Description of sequence: siRNA molecule: siELAVL2_10 formatted antisense (as) strand
  source 1..21: mol_type = other RNA
  modified_base 1: mod_base = um
  modified_base 2: mod_base = OTHER; note = 2'Fluoro-cytidine
  modified_base 3: mod_base = OTHER; note = 2'O-methyl-adenosine
  modified_base 4: mod_base = OTHER; note = 2'Fluoro-cytidine
  modified_base 5: mod_base = OTHER; note = 2'Fluoro-uridine
  modified_base 6: mod_base = OTHER; note = 2'Fluoro-adenosine
  modified_base 7: mod_base = gm
  modified_base 8: mod_base = OTHER; note = 2'Fluoro-guanosine
  modified_base 9: mod_base = um
  modified_base 10: mod_base = OTHER; note = 2'Fluoro-guanosine
  modified_base 11: mod_base = um
  modified_base 12: mod_base = OTHER; note = 2'Fluoro-cytidine
  modified_base 13: mod_base = OTHER; note = 2'O-methyl-adenosine
  modified_base 14: mod_base = OTHER; note = 2'Fluoro-uridine
  modified_base 15: mod_base = OTHER; note = 2'O-methyl-adenosine
  modified_base 16: mod_base = OTHER; note = 2'Fluoro-uridine
  modified_base 17: mod_base = gm
  modified_base 18: mod_base = OTHER; note = 2'O-methyl-adenosine
  modified_base 19: mod_base = OTHER; note = 2'O-methyl-adenosine
  modified_base 20: mod_base = gm
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tcactaggtgtcatatgaagt

SEQ ID NO: 109 - RNA, 21 nt, synthetic construct
  misc_feature 1..21: note = Description of sequence: siRNA molecule: siELAVL2_11 formatted antisense (as) strand
  source 1..21: mol_type = other RNA
  modified_base 1: mod_base = um
  modified_base 2: mod_base = OTHER; note = 2'Fluoro-guanosine
  modified_base 3: mod_base = um
  modified_base 4: mod_base = OTHER; note = 2'Fluoro-uridine
  modified_base 5: mod_base = OTHER; note = 2'Fluoro-adenosine
  modified_base 6: mod_base = OTHER; note = 2'Fluoro-adenosine
  modified_base 7: mod_base = OTHER; note = 2'O-methyl-adenosine
  modified_base 8: mod_base = OTHER; note = 2'Fluoro-uridine
  modified_base 9: mod_base = um
  modified_base 10: mod_base = OTHER; note = 2'Fluoro-uridine
  modified_base 11: mod_base = cm
  modified_base 12: mod_base = OTHER; note = 2'Fluoro-adenosine
  modified_base 13: mod_base = OTHER; note = 2'O-methyl-adenosine
  modified_base 14: mod_base = OTHER; note = 2'Fluoro-uridine
  modified_base 15: mod_base = OTHER; note = 2'O-methyl-adenosine
  modified_base 16: mod_base = OTHER; note = 2'Fluoro-guanosine
  modified_base 17: mod_base = cm
  modified_base 18: mod_base = OTHER; note = 2'O-methyl-adenosine
  modified_base 19: mod_base = um
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tgttaaatttcaatagcatat

SEQ ID NO: 110 - RNA, 21 nt, synthetic construct
  misc_feature 1..21: note = Description of sequence: siRNA molecule: siELAVL2_12 formatted antisense (as) strand
  source 1..21: mol_type = other RNA
  modified_base 1: mod_base = um
  modified_base 2: mod_base = OTHER; note = 2'Fluoro-guanosine
  modified_base 3: mod_base = um
  modified_base 4: mod_base = OTHER; note = 2'Fluoro-cytidine
  modified_base 5: mod_base = OTHER; note = 2'Fluoro-uridine
  modified_base 6: mod_base = OTHER; note = 2'Fluoro-uridine
  modified_base 7: mod_base = OTHER; note = 2'O-methyl-adenosine
  modified_base 8: mod_base = OTHER; note = 2'Fluoro-uridine
  modified_base 9: mod_base = um
  modified_base 10: mod_base = OTHER; note = 2'Fluoro-uridine
  modified_base 11: mod_base = OTHER; note = 2'O-methyl-adenosine
  modified_base 12: mod_base = OTHER; note = 2'Fluoro-cytidine
  modified_base 13: mod_base = OTHER; note = 2'O-methyl-adenosine
  modified_base 14: mod_base = OTHER; note = 2'Fluoro-uridine
  modified_base 15: mod_base = um
  modified_base 16: mod_base = OTHER; note = 2'Fluoro-adenosine
  modified_base 17: mod_base = cm
  modified_base 18: mod_base = OTHER; note = 2'O-methyl-adenosine
  modified_base 19: mod_base = cm
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tgtcttatttacattacacat

SEQ ID NO: 111 - RNA, 21 nt, synthetic construct
  misc_feature 1..21: note = Description of sequence: siRNA molecule: siELAVL2_13 formatted antisense (as) strand
  source 1..21: mol_type = other RNA
  modified_base 1: mod_base = um
  modified_base 2: mod_base = OTHER; note = 2'Fluoro-cytidine
  modified_base 3: mod_base = um
  modified_base 4: mod_base = OTHER; note = 2'Fluoro-uridine
  modified_base 5: mod_base = OTHER; note = 2'Fluoro-adenosine
  modified_base 6: mod_base = OTHER; note = 2'Fluoro-adenosine
  modified_base 7: mod_base = OTHER; note = 2'O-methyl-adenosine
  modified_base 8: mod_base = OTHER; note = 2'Fluoro-cytidine
  modified_base 9: mod_base = OTHER; note = 2'O-methyl-adenosine
  modified_base 10: mod_base = OTHER; note = 2'Fluoro-guanosine
  modified_base 11: mod_base = OTHER; note = 2'O-methyl-adenosine
  modified_base 12: mod_base = OTHER; note = 2'Fluoro-cytidine
  modified_base 13: mod_base = OTHER; note = 2'O-methyl-adenosine
  modified_base 14: mod_base = OTHER; note = 2'Fluoro-uridine
  modified_base 15: mod_base = OTHER; note = 2'O-methyl-adenosine
  modified_base 16: mod_base = OTHER; note = 2'Fluoro-adenosine
  modified_base 17: mod_base = cm
  modified_base 18: mod_base = OTHER; note = 2'O-methyl-adenosine
  modified_base 19: mod_base = gm
  modified_base 20: mod_base = cm
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tcttaaacagacataacagct

SEQ ID NO: 112 - RNA, 21 nt, synthetic construct
  misc_feature 1..21: note = Description of sequence: siRNA molecule: siELAVL2_14 formatted antisense (as) strand
  source 1..21: mol_type = other RNA
  modified_base 1: mod_base = um
  modified_base 2: mod_base = OTHER; note = 2'Fluoro-adenosine
  modified_base 3: mod_base = OTHER; note = 2'O-methyl-adenosine
  modified_base 4: mod_base = OTHER; note = 2'Fluoro-guanosine
  modified_base 5: mod_base = OTHER; note = 2'Fluoro-cytidine
  modified_base 6: mod_base = OTHER; note = 2'Fluoro-guanosine
  modified_base 7: mod_base = um
  modified_base 8: mod_base = OTHER; note = 2'Fluoro-uridine
  modified_base 9: mod_base = um
  modified_base 10: mod_base = OTHER; note = 2'Fluoro-adenosine
  modified_base 11: mod_base = um
  modified_base 12: mod_base = OTHER; note = 2'Fluoro-adenosine
  modified_base 13: mod_base = OTHER; note = 2'O-methyl-adenosine
  modified_base 14: mod_base = OTHER; note = 2'Fluoro-uridine
  modified_base 15: mod_base = gm
  modified_base 16: mod_base = OTHER; note = 2'Fluoro-uridine
  modified_base 17: mod_base = OTHER; note = 2'O-methyl-adenosine
  modified_base 18: mod_base = gm
  modified_base 19: mod_base = cm
  modified_base 20: mod_base = cm
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
taagcgtttataatgtagcct

SEQ ID NO: 113 - RNA, 21 nt, synthetic construct
  misc_feature 1..21: note = Description of sequence: siRNA molecule: siELAVL2_17 formatted antisense (as) strand
  source 1..21: mol_type = other RNA
  modified_base 1: mod_base = um
  modified_base 2: mod_base = OTHER; note = 2'Fluoro-adenosine
  modified_base 3: mod_base = um
  modified_base 4: mod_base = OTHER; note = 2'Fluoro-uridine
  modified_base 5: mod_base = OTHER; note = 2'Fluoro-cytidine
  modified_base 6: mod_base = OTHER; note = 2'Fluoro-uridine
  modified_base 7: mod_base = um
  modified_base 8: mod_base = OTHER; note = 2'Fluoro-cytidine
  modified_base 9: mod_base = OTHER; note = 2'O-methyl-adenosine
  modified_base 10: mod_base = OTHER; note = 2'Fluoro-adenosine
  modified_base 11: mod_base = OTHER; note = 2'O-methyl-adenosine
  modified_base 12: mod_base = OTHER; note = 2'Fluoro-cytidine
  modified_base 13: mod_base = OTHER; note = 2'O-methyl-adenosine
  modified_base 14: mod_base = OTHER; note = 2'Fluoro-adenosine
  modified_base 15: mod_base = um
  modified_base 16: mod_base = OTHER; note = 2'Fluoro-adenosine
  modified_base 17: mod_base = cm
  modified_base 18: mod_base = um
  modified_base 19: mod_base = gm
  modified_base 20: mod_base = cm
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tattcttcaaacaatactgct

SEQ ID NO: 114 - RNA, 21 nt, synthetic construct
  misc_feature 1..21: note = Description of sequence: siRNA molecule: siELAVL2_19 formatted antisense (as) strand
  source 1..21: mol_type = other RNA
  modified_base 1: mod_base = um
  modified_base 2: mod_base = OTHER; note = 2'Fluoro-guanosine
  modified_base 3: mod_base = um
  modified_base 4: mod_base = OTHER; note = 2'Fluoro-cytidine
  modified_base 5: mod_base = OTHER; note = 2'Fluoro-adenosine
  modified_base 6: mod_base = OTHER; note = 2'Fluoro-uridine
  modified_base 7: mod_base = OTHER; note = 2'O-methyl-adenosine
  modified_base 8: mod_base = OTHER; note = 2'Fluoro-guanosine
  modified_base 9: mod_base = um
  modified_base 10: mod_base = OTHER; note = 2'Fluoro-cytidine
  modified_base 11: mod_base = OTHER; note = 2'O-methyl-adenosine
  modified_base 12: mod_base = OTHER; note = 2'Fluoro-cytidine
  modified_base 13: mod_base = OTHER; note = 2'O-methyl-adenosine
  modified_base 14: mod_base = OTHER; note = 2'Fluoro-adenosine
  modified_base 15: mod_base = OTHER; note = 2'O-methyl-adenosine
  modified_base 16: mod_base = OTHER; note = 2'Fluoro-uridine
  modified_base 17: mod_base = cm
  modified_base 18: mod_base = cm
  modified_base 19: mod_base = OTHER; note = 2'O-methyl-adenosine
  modified_base 20: mod_base = OTHER; note = 2'O-methyl-adenosine
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tgtcatagtcacaaatccaat

SEQ ID NO: 115 - RNA, 21 nt, synthetic construct
  misc_feature 1..21: note = Description of sequence: siRNA molecule: siELAVL2_20 formatted antisense (as) strand
  source 1..21: mol_type = other RNA
  modified_base 1: mod_base = um
  modified_base 2: mod_base = OTHER; note = 2'Fluoro-adenosine
  modified_base 3: mod_base = cm
  modified_base 4: mod_base = OTHER; note = 2'Fluoro-adenosine
  modified_base 5: mod_base = OTHER; note = 2'Fluoro-adenosine
  modified_base 6: mod_base = OTHER; note = 2'Fluoro-adenosine
  modified_base 7: mod_base = gm
  modified_base 8: mod_base = OTHER; note = 2'Fluoro-cytidine
  modified_base 9: mod_base = um
  modified_base 10: mod_base = OTHER; note = 2'Fluoro-cytidine
  modified_base 11: mod_base = OTHER; note = 2'O-methyl-adenosine
  modified_base 12: mod_base = OTHER; note = 2'Fluoro-guanosine
  modified_base 13: mod_base = gm
  modified_base 14: mod_base = OTHER; note = 2'Fluoro-uridine
  modified_base 15: mod_base = gm
  modified_base 16: mod_base = OTHER; note = 2'Fluoro-uridine
  modified_base 17: mod_base = um
  modified_base 18: mod_base = OTHER; note = 2'O-methyl-adenosine
  modified_base 19: mod_base = gm
  modified_base 20: mod_base = cm
  modified_base 21: mod_base = OTHER; note = 2'Fluoro-uridine
  modified_base 1..3: mod_base = OTHER; note = Phosphorothioate linkage between positions 1^2, 2^3
  modified_base 14..21: mod_base = OTHER; note = Phosphorothioate linkage between positions 14^15, 15^16, 16^17, 17^18, 18^19, 19^20, 20^21
tacaaagctcaggtgttagct

SEQ ID NO: 116 - RNA, 16 nt, synthetic construct
  misc_feature 1..16: note = Description of sequence: siRNA molecule: siELAVL2_1 formatted sense strand
  source 1..16: mol_type = other RNA
  modified_base 1: mod_base = OTHER; note = 2'O-methyl-adenosine
  modified_base 2: mod_base = cm
  modified_base 3: mod_base = OTHER; note = 2'O-methyl-adenosine
  modified_base 4: mod_base = OTHER; note = 2'Fluoro-uridine
  modified_base 5: mod_base = um
  modified_base 6: mod_base = OTHER; note = 2'Fluoro-adenosine
  modified_base 7: mod_base = gm
  modified_base 8: mod_base = OTHER; note = 2'Fluoro-uridine
  modified_base 9: mod_base = OTHER; note = 2'O-methyl-adenosine
  modified_base 10: mod_base = OTHER; note = 2'Fluoro-adenosine
  modified_base 11: mod_base = OTHER; note = 2'O-methyl-adenosine
  modified_base 12: mod_base = um
  modified_base 13: mod_base = gm
  modified_base 14: mod_base = OTHER; note = 2'Fluoro-uridine
  modified_base 15: mod_base = cm
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
acattagtaaatgtca

SEQ ID NO: 117 - RNA, 16 nt, synthetic construct
  misc_feature 1..16: note = Description of sequence: siRNA molecule: siELAVL2_2 formatted sense strand
  source 1..16: mol_type = other RNA
  modified_base 1: mod_base = cm
  modified_base 2: mod_base = cm
  modified_base 3: mod_base = OTHER; note = 2'O-methyl-adenosine
  modified_base 4: mod_base = OTHER; note = 2'Fluoro-uridine
  modified_base 5: mod_base = gm
  modified_base 6: mod_base = OTHER; note = 2'Fluoro-uridine
  modified_base 7: mod_base = OTHER; note = 2'O-methyl-adenosine
  modified_base 8: mod_base = OTHER; note = 2'Fluoro-uridine
  modified_base 9: mod_base = gm
  modified_base 10: mod_base = OTHER; note = 2'Fluoro-adenosine
  modified_base 11: mod_base = OTHER; note = 2'O-methyl-adenosine
  modified_base 12: mod_base = OTHER; note = 2'O-methyl-adenosine
  modified_base 13: mod_base = gm
  modified_base 14: mod_base = OTHER; note = 2'Fluoro-adenosine
  modified_base 15: mod_base = OTHER; note = 2'O-methyl-adenosine
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
ccatgtatgaaagaaa

SEQ ID NO: 118 - RNA, 16 nt, synthetic construct
  misc_feature 1..16: note = Description of sequence: siRNA molecule: siELAVL2_3 formatted sense strand
  source 1..16: mol_type = other RNA
  modified_base 1: mod_base = gm
  modified_base 2: mod_base = um
  modified_base 3: mod_base = um
  modified_base 4: mod_base = OTHER; note = 2'Fluoro-uridine
  modified_base 5: mod_base = OTHER; note = 2'O-methyl-adenosine
  modified_base 6: mod_base = OTHER; note = 2'Fluoro-uridine
  modified_base 7: mod_base = OTHER; note = 2'O-methyl-adenosine
  modified_base 8: mod_base = OTHER; note = 2'Fluoro-adenosine
  modified_base 9: mod_base = OTHER; note = 2'O-methyl-adenosine
  modified_base 10: mod_base = OTHER; note = 2'Fluoro-uridine
  modified_base 11: mod_base = OTHER; note = 2'O-methyl-adenosine
  modified_base 12: mod_base = um
  modified_base 13: mod_base = gm
  modified_base 14: mod_base = OTHER; note = 2'Fluoro-uridine
  modified_base 15: mod_base = um
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
gtttataaatatgtta

SEQ ID NO: 119 - RNA, 16 nt, synthetic construct
  misc_feature 1..16: note = Description of sequence: siRNA molecule: siELAVL2_4 formatted sense strand
  source 1..16: mol_type = other RNA
  modified_base 1: mod_base = um
  modified_base 2: mod_base = um
  modified_base 3: mod_base = OTHER; note = 2'O-methyl-adenosine
  modified_base 4: mod_base = OTHER; note = 2'Fluoro-uridine
  modified_base 5: mod_base = OTHER; note = 2'O-methyl-adenosine
  modified_base 6: mod_base = OTHER; note = 2'Fluoro-adenosine
  modified_base 7: mod_base = OTHER; note = 2'O-methyl-adenosine
  modified_base 8: mod_base = OTHER; note = 2'Fluoro-uridine
  modified_base 9: mod_base = OTHER; note = 2'O-methyl-adenosine
  modified_base 10: mod_base = OTHER; note = 2'Fluoro-uridine
  modified_base 11: mod_base = gm
  modified_base 12: mod_base = um
  modified_base 13: mod_base = um
  modified_base 14: mod_base = OTHER; note = 2'Fluoro-uridine
  modified_base 15: mod_base = gm
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
ttataaatatgtttga

SEQ ID NO: 120 - RNA, 16 nt, synthetic construct
  misc_feature 1..16: note = Description of sequence: siRNA molecule: siELAVL2_5 formatted sense strand
  source 1..16: mol_type = other RNA
  modified_base 1: mod_base = OTHER; note = 2'O-methyl-adenosine
  modified_base 2: mod_base = um
  modified_base 3: mod_base = um
  modified_base 4: mod_base = OTHER; note = 2'Fluoro-adenosine
  modified_base 5: mod_base = gm
  modified_base 6: mod_base = OTHER; note = 2'Fluoro-cytidine
  modified_base 7: mod_base = OTHER; note = 2'O-methyl-adenosine
  modified_base 8: mod_base = OTHER; note = 2'Fluoro-uridine
  modified_base 9: mod_base = cm
  modified_base 10: mod_base = OTHER; note = 2'Fluoro-uridine
  modified_base 11: mod_base = OTHER; note = 2'O-methyl-adenosine
  modified_base 12: mod_base = gm
  modified_base 13: mod_base = cm
  modified_base 14: mod_base = OTHER; note = 2'Fluoro-uridine
  modified_base 15: mod_base = um
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
attagcatctagctta

SEQ ID NO: 121 - RNA, 16 nt, synthetic construct
  misc_feature 1..16: note = Description of sequence: siRNA molecule: siELAVL2_6 formatted sense strand
  source 1..16: mol_type = other RNA
  modified_base 1: mod_base = gm
  modified_base 2: mod_base = gm
  modified_base 3: mod_base = OTHER; note = 2'O-methyl-adenosine
  modified_base 4: mod_base = OTHER; note = 2'Fluoro-adenosine
  modified_base 5: mod_base = um
  modified_base 6: mod_base = OTHER; note = 2'Fluoro-guanosine
  modified_base 7: mod_base = OTHER; note = 2'O-methyl-adenosine
  modified_base 8: mod_base = OTHER; note = 2'Fluoro-cytidine
  modified_base 9: mod_base = cm
  modified_base 10: mod_base = OTHER; note = 2'Fluoro-adenosine
  modified_base 11: mod_base = gm
  modified_base 12: mod_base = um
  modified_base 13: mod_base = um
  modified_base 14: mod_base = OTHER; note = 2'Fluoro-uridine
  modified_base 15: mod_base = gm
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
ggaatgaccagtttga

SEQ ID NO: 122 - RNA, 16 nt, synthetic construct
  misc_feature 1..16: note = Description of sequence: siRNA molecule: siELAVL2_7 formatted sense strand
  source 1..16: mol_type = other RNA
  modified_base 1: mod_base = um
  modified_base 2: mod_base = um
  modified_base 3: mod_base = um
  modified_base 4: mod_base = OTHER; note = 2'Fluoro-adenosine
  modified_base 5: mod_base = um
  modified_base 6: mod_base = OTHER; note = 2'Fluoro-adenosine
  modified_base 7: mod_base = OTHER; note = 2'O-methyl-adenosine
  modified_base 8: mod_base = OTHER; note = 2'Fluoro-adenosine
  modified_base 9: mod_base = um
  modified_base 10: mod_base = OTHER; note = 2'Fluoro-adenosine
  modified_base 11: mod_base = um
  modified_base 12: mod_base = gm
  modified_base 13: mod_base = um
  modified_base 14: mod_base = OTHER; note = 2'Fluoro-uridine
  modified_base 15: mod_base = um
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
tttataaatatgttta

SEQ ID NO: 123 - RNA, 16 nt, synthetic construct
  misc_feature 1..16: note = Description of sequence: siRNA molecule: siELAVL2_8 formatted sense strand
  source 1..16: mol_type = other RNA
  modified_base 1: mod_base = um
  modified_base 2: mod_base = OTHER; note = 2'O-methyl-adenosine
  modified_base 3: mod_base = OTHER; note = 2'O-methyl-adenosine
  modified_base 4: mod_base = OTHER; note = 2'Fluoro-adenosine
  modified_base 5: mod_base = gm
  modified_base 6: mod_base = OTHER; note = 2'Fluoro-cytidine
  modified_base 7: mod_base = OTHER; note = 2'O-methyl-adenosine
  modified_base 8: mod_base = OTHER; note = 2'Fluoro-guanosine
  modified_base 9: mod_base = um
  modified_base 10: mod_base = OTHER; note = 2'Fluoro-adenosine
  modified_base 11: mod_base = cm
  modified_base 12: mod_base = cm
  modified_base 13: mod_base = um
  modified_base 14: mod_base = OTHER; note = 2'Fluoro-uridine
  modified_base 15: mod_base = gm
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
taaagcagtaccttga

SEQ ID NO: 124 - RNA, 16 nt, synthetic construct
  misc_feature 1..16: note = Description of sequence: siRNA molecule: siELAVL2_9 formatted sense strand
  source 1..16: mol_type = other RNA
  modified_base 1: mod_base = um
  modified_base 2: mod_base = OTHER; note = 2'O-methyl-adenosine
  modified_base 3: mod_base = OTHER; note = 2'O-methyl-adenosine
  modified_base 4: mod_base = OTHER; note = 2'Fluoro-guanosine
  modified_base 5: mod_base = gm
  modified_base 6: mod_base = OTHER; note = 2'Fluoro-guanosine
  modified_base 7: mod_base = OTHER; note = 2'O-methyl-adenosine
  modified_base 8: mod_base = OTHER; note = 2'Fluoro-adenosine
  modified_base 9: mod_base = gm
  modified_base 10: mod_base = OTHER; note = 2'Fluoro-adenosine
  modified_base 11: mod_base = cm
  modified_base 12: mod_base = OTHER; note = 2'O-methyl-adenosine
  modified_base 13: mod_base = um
  modified_base 14: mod_base = OTHER; note = 2'Fluoro-uridine
  modified_base 15: mod_base = um
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
taagggaagacattta

SEQ ID NO: 125 - RNA, 16 nt, synthetic construct
  misc_feature 1..16: note = Description of sequence: siRNA molecule: siELAVL2_10 formatted sense strand
  source 1..16: mol_type = other RNA
  modified_base 1: mod_base = OTHER; note = 2'O-methyl-adenosine
  modified_base 2: mod_base = um
  modified_base 3: mod_base = OTHER; note = 2'O-methyl-adenosine
  modified_base 4: mod_base = OTHER; note = 2'Fluoro-uridine
  modified_base 5: mod_base = gm
  modified_base 6: mod_base = OTHER; note = 2'Fluoro-adenosine
  modified_base 7: mod_base = cm
  modified_base 8: mod_base = OTHER; note = 2'Fluoro-adenosine
  modified_base 9: mod_base = cm
  modified_base 10: mod_base = OTHER; note = 2'Fluoro-cytidine
  modified_base 11: mod_base = um
  modified_base 12: mod_base = OTHER; note = 2'O-methyl-adenosine
  modified_base 13: mod_base = gm
  modified_base 14: mod_base = OTHER; note = 2'Fluoro-uridine
  modified_base 15: mod_base = gm
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
atatgacacctagtga

SEQ ID NO: 126 - RNA, 16 nt, synthetic construct
  misc_feature 1..16: note = Description of sequence: siRNA molecule: siELAVL2_11 formatted sense strand
  source 1..16: mol_type = other RNA
  modified_base 1: mod_base = cm
  modified_base 2: mod_base = um
  modified_base 3: mod_base = OTHER; note = 2'O-methyl-adenosine
  modified_base 4: mod_base = OTHER; note = 2'Fluoro-uridine
  modified_base 5: mod_base = um
  modified_base 6: mod_base = OTHER; note = 2'Fluoro-guanosine
  modified_base 7: mod_base = OTHER; note = 2'O-methyl-adenosine
  modified_base 8: mod_base = OTHER; note = 2'Fluoro-adenosine
  modified_base 9: mod_base = OTHER; note = 2'O-methyl-adenosine
  modified_base 10: mod_base = OTHER; note = 2'Fluoro-uridine
  modified_base 11: mod_base = um
  modified_base 12: mod_base = um
  modified_base 13: mod_base = OTHER; note = 2'O-methyl-adenosine
  modified_base 14: mod_base = OTHER; note = 2'Fluoro-adenosine
  modified_base 15: mod_base = cm
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
ctattgaaatttaaca

SEQ ID NO: 127 - RNA, 16 nt, synthetic construct
  misc_feature 1..16: note = Description of sequence: siRNA molecule: siELAVL2_12 formatted sense strand
  source 1..16: mol_type = other RNA
  modified_base 1: mod_base = um
  modified_base 2: mod_base = OTHER; note = 2'O-methyl-adenosine
  modified_base 3: mod_base = OTHER; note = 2'O-methyl-adenosine
  modified_base 4: mod_base = OTHER; note = 2'Fluoro-uridine
  modified_base 5: mod_base = gm
  modified_base 6: mod_base = OTHER; note = 2'Fluoro-uridine
  modified_base 7: mod_base = OTHER; note = 2'O-methyl-adenosine
  modified_base 8: mod_base = OTHER; note = 2'Fluoro-adenosine
  modified_base 9: mod_base = OTHER; note = 2'O-methyl-adenosine
  modified_base 10: mod_base = OTHER; note = 2'Fluoro-uridine
  modified_base 11: mod_base = OTHER; note = 2'O-methyl-adenosine
  modified_base 12: mod_base = OTHER; note = 2'O-methyl-adenosine
  modified_base 13: mod_base = gm
  modified_base 14: mod_base = OTHER; note = 2'Fluoro-adenosine
  modified_base 15: mod_base = cm
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
taatgtaaataagaca

SEQ ID NO: 128 - RNA, 16 nt, synthetic construct
  misc_feature 1..16: note = Description of sequence: siRNA molecule: siELAVL2_13 formatted sense strand
  source 1..16: mol_type = other RNA
  modified_base 1: mod_base = um
  modified_base 2: mod_base = um
  modified_base 3: mod_base = OTHER; note = 2'O-methyl-adenosine
  modified_base 4: mod_base = OTHER; note = 2'Fluoro-uridine
  modified_base 5: mod_base = gm
  modified_base 6: mod_base = OTHER; note = 2'Fluoro-uridine
  modified_base 7: mod_base = cm
  modified_base 8: mod_base = OTHER; note = 2'Fluoro-uridine
  modified_base 9: mod_base = gm
  modified_base 10: mod_base = OTHER; note = 2'Fluoro-uridine
  modified_base 11: mod_base = um
  modified_base 12: mod_base = um
  modified_base 13: mod_base = OTHER; note = 2'O-methyl-adenosine
  modified_base 14: mod_base = OTHER; note = 2'Fluoro-adenosine
  modified_base 15: mod_base = gm
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
ttatgtctgtttaaga

SEQ ID NO: 129 - RNA, 16 nt, synthetic construct
  misc_feature 1..16: note = Description of sequence: siRNA molecule: siELAVL2_14 formatted sense strand
  source 1..16: mol_type = other RNA
  modified_base 1: mod_base = OTHER; note = 2'O-methyl-adenosine
  modified_base 2: mod_base = cm
  modified_base 3: mod_base = OTHER; note = 2'O-methyl-adenosine
  modified_base 4: mod_base = OTHER; note = 2'Fluoro-uridine
  modified_base 5: mod_base = um
  modified_base 6: mod_base = OTHER; note = 2'Fluoro-adenosine
  modified_base 7: mod_base = um
  modified_base 8: mod_base = OTHER; note = 2'Fluoro-adenosine
  modified_base 9: mod_base = OTHER; note = 2'O-methyl-adenosine
  modified_base 10: mod_base = OTHER; note = 2'Fluoro-adenosine
  modified_base 11: mod_base = cm
  modified_base 12: mod_base = gm
  modified_base 13: mod_base = cm
  modified_base 14: mod_base = OTHER; note = 2'Fluoro-uridine
  modified_base 15: mod_base = um
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
acattataaacgctta

SEQ ID NO: 130 - RNA, 16 nt, synthetic construct
  misc_feature 1..16: note = Description of sequence: siRNA molecule: siELAVL2_17 formatted sense strand
  source 1..16: mol_type = other RNA
  modified_base 1: mod_base = um
  modified_base 2: mod_base = OTHER; note = 2'O-methyl-adenosine
  modified_base 3: mod_base = um
  modified_base 4: mod_base = OTHER; note = 2'Fluoro-uridine
  modified_base 5: mod_base = gm
  modified_base 6: mod_base = OTHER; note = 2'Fluoro-uridine
  modified_base 7: mod_base = um
  modified_base 8: mod_base = OTHER; note = 2'Fluoro-uridine
  modified_base 9: mod_base = gm
  modified_base 10: mod_base = OTHER; note = 2'Fluoro-adenosine
  modified_base 11: mod_base = OTHER; note = 2'O-methyl-adenosine
  modified_base 12: mod_base = gm
  modified_base 13: mod_base = OTHER; note = 2'O-methyl-adenosine
  modified_base 14: mod_base = OTHER; note = 2'Fluoro-adenosine
  modified_base 15: mod_base = um
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
tattgtttgaagaata

SEQ ID NO: 131 - RNA, 16 nt, synthetic construct
  misc_feature 1..16: note = Description of sequence: siRNA molecule: siELAVL2_19 formatted sense strand
  source 1..16: mol_type = other RNA
  modified_base 1: mod_base = OTHER; note = 2'O-methyl-adenosine
  modified_base 2: mod_base = um
  modified_base 3: mod_base = um
  modified_base 4: mod_base = OTHER; note = 2'Fluoro-uridine
  modified_base 5: mod_base = gm
  modified_base 6: mod_base = OTHER; note = 2'Fluoro-uridine
  modified_base 7: mod_base = gm
  modified_base 8: mod_base = OTHER; note = 2'Fluoro-adenosine
  modified_base 9: mod_base = cm
  modified_base 10: mod_base = OTHER; note = 2'Fluoro-uridine
  modified_base 11: mod_base = OTHER; note = 2'O-methyl-adenosine
  modified_base 12: mod_base = um
  modified_base 13: mod_base = gm
  modified_base 14: mod_base = OTHER; note = 2'Fluoro-adenosine
  modified_base 15: mod_base = cm
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
atttgtgactatgaca

SEQ ID NO: 132 - RNA, 16 nt, synthetic construct
  misc_feature 1..16: note = Description of sequence: siRNA molecule: siELAVL2_20 formatted sense strand
  source 1..16: mol_type = other RNA
  modified_base 1: mod_base = OTHER; note = 2'O-methyl-adenosine
  modified_base 2: mod_base = cm
  modified_base 3: mod_base = OTHER; note = 2'O-methyl-adenosine
  modified_base 4: mod_base = OTHER; note = 2'Fluoro-cytidine
  modified_base 5: mod_base = cm
  modified_base 6: mod_base = OTHER; note = 2'Fluoro-uridine
  modified_base 7: mod_base = gm
  modified_base 8: mod_base = OTHER; note = 2'Fluoro-adenosine
  modified_base 9: mod_base = gm
  modified_base 10: mod_base = OTHER; note = 2'Fluoro-cytidine
  modified_base 11: mod_base = um
  modified_base 12: mod_base = um
  modified_base 13: mod_base = um
  modified_base 14: mod_base = OTHER; note = 2'Fluoro-guanosine
  modified_base 15: mod_base = um
  modified_base 16: mod_base = OTHER; note = 2'O-methyl-adenosine
  modified_base 1..3: mod_base = OTHER; note = Phosphorothioate linkage between positions 1^2, 2^3
  modified_base 14..16: mod_base = OTHER; note = Phosphorothioate linkage between positions 14^15, 15^16
  misc_feature 16: note = 3' TEG-linked cholesterol conjugate
acacctgagctttgta

SEQ ID NO: 133 - DNA, 19 bp, synthetic construct
  misc_feature 1..19: note = Description of sequence: antisense oligo
  source 1..19: mol_type = other DNA
  modified_base 1: mod_base = OTHER; note = 2'-o-methoxy-ethyl-adenine
  modified_base 2: mod_base = OTHER; note = 2'-o-methoxy-ethyl-adenine
  modified_base 3: mod_base = OTHER; note = 2'-o-methoxy-ethyl-guanosine
  modified_base 4: mod_base = OTHER; note = 2'-o-methoxy-ethyl-cytidine
  modified_base 5: mod_base = OTHER; note = 2'-o-methoxy-ethyl-cytidine
  modified_base 15: mod_base = OTHER; note = 2'-o-methoxy-ethyl-adenine
  modified_base 16: mod_base = OTHER; note = 2'-o-methoxy-ethyl-guanosine
  modified_base 17: mod_base = OTHER; note = 2'-o-methoxy-ethyl-adenine
  modified_base 18: mod_base = OTHER; note = 2'-o-methoxy-ethyl-uridine
  modified_base 19: mod_base = OTHER; note = 2'-o-methoxy-ethyl-uridine
  modified_base 1..19: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19
  misc_feature 19: note = 3' TEG-linked cholesterol conjugate
aagccatattgagcagatt

SEQ ID NO: 134 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-adenine
  modified_base 2: mod_base = OTHER; note = 2'-o-methoxy-ethyl-guanosine
  modified_base 3: mod_base = OTHER; note = 2'-o-methoxy-ethyl-adenine
  modified_base 4: mod_base = OTHER; note = 2'-o-methoxy-ethyl-adenine
  modified_base 5: mod_base = OTHER; note = 2'-o-methoxy-ethyl-adenine
  modified_base 16: mod_base = OTHER; note = 2'-o-methoxy-ethyl-uridine
  modified_base 17: mod_base = OTHER; note = 2'-o-methoxy-ethyl-cytidine
  modified_base 18: mod_base = OTHER; note = 2'-o-methoxy-ethyl-cytidine
  modified_base 19: mod_base = OTHER; note = 2'-o-methoxy-ethyl-adenine
  modified_base 20: mod_base = OTHER; note = 2'-o-methoxy-ethyl-uridine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
agaaaacctctttactccat

SEQ ID NO: 135 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-adenine
  modified_base 2: mod_base = OTHER; note = 2'-o-methoxy-ethyl-cytidine
  modified_base 3: mod_base = OTHER; note = 2'-o-methoxy-ethyl-adenine
  modified_base 4: mod_base = OTHER; note = 2'-o-methoxy-ethyl-adenine
  modified_base 5: mod_base = OTHER; note = 2'-o-methoxy-ethyl-adenine
  modified_base 16: mod_base = OTHER; note = 2'-o-methoxy-ethyl-cytidine
  modified_base 17: mod_base = OTHER; note = 2'-o-methoxy-ethyl-cytidine
  modified_base 18: mod_base = OTHER; note = 2'-o-methoxy-ethyl-uridine
  modified_base 19: mod_base = OTHER; note = 2'-o-methoxy-ethyl-guanosine
  modified_base 20: mod_base = OTHER; note = 2'-o-methoxy-ethyl-uridine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
acaaatatacaccaccctgt

SEQ ID NO: 136 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-cytidine
  modified_base 2: mod_base = OTHER; note = 2'-o-methoxy-ethyl-cytidine
  modified_base 3: mod_base = OTHER; note = 2'-o-methoxy-ethyl-uridine
  modified_base 4: mod_base = OTHER; note = 2'-o-methoxy-ethyl-uridine
  modified_base 5: mod_base = OTHER; note = 2'-o-methoxy-ethyl-cytidine
  modified_base 16: mod_base = OTHER; note = 2'-o-methoxy-ethyl-cytidine
  modified_base 17: mod_base = OTHER; note = 2'-o-methoxy-ethyl-adenine
  modified_base 18: mod_base = OTHER; note = 2'-o-methoxy-ethyl-guanosine
  modified_base 19: mod_base = OTHER; note = 2'-o-methoxy-ethyl-cytidine
  modified_base 20: mod_base = OTHER; note = 2'-o-methoxy-ethyl-uridine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
ccttcacattggtgacagct

SEQ ID NO: 137 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-adenine
  modified_base 2: mod_base = OTHER; note = 2'-o-methoxy-ethyl-guanosine
  modified_base 3: mod_base = OTHER; note = 2'-o-methoxy-ethyl-uridine
  modified_base 4: mod_base = OTHER; note = 2'-o-methoxy-ethyl-uridine
  modified_base 5: mod_base = OTHER; note = 2'-o-methoxy-ethyl-uridine
  modified_base 16: mod_base = OTHER; note = 2'-o-methoxy-ethyl-cytidine
  modified_base 17: mod_base = OTHER; note = 2'-o-methoxy-ethyl-adenine
  modified_base 18: mod_base = OTHER; note = 2'-o-methoxy-ethyl-adenine
  modified_base 19: mod_base = OTHER; note = 2'-o-methoxy-ethyl-adenine
  modified_base 20: mod_base = OTHER; note = 2'-o-methoxy-ethyl-uridine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
agtttgtcatagtcacaaat

SEQ ID NO: 138 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-uridine
  modified_base 2: mod_base = OTHER; note = 2'-o-methoxy-ethyl-adenine
  modified_base 3: mod_base = OTHER; note = 2'-o-methoxy-ethyl-uridine
  modified_base 4: mod_base = OTHER; note = 2'-o-methoxy-ethyl-cytidine
  modified_base 5: mod_base = OTHER; note = 2'-o-methoxy-ethyl-cytidine
  modified_base 16: mod_base = OTHER; note = 2'-o-methoxy-ethyl-guanosine
  modified_base 17: mod_base = OTHER; note = 2'-o-methoxy-ethyl-cytidine
  modified_base 18: mod_base = OTHER; note = 2'-o-methoxy-ethyl-uridine
  modified_base 19: mod_base = OTHER; note = 2'-o-methoxy-ethyl-adenine
  modified_base 20: mod_base = OTHER; note = 2'-o-methoxy-ethyl-uridine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
tatccattgaggctagctat

SEQ ID NO: 139 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-uridine
  modified_base 2: mod_base = OTHER; note = 2'-o-methoxy-ethyl-guanosine
  modified_base 3: mod_base = OTHER; note = 2'-o-methoxy-ethyl-uridine
  modified_base 4: mod_base = OTHER; note = 2'-o-methoxy-ethyl-cytidine
  modified_base 5: mod_base = OTHER; note = 2'-o-methoxy-ethyl-uridine
  modified_base 16: mod_base = OTHER; note = 2'-o-methoxy-ethyl-cytidine
  modified_base 17: mod_base = OTHER; note = 2'-o-methoxy-ethyl-uridine
  modified_base 18: mod_base = OTHER; note = 2'-o-methoxy-ethyl-guanosine
  modified_base 19: mod_base = OTHER; note = 2'-o-methoxy-ethyl-cytidine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
tgtcttaaaggagacctgca

SEQ ID NO: 140 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-guanosine
  modified_base 2: mod_base = OTHER; note = 2'-o-methoxy-ethyl-adenine
  modified_base 3: mod_base = OTHER; note = 2'-o-methoxy-ethyl-cytidine
  modified_base 4: mod_base = OTHER; note = 2'-o-methoxy-ethyl-uridine
  modified_base 5: mod_base = OTHER; note = 2'-o-methoxy-ethyl-guanosine
  modified_base 16: mod_base = OTHER; note = 2'-o-methoxy-ethyl-cytidine
  modified_base 17: mod_base = OTHER; note = 2'-o-methoxy-ethyl-uridine
  modified_base 18: mod_base = OTHER; note = 2'-o-methoxy-ethyl-cytidine
  modified_base 19: mod_base = OTHER; note = 2'-o-methoxy-ethyl-adenine
  modified_base 20: mod_base = OTHER; note = 2'-o-methoxy-ethyl-uridine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
gactgaggacaagagctcat

SEQ ID NO: 141 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-guanosine
  modified_base 2: mod_base = OTHER; note = 2'-o-methoxy-ethyl-uridine
  modified_base 3: mod_base = OTHER; note = 2'-o-methoxy-ethyl-guanosine
  modified_base 4: mod_base = OTHER; note = 2'-o-methoxy-ethyl-cytidine
  modified_base 5: mod_base = OTHER; note = 2'-o-methoxy-ethyl-cytidine
  modified_base 16: mod_base = OTHER; note = 2'-o-methoxy-ethyl-adenine
  modified_base 17: mod_base = OTHER; note = 2'-o-methoxy-ethyl-adenine
  modified_base 18: mod_base = OTHER; note = 2'-o-methoxy-ethyl-uridine
  modified_base 19: mod_base = OTHER; note = 2'-o-methoxy-ethyl-uridine
  modified_base 20: mod_base = OTHER; note = 2'-o-methoxy-ethyl-cytidine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
gtgcccagggatattaattc

SEQ ID NO: 142 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-guanosine
  modified_base 2: mod_base = OTHER; note = 2'-o-methoxy-ethyl-cytidine
  modified_base 3: mod_base = OTHER; note = 2'-o-methoxy-ethyl-uridine
  modified_base 4: mod_base = OTHER; note = 2'-o-methoxy-ethyl-guanosine
  modified_base 5: mod_base = OTHER; note = 2'-o-methoxy-ethyl-guanosine
  modified_base 16: mod_base = OTHER; note = 2'-o-methoxy-ethyl-uridine
  modified_base 17: mod_base = OTHER; note = 2'-o-methoxy-ethyl-adenine
  modified_base 18: mod_base = OTHER; note = 2'-o-methoxy-ethyl-adenine
  modified_base 19: mod_base = OTHER; note = 2'-o-methoxy-ethyl-guanosine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
gctggatagttctcttaaga

SEQ ID NO: 143 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-cytidine
  modified_base 2: mod_base = OTHER; note = 2'-o-methoxy-ethyl-adenine
  modified_base 3: mod_base = OTHER; note = 2'-o-methoxy-ethyl-adenine
  modified_base 4: mod_base = OTHER; note = 2'-o-methoxy-ethyl-guanosine
  modified_base 5: mod_base = OTHER; note = 2'-o-methoxy-ethyl-adenine
  modified_base 16: mod_base = OTHER; note = 2'-o-methoxy-ethyl-adenine
  modified_base 17: mod_base = OTHER; note = 2'-o-methoxy-ethyl-adenine
  modified_base 18: mod_base = OTHER; note = 2'-o-methoxy-ethyl-guanosine
  modified_base 19: mod_base = OTHER; note = 2'-o-methoxy-ethyl-guanosine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
caagaaaaacaaaccaagga

SEQ ID NO: 144 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-cytidine
  modified_base 2: mod_base = OTHER; note = 2'-o-methoxy-ethyl-cytidine
  modified_base 3: mod_base = OTHER; note = 2'-o-methoxy-ethyl-cytidine
  modified_base 4: mod_base = OTHER; note = 2'-o-methoxy-ethyl-adenine
  modified_base 5: mod_base = OTHER; note = 2'-o-methoxy-ethyl-adenine
  modified_base 16: mod_base = OTHER; note = 2'-o-methoxy-ethyl-uridine
  modified_base 17: mod_base = OTHER; note = 2'-o-methoxy-ethyl-adenine
  modified_base 18: mod_base = OTHER; note = 2'-o-methoxy-ethyl-guanosine
  modified_base 19: mod_base = OTHER; note = 2'-o-methoxy-ethyl-uridine
  modified_base 20: mod_base = OTHER; note = 2'-o-methoxy-ethyl-uridine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
cccaaagagactctttagtt

SEQ ID NO: 145 - DNA, 19 bp, synthetic construct
  misc_feature 1..19: note = Description of sequence: antisense oligo
  source 1..19: mol_type = other DNA
  modified_base 1: mod_base = OTHER; note = 2'-o-methoxy-ethyl-adenine
  modified_base 2: mod_base = OTHER; note = 2'-o-methoxy-ethyl-adenine
  modified_base 3: mod_base = OTHER; note = 2'-o-methoxy-ethyl-cytidine
  modified_base 4: mod_base = OTHER; note = 2'-o-methoxy-ethyl-cytidine
  modified_base 5: mod_base = OTHER; note = 2'-o-methoxy-ethyl-adenine
  modified_base 15: mod_base = OTHER; note = 2'-o-methoxy-ethyl-guanosine
  modified_base 16: mod_base = OTHER; note = 2'-o-methoxy-ethyl-adenine
  modified_base 17: mod_base = OTHER; note = 2'-o-methoxy-ethyl-adenine
  modified_base 18: mod_base = OTHER; note = 2'-o-methoxy-ethyl-adenine
  modified_base 19: mod_base = OTHER; note = 2'-o-methoxy-ethyl-adenine
  modified_base 1..19: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19
  misc_feature 19: note = 3' TEG-linked cholesterol conjugate
aaccaaggaacaaagaaaa

SEQ ID NO: 146 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-adenine
  modified_base 2: mod_base = OTHER; note = 2'-o-methoxy-ethyl-guanosine
  modified_base 3: mod_base = OTHER; note = 2'-o-methoxy-ethyl-cytidine
  modified_base 4: mod_base = OTHER; note = 2'-o-methoxy-ethyl-adenine
  modified_base 5: mod_base = OTHER; note = 2'-o-methoxy-ethyl-adenine
  modified_base 16: mod_base = OTHER; note = 2'-o-methoxy-ethyl-adenine
  modified_base 17: mod_base = OTHER; note = 2'-o-methoxy-ethyl-cytidine
  modified_base 18: mod_base = OTHER; note = 2'-o-methoxy-ethyl-guanosine
  modified_base 19: mod_base = OTHER; note = 2'-o-methoxy-ethyl-cytidine
  modified_base 20: mod_base = OTHER; note = 2'-o-methoxy-ethyl-uridine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
agcaaataaaaagaaacgct

SEQ ID NO: 147 - DNA, 19 bp, synthetic construct
  misc_feature 1..19: note = Description of sequence: antisense oligo
  source 1..19: mol_type = other DNA
  modified_base 1: mod_base = OTHER; note = 2'-o-methoxy-ethyl-adenine
  modified_base 2: mod_base = OTHER; note = 2'-o-methoxy-ethyl-adenine
  modified_base 3: mod_base = OTHER; note = 2'-o-methoxy-ethyl-adenine
  modified_base 4: mod_base = OTHER; note = 2'-o-methoxy-ethyl-cytidine
  modified_base 5: mod_base = OTHER; note = 2'-o-methoxy-ethyl-adenine
  modified_base 15: mod_base = OTHER; note = 2'-o-methoxy-ethyl-adenine
  modified_base 16: mod_base = OTHER; note = 2'-o-methoxy-ethyl-cytidine
  modified_base 17: mod_base = OTHER; note = 2'-o-methoxy-ethyl-adenine
  modified_base 18: mod_base = OTHER; note = 2'-o-methoxy-ethyl-adenine
  modified_base 19: mod_base = OTHER; note = 2'-o-methoxy-ethyl-adenine
  modified_base 1..19: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19
  misc_feature 19: note = 3' TEG-linked cholesterol conjugate
aaacaaaccaaggaacaaa

SEQ ID NO: 148 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-uridine
  modified_base 2: mod_base = OTHER; note = 2'-o-methoxy-ethyl-adenine
  modified_base 3: mod_base = OTHER; note = 2'-o-methoxy-ethyl-uridine
  modified_base 4: mod_base = OTHER; note = 2'-o-methoxy-ethyl-cytidine
  modified_base 5: mod_base = OTHER; note = 2'-o-methoxy-ethyl-cytidine
  modified_base 16: mod_base = OTHER; note = 2'-o-methoxy-ethyl-guanosine
  modified_base 17: mod_base = OTHER; note = 2'-o-methoxy-ethyl-cytidine
  modified_base 18: mod_base = OTHER; note = 2'-o-methoxy-ethyl-uridine
  modified_base 19: mod_base = OTHER; note = 2'-o-methoxy-ethyl-adenine
  modified_base 20: mod_base = OTHER; note = 2'-o-methoxy-ethyl-uridine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
tatccattgaggctagctat

SEQ ID NO: 149 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-guanosine
  modified_base 2: mod_base = OTHER; note = 2'-o-methoxy-ethyl-cytidine
  modified_base 3: mod_base = OTHER; note = 2'-o-methoxy-ethyl-uridine
  modified_base 4: mod_base = OTHER; note = 2'-o-methoxy-ethyl-adenine
  modified_base 5: mod_base = OTHER; note = 2'-o-methoxy-ethyl-adenine
  modified_base 16: mod_base = OTHER; note = 2'-o-methoxy-ethyl-guanosine
  modified_base 17: mod_base = OTHER; note = 2'-o-methoxy-ethyl-adenine
  modified_base 18: mod_base = OTHER; note = 2'-o-methoxy-ethyl-adenine
  modified_base 19: mod_base = OTHER; note = 2'-o-methoxy-ethyl-adenine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
gctaaaagaaatggcgaaaa

SEQ ID NO: 150 - DNA, 20 bp, synthetic construct
  misc_feature 1..20: note = Description of sequence: antisense oligo
  source 1..20: mol_type = other DNA
  modified_base 1: mod_base = OTHER; note = 2'-o-methoxy-ethyl-guanosine
  modified_base 2: mod_base = OTHER; note = 2'-o-methoxy-ethyl-adenine
  modified_base 3: mod_base = OTHER; note = 2'-o-methoxy-ethyl-adenine
  modified_base 4: mod_base = OTHER; note = 2'-o-methoxy-ethyl-cytidine
  modified_base 5: mod_base = OTHER; note = 2'-o-methoxy-ethyl-adenine
  modified_base 16: mod_base = OTHER; note = 2'-o-methoxy-ethyl-adenine
  modified_base 17: mod_base = OTHER; note = 2'-o-methoxy-ethyl-cytidine
  modified_base 18: mod_base = OTHER; note = 2'-o-methoxy-ethyl-guanosine
  modified_base 19: mod_base = OTHER; note = 2'-o-methoxy-ethyl-adenine
  modified_base 20: mod_base = OTHER; note = 2'-o-methoxy-ethyl-adenine
  modified_base 1..20: mod_base = OTHER; note = Phosphorothioate linkage between positions 1^2, 2^3, 3^4, 4^5, 5^6, 6^7, 7^8, 8^9, 9^10, 10^11, 11^12, 12^13, 13^14, 14^15, 15^16, 16^17, 17^18, 18^19, 19^20
  misc_feature 20: note = 3' TEG-linked cholesterol conjugate
gaacaaagaaaagagacgaa

(19) United States
(12) Patent Application Publication
(10) Pub. No.: US 2026/0357253 A1
(43) Pub. Date: Nov. 12, 2026

(54) METHODS AND COMPOSITIONS FOR SILENCING ELAVL2 EXPRESSION FOR THE TREATMENT OF DISEASE

(71) Applicant: UNIVERSITY OF MASSACHUSETTS, Westborough, MA (US)

(72) Inventors: Fen-Biao GAO, Chestnut Hill, MA (US); Jonathan K. WATTS, Worcester, MA (US); Gopinath KRISHNAN, Worcester, MA (US)

(21) Appl. No.: 19/118,895

(22) PCT Filed: Oct. 6, 2023
(86) PCT No.: PCT/US2023/076274

§ 371 Date: Apr. 7, 2025

Related U.S. Application Data
(60) Provisional application No. 63/414,050, filed on Oct. 7, 2022.

(51) Int. Cl. C12N 15/113

(52) U.S. Cl. CPC C12N 15/113; C12N 2310/11; C12N 2310/14; C12N 2310/311; C12N 2310/312; C12N 2310/313; C12N 2310/314; C12N 2310/315; C12N 2310/316; C12N 2310/321; C12N 2310/322; C12N 2310/3231; C12N 2310/3233

(57) ABSTRACT

Disclosed are compositions and methods for reducing expression of ELAVL2. It has been determined that GGGGCC (G₄C₂) repeat expansion in the first intron of C9OR-F72 is the most commonly known genetic cause of amyotrophic lateral sclerosis (ALS) and frontotemporal dementia (FTD). Reducing expression of ELAVL2 reduces the downstream effect of the G₄C₂ repeat expansion, thereby providing methods of treatment for G₄C₂-related diseases and disorders.

### CROSS REFERENCE TO RELATED APPLICATIONS

This PCT application claims priority to, and the benefit of, U.S. Provisional Patent Application No. 63/414,050, filed Oct. 7, 2022, entitled “METHODS AND COMPOSITIONS FOR SILENCING ELAVL2 EXPRESSION FOR THE TREATMENT OF DISEASE,” which is incorporated by reference herein in its entirety.

### STATEMENT REGARDING FEDERALLY SPONSORED RESEARCH

This invention was made with government support under NS111990 awarded by the National Institutes of Health (NIH). The government has certain rights in the invention.

### REFERENCE TO SEQUENCE LISTING

The sequence listing submitted on Oct. 6, 2023, as an .XML file entitled “11579-004WO1_ST26” created on Oct. 5, 2023, and having a file size of 1,388,420 bytes is hereby incorporated by reference pursuant to 37 C.F.R. § 1.52 (e) (5).

### FIELD

The present disclosure relates to compositions and methods for reducing expression of ELAVL2.

### BACKGROUND

A GGGGCC (G₄C₂) repeat expansion in the first intron of C9ORF72 is the most commonly known genetic cause of amyotrophic lateral sclerosis (ALS) and frontotemporal dementia (FTD). RNAs transcribed from these repeats in both sense and antisense directions can be translated into five dipeptide repeat (DPR) proteins—poly(GR), poly(GA), poly(GP), poly(PR), and poly(PA). Among them, poly(GR) is a key neurotoxic species, as its expression strongly correlates with neurodegeneration in C9ORF72 patient brains. Moreover, poly(GR) is toxic in many cellular and animal models.

The use of antisense oligonucleotides (ASOs) is a contemplated approach to reduce the level of expanded G₄C₂ repeats-containing RNA, as shown in a mouse model of C9ORF72-ALS/FTD. Indeed, such a treatment in a single ALS patient using an ASO was reported to be safe. However, a clinical trial carried out by Biogen in 106 ALS patients with the expanded G₄C₂ repeats in the C9ORF72 gene halted due to lack of benefits. This failure is not surprising, because by the time these patients exhibit clinical symptoms, it is already too late to eliminate one of the most upstream toxic molecules that have initiated multiple disease cascades years or decades earlier.

What is needed are methods and compositions which target genetic suppressors of poly(GR) toxicity without affecting the level of toxic poly(GR) protein.

### SUMMARY

The present disclosure provides compositions and methods that target genetic suppressors of poly(GR) toxicity without affecting the level of toxic poly(GR) protein. Disclosed herein are gene silencers of these modifier genes and method of use thereof which offer clinical benefits by slowing down or blocking ongoing disease processes, even in the presence of disease-causing G₄C₂ repeats, poly(GR) and other dipeptide repeat (DPR) proteins.

Disclosed herein is a nucleic acid inhibitor which is capable of reducing expression of ELAVL2 by 10% or more compared to a control where the nucleic acid inhibitor is not present, wherein said nucleic acid inhibitor hybridizes to at least five nucleotides within SEQ ID NO: 1.

In some embodiments, the nucleic acid inhibitor hybridizes to at least five nucleotides within a region of SEQ ID NO: 2. In some embodiments, the nucleic acid inhibitor hybridizes to at least five nucleotides within a region of 1-450 or 2100-3983 of SEQ ID NO: 2.

In some embodiments, the nucleic acid inhibitor is 15-30 nucleotides long. In some embodiments, the nucleic acid inhibitor is an antisense oligonucleotide or an RNAi molecule. In some embodiments, the RNAi molecule is siRNA.

In some embodiments, the nucleic acid inhibitor comprises at least one modification. In some embodiments, the at least one modification comprises at least one modified sugar moiety; at least one modified inter-nucleotide linkage; and/or at least one modified nucleotide. In some embodiments, the at least one modification comprising an internucleotide linkage comprising: phosphorothioate, alkylphosphonate, phosphorodithioate, alkylphosphonothioate, phosphoramidate, carbamate, carbonate, phosphate triester, acetamidate, and/or carboxymethyl ester. In some embodiments, the at least one modification comprising a modified nucleotide comprises a peptide nucleic acid, a morpholino analogue, a locked nucleic acid (LNA) or other bridged or bicyclic nucleic acid analogue, and/or a combination thereof. In some embodiments, the at least one modification comprising a modified sugar moiety comprises: a 2′-fluoro sugar moiety, 2′-O-methoxyethyl modified sugar moiety, a 2′-methoxy modified sugar moiety, and/or another 2′-O-alkyl or alkoxyalkyl modified sugar moiety.

In some embodiments, the nucleic acid inhibitor further comprises a conjugate. In some embodiments, the conjugate comprises a therapeutic agent, a diagnostic agent, or an agent that increases cell uptake or penetration. In some embodiments, the conjugate comprises a peptide, a protein, an antibody or fragment thereof, a lipid, a neurotransmitter, a cationic polymer, a nanoparticle, a probe, or a carbohydrate.

In some embodiments, the conjugate is a multivalent conjugate. In some embodiments, the multivalent conjugate comprises a multimeric antisense oligonucleotide or a multivalent siRNA.

In one aspect, disclosed herein is a viral vector comprising the nucleic acid inhibitor of any preceding aspect, or which induces the intracellular expression of the nucleic acid inhibitor of any preceding aspect.

In one aspect, disclosed herein is a pharmaceutical composition comprising the nucleic acid inhibitor of any preceding aspect. In some embodiments, the pharmaceutical composition further comprises a vehicle for delivery of the nucleic acid inhibitor. In some embodiments, the vehicle comprises a nanoparticle. In some embodiments, the vehicle comprises a viral vector.

In one aspect, disclosed herein is a nucleic acid comprising 90% or more identity to any one of SEQ ID NOS: 3-50, SEQ ID NOS: 54-64, or SEQ ID NOS: 82-150.

In one aspect, disclosed herein is a nucleic acid comprising 95% or more identity to any one of SEQ ID NOS: 3-50, SEQ ID NOS: 54-64, or SEQ ID NOS: 82-150.

In one aspect, disclosed herein is a nucleic acid comprising SEQ ID NOS: 17, 21, or 24.

In one aspect, disclosed herein is a nucleic acid comprising SEQ ID NOS: 41, 45, and 48.

In one aspect, disclosed herein is a nucleic acid comprising SEQ ID NOS: 57, 58, 59, 60, 61, or 62.

In one aspect, disclosed herein is a nucleic acid comprising SEQ ID NOS: 63 or 64.

Further disclosed herein is a method of treating disorders and diseases associated with poly(GR) toxicity in neuronal cells in a subject in need thereof, the method comprising modulating expression of ELAVL2.

In some embodiments, the poly(GR) toxicity is caused by a GGGGCC (G₄C₂) repeat expansion in a first intron of C9ORF72. In some embodiments, the subject has, or is at risk of developing, amyotrophic lateral sclerosis (ALS) and/or frontotemporal dementia (FTD).

In some embodiments, the method comprises expression of ELAVL2 is reduced by 10% or more compared to a control wherein a modulator of ELAVL2 expression is not present. In some embodiments, one or more symptoms of a disease or disorder associated with poly(GR) toxicity is reduced by 10% or more compared to a control wherein a modulator of ELAVL2 expression is not present. In some embodiments, one or more symptoms of a disease or disorder associated with poly(GR) toxicity is prevented compared to a control wherein a modulator of ELAVL2 expression is not present.

In some embodiments, the method comprises modulation of expression of ELAVL2 is carried out by a nucleic acid inhibitor. In some embodiments, the nucleic acid inhibitor comprises an antisense oligonucleotide, short interfering RNA (siRNA), double stranded RNA (dsRNA), micro-RNA (miRNA), small nucleolar RNA (sno-RNA), Piwi-interacting RNA (piRNA), or short hairpin RNA (shRNA) molecules.

In some embodiments, the method comprises the nucleic acid inhibitor hybridizing to at least five nucleotides within SEQ ID NO: 1. In some embodiments, the method comprises the nucleic acid inhibitor hybridizing to at least five nucleotides within a region of SEQ ID NO: 2. In some embodiments, the method comprises the nucleic acid inhibitor hybridizing to at least five nucleotides within a region of 1-450 or 2100-3983 of SEQ ID NO: 2.

In some embodiments, the method comprises the nucleic acid inhibitor comprising 90% or more identity to any one of SEQ ID NOS: 3-50, SEQ ID NOS: 54-64, or SEQ ID NOS: 82-150. In some embodiments, the method comprises the nucleic acid inhibitor comprising 95% or more identity to any one of SEQ ID NOS: 3-50, SEQ ID NOS: 54-64, or SEQ ID NOS: 82-150. In some embodiments, the method comprises the nucleic acid inhibitor comprising SEQ ID NOS: 17, 21, or 24. In some embodiments, the method comprises the nucleic acid inhibitor comprising SEQ ID NOS: 41, 45, or 48. In some embodiments, the method comprises the nucleic acid inhibitor comprises SEQ ID NOS: 57, 58, 59, 60, 61, or 62. In some embodiments, the method comprises the nucleic acid inhibitor comprises SEQ ID NOS: 63 or 64.

Also disclosed herein is a method of screening for a nucleic acid inhibitor of ELAVL2, the method comprising contacting SEQ ID NO: 1 with complementary nucleic acids between 15-30 nucleotides long and determining which are effective at knocking down expression of ELAVL2.

In some embodiments, the screening takes place inside of a cell.

In one aspect, disclosed herein is a nucleic acid inhibitor identified by any preceding aspect. In some embodiments, the silencing RNA comprises an antisense oligonucleotide, short interfering RNA (siRNA), double stranded RNA (dsRNA), micro-RNA (miRNA), small nucleolar RNA (sno-RNA), Piwi-interacting RNA (piRNA), or short hairpin RNA (shRNA) molecules.

### BRIEF DESCRIPTION OF FIGURES

The accompanying figures, which are incorporated in and constitute a part of this specification, illustrate several aspects described below.

FIGS. 1A, 1B, and 1C show the Rbp9 (an ortholog of ELAVL2) knockdown (KD) in the eye also suppressed photoreceptor neuron degeneration and this suppressor effect is not due to a decrease in Poly (GR) levels in the fly eye. It was shown that Rbp9 knockdown by two independent RNAi suppressed poly(GR) toxicity in fly eye neurons (Panel A and B), confirming it is indeed a genetic suppressor. More importantly, rbp9 knockdown did not affect the expression level of poly(GR) (Panel C), showing rbp9 acts in a pathway downstream of poly(GR) expression. This result demonstrates that one can decrease C9ORF72-ALS/FTD disease phenotypes even in the presence of same amount of toxic proteins, a therapeutic approach different from previous attempts which tried to decrease the level of poly(GR) and other disease molecules (such as repeat RNA and poly(GA) etc.) by ASO targeting repeat-containing intronic RNA.

FIG. 2 shows the screening of ASOs identified those that could effectively knockdown the expression level of ELAVL2 in iPSC-derived human C9ORF72-ALS/FTD patient motor neurons. Quantitative real-time PCR analysis confirming ASO-mediated reduction of ELAVL-2 mRNA levels in human iPSC-derived motor neurons (3 weeks old). ASOs578-595 screened in C9 patient line40. ASOs 596-601 screened in C9patient line42. Expression of GAPDH mRNAs were used as endogenous controls. ELAVL-2 ASOs (5 μM) treated for 72 hr in two biologically independent experiment. Error bars represent mean±SD.

FIGS. 3A and 3B show confirmation of reduced ELAVL-2 mRNA and protein levels. FIG. 3A shows the quantitative real-time PCR analysis confirming ASO-mediated reduction of ELAVL-2 mRNA levels in human iPSC-derived motor neurons (3 weeks old). ELAVL-2 ASOs (5 μM) treated for one week in three biologically independent experiment. Error bars represent mean±SD. FIG. 3B shows the western blot analysis confirming reduction in ELAVL2 protein levels in motor neurons treated with ELAVL2-ASO #599.

FIG. 4 shows ELAV2 is localized in both the nucleus and cytosol in iPSC-derived motor neurons.

FIG. 5 shows the generation of CRISPR deletion of ELAVL2 gene in two C9ORF72 iPSC lines.

FIGS. 6A and 6B show the RNA-seq analysis of control (FIG. 6A) and C9ORF72 iPSC-derived (FIG. 6B) motor neurons with ELAVL2 knockdown.

FIGS. 7A and 7B show 24 specific antisense oligonucleotides (FIG. 7A) which were found to reduce expression of ELAVL2 (SEQ ID NOS: 3-24. Note that these correspond to the ASOs from FIG. 2); and the nucleic acid sequence encoding ELAVL2 (FIG. 7B), which shows where the specific ASOs from FIG. 7A bind.

FIG. 8 shows the quantitative real-time PCR analysis confirming siRNA-mediated reduction of ELAVL-2 mRNA levels in human iPSC-derived 2 weeks old motor neurons (isogenic control line). Expression of GAPDH mRNAs were used as endogenous controls. ELAVL-2 siRNAs (5 μM) treated for 72 h in one biologically independent experiment. Error bars represent mean±SD of two technical wells.

FIG. 9 shows the quantitative real-time PCR analysis confirming siRNA-mediated reduction of ELAVL-2 mRNA levels in human iPSC-derived 2 weeks old motor neurons. ASOs2240-2249 screened in healthy control lines. ASOs 2250-2259 screened in C9patient line. Expression of GAPDH mRNAs were used as endogenous controls. ELAVL-2 ASOs (5 μM) treated for 72 hr in two biologically independent experiment. Error bars represent mean±SD.

FIG. 10 shows the immunofluorescence analysis of ELAVL-2 protein cellular distribution in iPSC-derived motor neurons of 2 weeks old. Commercial ELAVL2 antibody used.

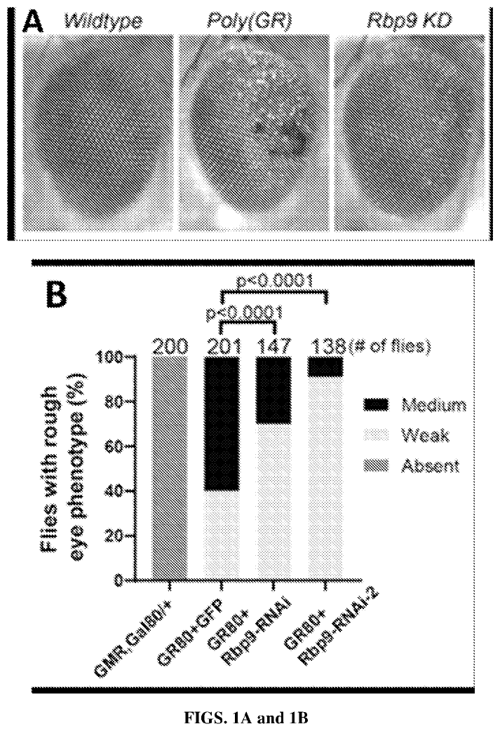

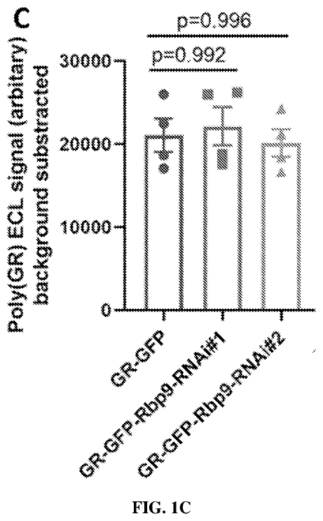

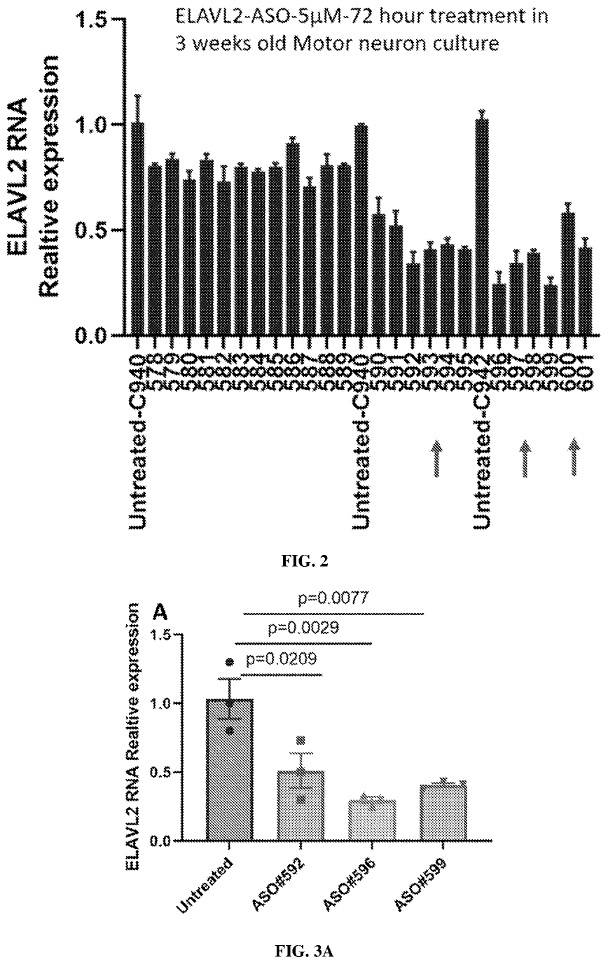

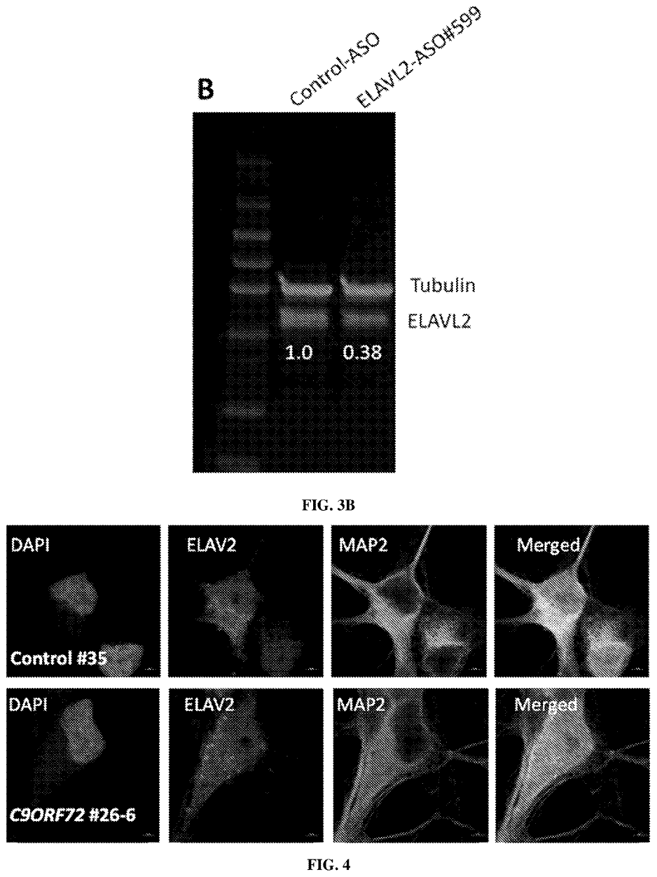

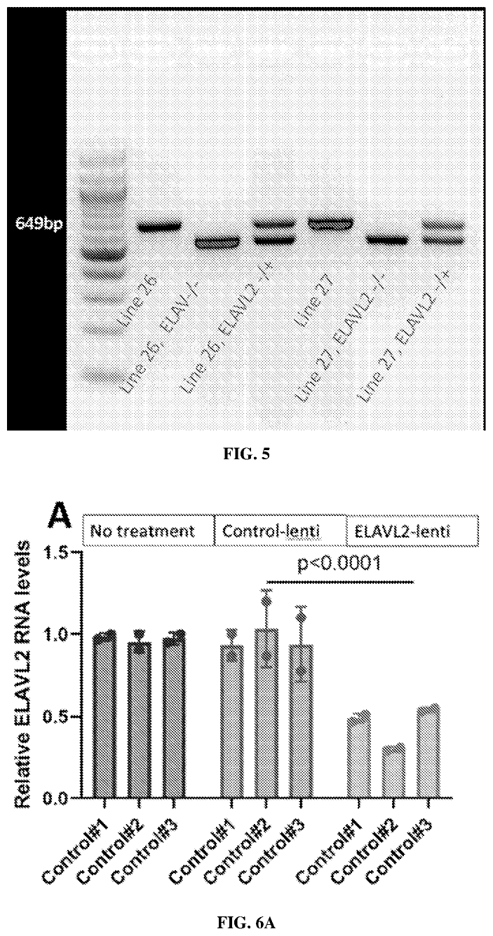

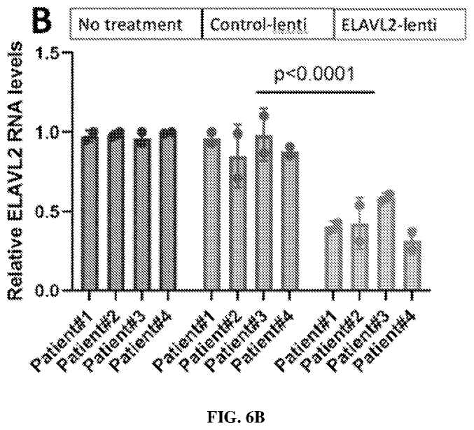

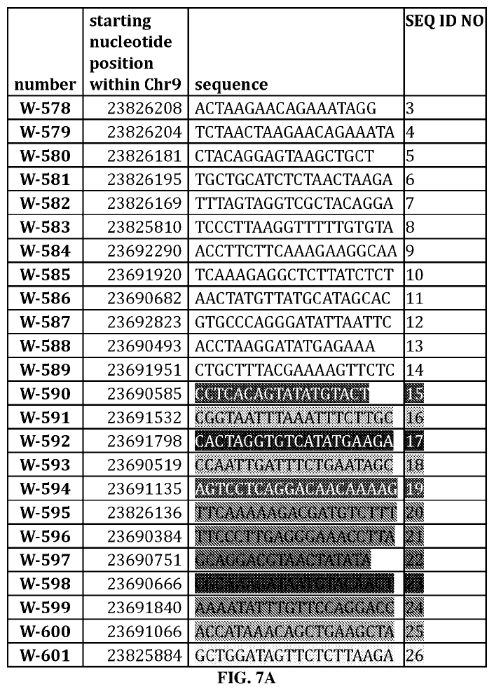

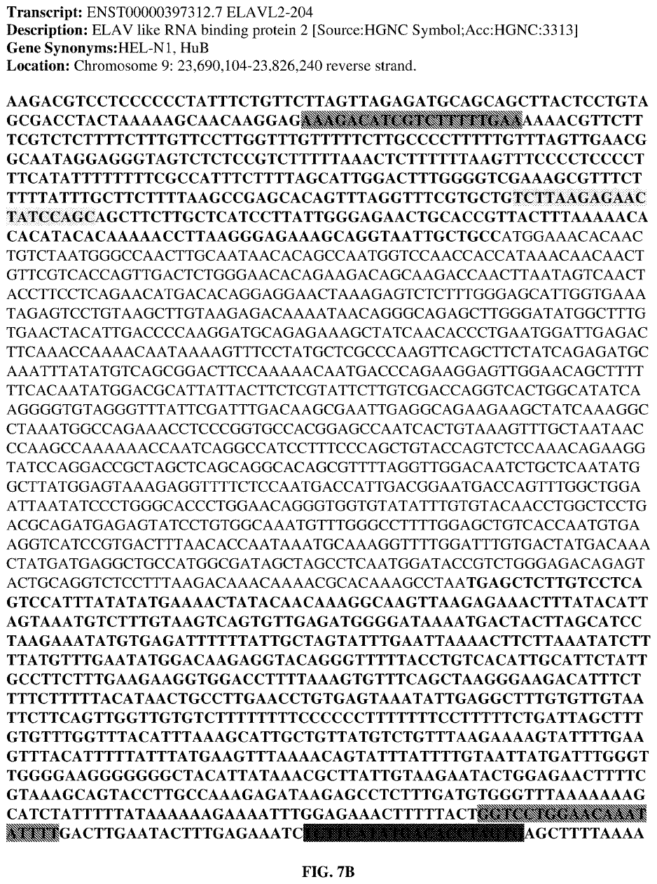

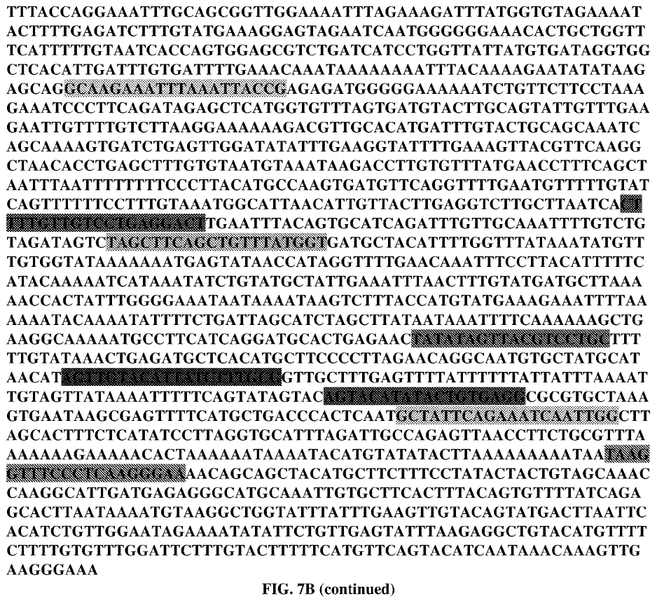

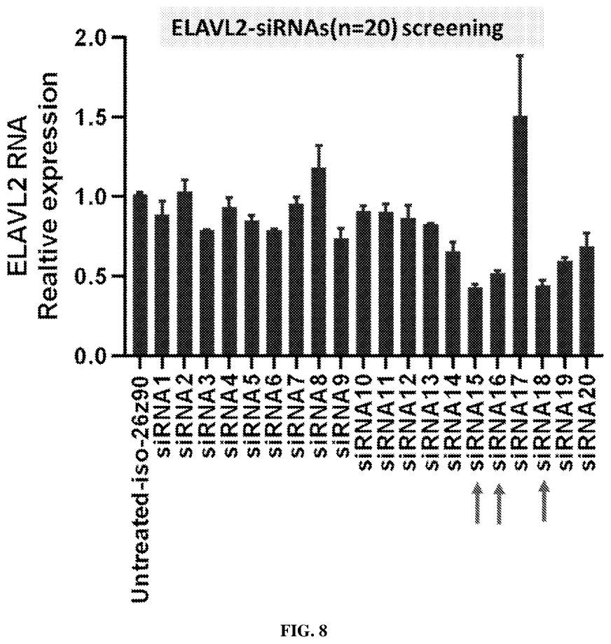

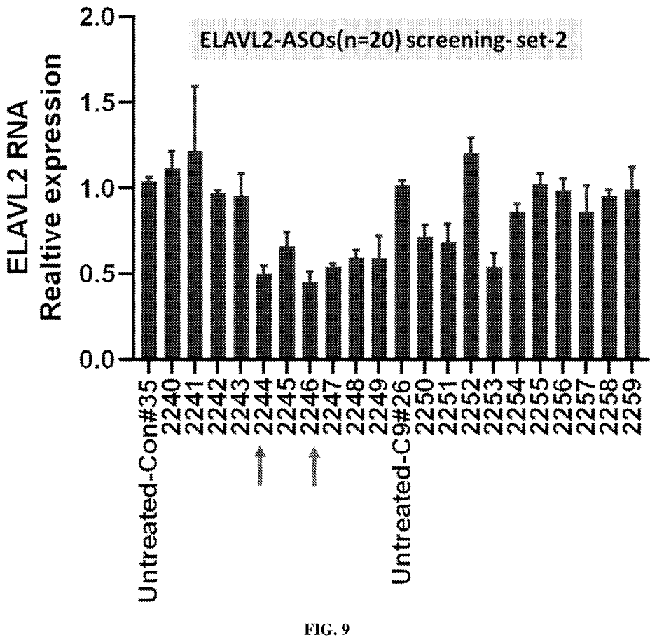

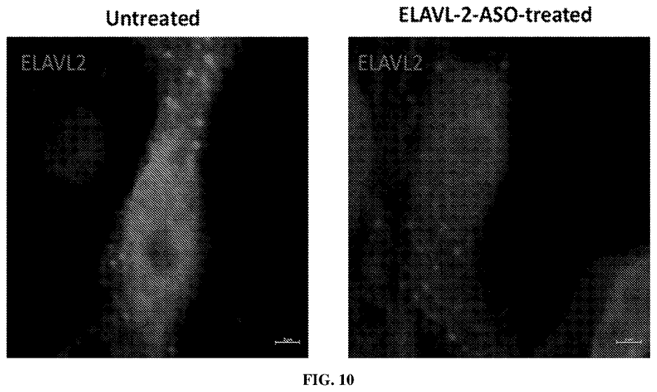

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### TERMINOLOGY

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce,” or “abrogate,” (used interchangeably) or other forms of the word, such as “reducing” or “reduction,” or “abrogating” or “abrogation” means lowering of an event or characteristic. It is understood that this is typically in relation to some standard or expected value, in other words it is relative, but that it is not always necessary for the standard or relative value to be referred to.

By “increase” or other forms of the word, such as “increasing,” means raising or elevating. It is understood that this is typically in relation to some standard or expected value, in other words it is relative, but that it is not always necessary for the standard or relative value to be referred to.

As used herein, by a “subject” means an individual. Thus, the “subject” can include domesticated animals (e.g., cats, dogs, etc.), livestock (e.g., cattle, horses, pigs, chickens, ducks, geese, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Detecting” is used herein to identify the existence, presence, or fact of something. General methods of detecting are known to the skilled artisan and may be supplemented with the protocols and reagents disclosed herein. For example, included herein are methods of detecting a nucleic acid molecule in sample. Detection can include a physical readout, such as fluorescence output.

The term “hybridization” is defined as forming base pairs between complementary regions of two strands of DNA, RNA, or between DNA and RNA, thereby forming a duplex molecule, for example. Hybridization conditions resulting in particular degrees of stringency will vary depending upon the nature of the hybridization method and the composition and length of the hybridizing nucleic acid sequences. Generally, the temperature of hybridization and the ionic strength (such as the Na+ concentration) of the hybridization buffer will determine the stringency of hybridization. Calculations regarding hybridization conditions for attaining particular degrees of stringency are discussed in Sambrook et al., (1989) Molecular Cloning, second edition, Cold Spring Harbor Laboratory, Plainview, N.Y. (chapters 9 and 11).

An “isolated” biological component (such as a nucleic acid molecule) has been substantially separated, produced apart from, or purified away from other biological components. Nucleic acid molecules which have been “isolated” include nucleic acids molecules purified by standard purification methods, as well as those chemically synthesized. Isolated does not require absolute purity, and can include nucleic acid molecules that are at least 50% isolated, such as at least 75%, 80%, 90%, 95%, 98%, 99% or even 100% isolated.

A “nucleic acid” is a deoxyribonucleotide or ribonucleotide polymer, which can include analogues of natural nucleotides that hybridize to nucleic acid molecules in a manner similar to naturally occurring nucleotides. In a particular example, a nucleic acid molecule is a single stranded (ss) DNA or RNA molecule, such as a probe or primer. In another particular example, a nucleic acid molecule is a double stranded (ds) nucleic acid, such as a target nucleic acid. Examples of modified nucleic acids are those with altered backbones, such as peptide nucleic acids (PNA).

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

Nucleotides include those nucleotides containing modified bases, modified sugar moieties and modified phosphate backbones, as known in the art and discussed in more detail below.

Examples of modified base moieties which can be used to modify nucleotides at any position on its structure include, but are not limited to: 7-deaza-8-azaguanosine, 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N-2-carboxypropyl) uracil, and 2,6-diaminopurine.

Examples of modified sugar moieties which may be used to modify nucleotides at any position on its structure include, but are not limited to: 2′-substituted ribose analogues, arabinose, 2-fluoroarabinose, xylose, and hexose; morpholino sugar analogues, or bicyclic or tricyclic sugar moieties. 2′-substituted ribose groups may include 2′-fluoro, 2′-amino, 2′-O-methyl, 2′-O-methoxyethyl, or other 2′-O-substitutions including alkyl, aryl, alkoxyalkyl, or aminoalkyl groups, among others.

Examples of modified components of the phosphate backbone include analogues such as a phosphorothioate, a phosphorodithioate, a phosphoramidothioate, a phosphoramidate, a phosphordiamidate, a methylphosphonate, an alkyl phosphotriester, or an analog thereof. The phosphate linkage analogue may not necessarily include a phosphorus atom, as formacetal, amide, triazole or other linkage groups may replace the phosphate group in an oligonucleotide.

The term “complementary binding” as used herein occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA. In this example, the sequence 5′-ATCG-3′ is the reverse complement of 3′-TAGC-5′. Nucleic acid molecules can be complementary to each other even without complete hydrogen-bonding of all bases of each molecule. For example, hybridization with a complementary nucleic acid sequence can occur under conditions of differing stringency in which a complement will bind at some but not all nucleotide positions.

In reference to the nucleic molecules of the present disclosure, the binding free energy for a nucleic acid molecule with its complementary sequence is sufficient to allow the relevant function of the nucleic acid molecule to proceed where there is a sufficient degree of complementarity to avoid non-specific binding of the nucleic acid molecule (e.g., antisense oligonucleotide) to non-target sequences under conditions in which specific binding is desired, i.e., under physiological conditions in the case of in vivo assays or therapeutic treatment, or under conditions in which the assays are performed in the case of in vitro assays (e.g., hybridization assays). Determination of binding free energies for nucleic acid molecules is well known in the art (see, e.g., Turner et ah, CSH Symp. Quant. Biol. LIL Wi, 1987; Frier et ah, Proc. Nat'l. Acad. Sci. USA 53:9373, 1986; Turner et ah, J. Am. Chem. Soc. 709:3783, 1987). Thus, “complementary” or “specifically hybridizable” or “specifically binds” are terms that indicate a sufficient degree of complementarity or precise pairing such that stable and specific binding occurs between a nucleic acid molecule and a DNA or RNA target. It is understood in the art that a nucleic acid molecule need not be 100% complementary to a target nucleic acid sequence to be specifically hybridizable or to specifically bind. That is, two or more nucleic acid molecules may be less than fully complementary and is indicated by a percentage of contiguous residues in a nucleic acid molecule that can form hydrogen bonds with a second nucleic acid molecule.

For example, a first nucleic acid molecule may have 10 nucleotides and a second nucleic acid molecule may have 10 nucleotides, then base pairing of 5, 6, 7, 8, 9, or 10 nucleotides between the first and second nucleic acid molecules, which may or may not form a contiguous double-stranded region, represents 50%, 60%, 70%, 80%, 90%, and 100% complementarity, respectively. In certain embodiments, complementary nucleic acid molecules may have wrongly paired bases—that is, bases that cannot form a traditional Watson-Crick base pair or other non-traditional types of pair (i.e., “mismatched” bases). For instance, complementary nucleic acid molecules may be identified as having a certain number of “mismatches,” such as zero or about 1, about 2, about 3, about 4 or about 5.

“Perfectly” or “fully” complementary nucleic acid molecules means those in which a certain number of nucleotides of a first nucleic acid molecule hydrogen bond (anneal) with the same number of residues in a second nucleic acid molecule to form a contiguous double-stranded region. For example, two or more fully complementary nucleic acid molecule strands can have the same number of nucleotides (i.e., have the same length and form one double-stranded region, with or without an overhang) or have a different number of nucleotides (e.g., one strand may be shorter than but fully contained within another strand or one strand may overhang the other strand).

A “polymorphism” is a variation in a gene sequence. The polymorphisms can be those variations (DNA sequence differences, e.g., substitutions, deletions, or insertions) which are generally found between individuals or different ethnic groups and geographic locations which, while having a different sequence, produce functionally equivalent gene products. Typically, the term can also refer to variants in the sequence which can lead to gene products that are not functionally equivalent. Polymorphisms also encompass variations which can be classified as alleles and/or mutations which can produce gene products which may have an altered function. Polymorphisms also encompass variations which can be classified as alleles and/or mutations which either produce no gene product or an inactive gene product or an active gene product produced at an abnormal rate or in an inappropriate tissue or in response to an inappropriate stimulus. Alleles are the alternate forms that occur at the polymorphism.

Polymorphisms can be referred to, for instance, by the nucleotide position at which the variation exists, by the change in amino acid sequence caused by the nucleotide variation, or by a change in some other characteristic of the nucleic acid molecule or protein that is linked to the variation.

The identity/similarity between two or more nucleic acid sequences is expressed in terms of the identity between the sequences. Sequence identity can be measured in terms of percentage identity; the higher the percentage, the more identical the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology (NCBI, National Library of Medicine, Building 38A, Room 8N805, Bethesda, Md. 20894) and on the Internet, for use in connection with the sequence analysis programs blastp, blastn, blastx, tblastn, and tblastx. Additional information can be found at the NCBI web site. BLASTN is used to compare nucleic acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75). One indication that two nucleic acid molecules are closely related is that the two molecules hybridize to each other under stringent conditions, as described above.

A “sample,” such as a biological sample, is a sample obtained from a subject. As used herein, biological samples include all clinical samples useful for detection of a methylated nucleotide, including, but not limited to, cells, tissues, and bodily fluids, such as: blood; derivatives and fractions of blood, such as serum; urine; sputum; or CVS samples. In a particular example, a sample includes blood obtained from a human subject, such as whole blood or serum.

A “test nucleic acid molecule” refers to a nucleic acid molecule whose detection, quantitation, qualitative detection, characterization, or a combination thereof, is intended. For example, the test nucleic acid molecule can be a defined region or particular portion of a nucleic acid molecule, for example a portion of a genome (such as a gene or a region of DNA or RNA containing a gene or portion thereof of interest). The nucleic acid molecule need not be in a purified form. Various other nucleic acid molecules can also be present with the test nucleic acid molecule. For example, the test nucleic acid molecule can be a specific nucleic acid molecule (which can include RNA or DNA), for which the detection of a particular polymorphism is intended. In some examples, a test nucleic acid includes a viral nucleic acid molecule, or a bacterial nucleic acid molecule. Purification or isolation of the test nucleic acid molecule, if needed, can be conducted by methods known to those in the art, such as by using a commercially available purification kit or the like.

By “contacting” means placement in direct physical association, for example solid, liquid or gaseous forms. Contacting includes, for example, direct physical association of fully- and partially-solvated molecules.

By “ribonucleic acid” or “RNA” is meant a nucleic acid molecule comprising at least one ribonucleotide molecule. The term RNA includes double-stranded (ds) RNA, single-stranded (ss) RNA, isolated RNA (such as partially purified RNA, essentially pure RNA, synthetic RNA, recombinantly produced RNA), altered RNA (which differs from naturally occurring RNA by the addition, deletion, substitution or alteration of one or more nucleotides), or any combination thereof. For example, such altered RNA can include addition of non-nucleotide material, such as at one or both ends of an RNA molecule, internally at one or more nucleotides of the RNA, or any combination thereof. Nucleotides in RNA molecules of the instant disclosure can also comprise non-standard nucleotides, such as naturally occurring nucleotides, non-naturally occurring nucleotides, chemically-modified nucleotides, deoxynucleotides, or any combination thereof. These altered RNAs may be referred to as analogs or analogs of RNA containing standard nucleotides (i.e., standard nucleotides, as used herein, are considered to be adenine, cytidine, guanidine, thymidine, and uridine).

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

The term “dsRNA” as used herein refers to any nucleic acid molecule comprising at least one ribonucleotide molecule and capable of inhibiting or down regulating gene expression, for example, by promoting RNA interference (“RNAi”) or gene silencing in a sequence-specific manner. The dsRNAs of the instant disclosure may be suitable substrates for Dicer or for association with RISC to mediate gene silencing by RNAi. One or both strands of the dsRNA can further comprise a terminal phosphate group, such as a 5′-phosphate or 5′,3′-diphosphate. As used herein, dsRNA molecules, in addition to at least one ribonucleotide, can further include substitutions, chemically-modified nucleotides, and non-nucleotides. In certain embodiments, dsRNA molecules comprise ribonucleotides up to about 100% of the nucleotide positions.

In addition, as used herein, the term dsRNA is meant to be equivalent to other terms used to describe nucleic acid molecules that are capable of mediating sequence specific RNAi, for example, meroduplex RNA (mdRNA), nicked dsRNA (ndsRNA), gapped dsRNA (gdsRNA), short interfering nucleic acid (siNA), siRNA, micro-RNA (miRNA), short hairpin RNA (shRNA), short interfering oligonucleotide, short interfering substituted oligonucleotide, short interfering modified oligonucleotide, chemically-modified dsRNA, post-transcriptional gene silencing RNA (ptgsRNA), or the like. A double-stranded structure may be formed by a self-complementary nucleic acid molecule or by annealing of two or more distinct complementary nucleic acid molecule strands.

In addition, as used herein, the term “RNAi” is meant to be equivalent to other terms used to describe sequence specific RNA interference, such as post transcriptional gene silencing, translational inhibition, or epigenetics. For example, dsRNA molecules of this disclosure can be used to epigenetically silence genes at the post-transcriptional level or the pre-transcriptional level or any combination thereof.

As used herein, “target nucleic acid” refers to any nucleic acid sequence whose expression or activity is to be altered (e.g., ELAVL2). The target nucleic acid can be DNA, RNA, or analogs thereof, and includes single, double, and multi-stranded forms. By “target site” or “target sequence” is meant a sequence within a target nucleic acid (e.g., mRNA) that, when present in an RNA molecule, is “targeted” for cleavage by RNAi and mediated by a dsRNA construct of this disclosure containing a sequence within the antisense strand that is complementary to the target site or sequence.

As used herein, “off-target effect” or “off-target profile” refers to the observed altered expression pattern of one or more genes in a cell or other biological sample not targeted, directly or indirectly, for gene silencing or antisense therapy. For example, an off-target effect can be quantified by using a DNA microarray to determine how many non-target genes have an expression level altered by about two-fold or more in the presence of a candidate antisense oligonucleotide or siRNA, or analog thereof specific for a target sequence, such as an ELAVL2 mRNA.

The term “gene” as used herein, especially in the context of “target gene” or “gene target” for gene silencing means a nucleic acid molecule that encodes an RNA or a transcription product of such gene, including a messenger RNA (mRNA, also referred to as structural genes that encode for a polypeptide), an mRNA splice variant of such gene, a functional RNA (fRNA), or non-coding RNA (ncRNA), such as small temporal RNA (stRNA), microRNA (miRNA), small nuclear RNA (snRNA), short interfering RNA (siRNA), small nucleolar RNA (snRNA), ribosomal RNA (rRNA), transfer RNA (tRNA) and precursor RNAs thereof.

As used herein, “gene silencing” refers to a partial or complete loss-of-function through targeted inhibition of gene expression in a cell, which may also be referred to as RNAi “knockdown,” “inhibition,” “down-regulation,” or “reduction” of expression of a target gene, such as a human FGF2 gene. Depending on the circumstances and the biological problem to be addressed, it may be preferable to partially reduce gene expression. Alternatively, it might be desirable to reduce gene expression as much as possible. The extent of silencing may be determined by methods described herein and known in the art (see, e.g., PCT Publication No. WO 99/32619; Elbashir et ah, EMBO J. 20: 6S77, 2001).

Depending on the assay, quantification of gene expression permits detection of various amounts of inhibition that may be desired in certain embodiments of this disclosure, including prophylactic and therapeutic methods, which will be capable of knocking down target gene expression, in terms of mRNA level or protein level or activity, for example, by equal to or greater than 10%, 20%, 30%, 40%, 50%, 60%, 70%, 75%, 80%, 85%, 90%, 95% or 99% of baseline (i.e., normal) or other control levels, including elevated expression levels as may be associated with particular disease states or other conditions targeted for therapy.

As used herein, the term “therapeutically effective amount” means an amount of antisense oligonucleotide(s) or RNAi that is sufficient to result in a decrease in severity of disease symptoms, an increase in frequency or duration of disease symptom-free periods, or a prevention of impairment or disability due to the disease, in the subject (e.g., human) to which it is administered. One of ordinary skill in the art would be able to determine such therapeutically effective amounts based on such factors as the subject's size, the severity of symptoms, and the particular composition or route of administration selected. The nucleic acid molecules of the instant disclosure, individually, or in combination or in conjunction with other drugs, can be used to treat diseases or conditions discussed herein. For example, to treat a particular disease, disorder, or condition, the nucleic acid molecules can be administered to a patient or can be administered to other appropriate cells evident to those skilled in the art, individually or in combination with one or more drugs, under conditions suitable for treatment.

### General Description

Amyotrophic lateral sclerosis (ALS) is a progressive, usually fatal motor neuron disorder that sometimes develops at the same time as frontotemporal dementia (FTD). ALS occurs in both familial (ELAF) and sporadic (ELAE) forms. A significant number of ELAF cases are associated with expansions of a non-coding G₄C₂ hexanucleotide repeats in the C9ORF72 gene. These expansions are also detected in 10-20% of familial frontotemporal dementia (FTD), 10% of sporadic FTD, and in ~5% of ELAE. These statistics define the G₄C₂ expansion of C9ORF72 as a common cause of ALS. In normal individuals, the G₄C₂ expansion ranges in size from 2 or 3 to more than 25 repeats; conversely, FTD/ALS patients have hundreds or even thousands of these repeats.

In the present disclosure, using an unbiased genetic screen in a fruit fly model of C9ORF72-ALS/FTD, many genetic suppressors of poly(GR) toxicity were identified without affecting the level of toxic poly(GR) protein. In particular, it was found that partial knockdown of Rbp9 suppressed poly(GR) toxicity in non-neuronal cells in a fly model. It was then confirmed that partial knockdown of Rbp9 also suppressed poly(GR) toxicity in neuronal cells in another fly model, and more importantly, this suppression was not due to a reduction in poly(GR) level.

The closest mammalian homolog of the fly gene rbp9 is ELAVL2. Multiple ASOs targeting human ELAVL2 were designed and screened to determine their knockdown efficiency in human iPSC-derived motor neurons. Those ASOs which were found to be effective are found in Table 1 that are SEQ ID NOS: 3-26 and 27-50, and Table 5 that are SEQ ID NOS: 63-64 and 133-150. Multiple siRNA's targeting ELAVL2 were also designed and screened to determine their knockdown efficiency. The siRNAs which were found to be effective are found in Table 3 that are SEQ ID NOS: 57-62 and 99-132.

Therefore, disclosed herein are nucleic acid inhibitors, and methods of using them, to target these modifier genes. This can offer clinical benefits because they may slow down or block ongoing disease processes, even in the presence of disease-causing G₄C₂ repeats RNA and DPR proteins. Also disclosed are methods of screening for other inhibitors of ELAVL2 which can be used to treat diseases and disorders related to G₄C₂ expansion.

### Compositions

Disclosed herein is a nucleic acid inhibitor which is capable of reducing expression of ELAVL2. This nucleic acid molecule can reduce expression by 10%, 15%, 20%, 25%, 30%, 35%, 40%, 45%, 50%, 55%, 60%, 65%, 70%, 75%, 80%, 85%, 90%, 95%, 100%, or any amount in between or below these numbers. This reduction in expression is compared to a control where the nucleic acid is not present. One of skill in the art will understand how to compare the results of inhibition by the nucleic acid inhibitor compared to a control where a negative control sequence or no nucleic acid inhibitor is present.

SEQ ID NO: 1 represents the genomic locus of chromosome 9 which encodes ELAVL2 and includes introns as well as exons (from human hg38 assembly, chr9: 23689906-23826667, reverse complement). SEQ ID NO: 2 represents the nucleic acid which specifically encodes ELAVL2. Contemplated herein is a nucleic acid inhibitor which hybridizes to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides within SEQ ID NO: 1 or SEQ ID NO: 2. Contemplated herein is a nucleic acid inhibitor which hybridizes to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides within SEQ ID NO: 1. Contemplated herein is a nucleic acid inhibitor which hybridizes to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides within SEQ ID NO: 2. In other words, the nucleic acid inhibitor does not need to hybridize completely within SEQ ID NO: 1 or SEQ ID NO: 2, but should hybridize substantially enough within this region as to cause an inhibition in expression of ELAVL2. This is referred to herein as “partial hybridization” within the specified region.

Also disclosed is a nucleic acid inhibitor which hybridizes completely or partially within certain areas of SEQ ID NO: 1 or 2. For example, there are certain regions of SEQ ID NO: 2 which can be considered “hot spots” so that nucleic acid inhibitors designed against those regions show very high probability of abrogating ELAVL2 expression. These regions can comprise, but are not limited to, between nucleotides 1-450 of SEQ ID NO: 2. Therefore, contemplated herein are nucleic acid inhibitors that hybridize to at least 5 contiguous nucleotides within the region of nucleotide number 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, and 450 of SEQ ID NO: 2.

These regions can also comprise, but are not limited to, between nucleotides 2100 and 3983 of SEQ ID NO: 2. Therefore, contemplated herein are nucleic acid inhibitors that hybridize to at least 5 contiguous nucleotides within the region of nucleotide number 2100, 2101, 2102, 2103, 2104, 2105, 2106, 2107, 2108, 2109, 2110, 2111, 2112, 2113, 2114, 2115, 2116, 2117, 2118, 2119, 2120, 2121, 2122, 2123, 2124, 2125, 2126, 2127, 2128, 2129, 2130, 2131, 2132, 2133, 2134, 2135, 2136, 2137, 2138, 2139, 2140, 2141, 2142, 2143, 2144, 2145, 2146, 2147, 2148, 2149, 2150, 2151, 2152, 2153, 2154, 2155, 2156, 2157, 2158, 2159, 2160, 2161, 2162, 2163, 2164, 2165, 2166, 2167, 2168, 2169, 2170, 2171, 2172, 2173, 2174, 2175, 2176, 2177, 2178, 2179, 2180, 2181, 2182, 2183, 2184, 2185, 2186, 2187, 2188, 2189, 2190, 2191, 2192, 2193, 2194, 2195, 2196, 2197, 2198, 2199, 2200, 2201, 2202, 2203, 2204, 2205, 2206, 2207, 2208, 2209, 2210, 2211, 2212, 2213, 2214, 2215, 2216, 2217, 2218, 2219, 2220, 2221, 2222, 2223, 2224, 2225, 2226, 2227, 2228, 2229, 2230, 2231, 2232, 2233, 2234, 2235, 2236, 2237, 2238, 2239, 2240, 2241, 2242, 2243, 2244, 2245, 2246, 2247, 2248, 2249, 2250, 2251, 2252, 2253, 2254, 2255, 2256, 2257, 2258, 2259, 2260, 2261, 2262, 2263, 2264, 2265, 2266, 2267, 2268, 2269, 2270, 2271, 2272, 2273, 2274, 2275, 2276, 2277, 2278, 2279, 2280, 2281, 2282, 2283, 2284, 2285, 2286, 2287, 2288, 2289, 2290, 2291, 2292, 2293, 2294, 2295, 2296, 2297, 2298, 2299, 2300, 2301, 2302, 2303, 2304, 2305, 2306, 2307, 2308, 2309, 2310, 2311, 2312, 2313, 2314, 2315, 2316, 2317, 2318, 2319, 2320, 2321, 2322, 2323, 2324, 2325, 2326, 2327, 2328, 2329, 2330, 2331, 2332, 2333, 2334, 2335, 2336, 2337, 2338, 2339, 2340, 2341, 2342, 2343, 2344, 2345, 2346, 2347, 2348, 2349, 2350, 2351, 2352, 2353, 2354, 2355, 2356, 2357, 2358, 2359, 2360, 2361, 2362, 2363, 2364, 2365, 2366, 2367, 2368, 2369, 2370, 2371, 2372, 2373, 2374, 2375, 2376, 2377, 2378, 2379, 2380, 2381, 2382, 2383, 2384, 2385, 2386, 2387, 2388, 2389, 2390, 2391, 2392, 2393, 2394, 2395, 2396, 2397, 2398, 2399, 2400, 2401, 2402, 2403, 2404, 2405, 2406, 2407, 2408, 2409, 2410, 2411, 2412, 2413, 2414, 2415, 2416, 2417, 2418, 2419, 2420, 2421, 2422, 2423, 2424, 2425, 2426, 2427, 2428, 2429, 2430, 2431, 2432, 2433, 2434, 2435, 2436, 2437, 2438, 2439, 2440, 2441, 2442, 2443, 2444, 2445, 2446, 2447, 2448, 2449, 2450, 2451, 2452, 2453, 2454, 2455, 2456, 2457, 2458, 2459, 2460, 2461, 2462, 2463, 2464, 2465, 2466, 2467, 2468, 2469, 2470, 2471, 2472, 2473, 2474, 2475, 2476, 2477, 2478, 2479, 2480, 2481, 2482, 2483, 2484, 2485, 2486, 2487, 2488, 2489, 2490, 2491, 2492, 2493, 2494, 2495, 2496, 2497, 2498, 2499, 2500, 2501, 2502, 2503, 2504, 2505, 2506, 2507, 2508, 2509, 2510, 2511, 2512, 2513, 2514, 2515, 2516, 2517, 2518, 2519, 2520, 2521, 2522, 2523, 2524, 2525, 2526, 2527, 2528, 2529, 2530, 2531, 2532, 2533, 2534, 2535, 2536, 2537, 2538, 2539, 2540, 2541, 2542, 2543, 2544, 2545, 2546, 2547, 2548, 2549, 2550, 2551, 2552, 2553, 2554, 2555, 2556, 2557, 2558, 2559, 2560, 2561, 2562, 2563, 2564, 2565, 2566, 2567, 2568, 2569, 2570, 2571, 2572, 2573, 2574, 2575, 2576, 2577, 2578, 2579, 2580, 2581, 2582, 2583, 2584, 2585, 2586, 2587, 2588, 2589, 2590, 2591, 2592, 2593, 2594, 2595, 2596, 2597, 2598, 2599, 2600, 2601, 2602, 2603, 2604, 2605, 2606, 2607, 2608, 2609, 2610, 2611, 2612, 2613, 2614, 2615, 2616, 2617, 2618, 2619, 2620, 2621, 2622, 2623, 2624, 2625, 2626, 2627, 2628, 2629, 2630, 2631, 2632, 2633, 2634, 2635, 2636, 2637, 2638, 2639, 2640, 2641, 2642, 2643, 2644, 2645, 2646, 2647, 2648, 2649, 2650, 2651, 2652, 2653, 2654, 2655, 2656, 2657, 2658, 2659, 2660, 2661, 2662, 2663, 2664, 2665, 2666, 2667, 2668, 2669, 2670, 2671, 2672, 2673, 2674, 2675, 2676, 2677, 2678, 2679, 2680, 2681, 2682, 2683, 2684, 2685, 2686, 2687, 2688, 2689, 2690, 2691, 2692, 2693, 2694, 2695, 2696, 2697, 2698, 2699, 2700, 2701, 2702, 2703, 2704, 2705, 2706, 2707, 2708, 2709, 2710, 2711, 2712, 2713, 2714, 2715, 2716, 2717, 2718, 2719, 2720, 2721, 2722, 2723, 2724, 2725, 2726, 2727, 2728, 2729, 2730, 2731, 2732, 2733, 2734, 2735, 2736, 2737, 2738, 2739, 2740, 2741, 2742, 2743, 2744, 2745, 2746, 2747, 2748, 2749, 2750, 2751, 2752, 2753, 2754, 2755, 2756, 2757, 2758, 2759, 2760, 2761, 2762, 2763, 2764, 2765, 2766, 2767, 2768, 2769, 2770, 2771, 2772, 2773, 2774, 2775, 2776, 2777, 2778, 2779, 2780, 2781, 2782, 2783, 2784, 2785, 2786, 2787, 2788, 2789, 2790, 2791, 2792, 2793, 2794, 2795, 2796, 2797, 2798, 2799, 2800, 2801, 2802, 2803, 2804, 2805, 2806, 2807, 2808, 2809, 2810, 2811, 2812, 2813, 2814, 2815, 2816, 2817, 2818, 2819, 2820, 2821, 2822, 2823, 2824, 2825, 2826, 2827, 2828, 2829, 2830, 2831, 2832, 2833, 2834, 2835, 2836, 2837, 2838, 2839, 2840, 2841, 2842, 2843, 2844, 2845, 2846, 2847, 2848, 2849, 2850, 2851, 2852, 2853, 2854, 2855, 2856, 2857, 2858, 2859, 2860, 2861, 2862, 2863, 2864, 2865, 2866, 2867, 2868, 2869, 2870, 2871, 2872, 2873, 2874, 2875, 2876, 2877, 2878, 2879, 2880, 2881, 2882, 2883, 2884, 2885, 2886, 2887, 2888, 2889, 2890, 2891, 2892, 2893, 2894, 2895, 2896, 2897, 2898, 2899, 2900, 2901, 2902, 2903, 2904, 2905, 2906, 2907, 2908, 2909, 2910, 2911, 2912, 2913, 2914, 2915, 2916, 2917, 2918, 2919, 2920, 2921, 2922, 2923, 2924, 2925, 2926, 2927, 2928, 2929, 2930, 2931, 2932, 2933, 2934, 2935, 2936, 2937, 2938, 2939, 2940, 2941, 2942, 2943, 2944, 2945, 2946, 2947, 2948, 2949, 2950, 2951, 2952, 2953, 2954, 2955, 2956, 2957, 2958, 2959, 2960, 2961, 2962, 2963, 2964, 2965, 2966, 2967, 2968, 2969, 2970, 2971, 2972, 2973, 2974, 2975, 2976, 2977, 2978, 2979, 2980, 2981, 2982, 2983, 2984, 2985, 2986, 2987, 2988, 2989, 2990, 2991, 2992, 2993, 2994, 2995, 2996, 2997, 2998, 2999, 3000, 3001, 3002, 3003, 3004, 3005, 3006, 3007, 3008, 3009, 3010, 3011, 3012, 3013, 3014, 3015, 3016, 3017, 3018, 3019, 3020, 3021, 3022, 3023, 3024, 3025, 3026, 3027, 3028, 3029, 3030, 3031, 3032, 3033, 3034, 3035, 3036, 3037, 3038, 3039, 3040, 3041, 3042, 3043, 3044, 3045, 3046, 3047, 3048, 3049, 3050, 3051, 3052, 3053, 3054, 3055, 3056, 3057, 3058, 3059, 3060, 3061, 3062, 3063, 3064, 3065, 3066, 3067, 3068, 3069, 3070, 3071, 3072, 3073, 3074, 3075, 3076, 3077, 3078, 3079, 3080, 3081, 3082, 3083, 3084, 3085, 3086, 3087, 3088, 3089, 3090, 3091, 3092, 3093, 3094, 3095, 3096, 3097, 3098, 3099, 3100, 3101, 3102, 3103, 3104, 3105, 3106, 3107, 3108, 3109, 3110, 3111, 3112, 3113, 3114, 3115, 3116, 3117, 3118, 3119, 3120, 3121, 3122, 3123, 3124, 3125, 3126, 3127, 3128, 3129, 3130, 3131, 3132, 3133, 3134, 3135, 3136, 3137, 3138, 3139, 3140, 3141, 3142, 3143, 3144, 3145, 3146, 3147, 3148, 3149, 3150, 3151, 3152, 3153, 3154, 3155, 3156, 3157, 3158, 3159, 3160, 3161, 3162, 3163, 3164, 3165, 3166, 3167, 3168, 3169, 3170, 3171, 3172, 3173, 3174, 3175, 3176, 3177, 3178, 3179, 3180, 3181, 3182, 3183, 3184, 3185, 3186, 3187, 3188, 3189, 3190, 3191, 3192, 3193, 3194, 3195, 3196, 3197, 3198, 3199, 3200, 3201, 3202, 3203, 3204, 3205, 3206, 3207, 3208, 3209, 3210, 3211, 3212, 3213, 3214, 3215, 3216, 3217, 3218, 3219, 3220, 3221, 3222, 3223, 3224, 3225, 3226, 3227, 3228, 3229, 3230, 3231, 3232, 3233, 3234, 3235, 3236, 3237, 3238, 3239, 3240, 3241, 3242, 3243, 3244, 3245, 3246, 3247, 3248, 3249, 3250, 3251, 3252, 3253, 3254, 3255, 3256, 3257, 3258, 3259, 3260, 3261, 3262, 3263, 3264, 3265, 3266, 3267, 3268, 3269, 3270, 3271, 3272, 3273, 3274, 3275, 3276, 3277, 3278, 3279, 3280, 3281, 3282, 3283, 3284, 3285, 3286, 3287, 3288, 3289, 3290, 3291, 3292, 3293, 3294, 3295, 3296, 3297, 3298, 3299, 3300, 3301, 3302, 3303, 3304, 3305, 3306, 3307, 3308, 3309, 3310, 3311, 3312, 3313, 3314, 3315, 3316, 3317, 3318, 3319, 3320, 3321, 3322, 3323, 3324, 3325, 3326, 3327, 3328, 3329, 3330, 3331, 3332, 3333, 3334, 3335, 3336, 3337, 3338, 3339, 3340, 3341, 3342, 3343, 3344, 3345, 3346, 3347, 3348, 3349, 3350, 3351, 3352, 3353, 3354, 3355, 3356, 3357, 3358, 3359, 3360, 3361, 3362, 3363, 3364, 3365, 3366, 3367, 3368, 3369, 3370, 3371, 3372, 3373, 3374, 3375, 3376, 3377, 3378, 3379, 3380, 3381, 3382, 3383, 3384, 3385, 3386, 3387, 3388, 3389, 3390, 3391, 3392, 3393, 3394, 3395, 3396, 3397, 3398, 3399, 3400, 3401, 3402, 3403, 3404, 3405, 3406, 3407, 3408, 3409, 3410, 3411, 3412, 3413, 3414, 3415, 3416, 3417, 3418, 3419, 3420, 3421, 3422, 3423, 3424, 3425, 3426, 3427, 3428, 3429, 3430, 3431, 3432, 3433, 3434, 3435, 3436, 3437, 3438, 3439, 3440, 3441, 3442, 3443, 3444, 3445, 3446, 3447, 3448, 3449, 3450, 3451, 3452, 3453, 3454, 3455, 3456, 3457, 3458, 3459, 3460, 3461, 3462, 3463, 3464, 3465, 3466, 3467, 3468, 3469, 3470, 3471, 3472, 3473, 3474, 3475, 3476, 3477, 3478, 3479, 3480, 3481, 3482, 3483, 3484, 3485, 3486, 3487, 3488, 3489, 3490, 3491, 3492, 3493, 3494, 3495, 3496, 3497, 3498, 3499, 3500, 3501, 3502, 3503, 3504, 3505, 3506, 3507, 3508, 3509, 3510, 3511, 3512, 3513, 3514, 3515, 3516, 3517, 3518, 3519, 3520, 3521, 3522, 3523, 3524, 3525, 3526, 3527, 3528, 3529, 3530, 3531, 3532, 3533, 3534, 3535, 3536, 3537, 3538, 3539, 3540, 3541, 3542, 3543, 3544, 3545, 3546, 3547, 3548, 3549, 3550, 3551, 3552, 3553, 3554, 3555, 3556, 3557, 3558, 3559, 3560, 3561, 3562, 3563, 3564, 3565, 3566, 3567, 3568, 3569, 3570, 3571, 3572, 3573, 3574, 3575, 3576, 3577, 3578, 3579, 3580, 3581, 3582, 3583, 3584, 3585, 3586, 3587, 3588, 3589, 3590, 3591, 3592, 3593, 3594, 3595, 3596, 3597, 3598, 3599, 3600, 3601, 3602, 3603, 3604, 3605, 3606, 3607, 3608, 3609, 3610, 3611, 3612, 3613, 3614, 3615, 3616, 3617, 3618, 3619, 3620, 3621, 3622, 3623, 3624, 3625, 3626, 3627, 3628, 3629, 3630, 3631, 3632, 3633, 3634, 3635, 3636, 3637, 3638, 3639, 3640, 3641, 3642, 3643, 3644, 3645, 3646, 3647, 3648, 3649, 3650, 3651, 3652, 3653, 3654, 3655, 3656, 3657, 3658, 3659, 3660, 3661, 3662, 3663, 3664, 3665, 3666, 3667, 3668, 3669, 3670, 3671, 3672, 3673, 3674, 3675, 3676, 3677, 3678, 3679, 3680, 3681, 3682, 3683, 3684, 3685, 3686, 3687, 3688, 3689, 3690, 3691, 3692, 3693, 3694, 3695, 3696, 3697, 3698, 3699, 3700, 3701, 3702, 3703, 3704, 3705, 3706, 3707, 3708, 3709, 3710, 3711, 3712, 3713, 3714, 3715, 3716, 3717, 3718, 3719, 3720, 3721, 3722, 3723, 3724, 3725, 3726, 3727, 3728, 3729, 3730, 3731, 3732, 3733, 3734, 3735, 3736, 3737, 3738, 3739, 3740, 3741, 3742, 3743, 3744, 3745, 3746, 3747, 3748, 3749, 3750, 3751, 3752, 3753, 3754, 3755, 3756, 3757, 3758, 3759, 3760, 3761, 3762, 3763, 3764, 3765, 3766, 3767, 3768, 3769, 3770, 3771, 3772, 3773, 3774, 3775, 3776, 3777, 3778, 3779, 3780, 3781, 3782, 3783, 3784, 3785, 3786, 3787, 3788, 3789, 3790, 3791, 3792, 3793, 3794, 3795, 3796, 3797, 3798, 3799, 3800, 3801, 3802, 3803, 3804, 3805, 3806, 3807, 3808, 3809, 3810, 3811, 3812, 3813, 3814, 3815, 3816, 3817, 3818, 3819, 3820, 3821, 3822, 3823, 3824, 3825, 3826, 3827, 3828, 3829, 3830, 3831, 3832, 3833, 3834, 3835, 3836, 3837, 3838, 3839, 3840, 3841, 3842, 3843, 3844, 3845, 3846, 3847, 3848, 3849, 3850, 3851, 3852, 3853, 3854, 3855, 3856, 3857, 3858, 3859, 3860, 3861, 3862, 3863, 3864, 3865, 3866, 3867, 3868, 3869, 3870, 3871, 3872, 3873, 3874, 3875, 3876, 3877, 3878, 3879, 3880, 3881, 3882, 3883, 3884, 3885, 3886, 3887, 3888, 3889, 3890, 3891, 3892, 3893, 3894, 3895, 3896, 3897, 3898, 3899, 3900, 3901, 3902, 3903, 3904, 3905, 3906, 3907, 3908, 3909, 3910, 3911, 3912, 3913, 3914, 3915, 3916, 3917, 3918, 3919, 3920, 3921, 3922, 3923, 3924, 3925, 3926, 3927, 3928, 3929, 3930, 3931, 3932, 3933, 3934, 3935, 3936, 3937, 3938, 3939, 3940, 3941, 3942, 3943, 3944, 3945, 3946, 3947, 3948, 3949, 3950, 3951, 3952, 3953, 3954, 3955, 3956, 3957, 3958, 3959, 3960, 3961, 3962, 3963, 3964, 3965, 3966, 3967, 3968, 3969, 3970, 3971, 3972, 3973, 3974, 3975, 3976, 3977, 3978, 3979, 3980, 3981, 3982, and 3983.

Specifically disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of SEQ ID NOS: 3-50, 63-64, and 133-150. SEQ ID NOS: 3-50 can be found in Table 1, and it is noted that SEQ ID NOS: 3-26 correspond to unmodified nucleic acid inhibitors, while SEQ ID NOS: 27-50 are examples of modified nucleic acid inhibitors. SEQ ID NOS: 63-64 and 133-150, which are also examples of modified nucleic acid inhibitors, can be found in Table 5. Referring to SEQ ID NOS: 27-50, 63-64, and 133-150, the nucleotides prefaced with “e” are 2′-O-MOE nucleotides, the nucleotides prefaced with “d” are deoxynucleotides, and # indicates phosphorothioate (PS) linkage. It is further noted that for in vivo use in the central nervous system (e.g. clinical use) mixed backbones can be used (i.e., reduced PS content, both with normal PO linkages and with next-generation linkages such as phosphoramidates). For example, for use in the central nervous system, an inhibitor can be synthesized with phosphorothioate or phosphoramidate linkages at the terminal positions, phosphodiester linkages between sugar-modified nucleotides, phosphorothioate linkages throughout the stretch of deoxynucleotides. A subset of positions within the deoxynucleotide stretch can be modified with a phosphonate or phosphoramidate linkage (for example 1, 2, or 3 linkages within the stretch of deoxynucleotides might typically be modified with a phosphonate or phosphoramidate linkage). Other patterns of backbone modifications are known to one of skill in the art.

Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of SEQ ID NOS: 54-62 and 82-132. SEQ ID NOS: 54-62 and 82-132 can be found in Table 3, and it is noted that SEQ ID NO: 54-56 and 82-98 are examples of unmodified nucleic acid inhibitors, SEQ ID NO: 57-59 and 99-115 are examples of modified nucleic acid inhibitors targeting an antisense (as) strand, and SEQ ID NO: 60-62 and 116-132 are examples of modified nucleic acid inhibitors targeting a sense strand.

Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 17. Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 21. Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 24.

Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 41. Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 45. Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 48.

Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 63. Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 64.

Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 54. Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 55. Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 56.

Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 57. Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 58. Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 59.

Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 60. Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 61. Further, disclosed are nucleic acid inhibitors comprising 80%, 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 62.

In some embodiments, the nucleic acid inhibitor comprises SEQ ID NO: 17. In some embodiments, the nucleic acid inhibitor comprises SEQ ID NO: 21. In some embodiments, the nucleic acid inhibitor comprises SEQ ID NO: 24.

In some embodiments, the nucleic acid inhibitor comprises SEQ ID NO: 41. In some embodiments, the nucleic acid inhibitor comprises SEQ ID NO: 45. In some embodiments, the nucleic acid inhibitor comprises SEQ ID NO: 48.

In some embodiments, the nucleic acid inhibitor comprises SEQ ID NO: 63. In some embodiments, the nucleic acid inhibitor comprises SEQ ID NO: 64.

In some embodiments, the nucleic acid inhibitor comprises SEQ ID NO: 54. In some embodiments, the nucleic acid inhibitor comprises SEQ ID NO: 55. In some embodiments, the nucleic acid inhibitor comprises SEQ ID NO: 56.

In some embodiments, the nucleic acid inhibitor comprises SEQ ID NO: 57. In some embodiments, the nucleic acid inhibitor comprises SEQ ID NO: 58. In some embodiments, the nucleic acid inhibitor comprises SEQ ID NO: 59.

In some embodiments, the nucleic acid inhibitor comprises SEQ ID NO: 60. In some embodiments, the nucleic acid inhibitor comprises SEQ ID NO: 61. In some embodiments, the nucleic acid inhibitor comprises SEQ ID NO: 62.

Referring to SEQ ID NOS: 57-62 and 99-132, the nucleotides prefaced with “m” are 2′-O-methyl, the nucleotides prefaced with “f” are 2′-fluoro, the strands prefaced with “P” are 5′-phosphate, and # indicates phosphorothioate (PS) linkage.

In some embodiments, 5′-phosphate analogues, for example vinylphosphate or substituted phosphates, can be used.

The nucleic acid inhibitor described herein can be any length. One of skill in the art will recognize that, for example, a typical antisense oligonucleotide is typically between 15 and 30 nucleotides long. However, contemplated herein are nucleic acids which are 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long, or longer.

The nucleic acid inhibitor described herein can be an antisense oligonucleotide (ASO) or an RNAi molecule, such as siRNA. Types of antisense and RNAi molecules are disclosed in Watts et al. (Watts J K, Corey D R. Silencing disease genes in the laboratory and the clinic. J Pathol. 2012 January; 226 (2): 365-79. doi: 10.1002/path.2993). Also contemplated herein is short interfering RNA (siRNA), double stranded RNA (dsRNA), micro-RNA (miRNA), small nucleolar RNA (sno-RNA), Piwi-interacting RNA (piRNA), or short hairpin RNA (shRNA) molecules.

When the nucleic acid inhibitor is an antisense oligonucleotide or an RNAi molecule, it can be modified. One of skill in the art will understand what types of modifications can be made to the antisense oligonucleotide or the RNAi molecule in order to provide stability, for example. Examples of modifications include, but are not limited to, at least one modified sugar moiety; at least one modified inter-nucleotide linkage; and/or at least one modified nucleotide.

Specifically, such modifications can include, for example, those which are internal or at the end(s) of the oligonucleotide molecule and include additions to the molecule of the internucleoside phosphate linkages, such as cholesteryl or diamine compounds with varying numbers of carbon residues between the amino groups and terminal ribose, deoxyribose and phosphate modifications which cleave, or crosslink to the opposite chains or to associated enzymes or other proteins. Examples of such modified oligonucleotides include oligonucleotides with a modified base and/or sugar such as arabinose instead of ribose, or a 3′, 5′-substituted oligonucleotide having a sugar which, at both its 3′ and 5′ positions is attached to a chemical group other than a hydroxyl group (at its 3′ position) and other than a phosphate group (at its 5′ position). Other modified oligonucleotides are capped with a nuclease resistance-conferring bulky substituent at their 3′ and/or 5′ end(s), or have a substitution in one nonbridging oxygen per nucleotide. Such modifications can be at some or all of the internucleoside linkages, as well as at either or both ends of the oligonucleotide and/or in the interior of the molecule. For the preparation of such modified oligonucleotides, see, e.g., Agrawal (1994) Methods in Molecular Biology 26; Uhlmann et al. (1990) Chem. Rev. 90:543-583). Oligonucleotides, which are self-stabilized are also considered to be modified oligonucleotides useful in the methods of the invention (Tang et al. (1993) Nucleic Acids Res. 20:2729-2735). These oligonucleotides comprise two regions: a target hybridizing region; and a self-complementary region having an oligonucleotide sequence complementary to a nucleic acid sequence that is within the self-stabilized oligonucleotide.

Such modifications can comprise an internucleotide linkage comprising: phosphorothioate, alkylphosphonate, phosphorodithioate, alkylphosphonothioate, phosphoramidate, carbamate, carbonate, phosphate triester, acetamidate, and/or carboxymethyl ester. The at least one modification can comprise a modified nucleotide comprises a peptide nucleic acid, a morpholino analogue, a locked nucleic acid (LNA) or other bridged or bicyclic nucleic acid analogue, and/or a combination thereof. The at least one modification can comprise a modified sugar moiety comprises: a 2′-O-methoxyethyl modified sugar moiety, a 2′-methoxy modified sugar moiety, and/or a 2′-O-alkyl modified sugar moiety.

The nucleic acid inhibitor described herein can include a conjugate. Such conjugates are known to those of skill in the art. Examples of modifications can be found in Winkler (Oligonucleotide conjugates for therapeutic applications. Ther Deliv. 2013 July; 4 (7): 791-809), herein incorporated by reference in its entirety for its teaching concerning conjugates. Nucleic acid inhibitors described herein can be conjugated to a nanoparticle, to enhance a property of the compositions, e.g., a pharmacokinetic parameter such as absorption, efficacy, bioavailability, and/or half-life. For example, the conjugate can comprise a therapeutic agent, a diagnostic agent, or an agent that increases cell uptake or penetration. More specifically, the conjugate can comprise a peptide, a protein, an antibody or fragment thereof, a lipid, a neurotransmitter, a cationic polymer, a nanoparticle, a probe, or a carbohydrate.

The conjugate can be a multivalent conjugate, such as a multimeric antisense oligonucleotide or multivalent siRNA, for example. In the case of multivalent siRNA, it can suppress one gene at several sites, or suppress multiple genes all at once. In the case of multivalent ASOs, ASO monomers to one or more targets can be co-synthesized as homo- or heterodimers or multimers via phosphodiester linkers, for example, that are stable outside of cells, but cleaved inside cells, releasing the active ASO monomers.

Also disclosed are vectors, such as viral vectors, which can be used to deliver the nucleic acid inhibitors. Those of skill in the art are apprised of how to engineer vectors to express nucleic acids of interest, such as ASOs. The viral vector can also be used to induce intracellular expression of the nucleic acid inhibitors.

Antisense sequences of the invention may be delivered alone in vivo or in association with a vector. In its broadest sense, a “vector” is any vehicle capable of facilitating the transfer of the antisense sequence to the cells and preferably cells expressing ELAVL2. Preferably, the vector transports the antisense sequence to cells with reduced degradation relative to the extent of degradation that would result in the absence of the vector. In general, the vectors useful in the invention include, but are not limited to, plasmids, phagemids, viruses, and other vehicles derived from viral or bacterial sources that have been manipulated by the insertion or incorporation of the ASO sequences. Viral vectors are a preferred type of vector and include, but are not limited to, nucleic acid sequences from the following viruses: lentivirus such as HIV-1, retrovirus, such as moloney murine leukemia virus, adenovirus, adeno-associated virus (AAV); SV40-type viruses; Herpes viruses such as HSV-1 and vaccinia virus. One can readily employ other vectors not named but known in the art.

Retrovirus-based and lentivirus-based vectors that are replication-deficient (i.e., capable of directing synthesis of the desired AON, but incapable of producing an infectious particle) have been approved for human gene therapy trials. They have the property to integrate into the target cell genome, thus allowing for a persistent transgene expression in the target cells and their progeny.

In one specific example, the ASO can be delivered using an AAV vector. The human parvovirus Adeno-Associated Virus (AAV) is a dependovirus that is naturally defective for replication which can be repurposed into a vector able to elicit prolonged expression of therapeutic cargo within infected cells. Accordingly, the present invention relates to an AAV vector comprising or expressing a nucleic acid inhibitor (antisense sequence or interfering RNA), targeting ELAVL2. Accordingly, the present invention relates to an AAV vector comprising an ASO as described above, targeting ELAVL2.

The nucleic acid inhibitors of the present invention can be presented in the form of a pharmaceutical composition. The pharmaceutical composition can comprise, for example, a vehicle for delivery of the nucleic acid inhibitor. In addition to the nucleic acid inhibitor (ASO or siRNA), a pharmaceutical composition of the present invention may also include a pharmaceutically or physiologically acceptable carrier such as saline, sodium phosphate, etc. The composition will generally be in the form of a liquid, although this needs not always to be the case. Suitable carriers, excipients and diluents include lactose, dextrose, sucrose, sorbitol, mannitol, starches, gum acacia, calcium phosphates, alginate, tragacanth, gelatin, calcium silicate, microcrystalline cellulose, polyvinylpyrrolidone, cellulose, water syrup, methyl cellulose, methyl and propylhydroxybenzoates, mineral oil, etc. The formulation can also include lubricating agents, wetting agents, emulsifying agents, preservatives, buffering agents, etc. In particular, the present invention involves the administration of a nucleic acid inhibitor and is thus somewhat akin to gene therapy. Those of skill in the art will recognize that nucleic acids are often delivered in conjunction with lipids (e.g. cationic lipids or neutral lipids, or mixtures of these), frequently in the form of liposomes or other suitable micro- or nano-structured material (e.g. micelles, lipocomplexes, dendrimers, emulsions, cubic phases, etc.).

The compositions of the invention are generally administered via enteral or parenteral routes, e.g. intravenously (i.v.), intra-arterially, subcutaneously, intramuscularly (i.m.), intracerebrally, intracerebroventricularly (i.e.v.), intrathecally (i.t.), intraperitoneally (i.p.), although other types of administration are not precluded, e.g. via inhalation, intranasally, topical, rectally, intraosseous, eye drops, ear drops administration, etc.

Injectable preparations, for example, sterile injectable aqueous or oleaginous suspensions may be formulated according to the known art using suitable dispensing or wetting agents and suspending agents. The sterile injectable preparation can also be a sterile injectable solution or suspension in a nontoxic parenterally acceptable diluent or solvent, for example, as a solution in 1,3-butanediol. While delivery may be either local (i.e. in situ, directly into tissue such as muscle tissue) or systemic, usually delivery will be local to affected muscle tissue, e.g. to skeletal muscle, smooth muscle, heart muscle, etc. Depending on the form of the AONs that are administered and the tissue or cell type that is targeted, techniques such as electroporation, sonoporation, a “gene gun” (delivering nucleic acid-coated gold particles), etc. may be employed.

### Methods of Treatment

Disclosed herein are methods of using nucleic acid inhibitors to treat disorders and diseases associated with poly(GR) toxicity in neuronal cells in a subject in need thereof. the method comprising modulating expression of ELAVL2. The poly(GR) toxicity can be caused by a GGGGCC (G₄C₂) repeat expansion in a first intron of C9ORF72. As described above, G₄C₂ repeat expansion is associated with ALS and FTD. Therefore, disclosed herein is a method of treating or preventing ALS or FTD by using nucleic acid inhibitors to modulate expression of ELAVL2. Further disclosed herein is a method of treating disorders and diseases other than those that are C9ORF72-related. For example, also contemplated are other diseases (such as non-C9ORF72-related ALS and FTD and a subset of patients with Alzheimer disease) in neuronal cells in a subject in need thereof, the method comprising modulating expression of ELAVL2.

By “treat” is meant to reduce the symptoms or clinical manifestations by 10%, 15%, 20%, 25%, 30%, 35%, 40%, 45%, 50%, 55%, 60%, 65%, 70%, 75%, 80%, 85%, 90%, 95%, or 100% compared to a control where a nucleic acid inhibitor of ELAVL2 is not given. One measurement to determine the effectiveness of treatment could be the reduction in the death of motor neurons in a subject being given the nucleic acid inhibitors of ELAVL2. The symptoms of ALS which can be reduced include, but are not limited to, muscle twitches in the arm, leg, shoulder, or tongue; muscle cramps; tight and stiff muscles (spasticity); muscle weakness affecting an arm, a leg, the neck, or diaphragm; slurred and nasal speech; or difficulty chewing or swallowing.

In FTD, the reduction in clinical manifestation can be measured by measuring the frontal and/or temporal lobes, as FTD is known to cause them to shrink. Therefore, the nucleic acid inhibitors disclosed herein can reduce the shrinkage of these lobes. Symptoms of FTD which can be ameliorated by the nucleic acid inhibitors disclosed herein include, but are not limited to, changes in their personalities and become socially inappropriate, impulsive or emotionally indifferent, or loss of language. These can be measured by a clinician, and effectiveness of the treatments disclosed herein can be determined by monitoring symptoms or by monitoring clinical manifestations.

One skilled in the art will recognize that the amount of a nucleic acid inhibitor to be administered will be an amount that is sufficient to induce amelioration of unwanted ALS or FTD symptoms. Such an amount may vary inter alia depending on such factors as the gender, age, weight, overall physical condition of the patient, etc. and may be determined on a case by case basis. The amount may also vary according to other components of a treatment protocol (e.g. administration of other medicaments, etc.).

When dosed directly into cerebrospinal fluid (e.g. by intrathecal or intraventricular injection, a suitable dose is generally in the range of from about 0.5 mg to about 500 mg, and more usually from about 5 mg to about 150 mg per dose. In a specific example, the dose can be between 8 mg to 100 mg. Such doses are typically repeated after several weeks to several months. In a specific example, the treatment is given every three to four months. Alternatively, nucleic acid inhibitors (ASOs or siRNAs, for example) may be delivered using conjugation or formulation approaches that allow delivery to the central nervous system through the bloodstream, but these approaches would typically require higher doses. If a viral-based delivery of the nucleic acid inhibitor is chosen, suitable doses will depend on different factors such as the virus that is employed, the route of delivery (intramuscular, intravenous, intra-arterial or other), but may typically range from 109 to 1015 viral particles/kg. Those of skill in the art will recognize that such parameters are normally worked out during clinical trials. Further, those of skill in the art will recognize that, while disease symptoms may be completely alleviated by the treatments described herein, this need not be the case. Even a partial or intermittent relief of symptoms may be of great benefit to the recipient. In addition, treatment of the patient may be a single event, or the patient is administered with the ASOs on multiple occasions, that may be, depending on the results obtained, several days apart, several weeks apart, or several months apart, or even several years apart.

### Methods of Screening

Also disclosed herein are methods of screening for a nucleic acid inhibitor of ELAVL2, the method comprising contacting SEQ ID NO: 1 or 2 with complementary nucleic acids between 15-30 nucleotides long, and determining which are effective at knocking down expression of ELAVL2. This screen can, in particular, be directed to the regions of the ELAVL2 locus described above, such as between nucleotides 1-450 and 2100-3983 of SEQ ID NO: 2. One of skill in the art will understand which tools can be used to screen for potential nucleic acid inhibitors. For example, Gagnon et al. (Gagnon K T, Corey D R. Guidelines for Experiments Using Antisense Oligonucleotides and Double-Stranded RNAs. Nucleic Acid Ther. 2019 June; 29 (3): 116-122) provides detailed examples of how to screen for ASOs, and this reference is incorporated in its entirety for its teaching concerning screening for ASOs. Also disclosed herein are nucleic acid inhibitors identified by these methods which are capable of targeting ELAVL2 nucleic acid.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1: Antisense Oligonucleotides (ASOs) Effective Against ELAVL2 (Set 1)

Table 1: SEQ ID NOS: 3-26 correspond to unmodified nucleic acid inhibitors, while SEQ ID NOS: 27-50 are examples of modified nucleic acid inhibitors. Referring to SEQ ID NOS: 27-50, the nucleotides prefaced with “e” are 2′-O-MOE nucleotides, and the nucleotides prefaced with “d” are deoxynucleotides. # indicates phosphorothioate (PS) linkage.

| | Chr9 | | | |
|---|---|---|---|---|
| | starting | | | |
| number | position | sequence | formatted sequence | name |
| W-578 | 23826208 | ACTAAGA | (eA)#(eC)#(eT)#(eA)#(eA)#(dG)#(dA)# | ElavL2_C1 |
| | | ACAGAAA | (dA)#(dC)#(dA)#(dG)#(dA)#(dA)#(eA)# | |
| | | TAGG (SEQ | (eT)#(eA)#(eG)#(eG) (SEQ ID NO: 27) | |
| | | ID NO: 3) | | |
| W-579 | 23826204 | TCTAACTA | (eT)#(eC)#(eT)#(eA)#(eA)#(dC)#(dT)#(dA)# | ElavL2_C2 |
| | | AGAACAG | (dA)#(dG)#(dA)#(dA)#(dC)#(dA)#(dG)# | |
| | | AAATA | (eA)#(eA)#(eA)#(eT)#(eA) | |
| | | (SEQ ID | (SEQ ID NO: 28) | |
| | | NO: 4) | | |
| W-580 | 23826181 | CTACAGG | (eC)#(eT)#(eA)#(eC)#(eA)#(dG)#(dG)# | ElavL2_C3 |
| | | AGTAAGC | (dA)#(dG)#(dT)#(dA)#(dA)#(dG)#(eC)# | |
| | | TGCT (SEQ | (eT)#(eG)#(eC)#(eT) (SEQ ID NO: 29) | |
| | | ID NO: 5) | | |
| W-581 | 23826195 | TGCTGCAT | (eT)#(eG)#(eC)#(eT)#(eG)#(dC)#(dA)#(dT)# | ElavL2_C4 |
| | | CTCTAACT | (dC)#(dT)#(dC)#(dT)#(dA)#(dA)#(dC)#(eT)# | |
| | | AAGA (SEQ | (eA)#(eA)#(eG)#(eA) (SEQ ID NO: 30) | |
| | | ID NO: 6) | | |
| W-582 | 23826169 | TTTAGTAG | (eT)#(eT)#(eT)#(eA)#(eG)#(dT)#(dA)#(dG)# | ElavL2_C5 |
| | | GTCGCTAC | (dG)#(dT)#(dC)#(dG)#(dC)#(dT)#(dA)#(eC)# | |
| | | AGGA (SEQ | (eA)#(eG)#(eG)#(eA) (SEQ ID NO: 31) | |
| | | ID NO: 7) | | |
| W-583 | 23825810 | TCCCTTAA | (eT)#(eC)#(eC)#(eC)#(eT)#(dT)#(dA)#(dA)# | ElavL2_6 |
| | | GGTTTTTG | (dG)#(dG)#(dT)#(dT)#(dT)#(dT)#(dT)#(eG)# | |
| | | TGTA (SEQ | (eT)#(eG)#(eT)#(eA) (SEQ ID NO: 32) | |
| | | ID NO: 8) | | |
| W-584 | 23692290 | ACCTTCTT | (eA)#(eC)#(eC)#(eT)#(eT)#(dC)#(dT)#(dT)# | ElavL2_7 |
| | | CAAAGAA | (dC)#(dA)#(dA)#(dA)#(dG)#(dA)#(dA)# | |
| | | GGCAA | (eG)#(eG)#(eC)#(eA)#(eA) | |
| | | (SEQ ID | (SEQ ID NO: 33) | |
| | | NO: 9) | | |
| W-585 | 23691920 | TCAAAGA | (eT)#(eC)#(eA)#(eA)#(eA)#(dG)#(dA)# | ElavL2_8 |
| | | GGCTCTTA | (dG)#(dG)#(dC)#(dT)#(dC)#(dT)#(dT)#(dA)# | |
| | | TCTCT | (eT)#(eC)#(eT)#(eC)#(eT) | |
| | | (SEQ ID | (SEQ ID NO: 34) | |
| | | NO: 10) | | |
| W-586 | 23690682 | AACTATGT | (eA)#(eA)#(eC)#(eT)#(eA)#(dT)#(dG)#(dT)# | ElavL2_9 |
| | | TATGCATA | (dT)#(dA)#(dT)#(dG)#(dC)#(dA)#(dT)#(eA)# | |
| | | GCAC (SEQ | (eG)#(eC)#(eA)#(eC) (SEQ ID NO: 35) | |
| | | ID NO: 11) | | |
| W-587 | 23692823 | GTGCCCA | (eG)#(eT)#(eG)#(eC)#(eC)#(dC)#(dA)# | ElavL2_10 |
| | | GGGATATT | (dG)#(dG)#(dG)#(dA)#(dT)#(dA)#(dT)#(dT)# | |
| | | AATTC | (eA)#(eA)#(eT)#(eT)#(eC) | |
| | | (SEQ ID | (SEQ ID NO: 36) | |
| | | NO: 12) | | |
| W-588 | 23690493 | ACCTAAG | (eA)#(eC)#(eC)#(eT)#(eA)#(dA)#(dG)# | ElavL2_11 |
| | | GATATGAG | (dG)#(dA)#(dT)#(dA)#(dT)#(dG)#(eA)#(eG)# | |
| | | AAA (SEQ | (eA)#(eA)#(eA) (SEQ ID NO: 37) | |
| | | ID NO: 13) | | |
| W-589 | 23691951 | CTGCTTTA | (eC)#(eT)#(eG)#(eC)#(eT)#(dT)#(dT)#(dA)# | ElavL2_12 |
| | | CGAAAAG | (dC)#(dG)#(dA)#(dA)#(dA)#(dA)#(dG)# | |
| | | TTCTC | (eT)#(eT)#(eC)#(eT)#(eC) | |
| | | (SEQ ID | (SEQ ID NO: 38) | |
| | | NO: 14) | | |
| W-590 | 23690585 | CCTCACA | (eC)#(eC)#(eT)#(eC)#(eA)#(dC)#(dA)# | ElavL2_13 |
| | | GTATATGT | (dG)#(dT)#(dA)#(dT)#(dA)#(dT)#(eG)#(eT)# | |
| | | ACT (SEQ | (eA)#(eC)#(eT) (SEQ ID NO: 39) | |
| | | ID NO: 15) | | |
| W-591 | 23691532 | CGGTAATT | (eC)#(eG)#(eG)#(eT)#(eA)#(dA)#(dT)#(dT)# | ElavL2_14 |
| | | TAAATTTC | (dT)#(dA)#(dA)#(dA)#(dT)#(dT)#(dT)#(eC)# | |
| | | TTGC (SEQ | (eT)#(eT)#(eG)#(eC) (SEQ ID NO: 40) | |
| | | ID NO: 16) | | |
| W-592 | 23691798 | CACTAGG | (eC)#(eA)#(eC)#(eT)#(eA)#(dG)#(dG)# | ElavL2_15 |
| | | TGTCATAT | (dT)#(dG)#(dT)#(dC)#(dA)#(dT)#(dA)#(dT)# | |
| | | GAAGA | (eG)#(eA)#(eA)#(eG)#(eA) | |
| | | (SEQ ID | (SEQ ID NO: 41) | |
| | | NO: 17) | | |
| W-593 | 23690519 | CCAATTG | (eC)#(eC)#(eA)#(eA)#(eT)#(dT)#(dG)# | ElavL2_16 |
| | | ATTTCTGA | (dA)#(dT)#(dT)#(dT)#(dC)#(dT)#(dG)#(dA)# | |
| | | ATAGC | (eA)#(eT)#(eA)#(eG)#(eC) | |
| | | (SEQ ID | (SEQ ID NO: 42) | |
| | | NO: 18) | | |
| W-594 | 23691135 | AGTCCTC | (eA)#(eG)#(eT)#(eC)#(eC)#(dT)#(dC)# | ElavL2_17 |
| | | AGGACAA | (dA)#(dG)#(dG)#(dA)#(dC)#(dA)#(dA)# | |
| | | CAAAAG | (dC)#(eA)#(eA)#(eA)#(eA)#(eG) | |
| | | (SEQ ID | (SEQ ID NO: 43) | |
| | | NO: 19) | | |
| W-595 | 23826136 | TTCAAAA | (eT)#(eT)#(eC)#(eA)#(eA)#(dA)#(dA)# | ElavL2_18 |
| | | AGACGAT | (dA)#(dG)#(dA)#(dC)#(dG)#(dA)#(dT)# | |
| | | GTCTTT | (dG)#(eT)#(eC)#(eT)#(eT)#(eT) | |
| | | (SEQ ID | (SEQ ID NO: 44) | |
| | | NO: 20) | | |
| W-596 | 23690384 | TTCCCTTG | (eT)#(eT)#(eC)#(eC)#(eC)#(dT)#(dT)#(dG)# | ElavL2_19 |
| | | AGGGAAA | (dA)#(dG)#(dG)#(dG)#(dA)#(dA)#(dA)# | |
| | | CCTTA | (eC)#(eC)#(eT)#(eT)#(eA) | |
| | | (SEQ ID | (SEQ ID NO: 45) | |
| | | NO: 21) | | |
| W-597 | 23690751 | GCAGGAC | (eG)#(eC)#(eA)#(eG)#(eG)#(dA)#(dC)# | ElavL2_20 |
| | | GTAACTAT | (dG)#(dT)#(dA)#(dA)#(dC)#(dT)#(eA)#(eT)# | |
| | | ATA (SEQ | (eA)#(eT)#(eA) (SEQ ID NO: 46) | |
| | | ID NO: 22) | | |
| W-598 | 23690666 | CGCAAAG | (eC)#(eG)#(eC)#(eA)#(eA)#(dA)#(dG)# | ElavL2_21 |
| | | ATAATGTA | (dA)#(dT)#(dA)#(dA)#(dT)#(dG)#(dT)#(dA)# | |
| | | CAACT | (eC)#(eA)#(eA)#(eC)#(eT) | |
| | | (SEQ ID | (SEQ ID NO: 47) | |
| | | NO: 23) | | |
| W-599 | 23691840 | AAAATATT | (eA)#(eA)#(eA)#(eA)#(eT)#(dA)#(dT)#(dT)# | ElavL2_22 |
| | | TGTTCCA | (dT)#(dG)#(dT)#(dT)#(dC)#(dC)#(dA)# | |
| | | GGACC | (eG)#(eG)#(eA)#(eC)#(eC) | |
| | | (SEQ ID | (SEQ ID NO: 48) | |
| | | NO: 24) | | |
| W-600 | 23691066 | ACCATAA | (eA)#(eC)#(eC)#(eA)#(eT)#(dA)#(dA)# | ElavL2_23 |
| | | ACAGCTG | (dA)#(dC)#(dA)#(dG)#(dC)#(dT)#(dG)# | |
| | | AAGCTA | (dA)#(eA)#(eG)#(eC)#(eT)#(eA) | |
| | | (SEQ ID | (SEQ ID NO: 49) | |
| | | NO: 25) | | |
| W-601 | 23825884 | GCTGGATA | (eG)#(eC)#(eT)#(eG)#(eG)#(dA)#(dT)#(dA)# | ElavL2_24 |
| | | GTTCTCTT | (dG)#(dT)#(dT)#(dC)#(dT)#(dC)#(dT)#(eT)# | |
| | | AAGA (SEQ | (eA)#(eA)#(eG)#(eA) | |
| | | ID NO: 26) | (SEQ ID NO: 50) | |

TABLE 2
Top 3 ELAVL2-ASO's (set 1) from Table 1.

| ID | formatted sequence |
|---|---|
| ElavL2_15 | (eC)#(eA)#(eC)#(eT)#(eA)#(dG)# |
| | (dG)#(dT)#(dG)#(dT)#(dC)#(dA)# |
| | (dT)#(dA)#(dT)#(eG)#(eA)#(eA)# |
| | (eG)#(eA) (SEQ ID NO: 41) |
| ElavL2_19 | (eT)#(eT)#(eC)#(eC)#(eC)#(dT)# |
| | (dT)#(dG)#(dA)#(dG)#(dG)#(dG)# |
| | (dA)#(dA)#(dA)#(eC)#(eC)#(eT)# |
| | (eT)#(eA) (SEQ ID NO: 45) |
| ElavL2_22 | (eA)#(eA)#(eA)#(eA)#(eT)#(dA)# |
| | (dT)#(dT)#(dT)#(dG)#(dT)#(dT)# |
| | (dC)#(dC)#(dA)#(eG)#(eG)#(eA)# |
| | (eC)#(eC) (SEQ ID NO: 48) |

### Example 2: Tables of Nucleotide Sequences of ELAVL2-siRNAs

Table 3: Screen of ELAVL2-siRNAs. SEQ ID NOS: 51-56 and 65-98 correspond to unmodified mRNA and siRNA inhibitors, while SEQ ID NOS: 57-62 and 99-132 are examples of modified anti-sense (as) and sense strand siRNAs. Referring to SEQ ID NOS: 57-62 and 99-132, the nucleotides prefaced with “m” are 2′-O-methyl, the nucleotides prefaced with “f” are 2′-fluoro, and as strands prefaced with “P” are 5′-phosphate. # indicates phosphorothioate (PS) linkage. TegChoI=a 3′ TEG-linked cholesterol conjugate used to improve delivery to cells in culture. In vivo these can be delivered, for example, as divalent asymmetric siRNAs 10 or C16-conjugated siRNAs. Sequences disclosed herein can be used with or without TegChoI.

| | mRNA | siRNA as | | | |
|---|---|---|---|---|---|
| ID | seq | seq | as strand | sense strand | |
| siELAVL2_ | UUAUAC | AGACAU | P(mU)#(fG)#( | (mA)#(mC)#(mA | activate |
| 1 | AUUAGU | UUACUA | mA)(fC)(fA)(f | )(fU)(mU)(fA)(m | between |
| | AAAUGU | AUGUAU | U)(mU)(fU)(m | G)(fU)(mA)(fA)( | human & |
| | CU (SEQ | AA | A)(fC)(mU)(f | mA)(mU)(mG)(f | mouse |
| | ID NO: | (SEQ ID | A)(mA)(fU)#( | U)#(mC)#(mA)- | |
| | 65) | NO: 82) | mG)#(fU)#(m | TegChol | |
| | | | A)#(mU)#(mA | (SEQ ID NO: | |
| | | | )#(mA)#(fU) | 116) | |
| | | | (SEQ ID NO: | | |
| | | | 99) | | |
| siELAVL2_ | UUUACC | UUUCUU | P(mU)#(fU)#( | (mC)#(mC)#(mA | activate |
| 2 | AUGUAU | UCAUAC | mU)(fC)(fU)(f | )(fU)(mG)(fU)(m | between |
| | GAAAGA | AUGGUA | U)(mU)(fC)(m | A)(fU)(mG)(fA)( | human & |
| | AA (SEQ | AA | A)(fU)(mA)(f | mA)(mA)(mG)(f | mouse |
| | ID NO: | (SEQ ID | C)(mA)(fU)#( | A)#(mA)#(mA)- | |
| | 66) | NO: 83) | mG)#(fG)#(m | TegChol | |
| | | | U)#(mA)#(mA | (SEQ ID NO: | |
| | | | )#(mA)#(fU) | 117) | |
| | | | (SEQ ID NO: | | |
| | | | 100) | | |
| siELAVL2_ | UUUGGU | AAACAU | P(mU)#(fA)#( | (mG)#(mU)#(mU | |
| 3 | UUAUAA | AUUUAU | mA)(fC)(fA)(f | )(fU)(mA)(fU)(m | |
| | AUAUGU | | U)(mA)(fU)(m | A)(fA)(mA)(fU)( | |
| | UU (SEQ | AAACCA | U)(fU)(mA)(f | mA)(mU)(mG)(f | |
| | ID NO: | AA | U)(mA)(fA)#( | U)#(mU)#(mA)- | |
| | 67) | (SEQ ID | mA)#(fC)#(m | TegChol | |
| | | NO: 84) | C)#(mA)#(mA | (SEQ ID NO: | |
| | | | )#(mA)#(fU) | 118) | |
| | | | (SEQ ID NO: | | |
| | | | 101) | | |
| siELAVL2_ | UGGUUU | ACAAAC | P(mU)#(fC)#( | (mU)#(mU)#(mA | |
| 4 | AUAAAU | AUAUUU | mA)(fA)(fA)(f | )(fU)(mA)(fA)(m | |
| | AUGUUU | AUAAAC | C)(mA)(fU)(m | A)(fU)(mA)(fU)( | |
| | GU (SEQ | CA | A)(fU)(mU)(f | mG)(mU)(mU)(f | |
| | ID NO: | (SEQ ID | U)(mA)(fU)#( | U)#(mG)#(mA)- | |
| | 68) | NO: 85) | mA)#(fA)#(m | TegChol | |
| | | | A)#(mC)#(mC | (SEQ ID NO: | |
| | | | )#(mA)#(fU) | 119) | |
| | | | (SEQ ID NO: | | |
| | | | 102) | | |
| siELAVL2_ | UCUGAU | UAAGCU | P(mU)#(fA)#( | (mA)#(mU)#(mU | activate |
| 5 | UAGCAU | AGAUGC | mA)(fG)(fC)(f | )(fA)(mG)(fC)(m | between |
| | CUAGCU | UAAUCA | U)(mA)(fG)(m | A)(fU)(mC)(fU)( | human & |
| | UA (SEQ | GA | A)(fU)(mG)(f | mA)(mG)(mC)(f | mouse |
| | ID NO: | (SEQ ID | C)(mU)(fA)#( | U)#(mU)#(mA)- | |
| | 69) | NO: 86) | mA)#(fU)#(m | TegChol | |
| | | | C)#(mA)#(mG | (SEQ ID NO: | |
| | | | )#(mA)#(fU) | 120) | |
| | | | (SEQ ID NO: | | |
| | | | 103) | | |
| siELAVL2_ | UGACGG | CCAAAC | P(mU)#(fC)#( | (mG)#(mG)#(mA | |
| 6 | AAUGAC | UGGUCA | mA)(fA)(fA)(f | )(fA)(mU)(fG)(m | |
| | CAGUUU | UUCCGU | C)(mU)(fG)(m | A)(fC)(mC)(fA)( | |
| | GG (SEQ | CA | G)(fU)(mC)(f | mG)(mU)(mU)(f | |
| | ID NO: | (SEQ ID | A)(mU)(fU)#( | U)#(mG)#(mA)- | |
| | 70) | NO: 87) | mC)#(fC)#(m | TegChol | |
| | | | G)#(mU)#(mC | (SEQ ID NO: | |
| | | | )#(mA)#(fU) | 121) | |
| | | | (SEQ ID NO: | | |
| | | | 104) | | |
| siELAVL2_ | UUGGUU | CAAACA | P(mU)#(fA)#( | (mU)#(mU)#(mU | |
| 7 | UAUAAA | UAUUUA | mA)(fA)(fC)(f | )(fA)(mU)(fA)(m | |
| | UAUGUU | UAAACC | A)(mU)(fA)(m | A)(fA)(mU)(fA)( | |
| | UG (SEQ | AA | U)(fU)(mU)(f | mU)(mG)(mU)(f | |
| | ID NO: | (SEQ ID | A)(mU)(fA)#( | U)#(mU)#(mA)- | |
| | 71) | NO: 88) | mA)#(fA)#(m | TegChol | |
| | | | C)#(mC)#(mA | (SEQ ID NO: | |
| | | | )#(mA)#(fU) | 122) | |
| | | | (SEQ ID NO: | | |
| | | | 105) | | |
| siELAVL2_ | UUCGUA | GCAAGG | P(mU)#(fC)#( | (mU)#(mA)#(mA | activate |
| 8 | AAGCAG | UACUGC | mA)(fA)(fG)(f | )(fA)(mG)(fC)(m | between |
| | UACCUU | UUUACG | G)(mU)(fA)(m | A)(fG)(mU)(fA)( | human & |
| | GC (SEQ | AA | C)(fU)(mG)(fC | mC)(mC)(mU)(f | mouse |
| | ID NO: | (SEQ ID | )(mU)(fU)#(m | U)#(mG)#(mA)- | |
| | 72) | NO: 89) | U)#(fA)#(mC) | TegChol | |
| | | | #(mG)#(mA)#( | (SEQ ID NO: | |
| | | | mA)#(fU) | 123) | |
| | | | (SEQ ID NO: | | |
| | | | 106) | | |
| siELAVL2_ | CAGCUA | GAAAUG | P(mU)#(fA)#( | (mU)#(mA)#(mA | activate |
| 9 | AGGGAA | UCUUCC | mA)(fA)(fU)(f | )(fG)(mG)(fG)(m | between |
| | GACAUU | CUUAGC | G)(mU)(fC)(m | A)(fA)(mG)(fA)( | human & |
| | UC (SEQ | UG | U)(fU)(mC)(fC | mC)(mA)(mU)(f | mouse |
| | ID NO: | (SEQ ID | )(mC)(fU)#(m | U)#(mU)#(mA)- | |
| | 73) | NO: 90) | U)#(fA)#(mG) | TegChol | |
| | | | #(mC)#(mU)#( | (SEQ ID NO: | |
| | | | mG)#(fU) | 124) | |
| | | | (SEQ ID NO: | | |
| | | | 107) | | |
| siELAVL2_ | CUUCAU | UCACUA | P(mU)#(fC)#( | (mA)#(mU)#(mA | activate |
| 10 | AUGACA | GGUGUC | mA)(fC)(fU)(f | )(fU)(mG)(fA)(m | between |
| | CCUAGU | AUAUGA | A)(mG)(fG)(m | C)(fA)(mC)(fC)( | human & |
| | GA (SEQ | AG | U)(fG)(mU)(f | mU)(mA)(mG)(f | mouse |
| | ID NO: | (SEQ ID | C)(mA)(fU)#( | U)#(mG)#(mA)- | |
| | 74) | NO: 91) | mA)#(fU)#(m | TegChol | |
| | | | G)#(mA)#(mA | (SEQ ID NO: | |
| | | | )#(mG)#(fU) | 125) | |
| | | | (SEQ ID NO: | | |
| | | | 108) | | |
| siELAVL2_ | UAUGCU | AGUUAA | P(mU)#(fG)#( | (mC)#(mU)#(mA | activate |
| 11 | AUUGAA | AUUUCA | mU)(fU)(fA)(f | )(fU)(mU)(fG)(m | between |
| | AUUUAA | AUAGCA | A)(mA)(fU)(m | A)(fA)(mA)(fU)( | human & |
| | CU (SEQ | UA | U)(fU)(mC)(f | mU)(mU)(mA)(f | mouse |
| | ID NO: | (SEQ ID | A)(mA)(fU)#( | A)#(mC)#(mA)- | |
| | 75) | NO: 92) | mA)#(fG)#(m | TegChol | |
| | | | C)#(mA)#(mU | (SEQ ID NO: | |
| | | | )#(mA)#(fU) | 126) | |
| | | | (SEQ ID NO: | | |
| | | | 109) | | |
| siELAVL2_ | UGUGUA | GGUCUU | P(mU)#(fG)#( | (mU)#(mA)#(mA | |
| 12 | AUGUAA | AUUUAC | mU)(fC)(fU)(f | )(fU)(mG)(fU)(m | |
| | AUAAGA | AUUACA | U)(mA)(fU)(m | A)(fA)(mA)(fU)( | |
| | CC (SEQ | CA | U)(fU)(mA)(f | mA)(mA)(mG)(f | |
| | ID NO: | (SEQ ID | C)(mA)(fU)#( | A)#(mC)#(mA)- | |
| | 76) | NO: 93) | mU)#(fA)#(m | TegChol | |
| | | | C)#(mA)#(mC | (SEQ ID NO: | |
| | | | )#(mA)#(fU) | 127) | |
| | | | (SEQ ID NO: | | |
| | | | 110) | | |
| siELAVL2_ | GCUGUU | UCUUAA | P(mU)#(fC)#( | (mU)#(mU)#(mA | |
| 13 | AUGUCU | ACAGAC | mU)(fU)(fA)(f | )(fU)(mG)(fU)(m | |
| | GUUUAA | AUAACA | A)(mA)(fC)(m | C)(fU)(mG)(fU)( | |
| | GA (SEQ | GC | A)(fG)(mA)(f | mU)(mU)(mA)(f | |
| | ID NO: | (SEQ ID | C)(mA)(fU)#( | A)#(mG)#(mA)- | |
| | 77) | NO: 94) | mA)#(fA)#(m | TegChol | |
| | | | C)#(mA)#(mG | (SEQ ID NO: | |
| | | | )#(mC)#(fU) | 128) | |
| | | | (SEQ ID NO: | | |
| | | | 111) | | |
| siELAVL2_ | GGCUAC | UAAGCG | P(mU)#(fA)#( | (mA)#(mC)#(mA | |
| 14 | AUUAUA | UUUAUA | mA)(fG)(fC)(f | )(fU)(mU)(fA)(m | |
| | AACGCU | AUGUAG | G)(mU)(fU)(m | U)(fA)(mA)(fA)( | |
| | UA (SEQ | CC | U)(fA)(mU)(f | mC)(mG)(mC)(f | |
| | ID NO: | (SEQ ID | A)(mA)(fU)#( | U)#(mU)#(mA)- | |
| | 78) | NO: 95) | mG)#(fU)#(m | TegChol | |
| | | | A)#(mG)#(mC | (SEQ ID NO: | |
| | | | )#(mC)#(fU) | 129) | |
| | | | (SEQ ID NO: | | |
| | | | 112) | | |
| siELAVL2_ | UUAGCA | CAUAUU | P(mU)#(fA)#( | (mC)#(mA)#(mU | activate |
| 15 | UCCUAA | UCUUAG | mU)(fA)(fU)(f | )(fC)(mC)(fU)(m | between |
| | GAAAUA | GAUGCU | U)(mU)(fC)(m | A)(fA)(mG)(fA)( | human & |
| | UG (SEQ | AA | U)(fU)(mA)(f | mA)(mA)(mU)(f | mouse |
| | ID NO: | (SEQ ID | G)(mG)(fA)#( | A)#(mU)#(mA)- | |
| | 51) | NO: 54) | mU)#(fG)#(m | TegChol | |
| | | | C)#(mU)#(mA | (SEQ ID NO: 60) | |
| | | | )#(mA)#(fU) | | |
| | | | (SEQ ID NO: | | |
| | | | 57) | | |
| siELAVL2_ | UCAGCU | AAAUGU | P(mU)#(fA)#( | (mC)#(mU)#(mA | activate |
| 16 | AAGGGA | CUUCCC | mA)(fU)(fG)(f | )(fA)(mG)(fG)(m | between |
| | AGACAU | UUAGCU | U)(mC)(fU)(m | G)(fA)(mA)(fG)( | human & |
| | UU (SEQ | GA | U)(fC)(mC)(fC | mA)(mC)(mA)(f | mouse |
| | ID NO: | (SEQ ID | )(m)(fU)#(m | U)#(mU)#(mA)- | |
| | 52) | NO: 55) | A)#(fG)#(mC) | TegChol | |
| | | | #(mU)#(mG)#( | (SEQ ID NO: 61) | |
| | | | mA)#(fU) | | |
| | | | (SEQ ID NO: | | |
| | | | 58) | | |
| siELAVL2_ | GCAGUA | AAUUCU | P(mU)#(fA)#( | (mU)#(mA)#(mU | |
| 17 | UUGUUU | UCAAAC | mU)(fU)(fC)(f | )(fU)(mG)(fU)(m | |
| | GAAGAA | AAUACU | U)(mU)(fC)(m | U)(fU)(mG)(fA)( | |
| | UU (SEQ | GC | A)(fA)(mA)(f | mA)(mG)(mA)(f | |
| | ID NO: | (SEQ ID | C)(mA)(fA)#( | A)#(mU)#(mA)- | |
| | 79) | NO: 96) | mU)#(fA)#(m | TegChol | |
| | | | C)#(mU)#(mG | (SEQ ID NO: | |
| | | | )#(mC)#(fU) | 130) | |
| | | | (SEQ ID NO: | | |
| | | | 113) | | |
| siELAVL2_ | UCAGAA | CUAAGC | P(mU)#(fU)#( | (mA)#(mA)#(mA | |
| 18 | AUCAAU | CAAUUG | mA)(fA)(fG)(f | )(fU)(mC)(fA)(m | |
| | UGGCUU | AUUUCU | C)(mC)(fA)(m | A)(fU)(mU)(fG)( | |
| | AG (SEQ | GA | A)(fU)(mU)(f | mG)(mC)(mU)(f | |
| | ID NO: | (SEQ ID | G)(mA)(fU)#( | U)#(mA)#(mA)- | |
| | 53) | NO: 56) | mU)#(fU)#(m | TegChol | |
| | | | C)#(mU)#(mG | (SEQ ID NO: 62) | |
| | | | )#(mA)#(fU) | | |
| | | | (SEQ ID NO: | | |
| | | | 59) | | |
| siELAVL2_ | UUGGAU | UGUCAU | P(mU)#(fG)#( | (mA)#(mU)#(mU | activate |
| 19 | UUGUGA | AGUCAC | mU)(fC)(fA)(f | )(fU)(mG)(fU)(m | between |
| | CUAUGA | AAAUCC | U)(mA)(fG)(m | G)(fA)(mC)(fU)( | human & |
| | CA (SEQ | AA | U)(fC)(mA)(fC | mA)(mU)(mG)(f | mouse |
| | ID NO: | (SEQ ID | )(mA)(fA)#(m | A)#(mC)#(mA)- | |
| | 80) | NO: 97) | A)#(fU)#(mC) | TegChol | |
| | | | #(mC)#(mA)#( | (SEQ ID NO: | |
| | | | mA)#(fU) | 131) | |
| | | | (SEQ ID NO: | | |
| | | | 114) | | |
| siELAVL2_ | GCUAAC | CACAAA | P(mU)#(fA)#( | (mA)#(mC)#(mA | activate |
| 20 | ACCUGA | GCUCAG | mC)(fA)(fA)(f | )(fC)(mC)(fU)(m | between |
| | GCUUUG | GUGUUA | A)(mG)(fC)(m | G)(fA)(mG)(fC)( | human & |
| | UG (SEQ | GC | U)(fC)(mA)(f | mU)(mU)(mU)(f | mouse |
| | ID NO: | (SEQ ID | G)(mG)(fU)#( | G)#(mU)#(mA)- | |
| | 81) | NO: 98) | mG)#(fU)#(m | TegChol | |
| | | | U)#(mA)#(mG | (SEQ ID NO: | |
| | | | )#(mC)#(fU) | 132) | |
| | | | (SEQ ID NO: | | |
| | | | 115) | | |

TABLE 4
Top 3 ELAVL2-siRNAs from Table 3.

| | | siRNA as | | | |
|---|---|---|---|---|---|
| ID | mRNA seq | seq | as strand | sense strand | |
| siELAVL2_15 | UUAGCA | CAUAUU | P(mU)#(fA)#(m | (mC)#(mA)#(mU | activate |
| | UCCUAA | UCUUAG | U)(fA)(fU)(fU)( | )(fC)(mC)(fU)(m | between |
| | GAAAUA | GAUGCU | mU)(fC)(mU)(fU | A)(fA)(mG)(fA)( | human & |
| | UG (SEQ | AA (SEQ | )(mA)(fG)(mG)(f | mA)(mA)(mU)(f | mouse |
| | ID NO: 51) | ID NO: 54) | A)#(mU)#(fG)#( | A)#(mU)#(mA)- | |
| | | | mC)#(mU)#(mA) | TegChol | |
| | | | #(mA)#(fU) | (SEQ ID NO: 60) | |
| | | | (SEQ ID NO: 57) | | |
| siELAVL2_16 | UCAGCU | AAAUGU | P(mU)#(fA)#(m | (mC)#(mU)#(mA | activate |
| | AAGGGA | CUUCCC | A)(fU)(fG)(fU)( | )(fA)(mG)(fG)(m | between |
| | AGACAU | UUAGCU | mC)(fU)(mU)(fC | G)(fA)(mA)(fG)( | human & |
| | UU(SEQ | GA (SEQ | )(mC)(fC)(mU)(f | mA)(mC)(mA)(f | mouse |
| | ID NO: 52) | ID NO: 55) | U)#(mA)#(fG)#( | U)#(mU)#(mA)- | |
| | | | mC)#(mU)#(mG) | TegChol | |
| | | | #(mA)#(fU) | (SEQ ID NO: 61) | |
| | | | (SEQ ID NO: 58) | | |
| siELAVL2_18 | UCAGAA | CUAAGC | P(mU)#(fU)#(m | (mA)#(mA)#(mA | |
| | AUCAAU | CAAUUG | A)(fA)(fG)(fC)( | )(fU)(mC)(fA)(m | |
| | UGGCUU | AUUUCU | mC)(fA)(mA)(fU | A)(fU)(mU)(fG)( | |
| | AG(SEQ | GA (SEQ | )(mU)(fG)(mA)(f | mG)(mC)(mU)(f | |
| | ID NO: 53) | ID NO: 56) | U)#(mU)#(fU)#( | U)#(mA)#(mA)- | |
| | | | mC)#(mU)#(mG) | TegChol | |
| | | | #(mA)#(fU) | (SEQ ID NO: 62) | |
| | | | (SEQ ID NO: 59) | | |

### Example 3: Tables of Nucleotide Sequences of ELAVL2-ASO's (Set 2)

Table 5: Screen of ELA VL2-ASO's (set 2). SEQ ID NOS: 133-150 are examples of modified nucleic acid inhibitors. Referring to SEQ ID NOS: 133-150, the nucleotides prefaced with “e” are 2′-O-MOE nucleotides, and the nucleotides prefaced with “d” are deoxynucleotides. # indicates phosphorothioate (PS) linkage.

| ID | formatted sequence |
|---|---|
| hELAV-1 | (eA)#(eA)#(eG)#(eC)#(eC)#(dA)#(dT)#(dA)#(dT)#(dT)#(dG)#(dA)#(dG)#(dC) |
| (w-2240) | #(eA)#(eG)#(eA)#(eU)#(eU) (SEQ ID NO: 133) |
| hELAV-2 | (eA)#(eG)#(eA)#(eA)#(eA)#(dA)#(dC)#(dC)#(dT)#(dC)#(dT)#(dT)#(dT)#(dA) |
| (w-2241) | #(dC)#(eU)#(eC)#(eC)#(eA)#(eU) (SEQ ID NO: 134) |
| hELAV-3 | (eA)#(eC)#(eA)#(eA)#(eA)#(dT)#(dA)#(dT)#(dA)#(dC)#(dA)#(dC)#(dC)#(dA) |
| (w-2242) | #(dC)#(eC)#(eC)#(eU)#(eG)#(eU) (SEQ ID NO: 135) |
| hELAV-4 | (eC)#(eC)#(eU)#(eU)#(eC)#(dA)#(dC)#(dA)#(dT)#(dT)#(dG)#(dG)#(dT)#(dG) |
| (w-2243) | #(dA)#(eC)#(eA)#(eG)#(eC)#(eU) (SEQ ID NO: 136) |
| hELAV-5 | (eC)#(eC)#(eA)#(eA)#(eA)#(dA)#(dC)#(dC)#(dT)#(dT)#(dT)#(dG)#(dC)#(dA) |
| (w-2244) | #(dT)#(eU)#(eU)#(eA)#(eU)#(eU) (SEQ ID NO: 63) |
| hELAV-6 | (eA)#(eG)#(eU)#(eU)#(eU)#(dG)#(dT)#(dC)#(dA)#(dT)#(dA)#(dG)#(dT)#(dC) |
| (w-2245) | #(dA)#(eC)#(eA)#(eA)#(eA)#(eU) (SEQ ID NO: 137) |
| hELAV-7 | (eC)#(eG)#(eC)#(eC)#(eA)#(dT)#(dG)#(dG)#(dC)#(dA)#(dG)#(dC)#(dC)#(dT) |
| (w-2246) | #(dC)#(eA)#(eU)#(eC)#(eA)#(eU) (SEQ ID NO: 64) |
| hELAV-8 | (eU)#(eA)#(eU)#(eC)#(eC)#(dA)#(dT)#(dT)#(dG)#(dA)#(dG)#(dG)#(dC)#(dT) |
| (w-2247) | #(dA)#(eG)#(eC)#(eU)#(eA)#(eU) (SEQ ID NO: 138) |
| hELAV-9 | (eU)#(eG)#(eU)#(eC)#(eU)#(dT)#(dA)#(dA)#(dA)#(dG)#(dG)#(dA)#(dG)#(dA |
| (w-2248) | )#(dC)#(eC)#(eU)#(eG)#(eC)#(eA) (SEQ ID NO: 139) |
| hELAV-10 | (eG)#(eA)#(eC)#(eU)#(eG)#(dA)#(dG)#(dG)#(dA)#(dC)#(dA)#(dA)#(dG)#(dA |
| (w-2249) | )#(dG)#(eC)#(eU)#(eC)#(eA)#(eU) (SEQ ID NO: 140) |
| hELAV-11 | (eG)#(eU)#(eG)#(eC)#(eC)#(dC)#(dA)#(dG)#(dG)#(dG)#(dA)#(dT)#(dA)#(dT) |
| (w-2250) | #(dT)#(eA)#(eA)#(eU)#(eU)#(eC) (SEQ ID NO: 141) |
| hELAV-12 | (eG)#(eC)#(eU)#(eG)#(eG)#(dA)#(dT)#(dA)#(dG)#(dT)#(dT)#(dC)#(dT)#(dC) |
| (w-2251) | #(dT)#(eU)#(eA)#(eA)#(eG)#(eA) (SEQ ID NO: 142) |
| hELAV-13 | (eC)#(eA)#(eA)#(eG)#(eA)#(dA)#(dA)#(dA)#(dA)#(dC)#(dA)#(dA)#(dA)#(dC |
| (w-2252) | )#(dC)#(eA)#(eA)#(eG)#(eG)#(eA) (SEQ ID NO: 143) |
| hELAV-14 | (eC)#(eC)#(eC)#(eA)#(eA)#(dA)#(dG)#(dA)#(dG)#(dA)#(dC)#(dT)#(dC)#(dT) |
| (w-2253) | #(dT)#(eU)#(eA)#(eG)#(eU)#(eU) (SEQ ID NO: 144) |
| hELAV-15 | (eA)#(eA)#(eC)#(eC)#(eA)#(dA)#(dG)#(dG)#(dA)#(dA)#(dC)#(dA)#(dA)#(dA |
| (w-2254) | )#(eG)#(eA)#(eA)#(eA)#(eA) (SEQ ID NO: 145) |
| hELAV-16 | (eA)#(eG)#(eC)#(eA)#(eA)#(dA)#(dT)#(dA)#(dA)#(dA)#(dA)#(dA)#(dG)#(dA |
| (w-2255) | )#(dA)#(eA)#(eC)#(eG)#(eC)#(eU) (SEQ ID NO: 146) |
| hELAV-17 | (eA)#(eA)#(eA)#(eC)#(eA)#(dA)#(dA)#(dC)#(dC)#(dA)#(dA)#(dG)#(dG)#(dA |
| (w-2256) | )#(eA)#(eC)#(eA)#(eA)#(eA) (SEQ ID NO: 147) |
| hELAV-18 | (eU)#(eA)#(eU)#(eC)#(eC)#(dA)#(dT)#(dT)#(dG)#(dA)#(dG)#(dG)#(dC)#(dT) |
| (w-2257) | #(dA)#(eG)#(eC)#(eU)#(eA)#(eU) (SEQ ID NO: 148) |
| hELAV-19 | (eG)#(eC)#(eU)#(eA)#(eA)#(dA)#(dA)#(dG)#(dA)#(dA)#(dA)#(dT)#(dG)#(dG |
| (w-2258) | )#(dC)#(eG)#(eA)#(eA)#(eA)#(eA) (SEQ ID NO: 149) |
| hELAV-20 | (eG)#(eA)#(eA)#(eC)#(eA)#(dA)#(dA)#(dG)#(dA)#(dA)#(dA)#(dA)#(dG)#(dA |
| (w-2259) | )#(dG)#(eA)#(eC)#(eG)#(eA)#(eA) (SEQ ID NO: 150) |

TABLE 6
Top 2 ELAVL2-ASO's (set 2) from Table 5.

| ID | formatted sequence |
|---|---|
| hELAV-5 | (eC)#(eC)#(eA)#(eA)#(eA)#(dA)# |
| (w-2244) | (dC)#(dC)#(dT)#(dT)#(dT)#(dG)# |
| | (dC)#(dA)#(dT)#(eU)#(eU)#(eA)# |
| | (eU)#(eU) (SEQ ID NO:63) |
| hELAV-7 | (eC)#(eG)#(eC)#(eC)#(eA)#(dT)# |
| (w-2246) | (dG)#(dG)#(dC)#(dA)#(dG)#(dC)# |
| | (dC)#(dT)#(dC)#(eA)#(eU)#(eC)# |
| | (eA)#(eU) (SEQ ID NO:64) |

### Example 4: Poly (GR) as a Neurotoxic Species

Among the dipeptide repeat (DPR) proteins, poly(GR) is a key neurotoxic species causing neurodegeneration in cellular and animal models. For example, it was found that poly(GR) expression in flies or human motor neurons are highly toxic. Moreover, an inducible mouse model was established in which (GR) 80 is expressed in the brain at ~5-15% of the level in C9ORF72 patient brains and demonstrated that low-level poly(GR) expression alone can elicit several FTD/ALS-like behavioral and cellular phenotypes.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A nucleic acid inhibitor which is capable of reducing expression of ELAVL2 by 10% or more compared to a control where the nucleic acid inhibitor is not present, wherein said nucleic acid inhibitor hybridizes to at least five nucleotides within SEQ ID NO: 1.

2. The nucleic acid inhibitor of claim 1, wherein the nucleic acid inhibitor hybridizes to at least five nucleotides within a region of SEQ ID NO: 2.

3. The nucleic acid inhibitor of claim 2, wherein the nucleic acid inhibitor hybridizes to at least five nucleotides within a region of 1-450 or 2100-3983 of SEQ ID NO: 2.

4. The nucleic acid inhibitor of claim 1, wherein the nucleic acid inhibitor is about 15-30 nucleotides long.

5. The nucleic acid inhibitor of claim 1, wherein the nucleic acid inhibitor is an antisense oligonucleotide or an RNAi molecule.

6. The nucleic acid inhibitor of claim 5, wherein the RNAi molecule is siRNA.

7. The nucleic acid inhibitor of claim 1, wherein the nucleic acid inhibitor comprises at least one modification.

8. The nucleic acid inhibitor of claim 7, wherein the at least one modification comprises at least one modified sugar moiety; at least one modified inter-nucleotide linkage; and/or at least one modified nucleotide.

9. The nucleic acid inhibitor of claim 8, wherein the at least one modification comprising an internucleotide linkage comprising: phosphorothioate, alkylphosphonate, phosphorodithioate, alkylphosphonothioate, phosphoramidate, carbamate, carbonate, phosphate triester, acetamidate, and/or carboxymethyl ester.

10. The nucleic acid inhibitor of claim 9, wherein the at least one modification comprising a modified nucleotide comprises a peptide nucleic acid, a morpholino analogue, a locked nucleic acid (LNA) or other bridged or bicyclic nucleic acid analogue, and/or a combination thereof.

11. The nucleic acid inhibitor of claim 9, wherein the at least one modification comprising a modified sugar moiety comprises: a 2′-fluoro sugar moiety, 2′-O-methoxyethyl modified sugar moiety, a 2′-methoxy modified sugar moiety, and/or another 2′-O-alkyl or alkoxyalkyl modified sugar moiety.

12.-16. (canceled)

17. A viral vector comprising the nucleic acid inhibitor of claim 1.

18. A pharmaceutical composition comprising the nucleic acid inhibitor of claim 1.

19. (canceled)

20. (canceled)

21. (canceled)

22. A nucleic acid comprising 90% or more identity to any one of SEQ ID NOS: 3-50, SEQ ID NOS: 54-64, or SEQ ID NOS: 82-150.

23. (canceled)

24. The nucleic acid of claim 22, comprising SEQ ID NOS: 17, 21, or 24.

25. The nucleic acid of claim 22, comprising SEQ ID NOS: 41, 45, or 48.

26. The nucleic acid of claim 22, comprising SEQ ID NOS: 54, 55, 56, 57, 58, 59, 60, 61, or 62.

27. The nucleic acid of claim 22, comprising SEQ ID NOS: 63 or 64.

28. A method of treating a disorder or disease associated with poly(GR) toxicity in neuronal cells in a subject in need thereof, the method comprising administering the nucleic acid of claim 22.

29.-44. (canceled)

45. A method of screening for a nucleic acid inhibitor of ELAVL2, the method comprising contacting SEQ ID NO: 1 with a complementary nucleic acid between about 15-30 nucleotides long, and determining the complementary nucleic acid is effective at knocking down expression of ELAVL2.

46. (canceled)

47. (canceled)

48. (canceled)
